(19) United States
(12) Patent Application Publication
(10) Pub. No.: US 2025/0228820 A1
(43) Pub. Date: Jul. 17, 2025

(54) METHODS FOR PREVENTING OR SLOWING THE PROGRESSION OF COGNITIVE DECLINE OR IMPAIRMENT IN SUBJECTS DISPLAYING NORMAL COGNITIVE PERFORMANCE

(71) Applicants: AGENEBIO, INC., Baltimore, MD (US); THE JOHNS HOPKINS UNIVERSITY, Baltimore, MD (US); THE MEDICAL COLLEGE OF WISCONSIN, Milwaukee, WI (US)

(72) Inventors: Shi-Jiang Li, Milwaukee, WI (US); Arnold Bakker, Baltimore, MD (US); Michela Gallagher, Baltimore, MD (US); Sharon Rosenzweig-Lipson, Baltimore, MD (US)

(21) Appl. No.: 18/852,724

(22) PCT Filed: Apr. 3, 2023
(86) PCT No.: PCT/US2023/017348

§ 371 Date: Sep. 30, 2024

Related U.S. Application Data
(60) Provisional application No. 63/326,773, filed on Apr. 1, 2022.

(51) Int. Cl. A61K 31/4015; A61K 9/00; A61K 9/28; A61P 25/28

(52) U.S. Cl. CPC A61K 31/4015; A61K 9/0004; A61K 9/0053; A61K 9/2813; A61K 9/2833; A61K 9/2866; A61P 25/28

(57) ABSTRACT

Methods for preventing or slowing the progression of cognitive impairment or preventing the development or reducing the rate of cognitive decline in a subject displaying or presenting with cognitive performance within the normal range for the subject's age. The methods comprise administering to the subject one or more of levetiracetam, brivaracetam or seletracetam, or a pharmaceutically acceptable salt thereof, a pharmaceutical composition comprising levetiracetam, brivaracetam or seletracetam or pharmaceutical salt thereof and a pharmaceutically acceptable carrier, a GABA_{A} α5 receptor agonist or a pharmaceutically acceptable salt, hydrate, solvate, isomer, or polymorph thereof, a pharmaceutical composition comprising a GABA_{A} α5 receptor agonist or a pharmaceutically acceptable salt, hydrate, solvate, isomer, or polymorph thereof, and a pharmaceutically acceptable carrier, or a combination or a composition comprising the levetiracetam, brivaracetam or seletracetam or pharmaceutical salt thereof and the GABA_{A} α5 receptor agonist or a pharmaceutically acceptable salt, hydrate, solvate, isomer, or polymorph. In some embodiments, the subjects have one or more risk factors that are predictive for or associated with the development or progression of cognitive impairment or decline.

### RELATED APPLICATIONS

This application claims the benefit of and priority from U.S. Provisional Application 63/326,773, filed Apr. 1, 2022, which is incorporated herein by reference in its entirety.

### STATEMENT OF GOVERNMENT SUPPORT

The subject matter of this disclosure was made with government support under Grant No. 1R21AG056882-01 awarded by the National Institutes of Health (NIH), and in particular, its National Institute on Aging (NIA) division, an agency of the United States Government. The United States Government has certain rights in the subject matter of this disclosure.

### FIELD OF THE DISCLOSURE

This disclosure relates to methods for preventing or slowing the progression of cognitive impairment or preventing the development or reducing the rate of cognitive decline in a subject displaying or presenting with cognitive performance within the normal range for the subject's age. The methods comprise administering to the subject one or more of levetiracetam, brivaracetam or seletracetam, or a pharmaceutically acceptable salt thereof, a pharmaceutical composition comprising levetiracetam, brivaracetam or seletracetam or pharmaceutical salt thereof and a pharmaceutically acceptable carrier, a GABA_{A} α5 receptor agonist or a pharmaceutically acceptable salt, hydrate, solvate, isomer, or polymorph thereof, a pharmaceutical composition comprising a GABA_{A} α5 receptor agonist or a pharmaceutically acceptable salt, hydrate, solvate, isomer, or polymorph thereof, and a pharmaceutically acceptable carrier, or a combination or a composition comprising the levetiracetam, brivaracetam or seletracetam or pharmaceutical salt thereof and the GABA_{A} α5 receptor agonist or a pharmaceutically acceptable salt, hydrate, solvate, isomer, or polymorph thereof.

In some aspects of this disclosure the subject has one or more risks that are predictive of or associated with developing cognitive decline or cognitive impairment or the progression of the decline or impairment. Such risks are associated with aging, with one or more genetic risks selected from the group of genomic variants, mutations, or polymorphs associated with a change in the expression of genes selected from the group consisting of ATP-binding cassette sub-family A member 7 (ABCA7), Clusterin (CLU), Complement receptor type 1 (CR1), Phosphatidylinositol binding clathrin assembly protein (PICALM), Phospholipase D3 (PLD3), Triggering receptor expressed on myeloid cells 2 (TREM2), and sortilin related receptor 1 (SORL1) in the genome of the subject; with the presence of at least one allele of the APOE4 gene in the genome of the subject, with the presence of one of more biofluid biomarkers selected from the group consisting of p-tau, t-tau, and amyloid β 42 in the subject, or with the presence of altered hippocampal functional connectivity in the subject.

### BACKGROUND OF THE DISCLOSURE

Cognitive ability may decline as a normal consequence of aging or may be associated with changes in hippocampal functional connectivity, with genomic variations, mutations, or polymorphs, or with various biofluid biomarkers associated with the development of cognitive impairment or decline in the context of CNS diseases and disorders, such as Alzheimer's disease. Although subjects may present or display with cognitive performance in the normal range for their age, they may still be at risk of developing cognitive impairment or decline.

In addition to aging, other such risk factors include genetic risk factors which can be characterized by genomic variants, polymorphs, and mutations associated with the development of CNS diseases or disorders associated with the development of cognitive impairment or decline. For example, Genome-Wide Association Studies (GWAS) have been used to associate variants of the ABCA7 gene with the development of Alzheimer's Disease. Indeed, those variants were reported to be enriched in Alzheimer's Disease patients (De Roeck A, Van Broeckhoven C, Sleegers K. The role of ABCA7 in Alzheimer's disease: evidence from genomics, transcriptomics and methylomics. Acta Neuropathol. 2019 August; 138 (2): 201-220). Similarly, CR1 polymorphisms and CLU variants have been reported to be linked to AD pathogenesis. Multiple polymorphisms within and around the PICALM gene, a rare missense variant in the PLD3 gene, and some variations of SORL1 have also been reported to be associated with the development of late onset AD. Similarly, variants of the TREM2 gene are associated with AD and induce partial loss of function of the TREM2 protein and alter the behavior of microglial cells, including their response to amyloid plaques.

Neuropathological, structural, and functional changes including increases in hippocampal functional connectivity, the presence of at least one allele of the APOE4 gene in the genome of the subject, or the presence of one of more biofluid biomarkers selected from the group consisting of p-tau, t-tau, and amyloid β42 in the subject may also be predictive or and associated with CNS diseases and disorders, including AD, that are associated with cognitive impairment or cognitive decline. These changes and genetic risks may begin insidiously, 20 to 30 years before the manifestation of cognitive symptoms and cognitive performance below normal age-based ranges.

Currently, no FDA-approved preventive therapy exists for subjects who display or present with cognitive performance within the normal range for the subject's age but who may be at risk for developing or progressing to cognitive impairment or cognitive decline.

There is, therefore, a need for effective clinical and therapeutic measures for preventing or slowing the progression of cognitive impairment or preventing the development or reducing the rate of cognitive decline in a subject who displays or presents with cognitive performance within the normal range for the subject's age but may be at risk for developing or progressing to cognitive impairment or cognitive decline.

### BRIEF SUMMARY OF THE DISCLOSURE

This disclosure relates to methods for preventing or slowing the progression of cognitive impairment or preventing the development or reducing the rate of cognitive decline in a subject displaying or presenting with cognitive performance within the normal range for the subject's age. The methods comprise administering to the subject one or more of levetiracetam, brivaracetam or seletracetam, or a pharmaceutically acceptable salt thereof, a pharmaceutical composition comprising levetiracetam, brivaracetam or seletracetam or pharmaceutical salt thereof and a pharmaceutically acceptable carrier, a GABA_{A} α5 receptor agonist or a pharmaceutically acceptable salt, hydrate, solvate, isomer, or polymorph thereof, a pharmaceutical composition comprising a GABA_{A} α5 receptor agonist or a pharmaceutically acceptable salt, hydrate, solvate, isomer, or polymorph thereof, and a pharmaceutically acceptable carrier, or a combination or a composition comprising the levetiracetam, brivaracetam or seletracetam or pharmaceutical salt thereof and the GABA_{A} α5 receptor agonist or a pharmaceutically acceptable salt, hydrate, solvate, isomer, or polymorph.

In some aspects of this disclosure the subject presents with one or more risk factors predictive of or associated with the development of cognitive decline or cognitive impairment or the progression of the decline or impairment. In some aspects of this disclosure, these risk factors may be associated with aging, with the presence of one or more variants, mutations, or polymorphs associated with a change in the expression of genes selected from the group consisting of ATP-binding cassette sub-family A member 7 (ABCA7), Clusterin (CLU), Complement receptor type 1 (CR1), Phosphatidylinositol binding clathrin assembly protein (PICALM), Phospholipase D3 (PLD3), Triggering receptor expressed on myeloid cells 2 (TREM2), and sortilin related receptor 1 (SORL1) in the genome of the subject, with the presence of at least one allele of the APOE4 gene in the genome of the subject, with the presence of one of more biofluid biomarkers selected from the group consisting of p-tau, t-tau, and amyloid β42 in the subject, or with the presence of altered hippocampal functional connectivity in the subject.

In some embodiments, the methods of this disclosure comprise administering to the subject levetiracetam, brivaracetam or seletracetam, or a pharmaceutically acceptable salt thereof at a daily dose of 0.7-350 mg or comprise administering to the subject a pharmaceutical composition comprising the daily dose of the levetiracetam, brivaracetam or seletracetam or pharmaceutical salt thereof, and a pharmaceutically acceptable carrier.

In some embodiments, the daily dose of the levetiracetam or seletracetam, or a pharmaceutically acceptable salt thereof is 7-350 mg. In some embodiments, the daily dose of the brivaracetam, or pharmaceutically acceptable salt thereof is 0.7-180 mg. In other embodiments, the daily dose of the levetiracetam or seletracetam, or pharmaceutically acceptable salt thereof is 125-250 mg. In some embodiments, the daily dose of the levetiracetam or seletracetam, or pharmaceutically acceptable salt thereof is 220 mg. In some embodiments, the daily dose of the levetiracetam or seletracetam or pharmaceutically acceptable salt thereof is 190 mg.

In some embodiments, the levetiracetam, brivaracetam or seletracetam of a pharmaceutically acceptable salt thereof is formulated in one or more of an oral form, an extended release form or a single-unit-dosage-form or for once-a-day administration. In some embodiments, the extended release form is a controlled release form, a prolonged release form, a sustained release form, a delayed release form, or a slow release form.

In some embodiments of this disclosure, the method comprises administering to the subject a daily dose of a pharmaceutical composition comprising 220 mg of levetiracetam or pharmaceutically acceptable salt thereof, 280 mg-350 mg of hydroxypropyl methylcellulose, 1.2 mg-1.4 mg of colloidal silicon dioxide, 92.8 mg-119.2 mg of silicified microcrystalline cellulose, and 6.0 mg-6.7 mg of magnesium stearate. In other embodiments, the daily dose of the levetiracetam or pharmaceutically acceptable salt thereof in the pharmaceutical composition is 220 mg and the pharmaceutical composition further comprises 280 mg of hydroxypropyl methylcellulose, 1.2 mg of colloidal silicon dioxide, 92.8 mg of silicified microcrystalline cellulose, and 6.0 mg of magnesium stearate. In other embodiments, the daily dose of the levetiracetam or pharmaceutically acceptable salt thereof in the pharmaceutical composition is 220 mg and the pharmaceutical composition further comprises 347.5 mg of hydroxypropyl methylcellulose, 1.4 mg of colloidal silicon dioxide, 119.2 mg of silicified microcrystalline cellulose, and 6.7 mg of magnesium stearate. In other embodiments, the hydroxypropyl methylcellulose is hypromellose 2208. In other embodiments, the silicified microcrystalline cellulose is silicified microcrystalline cellulose SMCC 90.

In some embodiments of this disclosure, the method comprises administering to the subject a daily dose of a pharmaceutical composition comprising 190 mg of levetiracetam or pharmaceutically acceptable salt thereof, 300 mg of hydroxypropyl methylcellulose, 1.2 mg of colloidal silicon dioxide, 102.8 mg of silicified microcrystalline cellulose or anhydrous dicalcium phosphate, and 6 mg of magnesium stearate. In other embodiments, the hydroxypropyl methylcellulose is hypromellose 2208. In other embodiments, the silicified microcrystalline cellulose is silicified microcrystalline cellulose SMCC 90.

In some embodiments of this disclosure, the method comprises administering a pharmaceutical composition comprising the daily dose of the levetiracetam or pharmaceutically acceptable salt thereof in extended release form, wherein the method provides a steady state plasma concentration of levetiracetam in a subject of between 1.9 μg/mL and 4.4 μg/mL within 3 hours after administration and extending for at least 8 hours of a 24-hour period after said administration. In some embodiments, the extended release pharmaceutical composition comprising the daily dose of the levetiracetam or pharmaceutically acceptable salt thereof provides a steady state plasma concentration of levetiracetam within 2 hours after said administration and extending for at least 13 hours of a 24-hour period after said administration. In some embodiments, the extended release pharmaceutical composition comprising the daily dose of the levetiracetam or pharmaceutically acceptable salt thereof provides a steady state plasma concentration of levetiracetam within 1 hour after said administration and extending for at least 13 hours of a 24-hour period after said administration. In other embodiments, the extended release pharmaceutical composition comprising the daily dose of the levetiracetam or pharmaceutically acceptable salt thereof provides said steady state plasma concentration of levetiracetam within 1 hour after administration and extending for at least 13 to 16 hours of a 24-hour period after said administration (See, FIG. 2 and WO2016191288, which is incorporated by reference herein in its entirety).

In some embodiments, the pharmaceutical composition of the levetiracetam, brivaracetam or seletracetam or a pharmaceutically acceptable salt thereof is formulated in one or more of an oral form, an extended release form or a single-unit-dosage-form or for once-a-day administration. In other embodiments, the extended release form is a controlled release form, a prolonged release form, a sustained release form, a delayed release form, or a slow release form.

In some embodiments, the GABA_{A} α5 receptor agonist, or the pharmaceutically acceptable salt, hydrate, solvate, isomer, or polymorph thereof or the GABA_{A} α5 receptor agonist, or the pharmaceutically acceptable salt, hydrate, solvate, isomer, or polymorph thereof of the pharmaceutical composition of this disclosure, is selected from the group consisting of:
i) a compound of formula II:

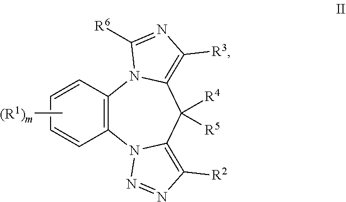

or a pharmaceutically acceptable salt, hydrate, solvate, polymorph, or isomer thereof,
wherein:
m is 0-3;
each occurrence of R¹, R², R⁴, and R⁵ are each independently selected from:
   halogen, —R, —OR, —NO₂, —NCS, —CN, —CF₃, —OCF₃, —SiR₃, —N(R)₂, —SR, —SOR, —SO₂R, —SO₂N(R)₂, —SO₃R, —(CR₂)₁₋₃R, —(CR₂)₁₋₃—OR, —(CR₂)₀₋₃—C(O)NR(CR₂)₀₋₃R, —(CR₂)₀₋₃—C(O)NR(CR₂)₀₋₃OR, —C(O)R, —C(O)C(O)R, —C(O)CH₂C(O)R, —C(S)R, —C(S)OR, —C(O)OR, —C(O)C(O)OR, —C(O)C(O)N(R)₂, —OC(O)R, —C(O)N(R)₂, —OC(O)N(R)₂, —C(S)N(R)₂, —(CR₂)₀₋₃NHC(O)R, —N(R)N(R)COR, —N(R)N(R)C(O)OR, —N(R)N(R)CON(R)₂, —N(R)SO₂R, —N(R)SO₂N(R)₂, —N(R)C(O)OR, —N(R)C(O)R, —N(R)C(S)R, —N(R)C(O)N(R)₂, —N(R)C(S)N(R)₂, —N(COR)COR, —N(OR)R, —C(═NH)N(R)₂, —C(O)N(OR)R, —C(═NOR)R, —OP(O)(OR)₂, —P(O)(R)₂, —P(O)(OR)₂, and —P(O)(H)(OR);
   R³ is selected from the group consisting of:
   halogen, —R, —OR, —NO₂, —NCS, —CN, —CF₃, —OCF₃, —SiR₃, —N(R)₂, —SR, —SOR, —SO₂R, —SO₂N(R)₂, —SO₃R, —(CR₂)₁₋₃R, —(CR₂)₁₋₃—OR, —(CR₂)₀₋₃—C(O)NR(CR₂)₀₋₃R, —(CR₂)₀₋₃—C(O)NR(CR₂)₀₋₃OR, —C(O)R, —C(O)C(O)R, —C(O)CH₂C(O)R, —C(S)R, —C(S)OR, —C(O)OR, —C(O)C(O)OR, —C(O)C(O)N(R)₂, —OC(O)R, —C(O)N(R)₂, —OC(O)N(R)₂, —C(S)N(R)₂, —(CR₂)₀₋₃NHC(O)R, —N(R)N(R)COR, —N(R)N(R)C(O)OR, —N(R)N(R)CON(R)₂, —N(R)SO₂R, —N(R)SO₂N(R)₂, —N(R)C(O)OR, —N(R)C(O)R, —N(R)C(S)R, —N(R)C(O)N(R)₂, —N(R)C(S)N(R)₂, —N(COR)COR, —N(OR)R, —C(═NH)N(R)₂, —C(O)N(OR)R, —C(═NOR)R, —OP(O)(OR)₂, —P(O)(R)₂, —P(O)(OR)₂, and —P(O)(H)(OR); —C≡CH, —C≡CR⁹, —(C1-C6)alkyl-C≡C—R¹⁰, —CH₂—O—R¹⁰, —CH₂—O—CH₂-R¹⁰

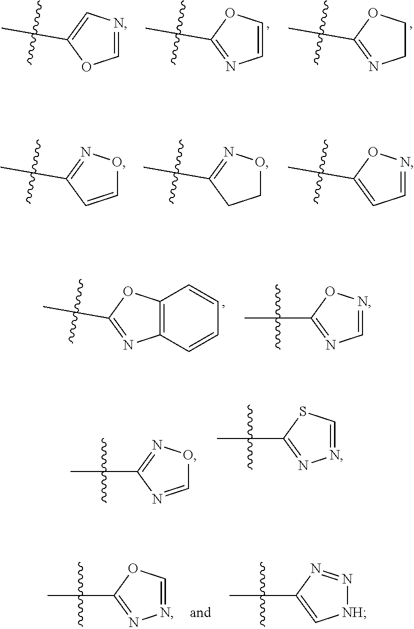

wherein each 5-member heterocycle or heteroaryl is substituted with 0-4 R₇;
wherein R³ is independently substituted with 0-5 R′;
R⁶ is selected from the group consisting of —H and —(C1-C6)alkyl;
wherein R₇ is selected from the group consisting of —(C1-C6)alkyl, —(C3-C6) cycloalkyl, -5 to 10 membered heteroaryl, —(C6-C10) aryl, (C6-C10) aryl-(C1-C6)alkyl-, -5 to 10 membered heteroaryl-(C1-C6)alkyl, and -5-10 membered heteroaryl; wherein each R₇ is independently substituted with 0-5 R′;
wherein each R⁸ is independently selected from the group consisting of —H, —(C1-C6)alkyl, —(C3-C6) cycloalkyl, —(C1-C6)alkyl-(C3-C6) cycloalkyl, —(C1-C6)alkyl-(C6-C10) aryl, —(C6-C10) aryl, -5-10 membered heteroaryl, and —(C1-C6)alkyl-5-10 membered heteroaryl;
   wherein each R⁸ excluding-H and —(C1-C6)alkyl is independently substituted by 0-5 of -halogen, —(C1-C6)alkyl, —CF₃, —OCF₃, or O—(C1-C6)alkyl;
wherein R⁹ is selected from the group consisting of —H, —(C1-C6)alkyl, —(C6-C10) aryl, -5-10 membered heteroaryl, —(C1-C6)alkyl-(C6-C10) aryl, —(C1-C6)alkyl-5-10 membered heteroaryl, —(C3-C6) cycloalkyl, —(C1-C6)alkyl-(C3-C6) cycloalkyl, —C(O)—(C6-C10) aryl, 5-10 membered heterocycle,

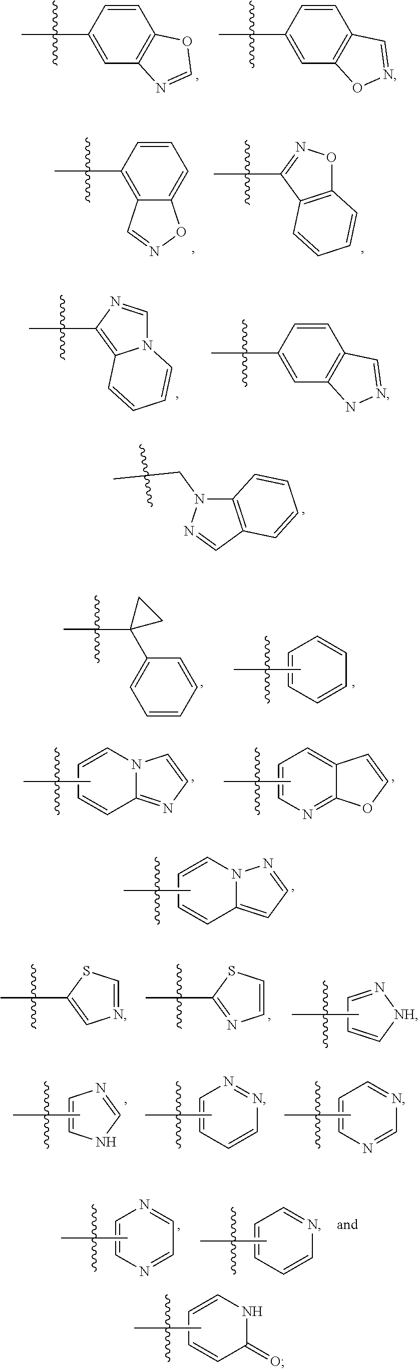

wherein each R⁹ is independently substituted with 0-5 R¹¹;
wherein R¹⁰ is selected from the group consisting of —H, halogen, —(C1-C6)alkyl, —(C6-C10) aryl, -5-10 membered heteroaryl, —(C3-C6) cycloalkyl, —CH₂—(C3-C6) cycloalkyl, —CH₂—(C6-C10) aryl, and —CH₂-5-10-membered heteroaryl,
   wherein each R¹⁰ is substituted with 0-5 R′;
wherein each occurrence of R¹¹ is independently selected from the group consisting of -halogen, —CN, SCH₃, —CF₃, —OH, —OCF₃, OCHF₂, —O(C1-C6)alkyl, —(C6-C10) aryl, —(C1-C6)alkyl, and -5 to 10 membered heteroaryl;
   each R is independently selected from the group consisting of:
      H—,
      (C1-C12)-aliphatic-,
      (C3-C10)-cycloalkyl-,
      (C3-C10)-cycloalkenyl-,
      [(C3-C10)-cycloalkyl]-(C1-C12)-aliphatic-,
      [(C3-C10)-cycloalkenyl]-(C1-C12)-aliphatic-,
      [(C3-C10)-cycloalkyl]-O—(C1-C12)-aliphatic-,
      [(C3-C10)-cycloalkenyl]-O—(C1-C12)-aliphatic-,
      (C6-C10)-aryl-,
      (C6-C10)-aryl-(C1-C12) aliphatic-,
      (C6-C10)-aryl-O—(C1-C12) aliphatic-,
      (C6-C10)-aryl-N(R″)—(C1-C12) aliphatic-,
      3- to 10-membered heterocyclyl-,
      (3- to 10-membered heterocyclyl)-(C1-C12) aliphatic-,
      (3- to 10-membered heterocyclyl)-O—(C1-C12) aliphatic-,
      (3- to 10-membered heterocyclyl)-N(R″)—(C1-C12) aliphatic-,
      5- to 10-membered heteroaryl-,
      (5- to 10-membered heteroaryl)-(C1-C12)-aliphatic-,
      (5- to 10-membered heteroaryl)-O—(C1-C12)-aliphatic-; and
      (5- to 10-membered heteroaryl)-N(R″)—(C1-C12)-aliphatic-;
wherein said heterocyclyl has 1-4 heteroatoms independently selected from the group consisting of N, NH, O, S, SO, and SO₂, and said heteroaryl has 1-4 heteroatoms independently selected from the group consisting of N, NH, O, and S;
   wherein each occurrence of R is independently substituted with 0-5 R′;
or when two R groups bound to the same atom, the two R groups may be taken together with the atom to which they are bound to form a 3- to 10-membered aromatic or non-aromatic ring having 0-4 heteroatoms independently selected from the group consisting of N, NH, O, S, SO, and SO₂, wherein said ring is optionally substituted with 0-5 R′, and wherein said ring is optionally fused to a (C6-C10) aryl, 5- to 10-membered heteroaryl, (C3-C10) cycloalkyl, or a 3- to 10-membered heterocyclyl;
wherein each occurrence of R′ is independently selected from the group consisting of halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂;
wherein each occurrence of R″ is independently selected from the group consisting of H, —(C1-C6)-alkyl, —(C1-C6)-aliphatic, (C3-C6)-cycloalkyl, 3- to 6-membered heterocyclyl, 5- to 10-membered heteroaryl-, (C6-C10)-aryl-, (5- to 10-membered heteroaryl)-(C1-C6)-alkyl-, (C6-C10)-aryl-(C1-C6)-alkyl-, (5- to 10-membered heteroaryl)-O—(C1-C6)-alkyl-, (C6-C10)-aryl-O—(C1-C6)-alkyl-, and (C6-C10)-aryl-O—(C1-C6)-alkyl-,
wherein each occurrence of R″ is independently substituted with 0-5 substituents selected from the group consisting of: halogen, —R^{o}, —OR^{o}, oxo, —CH₂OR^{o}, —CH₂N(R^{o})₂, —C(O)N(R^{o})₂, —C(O)OR^{o}, —NO₂, —NCS, —CN, —CF₃, —OCF₃ and —N(R^{o})₂, wherein each occurrence of R^{o} is independently selected from the group consisting of: —(C1-C6)-aliphatic, (C3-C6)-cycloalkyl, 3- to 6-membered heterocyclyl, 5- to 10-membered heteroaryl-, and (C6-C10)-aryl; and
   ii) a compound of formula IV:

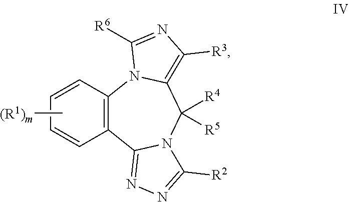

or a pharmaceutically acceptable salt, hydrate, solvate, polymorph, or isomer thereof,
wherein:
m is 0-3;
each R¹, R⁴ and R⁵ is independently selected from: each occurrence of R¹, R⁴, and R⁵ are each independently selected from:
   halogen, —R, —OR, —NO₂, —NCS, —CN, —CF₃, —OCF₂H—OCF₃, —SiR₃, —N(R)₂, —SR, —SOR, —SO₂R, —SO₂N(R)₂, —SO₃R, —(CR₂)₁₋₃R, —(CR₂)₁₋₃—OR, —(CR₂)₁₋₃—O(CR₂)₁₋₃—R, —(CR₂)₀₋₃—C(O)NR(CR₂)₀₋₃R, —(CR₂)₀₋₃—C(O)NR(CR₂)₀₋₃OR, —C(O)R, —C(O)C(O)R, —C(O)CH₂C(O)R, —C(S)R, —C(S)OR, —C(O)OR, —C(O)C(O)OR, —C(O)C(O)N(R)₂, —OC(O)R, —C(O)N(R)₂, —OC(O)N(R)₂, —C(S)N(R)₂, —(CR₂)₀₋₃NHC(O)R, —N(R)N(R)COR, —N(R)N(R)C(O)OR, —N(R)N(R)CON(R)₂, —N(R)SO₂R, —N(R)SO₂N(R)₂, —N(R)C(O)OR, —N(R)C(O)R, —N(R)C(S)R, —N(R)C(O)N(R)₂, —N(R)C(S)N(R)₂, —N(COR)COR, —N(OR)R, —C(═NH)N(R)₂, —C(O)N(OR)R, —C(═NOR)R, —OP(O)(OR)₂, —P(O)(R)₂, —P(O)(OR)₂, —P(O)(H)(OR), C≡C—R⁸, CH₂CF₃, or CHF₂;
R² is selected from —OR⁸, —SR⁸, —(CH₂)OR⁸, —(CH₂)ₙO(CH₂)ₙR⁸, —(CH₂)ₚR⁸ or —(CH₂)ₙN(R″)R¹⁰, wherein n is an integer selected from 0-4; p is an integer selected from 2-4;
   wherein R² is independently substituted with 0-5 R′;
each R³ is independently selected from:
   —H, —CN, halogen, —(C1-C6) aliphatic, —CH═CR⁹, —C≡CR⁹, —SO₂((C1-C6)alkyl), —C(O)N((C1-C6)alkyl)₂), —C(O)NH((C1-C6) aliphatic), (C6-C10)-aryl-(C1-C12) aliphatic-, —C(O)((C1-C6)alkyl), —C(O)O((C1-C6)alkyl), 5- or 6-membered heterocyclyl, 5- or 6-membered heteroaryl, —(C1-C6)alkyl-C≡C—R¹⁰, —CH₂—O—R¹⁰, —CH₂—O—CH₂—R¹⁰

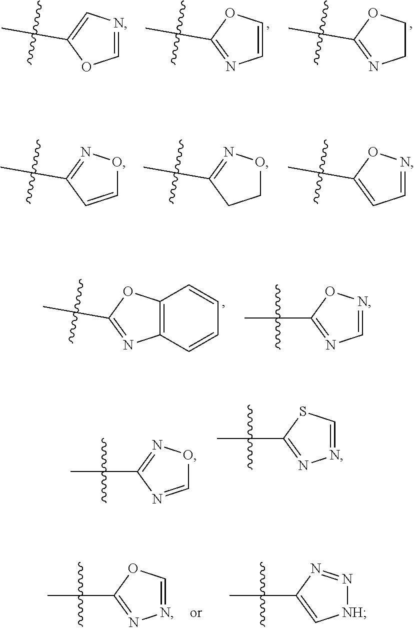

wherein each 5-10-membered heterocycle or heteroaryl are substituted with 0-3 R₇;
   wherein R³ is independently substituted with 0-5 R′;
R⁶ is selected from the group consisting of —H and —(C1-C6)alkyl;
R₇ is selected from the group consisting of —(C1-C6)alkyl, —(C3-C6) cycloalkyl, -5 to 10 membered heteroaryl, —(C6-C10) aryl, —(C6-C10) aryl-(C1-C6)alkyl, and -5 to 10 membered heteroaryl-(C1-C6)alkyl, and -5-10 membered heteroaryl;
   wherein each R₇ is independently substituted with 0-5 R′;
R⁸ is independently selected from the group consisting of —H, —(C1-C6)alkyl, —(C3-C10)-cycloalkyl, (C6-C10)-aryl, or 5- to 10-membered heteroaryl, 5-10 membered heteroaryl-(C1-C6)alkyl-, —(C1-C6)alkyl-(C6-C10) aryl, and —(C1-C6)alkyl-(C3-C6) cycloalkyl;
   wherein each occurrence of R⁸ is independently substituted with 0-5 R′;
wherein R⁹ is selected from the group consisting of —H, —(C1-C6)alkyl, —(C3-C6) cycloalkyl, —(C1-C6)alkyl-(C3-C6) cycloalkyl, —(C1-C6)alkyl-(C6-C10) aryl, —(C6-C10) aryl, -5-10 membered heteroaryl, —(C1-C6)alkyl-5-10 membered heteroaryl, 5-10 membered heterocycle, —C(O)—(C6-C10) aryl,

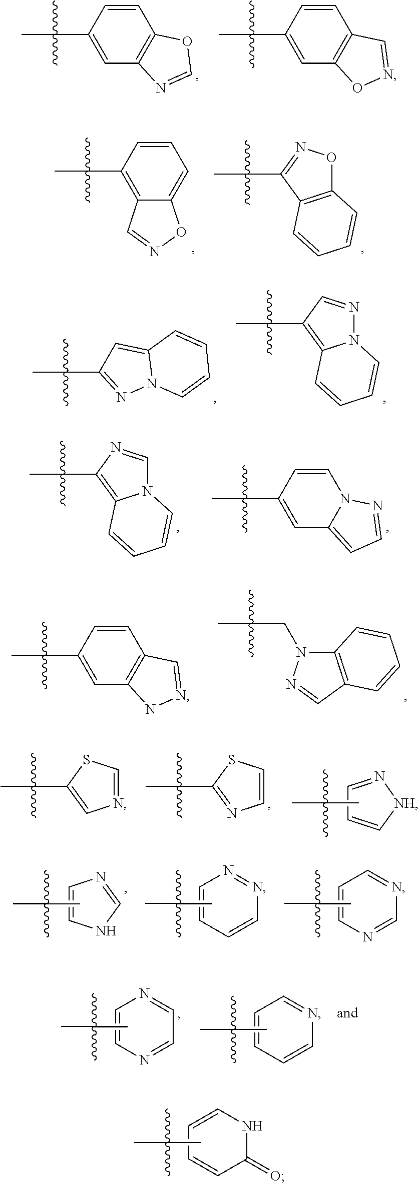

wherein each wherein each R⁹ is independently substituted with 0-5 R¹¹;
R¹⁰ is selected from the group consisting of —H, —(C1-C6)alkyl, —(C3-C10)-cycloalkyl, 3- to 10-membered heterocyclyl-, (C6-C10)-aryl, 5- to 10-membered heteroaryl, —CH₂—(C3-C6) cycloalkyl, —CH₂—(C6-C10) aryl, and —CH₂-5-10-membered heteroaryl,
   wherein each occurrence of R¹⁰ is independently substituted with 0-5 R′;
wherein each occurrence of R¹¹ is independently selected from the group consisting of -halogen, —CF₃, —OCF₃, —OH, OCF₂H, —O—(C1-C6)alkyl, —(C6-C10) aryl, —(C1-C6)alkyl, —O—CH₂—(C3-C6) cycloalkyl, —CN, and -5 to 10 membered heteroaryl;
wherein each occurrence of R′ is independently selected from the group consisting of halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂;
wherein each occurrence of R″ is independently selected from the group consisting of H, —(C1-C6)-aliphatic, —(C1-C6)-alkyl, (C3-C6)-cycloalkyl, 3- to 6-membered heterocyclyl, 5- to 10-membered heteroaryl-, (C6-C10)-aryl-, (5- to 10-membered heteroaryl)-(C1-C6)-alkyl-, (C6-C10)-aryl-(C1-C6)-alkyl-, (5- to 10-membered heteroaryl)-O—(C1-C6)-alkyl-, and (C6-C10)-aryl-O—(C1-C6)-alkyl-;
wherein each occurrence of R″ is independently substituted with 0-5 R^{t} independently selected from the group consisting of: halogen, —R^{o}, —OR^{o}, oxo, —CH₂OR^{o}, —CH₂N(R^{o})₂, —C(O)N(R^{o})₂, —C(O)OR^{o}, —NO₂, —NCS, —CN, —CF₃, —OCF₃ and —N(R^{o})₂, wherein
   each occurrence of R^{o} is independently selected from: —(C1-C6)-aliphatic, (C3-C6)-cycloalkyl, 3- to 6-membered heterocyclyl, 5- to 10-membered heteroaryl-, and (C6-C10)-aryl-.

In some embodiments, the GABA_{A} α5 receptor agonist, or the pharmaceutically acceptable salt, hydrate, solvate, isomer, or polymorph thereof or the GABA_{A} α5 receptor agonist of this disclosure, or the pharmaceutically acceptable salt, hydrate, solvate, isomer, or polymorph thereof of the pharmaceutical composition of this disclosure, is selected from the group consisting of:
i) a compound of formula II:

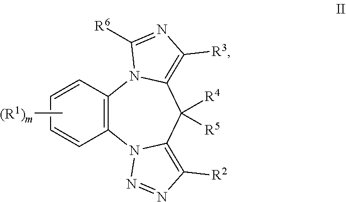

or a pharmaceutically acceptable salt, hydrate, solvate, polymorph, or isomer thereof,
wherein:
m is 0-3;
each R¹ is independently selected from the group consisting of: halogen, —H, —(C1-C6)alkyl, —OH, —O((C1-C6)alkyl), —NO₂, —CN, —CF₃, —OCF₃, —OCHF₂, —OMe, —C≡C—R⁸, —CHF₂, —CH₂CF₃, —(C6-C10) aryl, —(C1-C6)alkyl-(C6-C10) aryl, -5-10 membered heteroaryl, —(C1-C6)alkyl-5-10 membered heteroaryl, and —(C3-C6) cycloalkyl; wherein R¹ is independently substituted with 0-5 R′;
R² is selected from the group consisting of:
   —H, halogen, —OH, —(C1-C6) aliphatic, —O((C1-C6)alkyl), —C(O)O((C1-C6)alkyl), —C(O)NR₂, —(CR₂)₁₋₃—OR, —(CR₂)₁₋₃—O(CR₂)₁₋₃—R, —OR⁹, —C(O)R⁸, —CH₂R⁸, —CH₃, —CH₂—OR⁸, (C6-C10)-aryl-,
      (C6-C10)-aryl-(C1-C12) aliphatic-,
      (C6-C10)-aryl-O—(C1-C12) aliphatic-,
      (C6-C10)-aryl-N(R″)—(C1-C12) aliphatic-,
      (5- to 10-membered heteroaryl)-(C1-C12) aliphatic-,
      (5- to 10-membered heteroaryl)-O—(C1-C12) aliphatic-,
      (5- to 10-membered heteroaryl)-N(R″)—(C1-C12) aliphatic-,
      (3- to 10-membered heterocyclyl)-(C1-C12) aliphatic-,
      (3- to 10-membered heterocyclyl)-O—(C1-C12) aliphatic-, and
      (3- to 10-membered heterocyclyl)-N(R″)—(C1-C12) aliphatic-,
      wherein R² is independently substituted with 0-5 R′;
R³ is selected from the group consisting of:
   —(C1-C6)alkyl, —(C2-C6) alkenyl, —C≡CH, —C≡CR⁹, —CN, halogen, —SO₂((C6-C10)-aryl), —SO₂((C1-C6)alkyl), —C(O)N((C1-C6)alkyl)₂, —C(O)NH₂, —C(O)O((C1-C6)alkyl), —C(O)((C1-C6)alkyl), —(C6-C10) aryl, 5- to 10-membered heteroaryl, 5- to 10-membered heterocyclyl, —(C1-C6)alkyl-C≡C—R¹⁰, —CH₂—O—R¹⁰, —CH₂—O—CH₂-R¹⁰

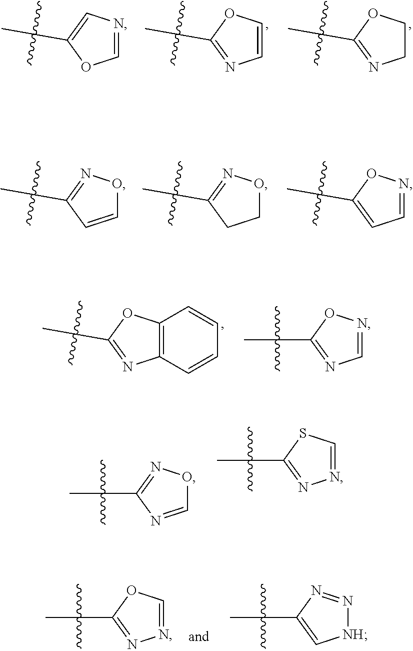

wherein each 5-member heterocycle or heteroaryl is substituted with 0-4 R₇;
wherein R³ is independently substituted with 0-5 R′;
R⁴ and R⁵ are each independently selected from the group consisting of —H, halogen, —(C1-C6)alkyl, or —(C1-C6)alkyl-(C6-C10) aryl; the (C6-C10) aryl being independently substituted with 0-5 halogen;
   R⁶ is selected from the group consisting of —H and —(C1-C6)alkyl;
wherein R₇ is selected from the group consisting of (C1-C6)alkyl, —(C3-C6) cycloalkyl, -5 to 10 membered heteroaryl, —(C6-C10) aryl, (C6-C10) aryl-(C1-C6)alkyl-, -5 to 10 membered heteroaryl-(C1-C6)alkyl, and -5-10 membered heteroaryl; wherein each R₇ is independently substituted with 0-5 R′;
wherein each R⁸ is independently selected from the group consisting of —H, —(C1-C6)alkyl, —(C3-C6) cycloalkyl, —(C1-C6)alkyl-(C3-C6) cycloalkyl, —(C1-C6)alkyl-(C6-C10) aryl, —(C6-C10) aryl, -5-10 membered heteroaryl, and —(C1-C6)alkyl-5-10 membered heteroaryl;
wherein each R⁸ excluding-H and —(C1-C6)alkyl is independently substituted by 0-5 of -halogen, —(C1-C6)alkyl, —CF₃, —OCF₃, or O—(C1-C6)alkyl;
wherein R⁹ is selected from the group consisting of —H, —(C1-C6)alkyl, —(C6-C10) aryl, -5-10 membered heteroaryl, —(C1-C6)alkyl-(C6-C10) aryl, —(C1-C6)alkyl-5-10 membered heteroaryl, —(C3-C6) cycloalkyl, —(C1-C6)alkyl-(C3-C6) cycloalkyl, —C(O)—(C6-C10) aryl, 5-10 membered heterocycle,

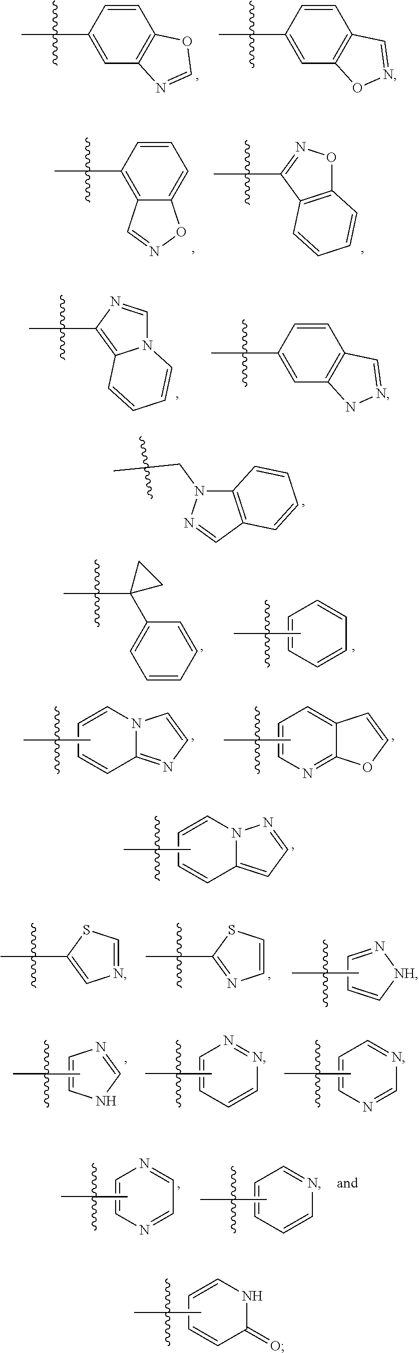

wherein each R′ is independently substituted with 0-5 R¹¹;
wherein R¹⁰ is selected from the group consisting of —H, halogen, —(C1-C6)alkyl, —(C6-C10) aryl, -5-10 membered heteroaryl, —(C3-C6) cycloalkyl, —CH₂—(C3-C6) cycloalkyl, —CH₂—(C6-C10) aryl, and —CH₂-5-10-membered heteroaryl,
   wherein each R¹⁰ is substituted with 0-5 R′;
wherein each occurrence of R¹¹ is independently selected from the group consisting of -halogen, —CN, SCH₃, —CF₃, —OH, —OCF₃, OCHF₂, —O(C1-C6)alkyl, —(C6-C10) aryl, —(C1-C6)alkyl, and -5 to 10 membered heteroaryl;
   each R is independently selected from the group consisting of:
      H—,
      (C1-C12)-aliphatic-,
      (C3-C10)-cycloalkyl-,
      (C3-C10)-cycloalkenyl-,
      [(C3-C10)-cycloalkyl]-(C1-C12)-aliphatic-,
      [(C3-C10)-cycloalkenyl]-(C1-C12)-aliphatic-,
      [(C3-C10)-cycloalkyl]-O—(C1-C12)-aliphatic-,
      [(C3-C10)-cycloalkenyl]-O—(C1-C12)-aliphatic-,
      (C6-C10)-aryl-,
      (C6-C10)-aryl-(C1-C12) aliphatic-,
      (C6-C10)-aryl-O—(C1-C12) aliphatic-,
      (C6-C10)-aryl-N(R″)—(C1-C12) aliphatic-,
      3- to 10-membered heterocyclyl-,
      (3- to 10-membered heterocyclyl)-(C1-C12) aliphatic-,
      (3- to 10-membered heterocyclyl)-O—(C1-C12) aliphatic-,
      (3- to 10-membered heterocyclyl)-N(R″)—(C1-C12) aliphatic-,
      5- to 10-membered heteroaryl-,
      (5- to 10-membered heteroaryl)-(C1-C12)-aliphatic-,
      (5- to 10-membered heteroaryl)-O—(C1-C12)-aliphatic-; and
      (5- to 10-membered heteroaryl)-N(R″)—(C1-C12)-aliphatic-;
wherein said heterocyclyl has 1-4 heteroatoms independently selected from the group consisting of N, NH, O, S, SO, and SO₂, and said heteroaryl has 1-4 heteroatoms independently selected from the group consisting of N, NH, O, and S;
   wherein each occurrence of R is independently substituted with 0-5 R′;
   or when two R groups bound to the same atom, the two R groups may be taken together with the atom to which they are bound to form a 3- to 10-membered aromatic or non-aromatic ring having 0-4 heteroatoms independently selected from the group consisting of N, NH, O, S, SO, and SO₂, wherein said ring is optionally substituted with 0-5 R′, and
   wherein said ring is optionally fused to a (C6-C10) aryl, 5- to 10-membered heteroaryl, (C3-C10) cycloalkyl, or a 3- to 10-membered heterocyclyl;
wherein each occurrence of R′ is independently selected from the group consisting of halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂;
wherein each occurrence of R″ is independently selected from the group consisting of H, —(C1-C6)-alkyl, —(C1-C6)-aliphatic, (C3-C6)-cycloalkyl, 3- to 6-membered heterocyclyl, 5- to 10-membered heteroaryl-, (C6-C10)-aryl-, (5- to 10-membered heteroaryl)-(C1-C6)-alkyl-, (C6-C10)-aryl-(C1-C6)-alkyl-, (5- to 10-membered heteroaryl)-O—(C1-C6)-alkyl-, (C6-C10)-aryl-O—(C1-C6)-alkyl-, and (C6-C10)-aryl-O—(C1-C6)-alkyl-,
wherein each occurrence of R″ is independently substituted with 0-5 substituents selected from the group consisting of: halogen, —R^{o}, —OR^{o}, oxo, —CH₂OR^{o}, —CH₂N(R^{o})₂, —C(O)N(R^{o})₂, —C(O)OR^{o}, —NO₂, —NCS, —CN, —CF₃, —OCF₃ and —N(R^{o})₂, wherein each occurrence of R^{o} is independently selected from the group consisting of: —(C1-C6)-aliphatic, (C3-C6)-cycloalkyl, 3- to 6-membered heterocyclyl, 5- to 10-membered heteroaryl-, and (C6-C10)-aryl; and
   ii) a compound of formula IV:

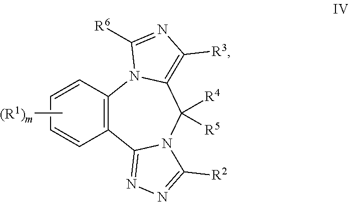

or a pharmaceutically acceptable salt, hydrate, solvate, polymorph, or isomer thereof,
wherein:
m is 0-3;
each R¹ is independently selected from the group consisting of: halogen, —H, —(C1-C6)alkyl, —C≡C—R⁹, —OH, —O((C1-C6)alkyl), —NO₂, —CN, —CF₃, —OCF₃, —CHF₂, —CH₂CF₃, —(C6-C10) aryl, —(C1-C6)alkyl-(C6-C10) aryl, -5-10 membered heteroaryl, —(C1-C6)alkyl-5-10 membered heteroaryl, and —(C3-C6) cycloalkyl;
   wherein R¹ is independently substituted with 0-5 R′;
R² is selected from the group consisting of —OR⁸, —SR⁸, —(CH₂)ₙOR8, —(CH₂)ₙO(CH₂)ₙR⁸, —(CH₂)ₚR⁸ and —(CH₂)ₙN(R″)R¹⁰, wherein n is an integer selected from 0-4; p is an integer selected from 2-4;
   wherein R² is independently substituted with 0-5 R′;
each R³ is independently selected from the group consisting of:
   —H, —CN, halogen, —(C1-C6) aliphatic, —CH═CR⁹, —C≡CR⁹, —SO₂((C1-C6)alkyl), —C(O)N((C1-C6)alkyl)₂), —C(O)NH((C1-C6) aliphatic), (C6-C10)-aryl-(C1-C12) aliphatic-, —C(O)((C1-C6)alkyl), —C(O)O((C1-C6)alkyl), 5- or 6-membered heterocyclyl, 5- or 6-membered heteroaryl, —CH₂—O—R¹⁰, —CH₂—O—CH₂-R¹⁰

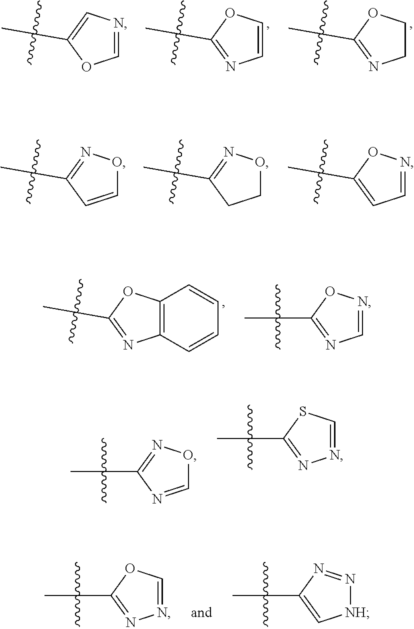

wherein each 5-10-membered heterocycle or heteroaryl are substituted with 0-3 R₇;
wherein R³ is independently substituted with 0-5 R′;
R⁴ and R⁵ are each independently selected from the group consisting of —H, halogen and —(C1-C6)alkyl;
R⁶ is selected from the group consisting of —H and —(C1-C6)alkyl;
R₇ is selected from the group consisting of —(C1-C6)alkyl, —(C3-C6) cycloalkyl, -5 to 10 membered heteroaryl, —(C6-C10) aryl, —(C6-C10) aryl-(C1-C6)alkyl, and -5 to 10 membered heteroaryl-(C1-C6)alkyl, and -5-10 membered heteroaryl;
   wherein each R₇ is independently substituted with 0-5 R′;
R⁸ is independently selected from the group consisting of —H, —(C1-C6)alkyl, —(C3-C10)-cycloalkyl, (C6-C10)-aryl, or 5- to 10-membered heteroaryl, 5-10 membered heteroaryl-(C1-C6)alkyl-, —(C1-C6)alkyl-(C6-C10) aryl, and —(C1-C6)alkyl-(C3-C6) cycloalkyl;
   wherein each occurrence of R⁸ is independently substituted with 0-5 R′;
wherein R⁹ is selected from the group consisting of —H, —(C1-C6)alkyl, —(C3-C6) cycloalkyl, —(C1-C6)alkyl-(C3-C6) cycloalkyl, —(C1-C6)alkyl-(C6-C10) aryl, —(C6-C10) aryl, -5-10 membered heteroaryl, —(C1-C6)alkyl-5-10 membered heteroaryl, 5-10 membered heterocycle, —C(O)—(C6-C10) aryl,

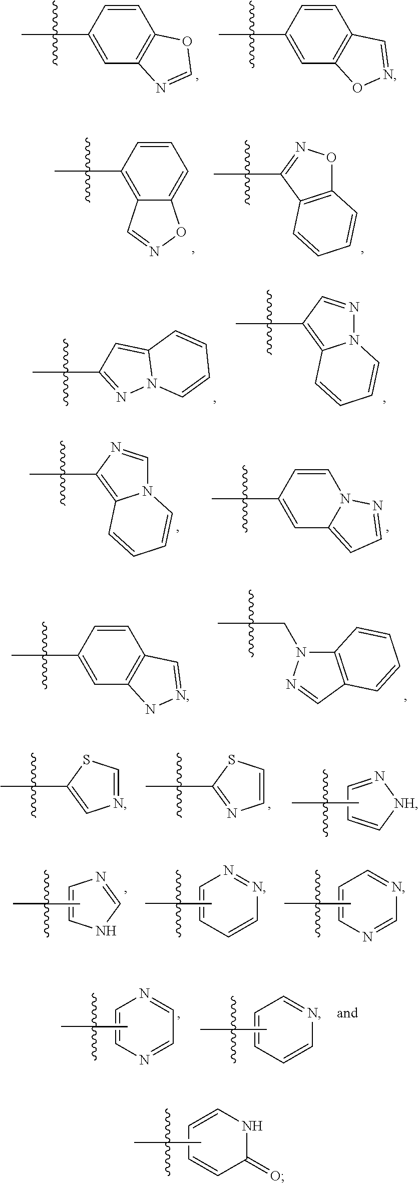

wherein each wherein each R⁹ is independently substituted with 0-5 R¹¹;
R¹⁰ is selected from the group consisting of —H, —(C1-C6)alkyl, —(C3-C10)-cycloalkyl, 3- to 10-membered heterocyclyl-, (C6-C10)-aryl, 5- to 10-membered heteroaryl, —CH₂—(C3-C6) cycloalkyl, —CH₂—(C6-C10) aryl, and —CH₂-5-10-membered heteroaryl,
   wherein each occurrence of R¹⁰ is independently substituted with 0-5 R′;
wherein each occurrence of R¹¹ is independently selected from the group consisting of -halogen, —CF₃, —OCF₃, OCF₂H, —O—(C1-C6)alkyl, —(C6-C10) aryl, —(C1-C6)alkyl, —O—CH₂—(C3-C6) cycloalkyl, and -5 to 10 membered heteroaryl;
wherein each occurrence of R′ is independently selected from the group consisting of halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂;
wherein each occurrence of R″ is independently selected from the group consisting of H, —(C1-C6)-aliphatic, —(C1-C6)-alkyl, (C3-C6)-cycloalkyl, 3- to 6-membered heterocyclyl, 5- to 10-membered heteroaryl-, (C6-C10)-aryl-, (5- to 10-membered heteroaryl)-(C1-C6)-alkyl-, (C6-C10)-aryl-(C1-C6)-alkyl-, (5- to 10-membered heteroaryl)-O—(C1-C6)-alkyl-, and (C6-C10)-aryl-O—(C1-C6)-alkyl-;
wherein each occurrence of R″ is independently substituted with 0-5 R^{t} independently selected from the group consisting of: halogen, —R^{o}, —OR^{o}, oxo, —CH₂OR^{o}, —CH₂N(R^{o})₂, —C(O)N(R^{o})₂, —C(O)OR^{o}, —NO₂, —NCS, —CN, —CF₃, —OCF₃ and —N(R^{o})₂, wherein
   each occurrence of R^{o} is independently selected from: —(C1-C6)-aliphatic, (C3-C6)-cycloalkyl, 3- to 6-membered heterocyclyl, 5- to 10-membered heteroaryl-, and (C6-C10)-aryl-.

In some embodiments, the GABA_{A} α5 receptor agonist, or the pharmaceutically acceptable salt, hydrate, solvate, polymorph, or isomer thereof or the GABA_{A} α5 receptor agonist of this disclosure, or the pharmaceutically acceptable salt, hydrate, solvate, isomer, or polymorph thereof of the pharmaceutical composition of this disclosure, is a compound of Formula II as recited in paragraph 19 or is a compound of Formula II as recited in paragraph 20, or a pharmaceutically acceptable salt, hydrate, solvate, polymorph, or isomer thereof. In other embodiments, the GABA_{A} α5 receptor agonist, or the pharmaceutically acceptable salt, hydrate, solvate, polymorph, or isomer thereof or the GABA_{A} α5 receptor agonist of this disclosure, or the pharmaceutically acceptable salt, hydrate, solvate, isomer, or polymorph thereof of the pharmaceutical composition of this disclosure, is a compound of Formula IV as recited in paragraph 19 or is a compound of Formula IV as recited in paragraph 20, or a pharmaceutically acceptable salt, hydrate, solvate, polymorph, or isomer thereof.

In some embodiments, the GABA_{A} α5 receptor agonist or the GABA_{A} α5 receptor agonist of the pharmaceutical composition of this disclosure is selected from the group consisting of compounds 1-12, 44-56, 101-268, 270-644, 646-687, 689-698, 700-703, 705, 707-721, and 723-740, or their pharmaceutically acceptable salts, hydrates, solvates, polymorphs, or isomers. See, e.g., WO2018130868, WO2018130869, WO2019246300, WO2021127543, and WO2022011318. Each of these published documents is incorporated by reference herein in its entirety and in particular in the context of the recited compounds, their synthesis and properties.

In some embodiments, the GABA_{A} α5 receptor agonist or the GABA_{A} α5 receptor agonist of the pharmaceutical composition of this disclosure is selected from the group consisting of:

| Compound | Structure |
|---|---|
| 1 | 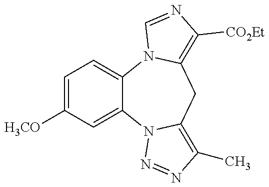 [SMILES: CCOC(=O)c1ncn2c1Cc1c(C)nnn1-c1cc(OC)ccc1-2] |
| 2 | 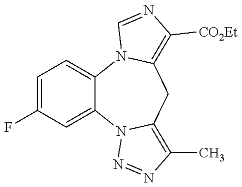 [SMILES: CCOC(=O)c1ncn2c1Cc1c(C)nnn1-c1cc(F)ccc1-2] |
| 3 | 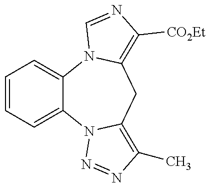 [SMILES: CCOC(=O)c1ncn2c1Cc1c(C)nnn1-c1ccccc1-2] |
| 4 | 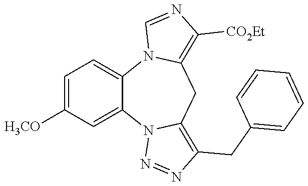 [SMILES: CCOC(=O)c1ncn2c1Cc1c(Cc3ccccc3)nnn1-c1cc(OC)ccc1-2] |
| 5 | 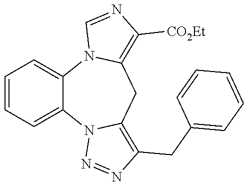 [SMILES: CCOC(=O)c1ncn2c1Cc1c(Cc3ccccc3)nnn1-c1ccccc1-2] |
| 6 | 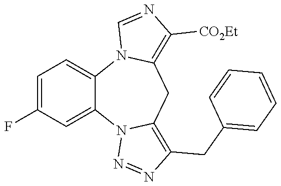 [SMILES: CCOC(=O)c1ncn2c1Cc1c(Cc3ccccc3)nnn1-c1cc(F)ccc1-2] |
| 7 | 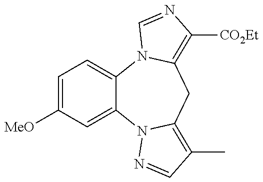 [SMILES: CCOC(=O)c1ncn2c1Cc1c(C)cnn1-c1cc(OC)ccc1-2] |
| 8 | 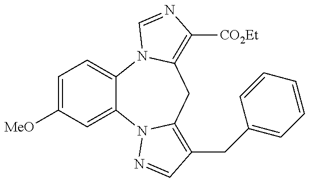 [SMILES: CCOC(=O)c1ncn2c1Cc1c(Cc3ccccc3)cnn1-c1cc(OC)ccc1-2] |
| 9 | 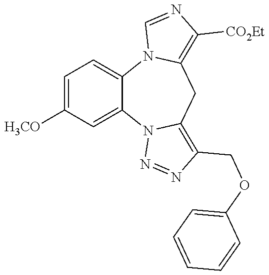 [SMILES: CCOC(=O)c1ncn2c1Cc1c(COc3ccccc3)nnn1-c1cc(OC)ccc1-2] |
| 10 | 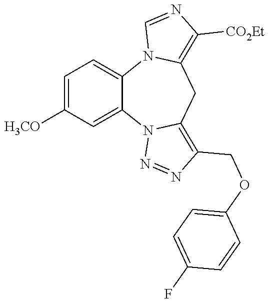 [SMILES: CCOC(=O)c1ncn2c1Cc1c(COc3ccc(F)cc3)nnn1-c1cc(OC)ccc1-2] |
| 11 | 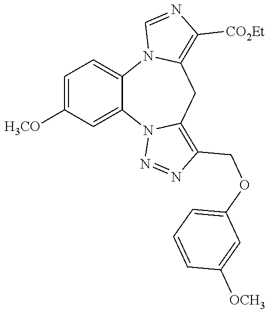 [SMILES: CCOC(=O)c1ncn2c1Cc1c(COc3cccc(OC)c3)nnn1-c1cc(OC)ccc1-2] |
| 12 | 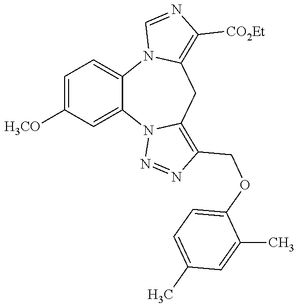 [SMILES: CCOC(=O)c1ncn2c1Cc1c(COc3ccc(C)cc3C)nnn1-c1cc(OC)ccc1-2] |
| 44 | 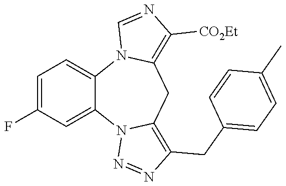 [SMILES: CCOC(=O)c1ncn2c1Cc1c(Cc3ccc(C)cc3)nnn1-c1cc(F)ccc1-2] |
| 45 | 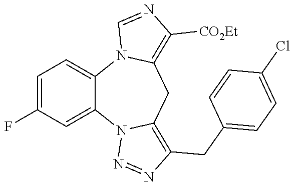 [SMILES: CCOC(=O)c1ncn2c1Cc1c(Cc3ccc(Cl)cc3)nnn1-c1cc(F)ccc1-2] |
| 46 | 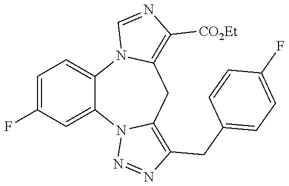 [SMILES: CCOC(=O)c1ncn2c1Cc1c(Cc3ccc(F)cc3)nnn1-c1cc(F)ccc1-2] |
| 47 | 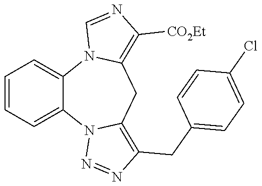 [SMILES: CCOC(=O)c1ncn2c1Cc1c(Cc3ccc(Cl)cc3)nnn1-c1ccccc1-2] |
| 48 | 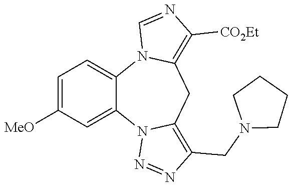 [SMILES: CCOC(=O)c1ncn2c1Cc1c(CN3CCCC3)nnn1-c1cc(OC)ccc1-2] |
| 49 | 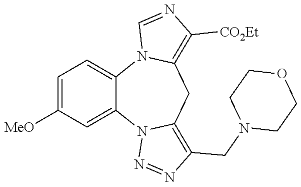 [SMILES: CCOC(=O)c1ncn2c1Cc1c(CN3CCOCC3)nnn1-c1cc(OC)ccc1-2] |
| 50 | 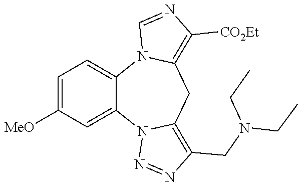 [SMILES: CCOC(=O)c1ncn2c1Cc1c(CN(CC)CC)nnn1-c1cc(OC)ccc1-2] |
| 51 | 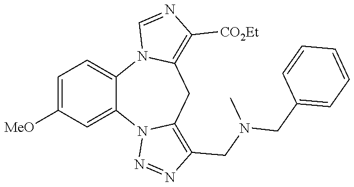 [SMILES: CCOC(=O)c1ncn2c1Cc1c(CN(C)Cc3ccccc3)nnn1-c1cc(OC)ccc1-2] |
| 52 | 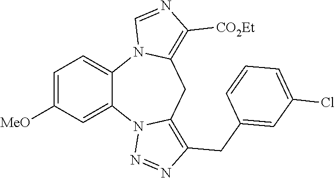 [SMILES: CCOC(=O)c1ncn2c1Cc1c(Cc3cccc(Cl)c3)nnn1-c1cc(OC)ccc1-2] |
| 53 | 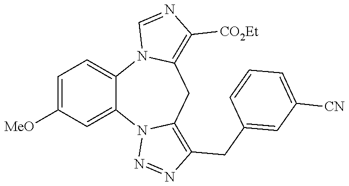 [SMILES: CCOC(=O)c1ncn2c1Cc1c(Cc3cccc(C#N)c3)nnn1-c1cc(OC)ccc1-2] |
| 54 | 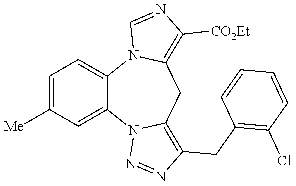 [SMILES: CCOC(=O)c1ncn2c1Cc1c(Cc3ccccc3Cl)nnn1-c1cc(C)ccc1-2] |
| 55 | 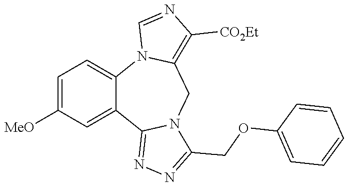 [SMILES: CCOC(=O)c1ncn2c1Cn1c(COc3ccccc3)nnc1-c1cc(OC)ccc1-2] |
| 56 | 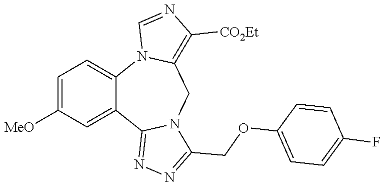 [SMILES: CCOC(=O)c1ncn2c1Cn1c(COc3ccc(F)cc3)nnc1-c1cc(OC)ccc1-2] |
| 101 | 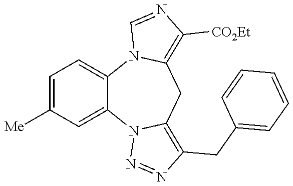 [SMILES: CCOC(=O)c1ncn2c1Cc1c(Cc3ccccc3)nnn1-c1cc(C)ccc1-2] |
| 102 | 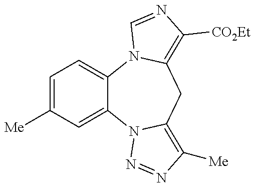 [SMILES: CCOC(=O)c1ncn2c1Cc1c(C)nnn1-c1cc(C)ccc1-2] |
| 103 | 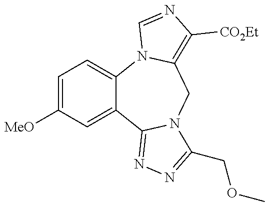 [SMILES: CCOC(=O)c1ncn2c1Cn1c(COC)nnc1-c1cc(OC)ccc1-2] |
| 104 | 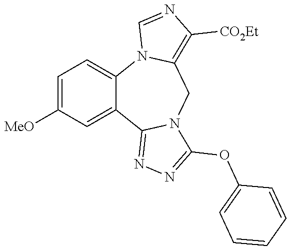 [SMILES: CCOC(=O)c1ncn2c1Cn1c(Oc3ccccc3)nnc1-c1cc(OC)ccc1-2] |
| 105 | 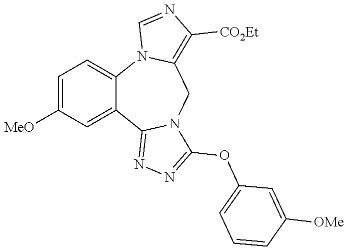 [SMILES: CCOC(=O)c1ncn2c1Cn1c(Oc3cccc(OC)c3)nnc1-c1cc(OC)ccc1-2] |
| 106 | 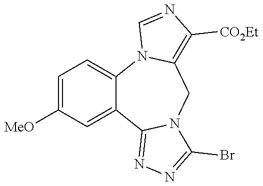 [SMILES: CCOC(=O)c1ncn2c1Cn1c(Br)nnc1-c1cc(OC)ccc1-2] |
| 107 | 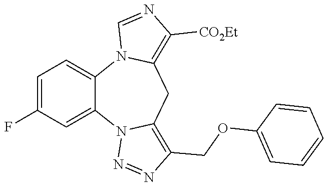 [SMILES: CCOC(=O)c1ncn2c1Cc1c(COc3ccccc3)nnn1-c1cc(F)ccc1-2] |
| 108 | 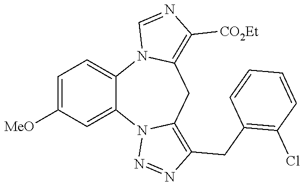 [SMILES: CCOC(=O)c1ncn2c1Cc1c(Cc3ccccc3Cl)nnn1-c1cc(OC)ccc1-2] |
| 109 | 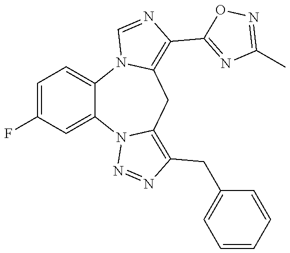 [SMILES: Cc1noc(-c2ncn3c2Cc2c(Cc4ccccc4)nnn2-c2cc(F)ccc2-3)n1] |
| 110 | 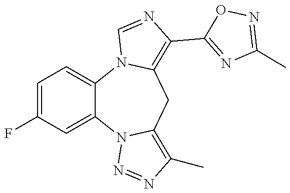 [SMILES: Cc1noc(-c2ncn3c2Cc2c(C)nnn2-c2cc(F)ccc2-3)n1] |
| 111 | 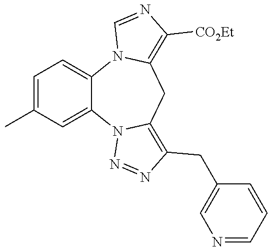 [SMILES: CCOC(=O)c1ncn2c1Cc1c(Cc3cccnc3)nnn1-c1cc(C)ccc1-2] |
| 112 | 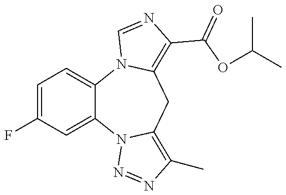 [SMILES: Cc1nnn2c1Cc1c(C(=O)OC(C)C)ncn1-c1ccc(F)cc1-2] |
| 113 | 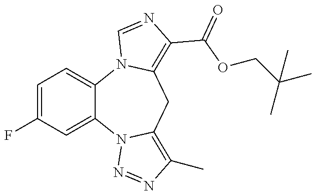 [SMILES: Cc1nnn2c1Cc1c(C(=O)OCC(C)(C)C)ncn1-c1ccc(F)cc1-2] |
| 114 | 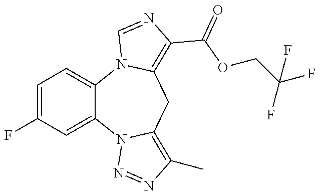 [SMILES: Cc1nnn2c1Cc1c(C(=O)OCC(F)(F)F)ncn1-c1ccc(F)cc1-2] |
| 115 | 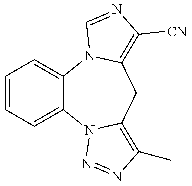 [SMILES: Cc1nnn2c1Cc1c(C#N)ncn1-c1ccccc1-2] |
| 116 | 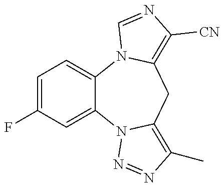 [SMILES: Cc1nnn2c1Cc1c(C#N)ncn1-c1ccc(F)cc1-2] |
| 117 | 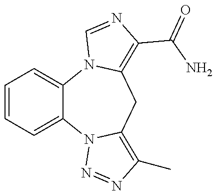 [SMILES: Cc1nnn2c1Cc1c(C(N)=O)ncn1-c1ccccc1-2] |
| 118 | 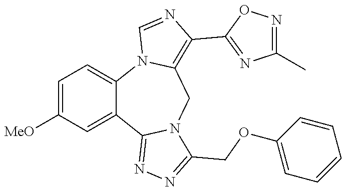 [SMILES: COc1ccc2c(c1)-c1nnc(COc3ccccc3)n1Cc1c(-c3nc(C)no3)ncn1-2] |
| 119 | 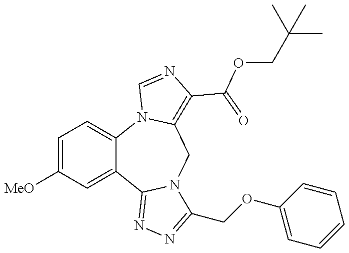 [SMILES: COc1ccc2c(c1)-c1nnc(COc3ccccc3)n1Cc1c(C(=O)OCC(C)(C)C)ncn1-2] |
| 120 | 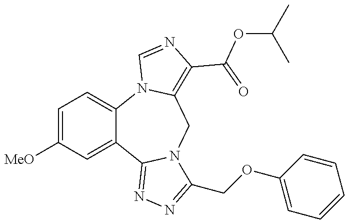 [SMILES: COc1ccc2c(c1)-c1nnc(COc3ccccc3)n1Cc1c(C(=O)OC(C)C)ncn1-2] |
| 121 | 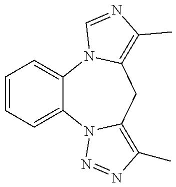 [SMILES: Cc1ncn2c1Cc1c(C)nnn1-c1ccccc1-2] |
| 122 | 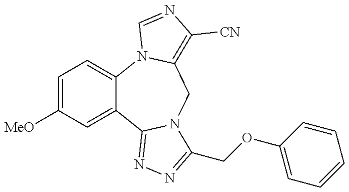 [SMILES: COc1ccc2c(c1)-c1nnc(COc3ccccc3)n1Cc1c(C#N)ncn1-2] |
| 123 | 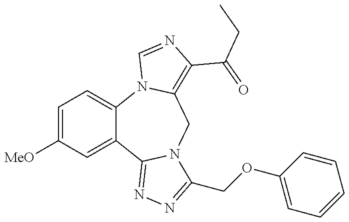 [SMILES: CCC(=O)c1ncn2c1Cn1c(COc3ccccc3)nnc1-c1cc(OC)ccc1-2] |
| 124 | 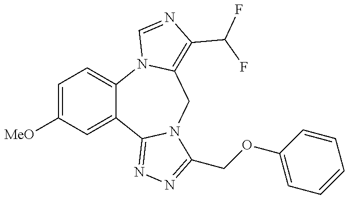 [SMILES: COc1ccc2c(c1)-c1nnc(COc3ccccc3)n1Cc1c(C(F)F)ncn1-2] |
| 125 | 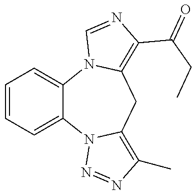 [SMILES: CCC(=O)c1ncn2c1Cc1c(C)nnn1-c1ccccc1-2] |
| 126 | 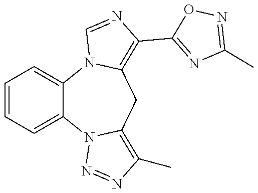 [SMILES: Cc1noc(-c2ncn3c2Cc2c(C)nnn2-c2ccccc2-3)n1] |
| 127 | 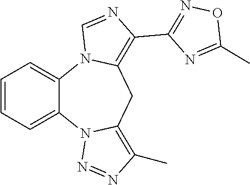 [SMILES: Cc1nc(-c2ncn3c2Cc2c(C)nnn2-c2ccccc2-3)no1] |
| 128 | 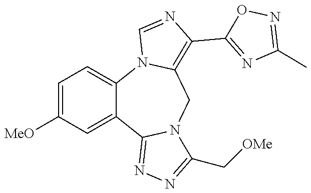 [SMILES: COCc1nnc2n1Cc1c(-c3nc(C)no3)ncn1-c1ccc(OC)cc1-2] |
| 129 | 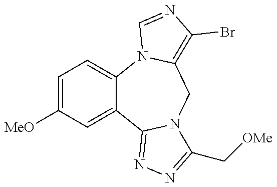 [SMILES: COCc1nnc2n1Cc1c(Br)ncn1-c1ccc(OC)cc1-2] |
| 130 | 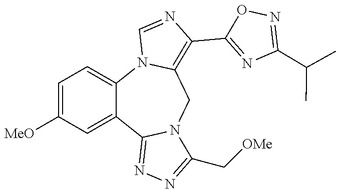 [SMILES: COCc1nnc2n1Cc1c(-c3nc(C(C)C)no3)ncn1-c1ccc(OC)cc1-2] |
| 131 | 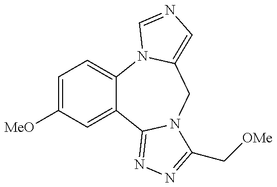 [SMILES: COCc1nnc2n1Cc1cncn1-c1ccc(OC)cc1-2] |
| 132 | 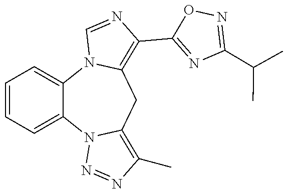 [SMILES: Cc1nnn2c1Cc1c(-c3nc(C(C)C)no3)ncn1-c1ccccc1-2] |
| 133 | 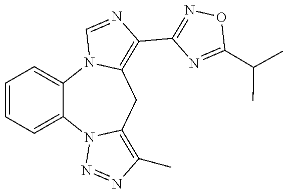 [SMILES: Cc1nnn2c1Cc1c(-c3noc(C(C)C)n3)ncn1-c1ccccc1-2] |
| 134 | 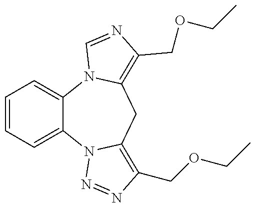 [SMILES: CCOCc1ncn2c1Cc1c(COCC)nnn1-c1ccccc1-2] |
| 135 | 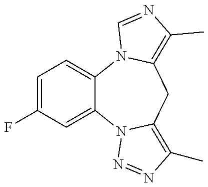 [SMILES: Cc1ncn2c1Cc1c(C)nnn1-c1cc(F)ccc1-2] |
| 136 | 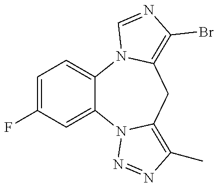 [SMILES: Cc1nnn2c1Cc1c(Br)ncn1-c1ccc(F)cc1-2] |
| 137 | 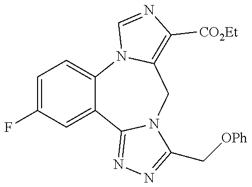 [SMILES: CCOC(=O)c1ncn2c1Cn1c(COc3ccccc3)nnc1-c1cc(F)ccc1-2] |
| 138 | 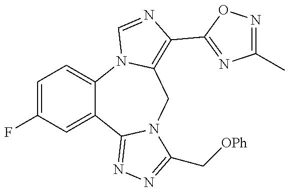 [SMILES: Cc1noc(-c2ncn3c2Cn2c(COc4ccccc4)nnc2-c2cc(F)ccc2-3)n1] |
| 139 | 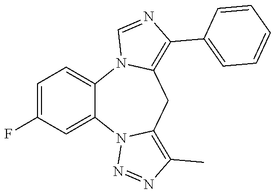 [SMILES: Cc1nnn2c1Cc1c(-c3ccccc3)ncn1-c1ccc(F)cc1-2] |
| 140 | 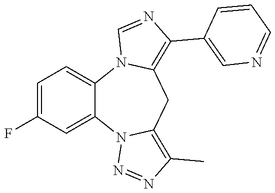 [SMILES: Cc1nnn2c1Cc1c(-c3cccnc3)ncn1-c1ccc(F)cc1-2] |
| 141 | 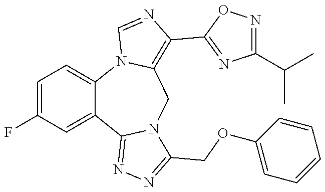 [SMILES: CC(C)c1noc(-c2ncn3c2Cn2c(COc4ccccc4)nnc2-c2cc(F)ccc2-3)n1] |
| 142 | 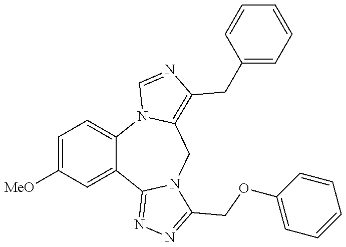 [SMILES: COc1ccc2c(c1)-c1nnc(COc3ccccc3)n1Cc1c(Cc3ccccc3)ncn1-2] |
| 143 | 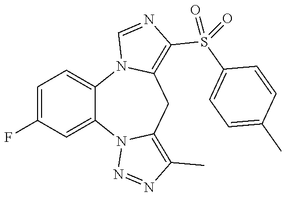 [SMILES: Cc1ccc(S(=O)(=O)c2ncn3c2Cc2c(C)nnn2-c2cc(F)ccc2-3)cc1] |
| 144 | 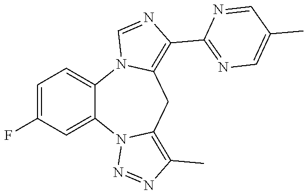 [SMILES: Cc1cnc(-c2ncn3c2Cc2c(C)nnn2-c2cc(F)ccc2-3)nc1] |
| 145 | 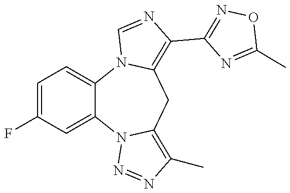 [SMILES: Cc1nc(-c2ncn3c2Cc2c(C)nnn2-c2cc(F)ccc2-3)no1] |
| 146 | 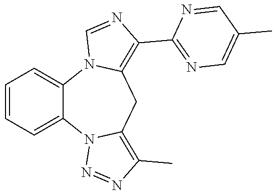 [SMILES: Cc1cnc(-c2ncn3c2Cc2c(C)nnn2-c2ccccc2-3)nc1] |
| 147 | 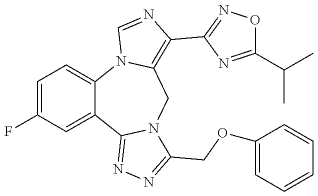 [SMILES: CC(C)c1nc(-c2ncn3c2Cn2c(COc4ccccc4)nnc2-c2cc(F)ccc2-3)no1] |
| 148 | 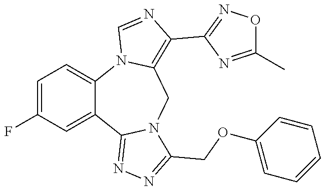 [SMILES: Cc1nc(-c2ncn3c2Cn2c(COc4ccccc4)nnc2-c2cc(F)ccc2-3)no1] |
| 149 | 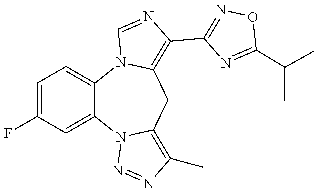 [SMILES: Cc1nnn2c1Cc1c(-c3noc(C(C)C)n3)ncn1-c1ccc(F)cc1-2] |
| 150 | 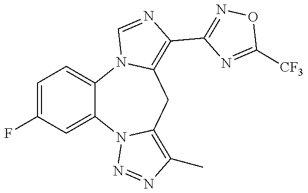 [SMILES: Cc1nnn2c1Cc1c(-c3noc(C(F)(F)F)n3)ncn1-c1ccc(F)cc1-2] |
| 151 | 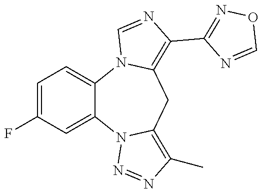 [SMILES: Cc1nnn2c1Cc1c(-c3ncon3)ncn1-c1ccc(F)cc1-2] |
| 152 | 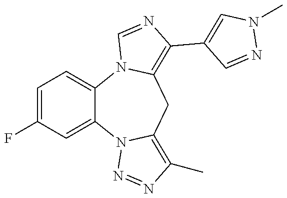 [SMILES: Cc1nnn2c1Cc1c(-c3cnn(C)c3)ncn1-c1ccc(F)cc1-2] |
| 153 | 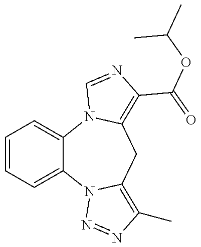 [SMILES: Cc1nnn2c1Cc1c(C(=O)OC(C)C)ncn1-c1ccccc1-2] |
| 154 | 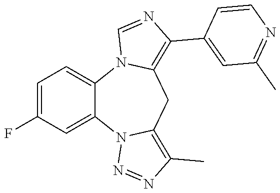 [SMILES: Cc1cc(-c2ncn3c2Cc2c(C)nnn2-c2cc(F)ccc2-3)ccn1] |
| 155 | 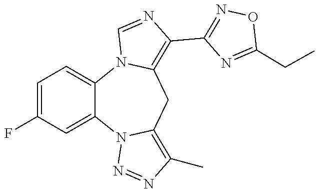 [SMILES: CCc1nc(-c2ncn3c2Cc2c(C)nnn2-c2cc(F)ccc2-3)no1] |
| 156 | 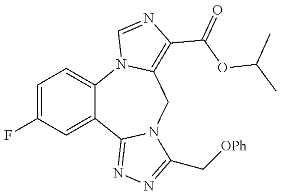 [SMILES: CC(C)OC(=O)c1ncn2c1Cn1c(COc3ccccc3)nnc1-c1cc(F)ccc1-2] |
| 157 | 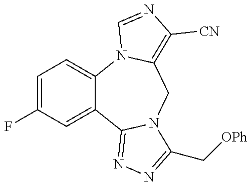 [SMILES: N#Cc1ncn2c1Cn1c(COc3ccccc3)nnc1-c1cc(F)ccc1-2] |
| 158 | 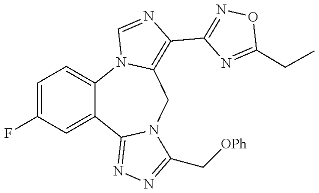 [SMILES: CCc1nc(-c2ncn3c2Cn2c(COc4ccccc4)nnc2-c2cc(F)ccc2-3)no1] |
| 159 | 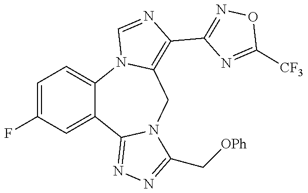 [SMILES: Fc1ccc2c(c1)-c1nnc(COc3ccccc3)n1Cc1c(-c3noc(C(F)(F)F)n3)ncn1-2] |
| 160 | 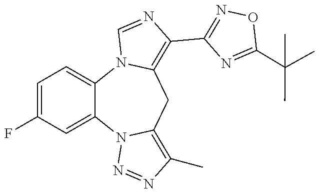 [SMILES: Cc1nnn2c1Cc1c(-c3noc(C(C)(C)C)n3)ncn1-c1ccc(F)cc1-2] |
| 161 | 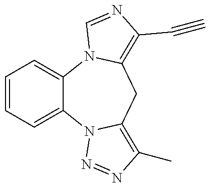 [SMILES: C#Cc1ncn2c1Cc1c(C)nnn1-c1ccccc1-2] |
| 162 | 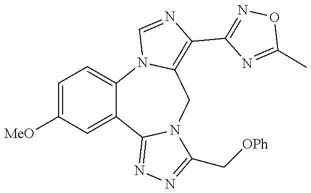 [SMILES: COc1ccc2c(c1)-c1nnc(COc3ccccc3)n1Cc1c(-c3noc(C)n3)ncn1-2] |
| 163 | 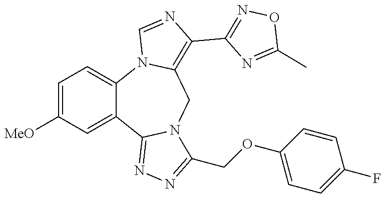 [SMILES: COc1ccc2c(c1)-c1nnc(COc3ccc(F)cc3)n1Cc1c(-c3noc(C)n3)ncn1-2] |
| 164 | 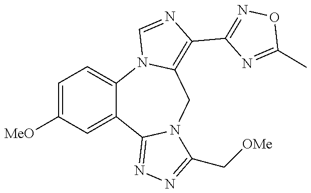 [SMILES: COCc1nnc2n1Cc1c(-c3noc(C)n3)ncn1-c1ccc(OC)cc1-2] |
| 165 | 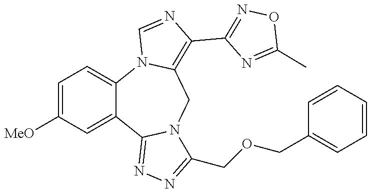 [SMILES: COc1ccc2c(c1)-c1nnc(COCc3ccccc3)n1Cc1c(-c3noc(C)n3)ncn1-2] |
| 166 | 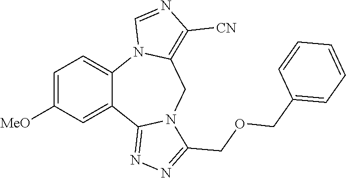 [SMILES: COc1ccc2c(c1)-c1nnc(COCc3ccccc3)n1Cc1c(C#N)ncn1-2] |
| 167 | 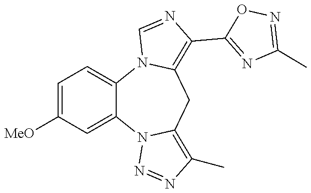 [SMILES: COc1ccc2c(c1)-n1nnc(C)c1Cc1c(-c3nc(C)no3)ncn1-2] |
| 168 | 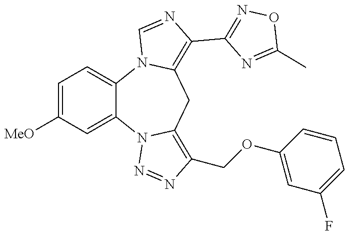 [SMILES: COc1ccc2c(c1)-n1nnc(COc3cccc(F)c3)c1Cc1c(-c3noc(C)n3)ncn1-2] |
| 169 | 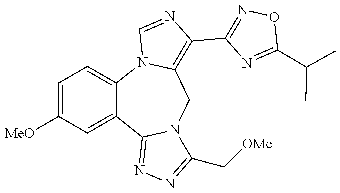 [SMILES: COCc1nnc2n1Cc1c(-c3noc(C(C)C)n3)ncn1-c1ccc(OC)cc1-2] |
| 170 | 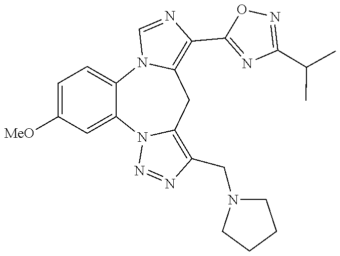 [SMILES: COc1ccc2c(c1)-n1nnc(CN3CCCC3)c1Cc1c(-c3nc(C(C)C)no3)ncn1-2] |
| 171 | 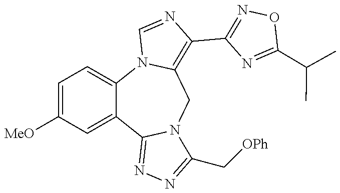 [SMILES: COc1ccc2c(c1)-c1nnc(COc3ccccc3)n1Cc1c(-c3noc(C(C)C)n3)ncn1-2] |
| 172 | 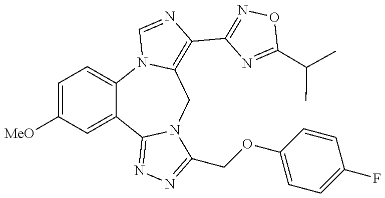 [SMILES: COc1ccc2c(c1)-c1nnc(COc3ccc(F)cc3)n1Cc1c(-c3noc(C(C)C)n3)ncn1-2] |
| 173 | 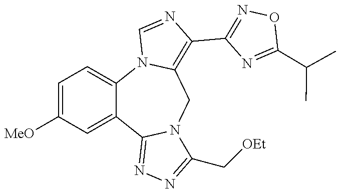 [SMILES: CCOCc1nnc2n1Cc1c(-c3noc(C(C)C)n3)ncn1-c1ccc(OC)cc1-2] |
| 174 | 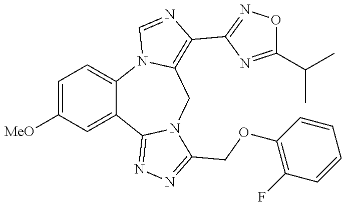 [SMILES: COc1ccc2c(c1)-c1nnc(COc3ccccc3F)n1Cc1c(-c3noc(C(C)C)n3)ncn1-2] |
| 175 | 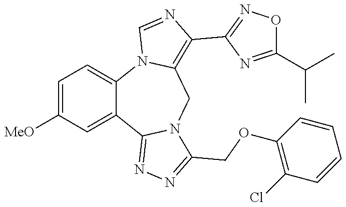 [SMILES: COc1ccc2c(c1)-c1nnc(COc3ccccc3Cl)n1Cc1c(-c3noc(C(C)C)n3)ncn1-2] |
| 176 | 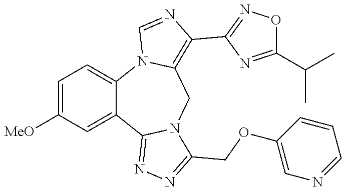 [SMILES: COc1ccc2c(c1)-c1nnc(COc3cccnc3)n1Cc1c(-c3noc(C(C)C)n3)ncn1-2] |
| 177 | 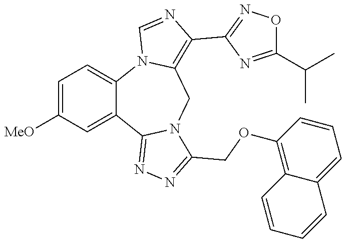 [SMILES: COc1ccc2c(c1)-c1nnc(COc3cccc4ccccc34)n1Cc1c(-c3noc(C(C)C)n3)ncn1-2] |
| 178 | 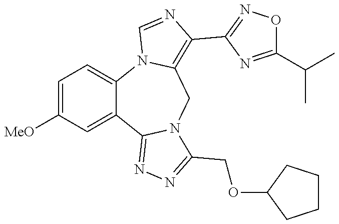 [SMILES: COc1ccc2c(c1)-c1nnc(COC3CCCC3)n1Cc1c(-c3noc(C(C)C)n3)ncn1-2] |
| 179 | 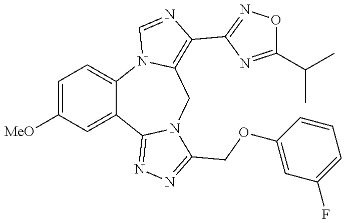 [SMILES: COc1ccc2c(c1)-c1nnc(COc3cccc(F)c3)n1Cc1c(-c3noc(C(C)C)n3)ncn1-2] |
| 180 | 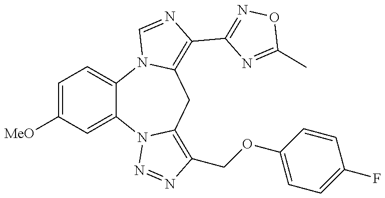 [SMILES: COc1ccc2c(c1)-n1nnc(COc3ccc(F)cc3)c1Cc1c(-c3noc(C)n3)ncn1-2] |
| 181 | 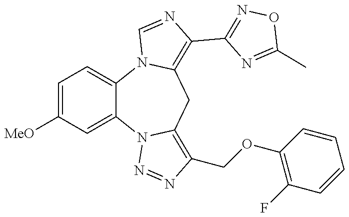 [SMILES: COc1ccc2c(c1)-n1nnc(COc3ccccc3F)c1Cc1c(-c3noc(C)n3)ncn1-2] |
| 182 | 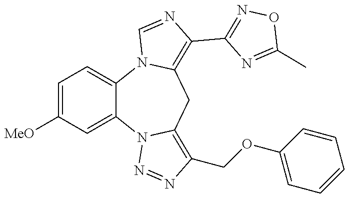 [SMILES: COc1ccc2c(c1)-n1nnc(COc3ccccc3)c1Cc1c(-c3noc(C)n3)ncn1-2] |
| 183 | 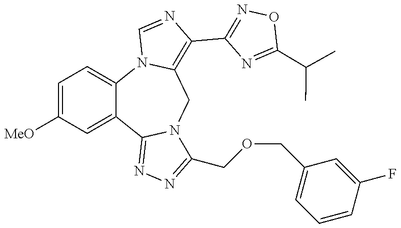 [SMILES: COc1ccc2c(c1)-c1nnc(COCc3cccc(F)c3)n1Cc1c(-c3noc(C(C)C)n3)ncn1-2] |
| 184 | 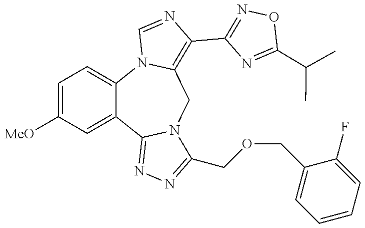 [SMILES: COc1ccc2c(c1)-c1nnc(COCc3ccccc3F)n1Cc1c(-c3noc(C(C)C)n3)ncn1-2] |
| 185 | 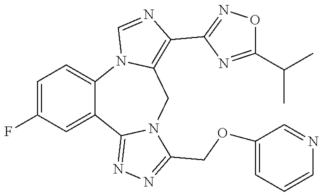 [SMILES: CC(C)c1nc(-c2ncn3c2Cn2c(COc4cccnc4)nnc2-c2cc(F)ccc2-3)no1] |
| 186 | 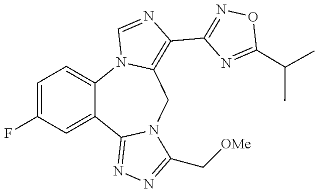 [SMILES: COCc1nnc2n1Cc1c(-c3noc(C(C)C)n3)ncn1-c1ccc(F)cc1-2] |
| 187 | 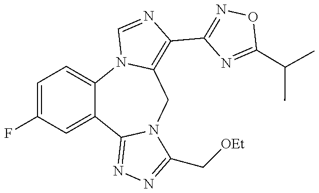 [SMILES: CCOCc1nnc2n1Cc1c(-c3noc(C(C)C)n3)ncn1-c1ccc(F)cc1-2] |
| 188 | 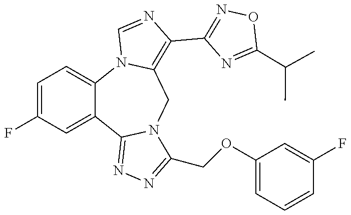 [SMILES: CC(C)c1nc(-c2ncn3c2Cn2c(COc4cccc(F)c4)nnc2-c2cc(F)ccc2-3)no1] |
| 189 | 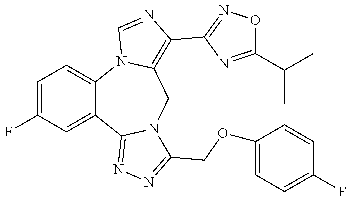 [SMILES: CC(C)c1nc(-c2ncn3c2Cn2c(COc4ccc(F)cc4)nnc2-c2cc(F)ccc2-3)no1] |
| 190 | 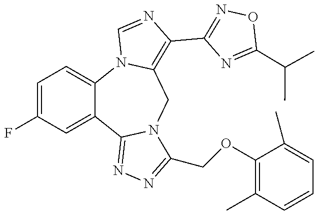 [SMILES: Cc1cccc(C)c1OCc1nnc2n1Cc1c(-c3noc(C(C)C)n3)ncn1-c1ccc(F)cc1-2] |
| 191 | 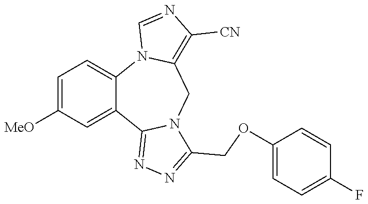 [SMILES: COc1ccc2c(c1)-c1nnc(COc3ccc(F)cc3)n1Cc1c(C#N)ncn1-2] |
| 192 | 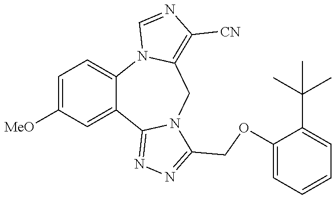 [SMILES: COc1ccc2c(c1)-c1nnc(COc3ccccc3C(C)(C)C)n1Cc1c(C#N)ncn1-2] |
| 193 | 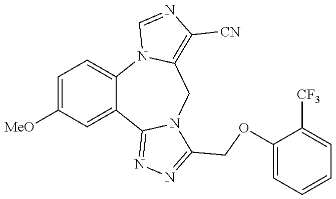 [SMILES: COc1ccc2c(c1)-c1nnc(COc3ccccc3C(F)(F)F)n1Cc1c(C#N)ncn1-2] |
| 194 | 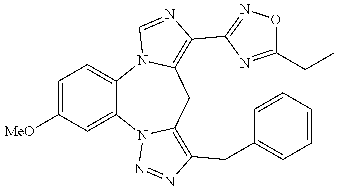 [SMILES: CCc1nc(-c2ncn3c2Cc2c(Cc4ccccc4)nnn2-c2cc(OC)ccc2-3)no1] |
| 195 | 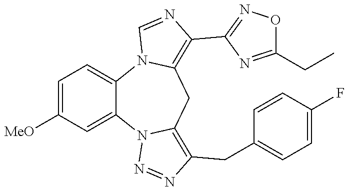 [SMILES: CCc1nc(-c2ncn3c2Cc2c(Cc4ccc(F)cc4)nnn2-c2cc(OC)ccc2-3)no1] |
| 196 | 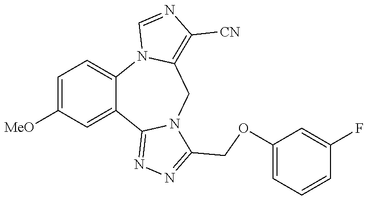 [SMILES: COc1ccc2c(c1)-c1nnc(COc3cccc(F)c3)n1Cc1c(C#N)ncn1-2] |
| 197 | 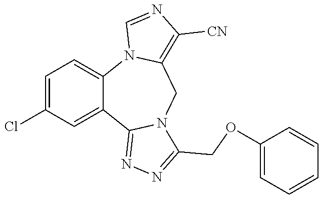 [SMILES: N#Cc1ncn2c1Cn1c(COc3ccccc3)nnc1-c1cc(Cl)ccc1-2] |
| 198 | 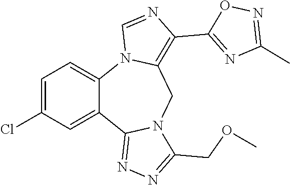 [SMILES: COCc1nnc2n1Cc1c(-c3nc(C)no3)ncn1-c1ccc(Cl)cc1-2] |
| 199 | 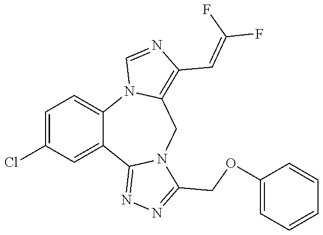 [SMILES: FC(F)=Cc1ncn2c1Cn1c(COc3ccccc3)nnc1-c1cc(Cl)ccc1-2] |
| 200 | 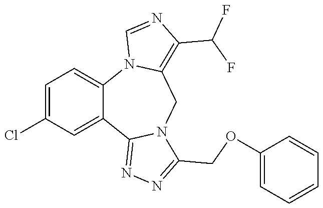 [SMILES: FC(F)c1ncn2c1Cn1c(COc3ccccc3)nnc1-c1cc(Cl)ccc1-2] |
| 201 | 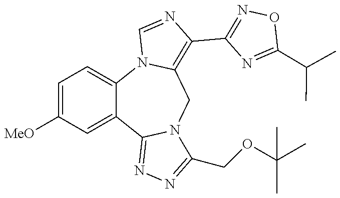 [SMILES: COc1ccc2c(c1)-c1nnc(COC(C)(C)C)n1Cc1c(-c3noc(C(C)C)n3)ncn1-2] |
| 202 | 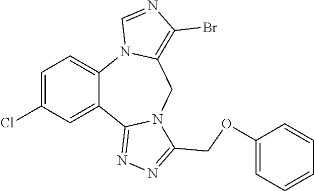 [SMILES: Clc1ccc2c(c1)-c1nnc(COc3ccccc3)n1Cc1c(Br)ncn1-2] |
| 203 | 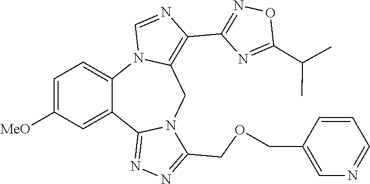 [SMILES: COc1ccc2c(c1)-c1nnc(COCc3cccnc3)n1Cc1c(-c3noc(C(C)C)n3)ncn1-2] |
| 204 | 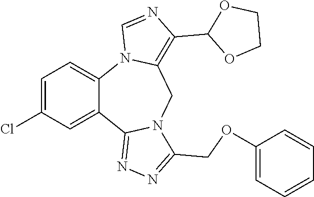 [SMILES: Clc1ccc2c(c1)-c1nnc(COc3ccccc3)n1Cc1c(C3OCCO3)ncn1-2] |
| 205 | 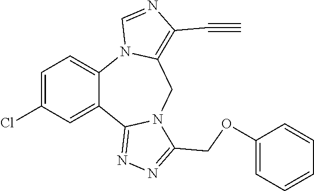 [SMILES: C#Cc1ncn2c1Cn1c(COc3ccccc3)nnc1-c1cc(Cl)ccc1-2] |
| 206 | 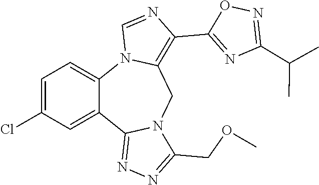 [SMILES: COCc1nnc2n1Cc1c(-c3nc(C(C)C)no3)ncn1-c1ccc(Cl)cc1-2] |
| 207 | 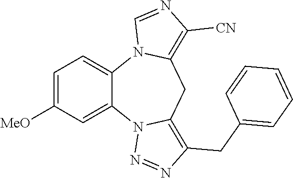 [SMILES: COc1ccc2c(c1)-n1nnc(Cc3ccccc3)c1Cc1c(C#N)ncn1-2] |
| 208 | 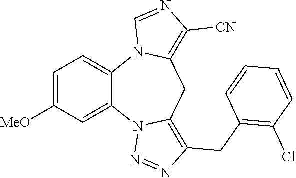 [SMILES: COc1ccc2c(c1)-n1nnc(Cc3ccccc3Cl)c1Cc1c(C#N)ncn1-2] |
| 209 | 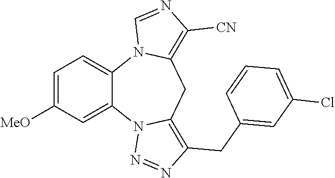 [SMILES: COc1ccc2c(c1)-n1nnc(Cc3cccc(Cl)c3)c1Cc1c(C#N)ncn1-2] |
| 210 | 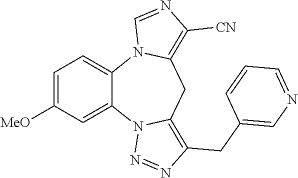 [SMILES: COc1ccc2c(c1)-n1nnc(Cc3cccnc3)c1Cc1c(C#N)ncn1-2] |
| 211 | 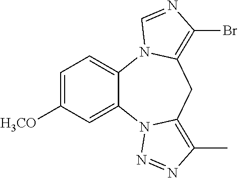 [SMILES: COc1ccc2c(c1)-n1nnc(C)c1Cc1c(Br)ncn1-2] |
| 212 | 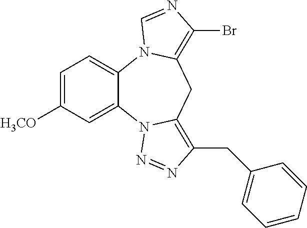 [SMILES: COc1ccc2c(c1)-n1nnc(Cc3ccccc3)c1Cc1c(Br)ncn1-2] |
| 213 | 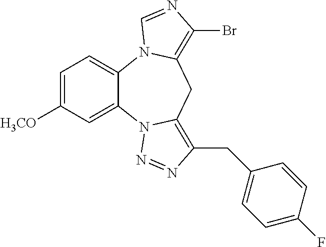 [SMILES: COc1ccc2c(c1)-n1nnc(Cc3ccc(F)cc3)c1Cc1c(Br)ncn1-2] |
| 214 | 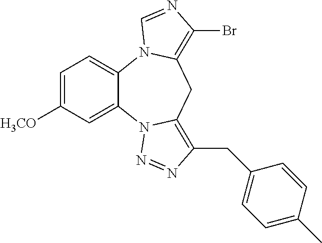 [SMILES: COc1ccc2c(c1)-n1nnc(Cc3ccc(C)cc3)c1Cc1c(Br)ncn1-2] |
| 215 | 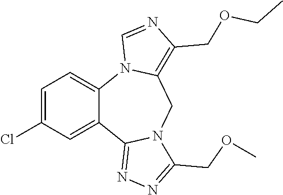 [SMILES: CCOCc1ncn2c1Cn1c(COC)nnc1-c1cc(Cl)ccc1-2] |
| 216 | 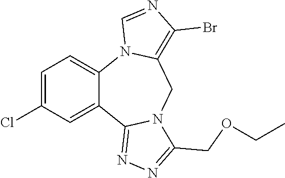 [SMILES: CCOCc1nnc2n1Cc1c(Br)ncn1-c1ccc(Cl)cc1-2] |
| 217 | 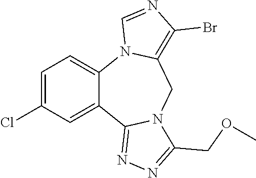 [SMILES: COCc1nnc2n1Cc1c(Br)ncn1-c1ccc(Cl)cc1-2] |
| 218 | 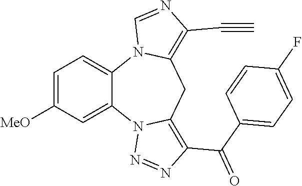 [SMILES: C#Cc1ncn2c1Cc1c(C(=O)c3ccc(F)cc3)nnn1-c1cc(OC)ccc1-2] |
| 219 | 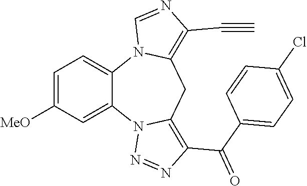 [SMILES: C#Cc1ncn2c1Cc1c(C(=O)c3ccc(Cl)cc3)nnn1-c1cc(OC)ccc1-2] |
| 220 | 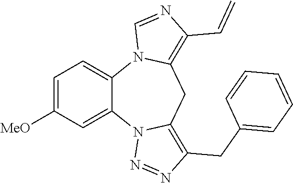 [SMILES: C=Cc1ncn2c1Cc1c(Cc3ccccc3)nnn1-c1cc(OC)ccc1-2] |
| 221 | 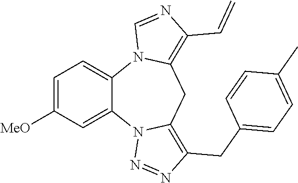 [SMILES: C=Cc1ncn2c1Cc1c(Cc3ccc(C)cc3)nnn1-c1cc(OC)ccc1-2] |
| 222 | 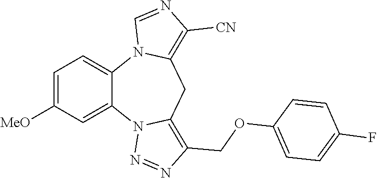 [SMILES: COc1ccc2c(c1)-n1nnc(COc3ccc(F)cc3)c1Cc1c(C#N)ncn1-2] |
| 223 | 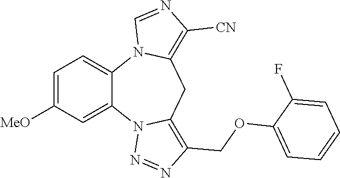 [SMILES: COc1ccc2c(c1)-n1nnc(COc3ccccc3F)c1Cc1c(C#N)ncn1-2] |
| 224 | 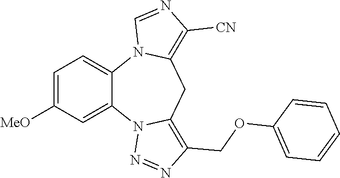 [SMILES: COc1ccc2c(c1)-n1nnc(COc3ccccc3)c1Cc1c(C#N)ncn1-2] |
| 225 | 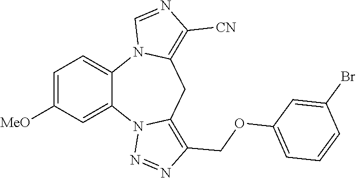 [SMILES: COc1ccc2c(c1)-n1nnc(COc3cccc(Br)c3)c1Cc1c(C#N)ncn1-2] |
| 226 | 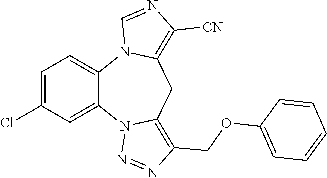 [SMILES: N#Cc1ncn2c1Cc1c(COc3ccccc3)nnn1-c1cc(Cl)ccc1-2] |
| 227 | 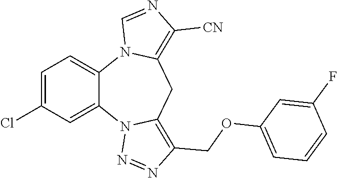 [SMILES: N#Cc1ncn2c1Cc1c(COc3cccc(F)c3)nnn1-c1cc(Cl)ccc1-2] |
| 228 | 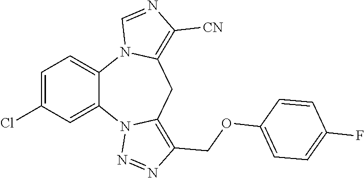 [SMILES: N#Cc1ncn2c1Cc1c(COc3ccc(F)cc3)nnn1-c1cc(Cl)ccc1-2] |
| 229 | 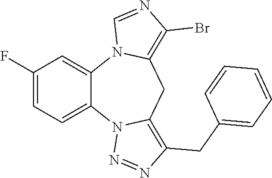 [SMILES: Fc1ccc2c(c1)-n1cnc(Br)c1Cc1c(Cc3ccccc3)nnn1-2] |
| 230 | 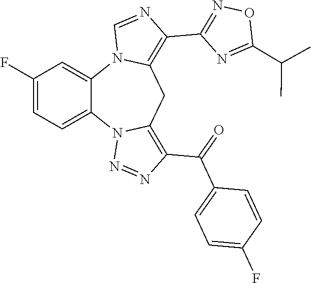 [SMILES: CC(C)c1nc(-c2ncn3c2Cc2c(C(=O)c4ccc(F)cc4)nnn2-c2ccc(F)cc2-3)no1] |
| 231 | 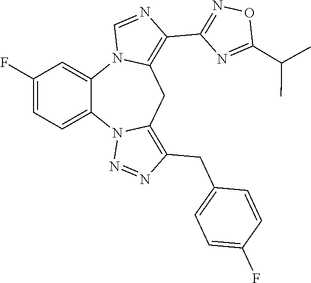 [SMILES: CC(C)c1nc(-c2ncn3c2Cc2c(Cc4ccc(F)cc4)nnn2-c2ccc(F)cc2-3)no1] |
| 232 | 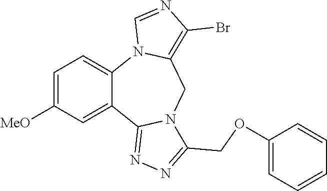 [SMILES: COc1ccc2c(c1)-c1nnc(COc3ccccc3)n1Cc1c(Br)ncn1-2] |
| 233 | 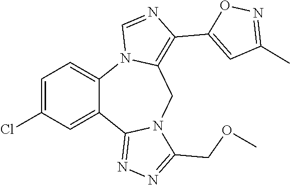 [SMILES: COCc1nnc2n1Cc1c(-c3cc(C)no3)ncn1-c1ccc(Cl)cc1-2] |
| 234 | 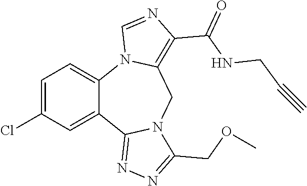 [SMILES: C#CCNC(=O)c1ncn2c1Cn1c(COC)nnc1-c1cc(Cl)ccc1-2] |
| 235 | 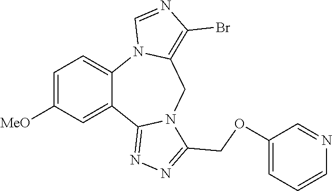 [SMILES: COc1ccc2c(c1)-c1nnc(COc3cccnc3)n1Cc1c(Br)ncn1-2] |
| 236 | 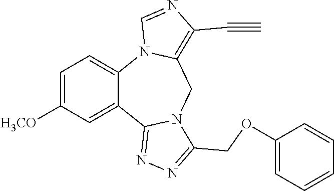 [SMILES: C#Cc1ncn2c1Cn1c(COc3ccccc3)nnc1-c1cc(OC)ccc1-2] |
| 237 | 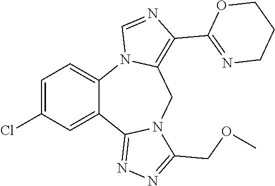 [SMILES: COCc1nnc2n1Cc1c(C3=NCCCO3)ncn1-c1ccc(Cl)cc1-2] |
| 238 | 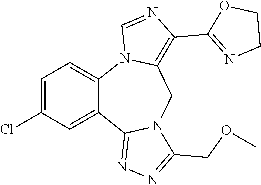 [SMILES: COCc1nnc2n1Cc1c(C3=NCCO3)ncn1-c1ccc(Cl)cc1-2] |
| 239 | 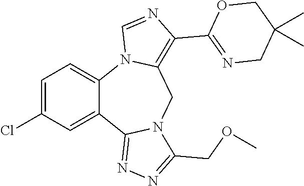 [SMILES: COCc1nnc2n1Cc1c(C3=NCC(C)(C)CO3)ncn1-c1ccc(Cl)cc1-2] |
| 240 | 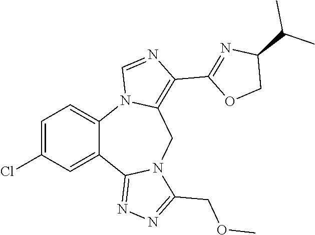 [SMILES: COCc1nnc2n1Cc1c(C3=N[C@@H](C(C)C)CO3)ncn1-c1ccc(Cl)cc1-2] |
| 241 | 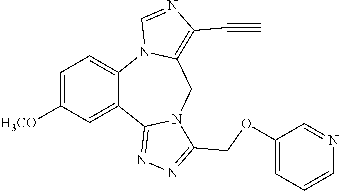 [SMILES: C#Cc1ncn2c1Cn1c(COc3cccnc3)nnc1-c1cc(OC)ccc1-2] |
| 242 | 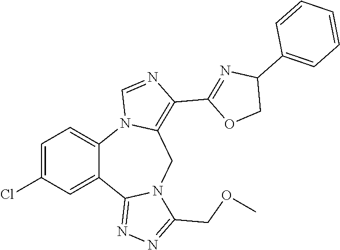 [SMILES: COCc1nnc2n1Cc1c(C3=NC(c4ccccc4)CO3)ncn1-c1ccc(Cl)cc1-2] |
| 243 | 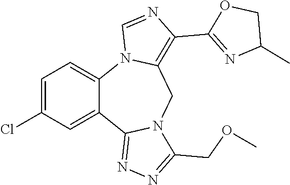 [SMILES: COCc1nnc2n1Cc1c(C3=NC(C)CO3)ncn1-c1ccc(Cl)cc1-2] |
| 244 | 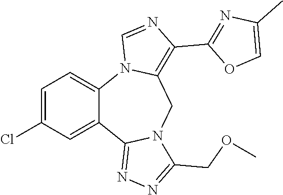 [SMILES: COCc1nnc2n1Cc1c(-c3nc(C)co3)ncn1-c1ccc(Cl)cc1-2] |
| 245 | 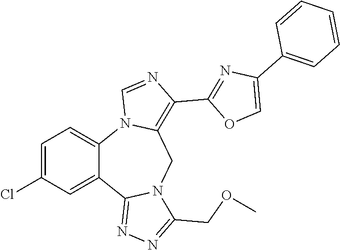 [SMILES: COCc1nnc2n1Cc1c(-c3nc(-c4ccccc4)co3)ncn1-c1ccc(Cl)cc1-2] |
| 246 | 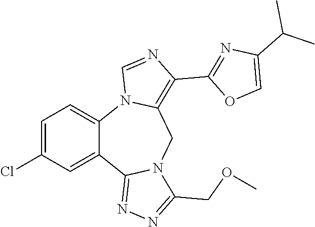 [SMILES: COCc1nnc2n1Cc1c(-c3nc(C(C)C)co3)ncn1-c1ccc(Cl)cc1-2] |
| 247 | 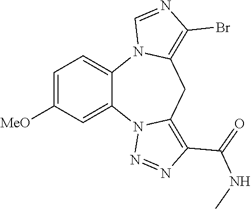 [SMILES: CNC(=O)c1nnn2c1Cc1c(Br)ncn1-c1ccc(OC)cc1-2] |
| 248 | 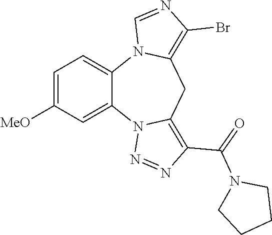 [SMILES: COc1ccc2c(c1)-n1nnc(C(=O)N3CCCC3)c1Cc1c(Br)ncn1-2] |
| 249 | 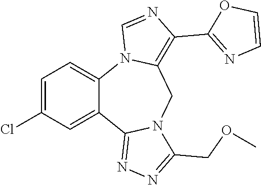 [SMILES: COCc1nnc2n1Cc1c(-c3ncco3)ncn1-c1ccc(Cl)cc1-2] |
| 250 | 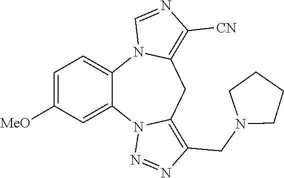 [SMILES: COc1ccc2c(c1)-n1nnc(CN3CCCC3)c1Cc1c(C#N)ncn1-2] |
| 251 | 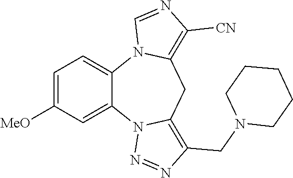 [SMILES: COc1ccc2c(c1)-n1nnc(CN3CCCCC3)c1Cc1c(C#N)ncn1-2] |
| 252 | 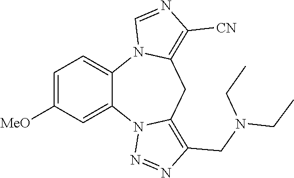 [SMILES: CCN(CC)Cc1nnn2c1Cc1c(C#N)ncn1-c1ccc(OC)cc1-2] |
| 253 | 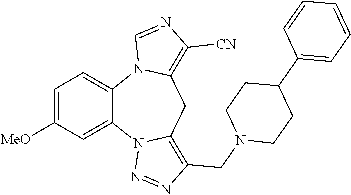 [SMILES: COc1ccc2c(c1)-n1nnc(CN3CCC(c4ccccc4)CC3)c1Cc1c(C#N)ncn1-2] |
| 254 | 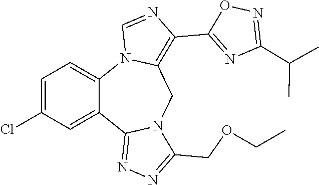 [SMILES: CCOCc1nnc2n1Cc1c(-c3nc(C(C)C)no3)ncn1-c1ccc(Cl)cc1-2] |
| 255 | 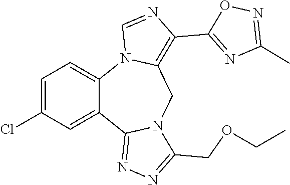 [SMILES: CCOCc1nnc2n1Cc1c(-c3nc(C)no3)ncn1-c1ccc(Cl)cc1-2] |
| 256 | 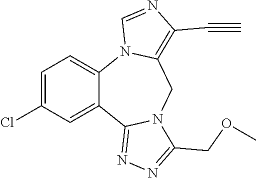 [SMILES: C#Cc1ncn2c1Cn1c(COC)nnc1-c1cc(Cl)ccc1-2] |
| 257 | 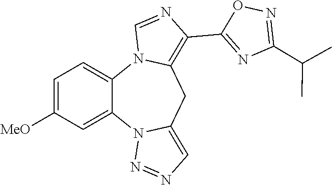 [SMILES: COc1ccc2c(c1)-n1nncc1Cc1c(-c3nc(C(C)C)no3)ncn1-2] |
| 258 | 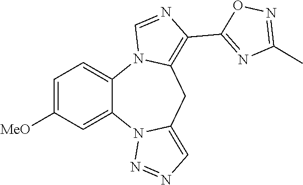 [SMILES: COc1ccc2c(c1)-n1nncc1Cc1c(-c3nc(C)no3)ncn1-2] |
| 259 | 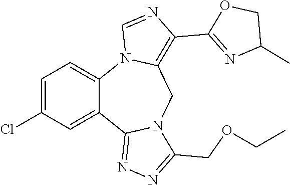 [SMILES: CCOCc1nnc2n1Cc1c(C3=NC(C)CO3)ncn1-c1ccc(Cl)cc1-2] |
| 260 | 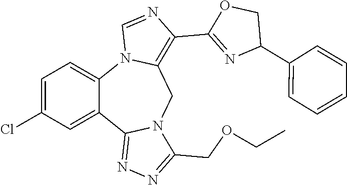 [SMILES: CCOCc1nnc2n1Cc1c(C3=NC(c4ccccc4)CO3)ncn1-c1ccc(Cl)cc1-2] |
| 261 | 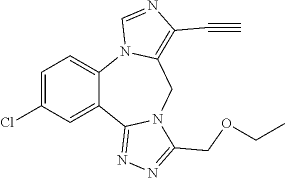 [SMILES: C#Cc1ncn2c1Cn1c(COCC)nnc1-c1cc(Cl)ccc1-2] |
| 262 | 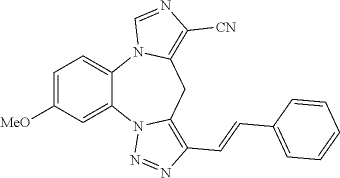 [SMILES: COc1ccc2c(c1)-n1nnc(/C=C/c3ccccc3)c1Cc1c(C#N)ncn1-2] |
| 263 | 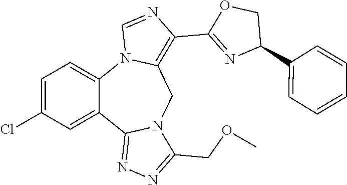 [SMILES: COCc1nnc2n1Cc1c(C3=N[C@H](c4ccccc4)CO3)ncn1-c1ccc(Cl)cc1-2] |
| 264 | 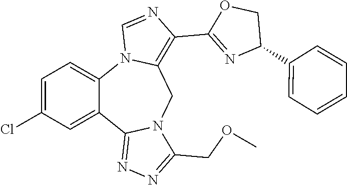 [SMILES: COCc1nnc2n1Cc1c(C3=N[C@@H](c4ccccc4)CO3)ncn1-c1ccc(Cl)cc1-2] |
| 265 | 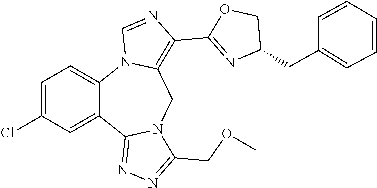 [SMILES: COCc1nnc2n1Cc1c(C3=N[C@@H](Cc4ccccc4)CO3)ncn1-c1ccc(Cl)cc1-2] |
| 266 | 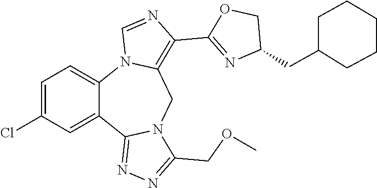 [SMILES: COCc1nnc2n1Cc1c(C3=N[C@@H](CC4CCCCC4)CO3)ncn1-c1ccc(Cl)cc1-2] |
| 267 | 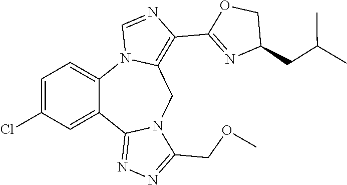 [SMILES: COCc1nnc2n1Cc1c(C3=N[C@H](CC(C)C)CO3)ncn1-c1ccc(Cl)cc1-2] |
| 268 | 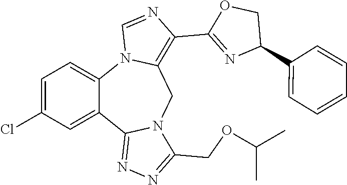 [SMILES: CC(C)OCc1nnc2n1Cc1c(C3=N[C@H](c4ccccc4)CO3)ncn1-c1ccc(Cl)cc1-2] |
| 270 | 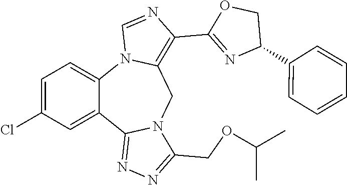 [SMILES: CC(C)OCc1nnc2n1Cc1c(C3=N[C@@H](c4ccccc4)CO3)ncn1-c1ccc(Cl)cc1-2] |
| 271 | 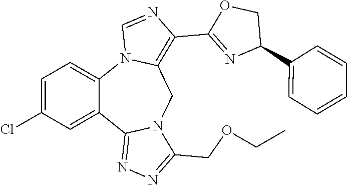 [SMILES: CCOCc1nnc2n1Cc1c(C3=N[C@H](c4ccccc4)CO3)ncn1-c1ccc(Cl)cc1-2] |
| 272 | 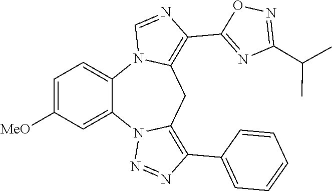 [SMILES: COc1ccc2c(c1)-n1nnc(-c3ccccc3)c1Cc1c(-c3nc(C(C)C)no3)ncn1-2] |
| 273 | 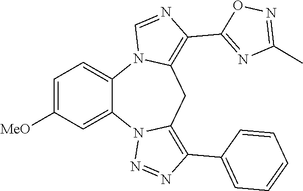 [SMILES: COc1ccc2c(c1)-n1nnc(-c3ccccc3)c1Cc1c(-c3nc(C)no3)ncn1-2] |
| 274 | 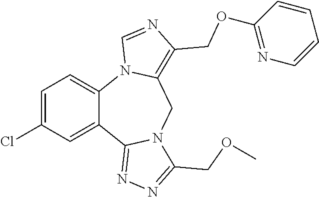 [SMILES: COCc1nnc2n1Cc1c(COc3ccccn3)ncn1-c1ccc(Cl)cc1-2] |
| 275 | 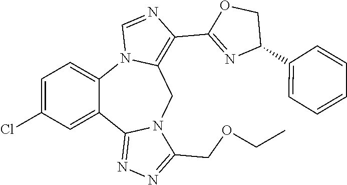 [SMILES: CCOCc1nnc2n1Cc1c(C3=N[C@@H](c4ccccc4)CO3)ncn1-c1ccc(Cl)cc1-2] |
| 276 | 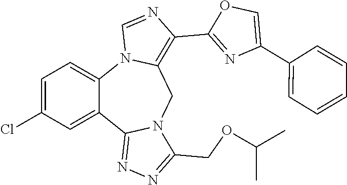 [SMILES: CC(C)OCc1nnc2n1Cc1c(-c3nc(-c4ccccc4)co3)ncn1-c1ccc(Cl)cc1-2] |
| 277 | 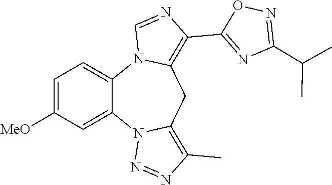 [SMILES: COc1ccc2c(c1)-n1nnc(C)c1Cc1c(-c3nc(C(C)C)no3)ncn1-2] |
| 278 | 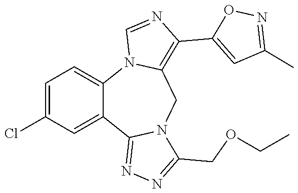 [SMILES: CCOCc1nnc2n1Cc1c(-c3cc(C)no3)ncn1-c1ccc(Cl)cc1-2] |
| 279 | 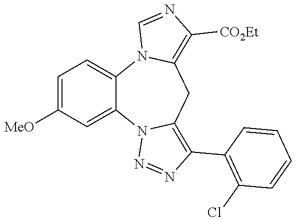 [SMILES: CCOC(=O)c1ncn2c1Cc1c(-c3ccccc3Cl)nnn1-c1cc(OC)ccc1-2] |
| 280 | 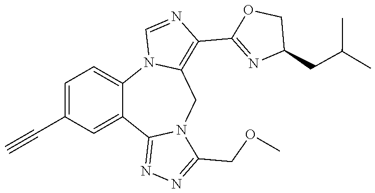 [SMILES: C#Cc1ccc2c(c1)-c1nnc(COC)n1Cc1c(C3=N[C@H](CC(C)C)CO3)ncn1-2] |
| 281 | 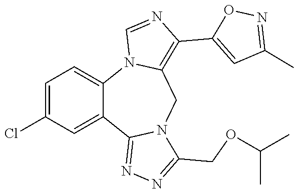 [SMILES: Cc1cc(-c2ncn3c2Cn2c(COC(C)C)nnc2-c2cc(Cl)ccc2-3)on1] |
| 282 | 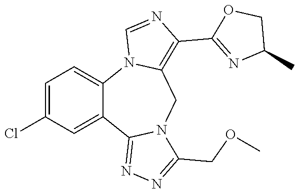 [SMILES: COCc1nnc2n1Cc1c(C3=N[C@H](C)CO3)ncn1-c1ccc(Cl)cc1-2] |
| 283 | 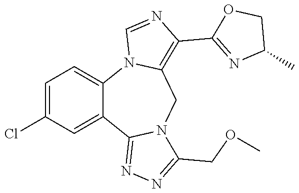 [SMILES: COCc1nnc2n1Cc1c(C3=N[C@@H](C)CO3)ncn1-c1ccc(Cl)cc1-2] |
| 284 | 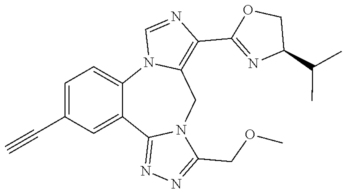 [SMILES: C#Cc1ccc2c(c1)-c1nnc(COC)n1Cc1c(C3=N[C@H](C(C)C)CO3)ncn1-2] |
| 285 | 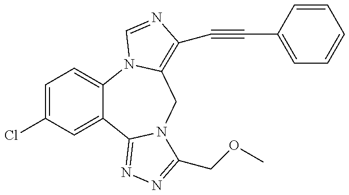 [SMILES: COCc1nnc2n1Cc1c(C#Cc3ccccc3)ncn1-c1ccc(Cl)cc1-2] |
| 286 | 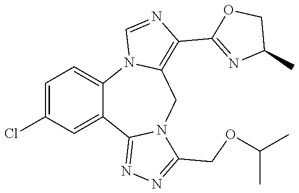 [SMILES: CC(C)OCc1nnc2n1Cc1c(C3=N[C@H](C)CO3)ncn1-c1ccc(Cl)cc1-2] |
| 287 | 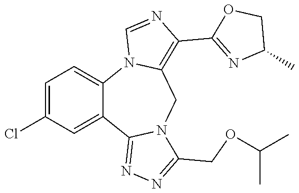 [SMILES: CC(C)OCc1nnc2n1Cc1c(C3=N[C@@H](C)CO3)ncn1-c1ccc(Cl)cc1-2] |
| 288 | 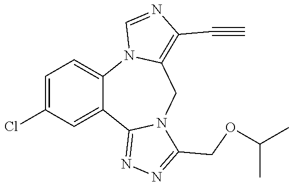 [SMILES: C#Cc1ncn2c1Cn1c(COC(C)C)nnc1-c1cc(Cl)ccc1-2] |
| 289 | 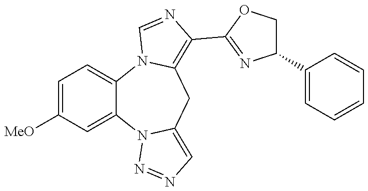 [SMILES: COc1ccc2c(c1)-n1nncc1Cc1c(C3=N[C@@H](c4ccccc4)CO3)ncn1-2] |
| 290 | 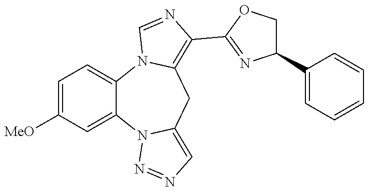 [SMILES: COc1ccc2c(c1)-n1nncc1Cc1c(C3=N[C@H](c4ccccc4)CO3)ncn1-2] |
| 291 | 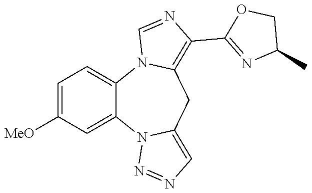 [SMILES: COc1ccc2c(c1)-n1nncc1Cc1c(C3=N[C@H](C)CO3)ncn1-2] |
| 292 | 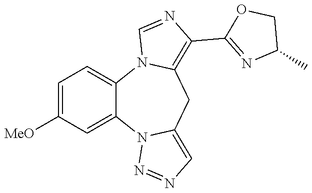 [SMILES: COc1ccc2c(c1)-n1nncc1Cc1c(C3=N[C@@H](C)CO3)ncn1-2] |
| 293 | 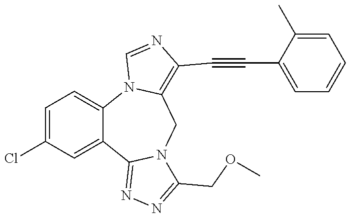 [SMILES: COCc1nnc2n1Cc1c(C#Cc3ccccc3C)ncn1-c1ccc(Cl)cc1-2] |
| 294 | 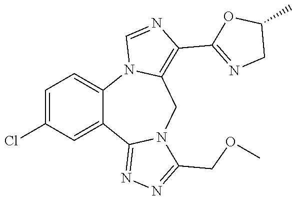 [SMILES: COCc1nnc2n1Cc1c(C3=NC[C@@H](C)O3)ncn1-c1ccc(Cl)cc1-2] |
| 295 | 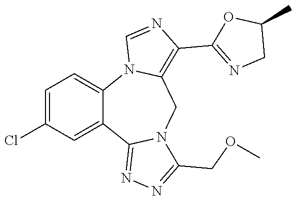 [SMILES: COCc1nnc2n1Cc1c(C3=NC[C@H](C)O3)ncn1-c1ccc(Cl)cc1-2] |
| 296 | 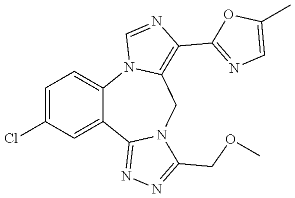 [SMILES: COCc1nnc2n1Cc1c(-c3ncc(C)o3)ncn1-c1ccc(Cl)cc1-2] |
| 297 | 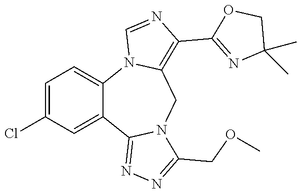 [SMILES: COCc1nnc2n1Cc1c(C3=NC(C)(C)CO3)ncn1-c1ccc(Cl)cc1-2] |
| 298 | 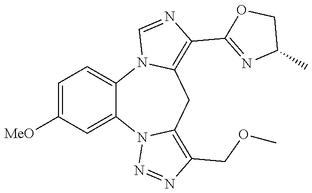 [SMILES: COCc1nnn2c1Cc1c(C3=N[C@@H](C)CO3)ncn1-c1ccc(OC)cc1-2] |
| 299 | 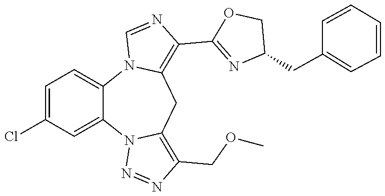 [SMILES: COCc1nnn2c1Cc1c(C3=N[C@@H](Cc4ccccc4)CO3)ncn1-c1ccc(Cl)cc1-2] |
| 300 | 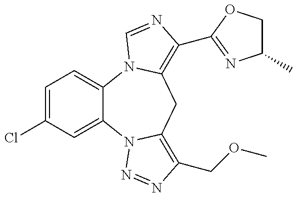 [SMILES: COCc1nnn2c1Cc1c(C3=N[C@@H](C)CO3)ncn1-c1ccc(Cl)cc1-2] |
| 301 | 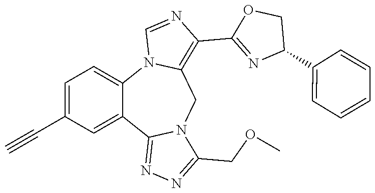 [SMILES: C#Cc1ccc2c(c1)-c1nnc(COC)n1Cc1c(C3=N[C@@H](c4ccccc4)CO3)ncn1-2] |
| 302 | 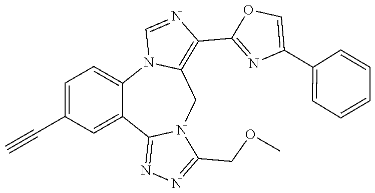 [SMILES: C#Cc1ccc2c(c1)-c1nnc(COC)n1Cc1c(-c3nc(-c4ccccc4)co3)ncn1-2] |
| 303 | 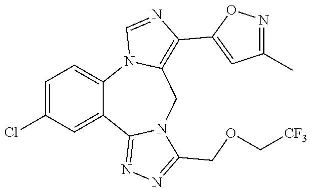 [SMILES: Cc1cc(-c2ncn3c2Cn2c(COCC(F)(F)F)nnc2-c2cc(Cl)ccc2-3)on1] |
| 304 | 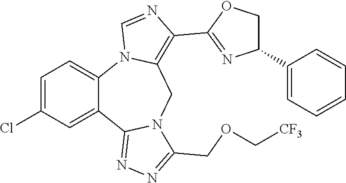 [SMILES: FC(F)(F)COCc1nnc2n1Cc1c(C3=N[C@@H](c4ccccc4)CO3)ncn1-c1ccc(Cl)cc1-2] |
| 305 | 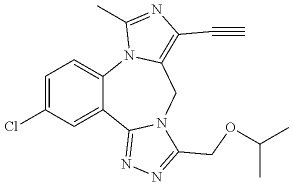 [SMILES: C#Cc1nc(C)n2c1Cn1c(COC(C)C)nnc1-c1cc(Cl)ccc1-2] |
| 306 | 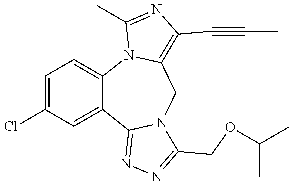 [SMILES: CC#Cc1nc(C)n2c1Cn1c(COC(C)C)nnc1-c1cc(Cl)ccc1-2] |
| 307 | 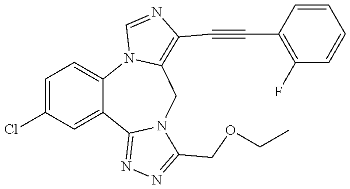 [SMILES: CCOCc1nnc2n1Cc1c(C#Cc3ccccc3F)ncn1-c1ccc(Cl)cc1-2] |
| 308 | 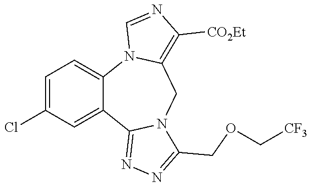 [SMILES: CCOC(=O)c1ncn2c1Cn1c(COCC(F)(F)F)nnc1-c1cc(Cl)ccc1-2] |
| 309 | 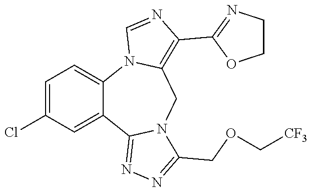 [SMILES: FC(F)(F)COCc1nnc2n1Cc1c(C3=NCCO3)ncn1-c1ccc(Cl)cc1-2] |
| 310 | 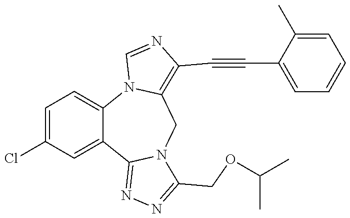 [SMILES: Cc1ccccc1C#Cc1ncn2c1Cn1c(COC(C)C)nnc1-c1cc(Cl)ccc1-2] |
| 311 | 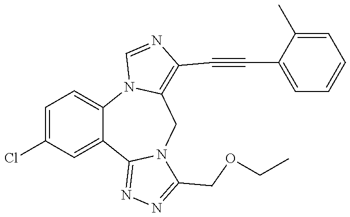 [SMILES: CCOCc1nnc2n1Cc1c(C#Cc3ccccc3C)ncn1-c1ccc(Cl)cc1-2] |
| 312 | 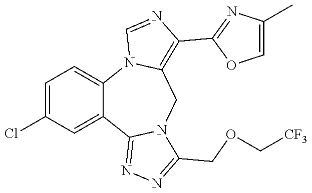 [SMILES: Cc1coc(-c2ncn3c2Cn2c(COCC(F)(F)F)nnc2-c2cc(Cl)ccc2-3)n1] |
| 313 | 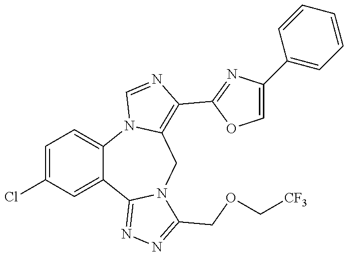 [SMILES: FC(F)(F)COCc1nnc2n1Cc1c(-c3nc(-c4ccccc4)co3)ncn1-c1ccc(Cl)cc1-2] |
| 314 | 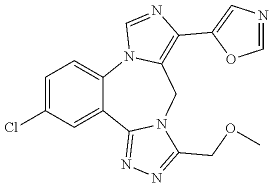 [SMILES: COCc1nnc2n1Cc1c(-c3cnco3)ncn1-c1ccc(Cl)cc1-2] |
| 315 | 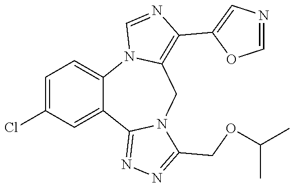 [SMILES: CC(C)OCc1nnc2n1Cc1c(-c3cnco3)ncn1-c1ccc(Cl)cc1-2] |
| 316 | 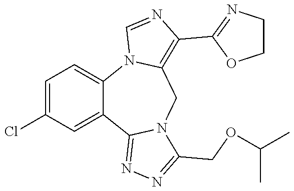 [SMILES: CC(C)OCc1nnc2n1Cc1c(C3=NCCO3)ncn1-c1ccc(Cl)cc1-2] |
| 317 | 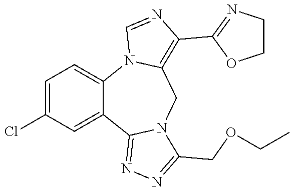 [SMILES: CCOCc1nnc2n1Cc1c(C3=NCCO3)ncn1-c1ccc(Cl)cc1-2] |
| 318 | 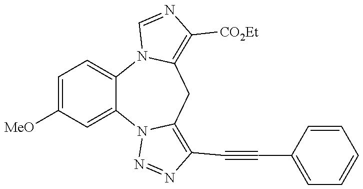 [SMILES: CCOC(=O)c1ncn2c1Cc1c(C#Cc3ccccc3)nnn1-c1cc(OC)ccc1-2] |
| 319 | 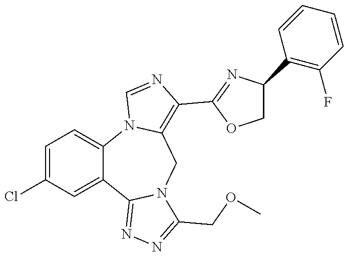 [SMILES: COCc1nnc2n1Cc1c(C3=N[C@@H](c4ccccc4F)CO3)ncn1-c1ccc(Cl)cc1-2] |
| 320 | 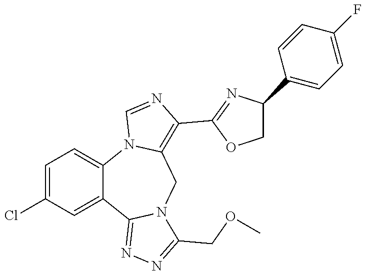 [SMILES: COCc1nnc2n1Cc1c(C3=N[C@@H](c4ccc(F)cc4)CO3)ncn1-c1ccc(Cl)cc1-2] |
| 321 | 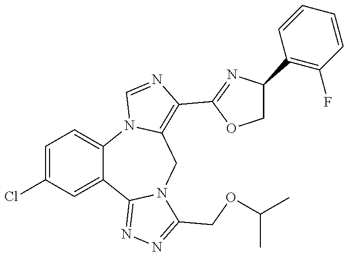 [SMILES: CC(C)OCc1nnc2n1Cc1c(C3=N[C@@H](c4ccccc4F)CO3)ncn1-c1ccc(Cl)cc1-2] |
| 322 | 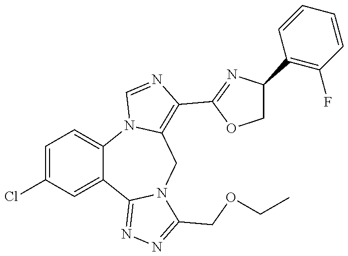 [SMILES: CCOCc1nnc2n1Cc1c(C3=N[C@@H](c4ccccc4F)CO3)ncn1-c1ccc(Cl)cc1-2] |
| 323 | 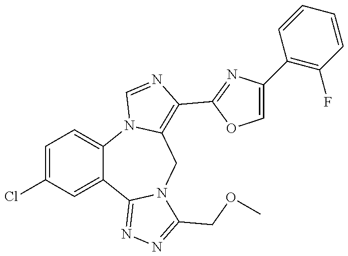 [SMILES: COCc1nnc2n1Cc1c(-c3nc(-c4ccccc4F)co3)ncn1-c1ccc(Cl)cc1-2] |
| 324 | 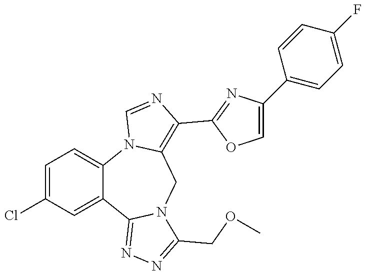 [SMILES: COCc1nnc2n1Cc1c(-c3nc(-c4ccc(F)cc4)co3)ncn1-c1ccc(Cl)cc1-2] |
| 325 | 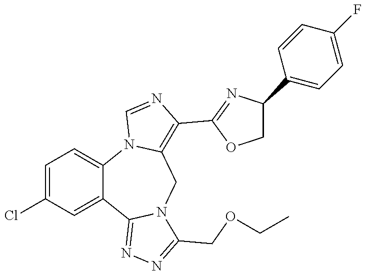 [SMILES: CCOCc1nnc2n1Cc1c(C3=N[C@@H](c4ccc(F)cc4)CO3)ncn1-c1ccc(Cl)cc1-2] |
| 326 | 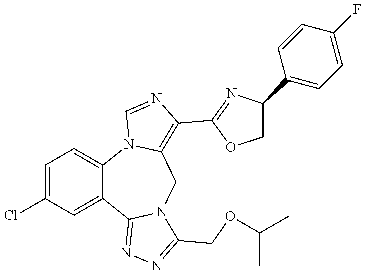 [SMILES: CC(C)OCc1nnc2n1Cc1c(C3=N[C@@H](c4ccc(F)cc4)CO3)ncn1-c1ccc(Cl)cc1-2] |
| 327 | 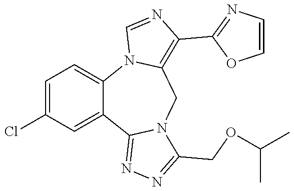 [SMILES: CC(C)OCc1nnc2n1Cc1c(-c3ncco3)ncn1-c1ccc(Cl)cc1-2] |
| 328 | 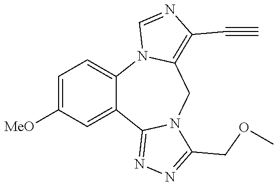 [SMILES: C#Cc1ncn2c1Cn1c(COC)nnc1-c1cc(OC)ccc1-2] |
| 329 | 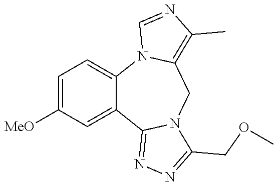 [SMILES: COCc1nnc2n1Cc1c(C)ncn1-c1ccc(OC)cc1-2] |
| 330 | 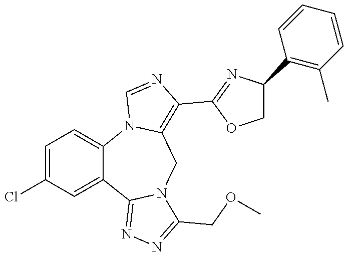 [SMILES: COCc1nnc2n1Cc1c(C3=N[C@@H](c4ccccc4C)CO3)ncn1-c1ccc(Cl)cc1-2] |
| 331 | 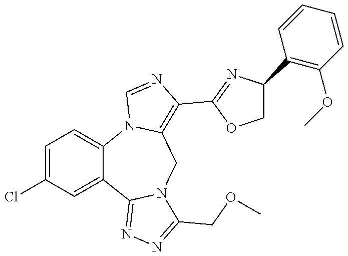 [SMILES: COCc1nnc2n1Cc1c(C3=N[C@@H](c4ccccc4OC)CO3)ncn1-c1ccc(Cl)cc1-2] |
| 332 | 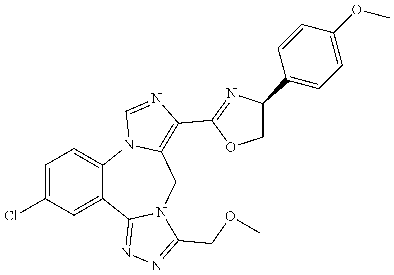 [SMILES: COCc1nnc2n1Cc1c(C3=N[C@@H](c4ccc(OC)cc4)CO3)ncn1-c1ccc(Cl)cc1-2] |
| 333 | 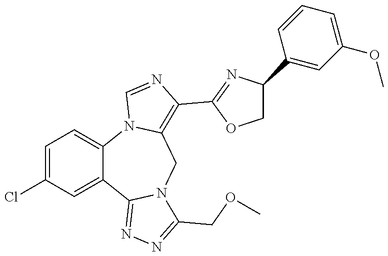 [SMILES: COCc1nnc2n1Cc1c(C3=N[C@@H](c4cccc(OC)c4)CO3)ncn1-c1ccc(Cl)cc1-2] |
| 334 | 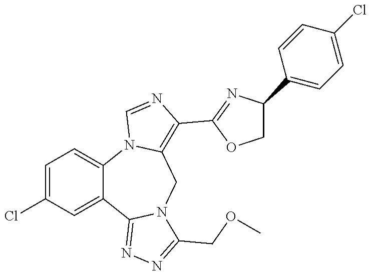 [SMILES: COCc1nnc2n1Cc1c(C3=N[C@@H](c4ccc(Cl)cc4)CO3)ncn1-c1ccc(Cl)cc1-2] |
| 335 | 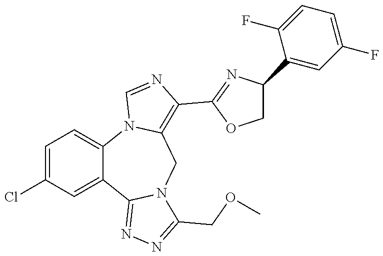 [SMILES: COCc1nnc2n1Cc1c(C3=N[C@@H](c4cc(F)ccc4F)CO3)ncn1-c1ccc(Cl)cc1-2] |
| 336 | 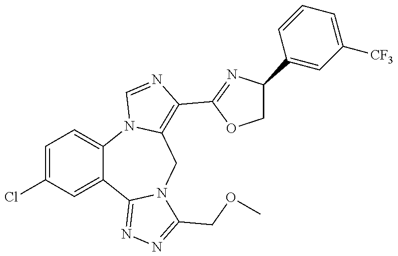 [SMILES: COCc1nnc2n1Cc1c(C3=N[C@@H](c4cccc(C(F)(F)F)c4)CO3)ncn1-c1ccc(Cl)cc1-2] |
| 337 | 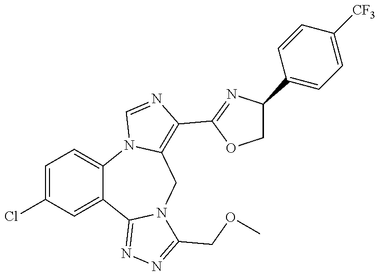 [SMILES: COCc1nnc2n1Cc1c(C3=N[C@@H](c4ccc(C(F)(F)F)cc4)CO3)ncn1-c1ccc(Cl)cc1-2] |
| 338 | 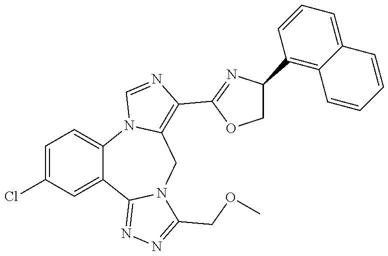 [SMILES: COCc1nnc2n1Cc1c(C3=N[C@@H](c4cccc5ccccc45)CO3)ncn1-c1ccc(Cl)cc1-2] |
| 339 | 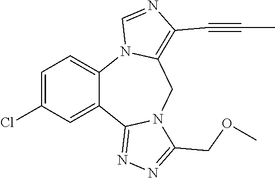 [SMILES: CC#Cc1ncn2c1Cn1c(COC)nnc1-c1cc(Cl)ccc1-2] |
| 340 | 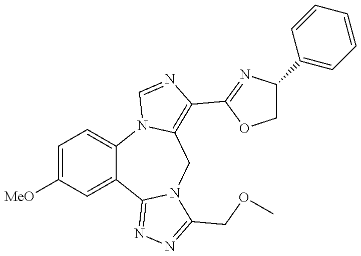 [SMILES: COCc1nnc2n1Cc1c(C3=N[C@H](c4ccccc4)CO3)ncn1-c1ccc(OC)cc1-2] |
| 341 | 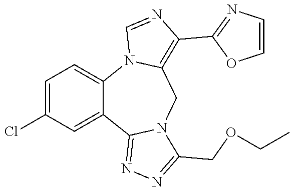 [SMILES: CCOCc1nnc2n1Cc1c(-c3ncco3)ncn1-c1ccc(Cl)cc1-2] |
| 342 | 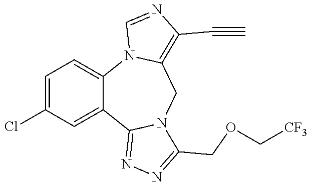 [SMILES: C#Cc1ncn2c1Cn1c(COCC(F)(F)F)nnc1-c1cc(Cl)ccc1-2] |
| 343 | 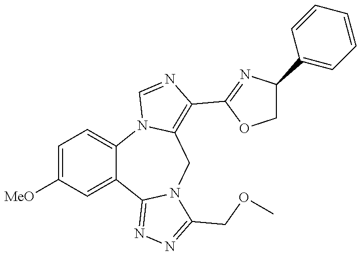 [SMILES: COCc1nnc2n1Cc1c(C3=N[C@@H](c4ccccc4)CO3)ncn1-c1ccc(OC)cc1-2] |
| 344 | 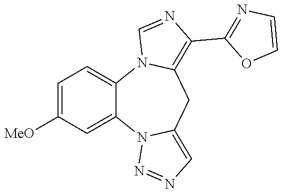 [SMILES: COc1ccc2c(c1)-n1nncc1Cc1c(-c3ncco3)ncn1-2] |
| 345 | 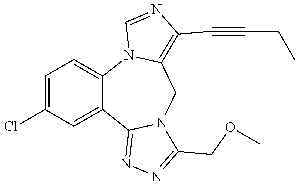 [SMILES: CCC#Cc1ncn2c1Cn1c(COC)nnc1-c1cc(Cl)ccc1-2] |
| 346 | 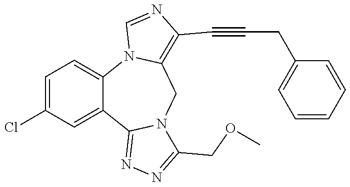 [SMILES: COCc1nnc2n1Cc1c(C#CCc3ccccc3)ncn1-c1ccc(Cl)cc1-2] |
| 347 | 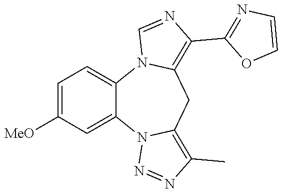 [SMILES: COc1ccc2c(c1)-n1nnc(C)c1Cc1c(-c3ncco3)ncn1-2] |
| 348 | 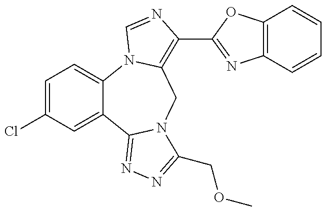 [SMILES: COCc1nnc2n1Cc1c(-c3nc4ccccc4o3)ncn1-c1ccc(Cl)cc1-2] |
| 349 | 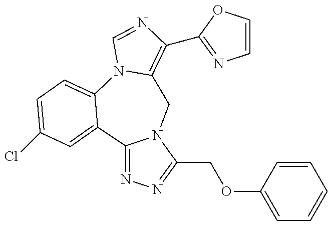 [SMILES: Clc1ccc2c(c1)-c1nnc(COc3ccccc3)n1Cc1c(-c3ncco3)ncn1-2] |
| 350 | 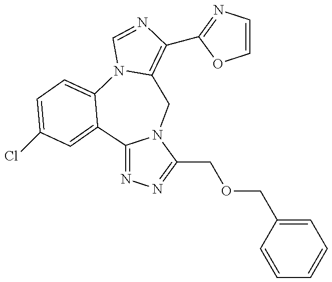 [SMILES: Clc1ccc2c(c1)-c1nnc(COCc3ccccc3)n1Cc1c(-c3ncco3)ncn1-2] |
| 351 | 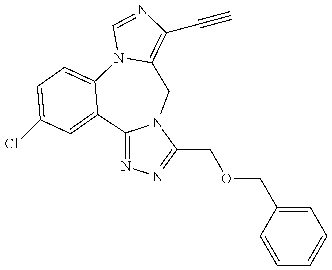 [SMILES: C#Cc1ncn2c1Cn1c(COCc3ccccc3)nnc1-c1cc(Cl)ccc1-2] |
| 352 | 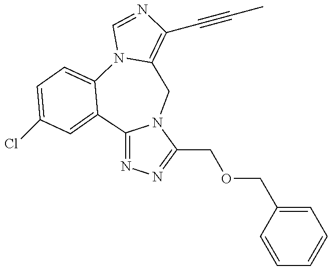 [SMILES: CC#Cc1ncn2c1Cn1c(COCc3ccccc3)nnc1-c1cc(Cl)ccc1-2] |
| 353 | 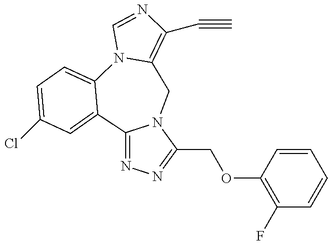 [SMILES: C#Cc1ncn2c1Cn1c(COc3ccccc3F)nnc1-c1cc(Cl)ccc1-2] |
| 354 | 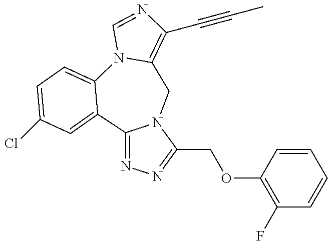 [SMILES: CC#Cc1ncn2c1Cn1c(COc3ccccc3F)nnc1-c1cc(Cl)ccc1-2] |
| 355 | 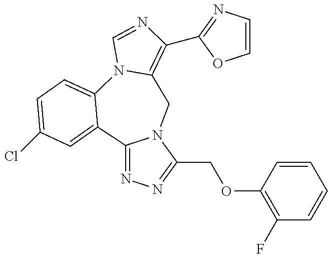 [SMILES: Fc1ccccc1OCc1nnc2n1Cc1c(-c3ncco3)ncn1-c1ccc(Cl)cc1-2] |
| 356 | 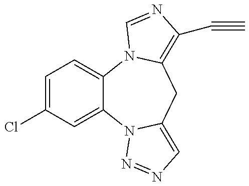 [SMILES: C#Cc1ncn2c1Cc1cnnn1-c1cc(Cl)ccc1-2] |
| 357 | 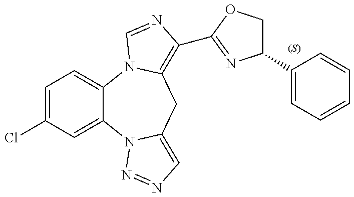 |
| 358 | 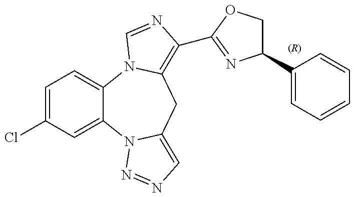 |
| 359 | 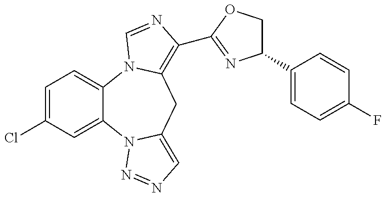 [SMILES: Fc1ccc([C@H]2COC(c3ncn4c3Cc3cnnn3-c3cc(Cl)ccc3-4)=N2)cc1] |
| 360 | 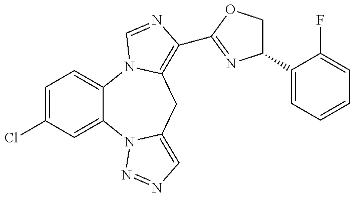 [SMILES: Fc1ccccc1[C@H]1COC(c2ncn3c2Cc2cnnn2-c2cc(Cl)ccc2-3)=N1] |
| 361 | 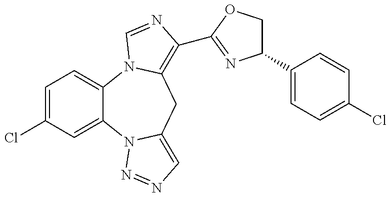 [SMILES: Clc1ccc([C@H]2COC(c3ncn4c3Cc3cnnn3-c3cc(Cl)ccc3-4)=N2)cc1] |
| 362 | 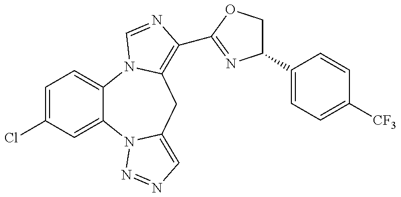 [SMILES: FC(F)(F)c1ccc([C@H]2COC(c3ncn4c3Cc3cnnn3-c3cc(Cl)ccc3-4)=N2)cc1] |
| 363 | 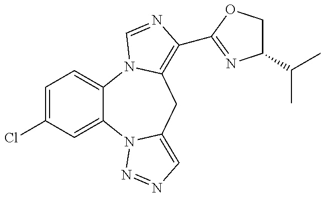 [SMILES: CC(C)[C@H]1COC(c2ncn3c2Cc2cnnn2-c2cc(Cl)ccc2-3)=N1] |
| 364 | 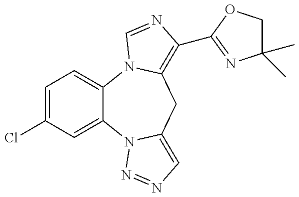 [SMILES: CC1(C)COC(c2ncn3c2Cc2cnnn2-c2cc(Cl)ccc2-3)=N1] |
| 365 | 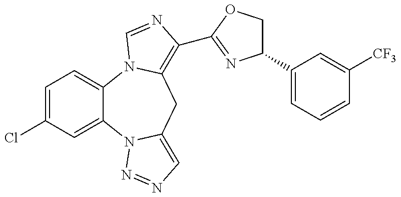 [SMILES: FC(F)(F)c1cccc([C@H]2COC(c3ncn4c3Cc3cnnn3-c3cc(Cl)ccc3-4)=N2)c1] |
| 366 | 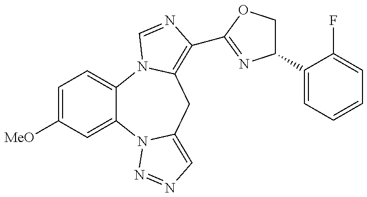 [SMILES: COc1ccc2c(c1)-n1nncc1Cc1c(C3=N[C@@H](c4ccccc4F)CO3)ncn1-2] |
| 367 | 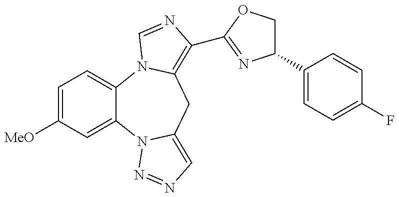 [SMILES: COc1ccc2c(c1)-n1nncc1Cc1c(C3=N[C@@H](c4ccc(F)cc4)CO3)ncn1-2] |
| 368 | 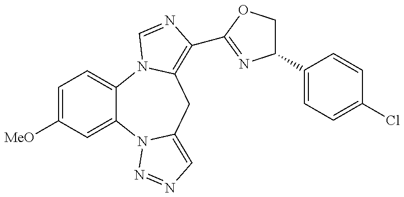 [SMILES: COc1ccc2c(c1)-n1nncc1Cc1c(C3=N[C@@H](c4ccc(Cl)cc4)CO3)ncn1-2] |
| 369 | 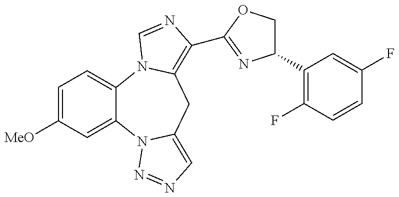 [SMILES: COc1ccc2c(c1)-n1nncc1Cc1c(C3=N[C@@H](c4cc(F)ccc4F)CO3)ncn1-2] |
| 370 | 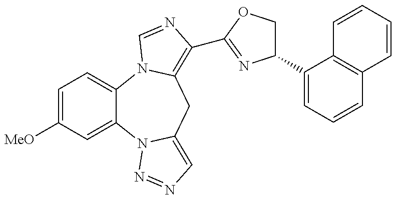 [SMILES: COc1ccc2c(c1)-n1nncc1Cc1c(C3=N[C@@H](c4cccc5ccccc45)CO3)ncn1-2] |
| 371 | 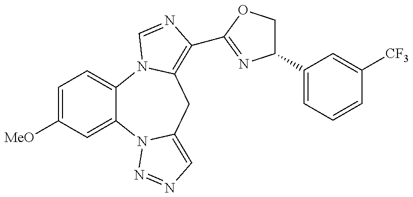 [SMILES: COc1ccc2c(c1)-n1nncc1Cc1c(C3=N[C@@H](c4cccc(C(F)(F)F)c4)CO3)ncn1-2] |
| 372 | 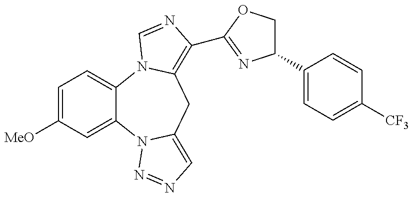 [SMILES: COc1ccc2c(c1)-n1nncc1Cc1c(C3=N[C@@H](c4ccc(C(F)(F)F)cc4)CO3)ncn1-2] |
| 373 | 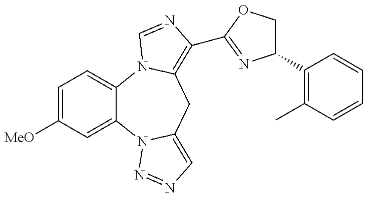 [SMILES: COc1ccc2c(c1)-n1nncc1Cc1c(C3=N[C@@H](c4ccccc4C)CO3)ncn1-2] |
| 374 | 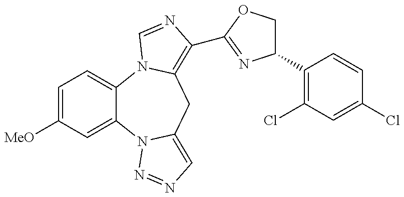 [SMILES: COc1ccc2c(c1)-n1nncc1Cc1c(C3=N[C@@H](c4ccc(Cl)cc4Cl)CO3)ncn1-2] |
| 375 | 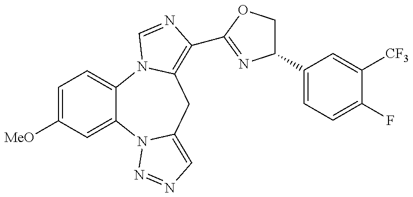 [SMILES: COc1ccc2c(c1)-n1nncc1Cc1c(C3=N[C@@H](c4ccc(F)c(C(F)(F)F)c4)CO3)ncn1-2] |
| 376 | 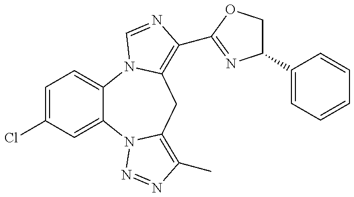 [SMILES: Cc1nnn2c1Cc1c(C3=N[C@@H](c4ccccc4)CO3)ncn1-c1ccc(Cl)cc1-2] |
| 377 | 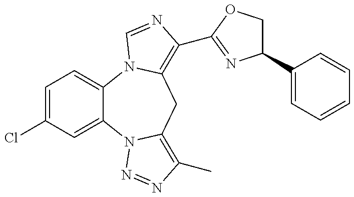 [SMILES: Cc1nnn2c1Cc1c(C3=N[C@H](c4ccccc4)CO3)ncn1-c1ccc(Cl)cc1-2] |
| 378 | 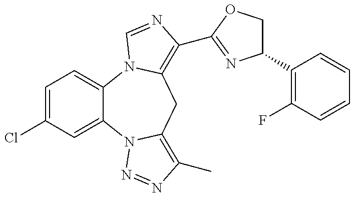 [SMILES: Cc1nnn2c1Cc1c(C3=N[C@@H](c4ccccc4F)CO3)ncn1-c1ccc(Cl)cc1-2] |
| 379 | 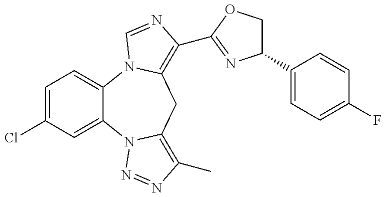 [SMILES: Cc1nnn2c1Cc1c(C3=N[C@@H](c4ccc(F)cc4)CO3)ncn1-c1ccc(Cl)cc1-2] |
| 380 | 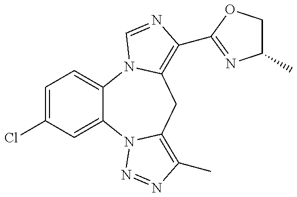 [SMILES: Cc1nnn2c1Cc1c(C3=N[C@@H](C)CO3)ncn1-c1ccc(Cl)cc1-2] |
| 381 | 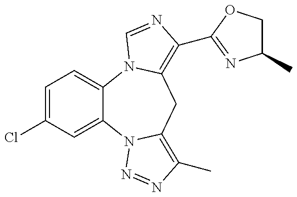 [SMILES: Cc1nnn2c1Cc1c(C3=N[C@H](C)CO3)ncn1-c1ccc(Cl)cc1-2] |
| 382 | 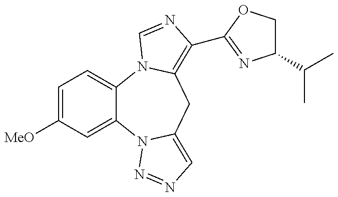 [SMILES: COc1ccc2c(c1)-n1nncc1Cc1c(C3=N[C@@H](C(C)C)CO3)ncn1-2] |
| 383 | 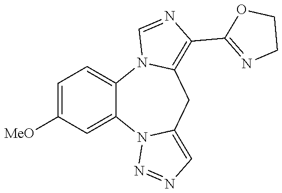 [SMILES: COc1ccc2c(c1)-n1nncc1Cc1c(C3=NCCO3)ncn1-2] |
| 384 | 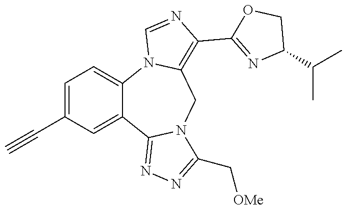 [SMILES: C#Cc1ccc2c(c1)-c1nnc(COC)n1Cc1c(C3=N[C@@H](C(C)C)CO3)ncn1-2] |
| 385 | 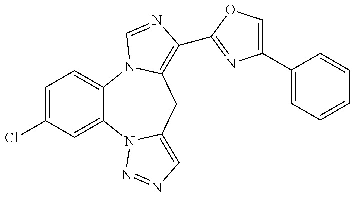 [SMILES: Clc1ccc2c(c1)-n1nncc1Cc1c(-c3nc(-c4ccccc4)co3)ncn1-2] |
| 386 | 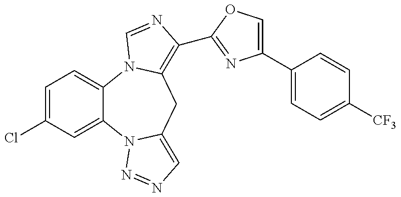 [SMILES: FC(F)(F)c1ccc(-c2coc(-c3ncn4c3Cc3cnnn3-c3cc(Cl)ccc3-4)n2)cc1] |
| 387 | 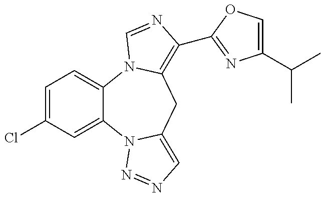 [SMILES: CC(C)c1coc(-c2ncn3c2Cc2cnnn2-c2cc(Cl)ccc2-3)n1] |
| 388 | 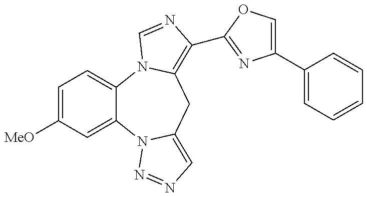 [SMILES: COc1ccc2c(c1)-n1nncc1Cc1c(-c3nc(-c4ccccc4)co3)ncn1-2] |
| 389 | 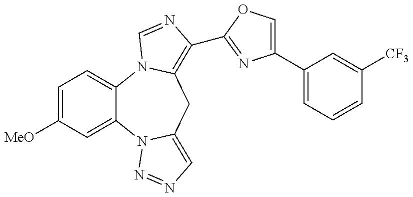 [SMILES: COc1ccc2c(c1)-n1nncc1Cc1c(-c3nc(-c4cccc(C(F)(F)F)c4)co3)ncn1-2] |
| 390 | 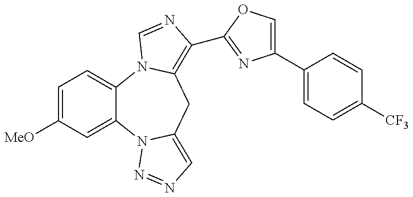 [SMILES: COc1ccc2c(c1)-n1nncc1Cc1c(-c3nc(-c4ccc(C(F)(F)F)cc4)co3)ncn1-2] |
| 391 | 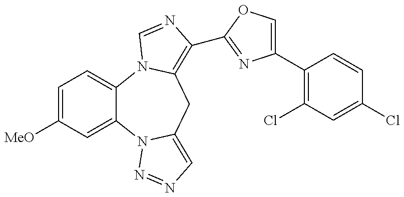 [SMILES: COc1ccc2c(c1)-n1nncc1Cc1c(-c3nc(-c4ccc(Cl)cc4Cl)co3)ncn1-2] |
| 392 | 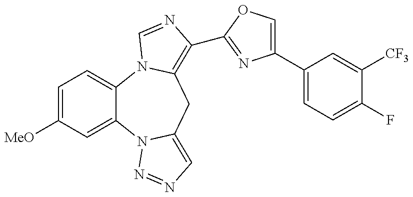 [SMILES: COc1ccc2c(c1)-n1nncc1Cc1c(-c3nc(-c4ccc(F)c(C(F)(F)F)c4)co3)ncn1-2] |
| 393 | 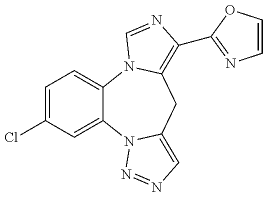 [SMILES: Clc1ccc2c(c1)-n1nncc1Cc1c(-c3ncco3)ncn1-2] |
| 394 | 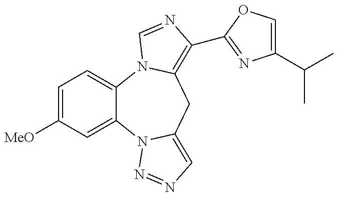 [SMILES: COc1ccc2c(c1)-n1nncc1Cc1c(-c3nc(C(C)C)co3)ncn1-2] |
| 395 | 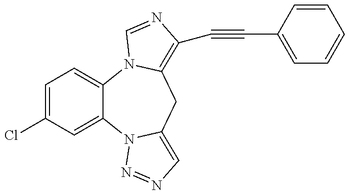 [SMILES: Clc1ccc2c(c1)-n1nncc1Cc1c(C#Cc3ccccc3)ncn1-2] |
| 396 | 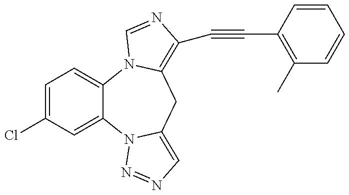 [SMILES: Cc1ccccc1C#Cc1ncn2c1Cc1cnnn1-c1cc(Cl)ccc1-2] |
| 397 | 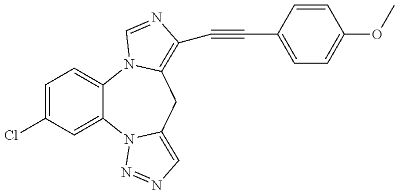 [SMILES: COc1ccc(C#Cc2ncn3c2Cc2cnnn2-c2cc(Cl)ccc2-3)cc1] |
| 398 | 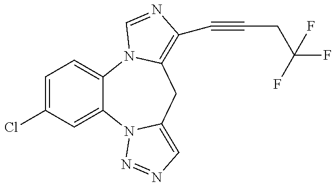 [SMILES: FC(F)(F)CC#Cc1ncn2c1Cc1cnnn1-c1cc(Cl)ccc1-2] |
| 399 | 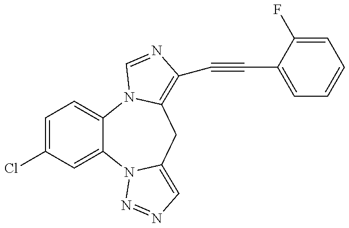 [SMILES: Fc1ccccc1C#Cc1ncn2c1Cc1cnnn1-c1cc(Cl)ccc1-2] |
| 400 | 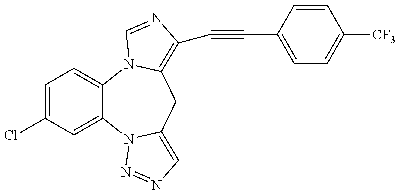 [SMILES: FC(F)(F)c1ccc(C#Cc2ncn3c2Cc2cnnn2-c2cc(Cl)ccc2-3)cc1] |
| 401 | 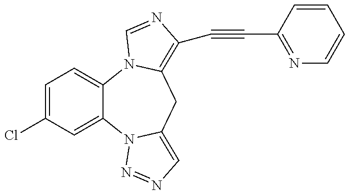 [SMILES: Clc1ccc2c(c1)-n1nncc1Cc1c(C#Cc3ccccn3)ncn1-2] |
| 402 | 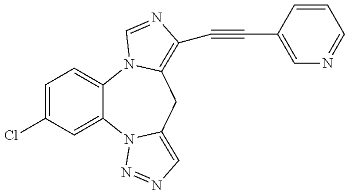 [SMILES: Clc1ccc2c(c1)-n1nncc1Cc1c(C#Cc3cccnc3)ncn1-2] |
| 403 | 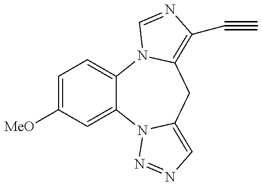 [SMILES: C#Cc1ncn2c1Cc1cnnn1-c1cc(OC)ccc1-2] |
| 404 | 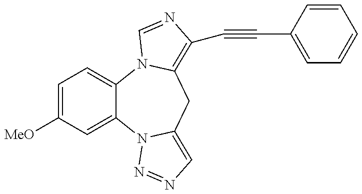 [SMILES: COc1ccc2c(c1)-n1nncc1Cc1c(C#Cc3ccccc3)ncn1-2] |
| 405 | 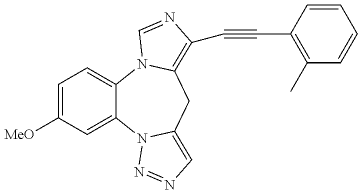 [SMILES: COc1ccc2c(c1)-n1nncc1Cc1c(C#Cc3ccccc3C)ncn1-2] |
| 406 | 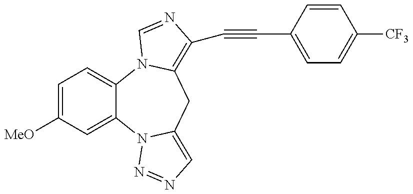 [SMILES: COc1ccc2c(c1)-n1nncc1Cc1c(C#Cc3ccc(C(F)(F)F)cc3)ncn1-2] |
| 407 | 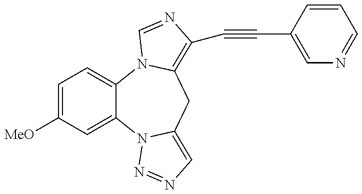 [SMILES: COc1ccc2c(c1)-n1nncc1Cc1c(C#Cc3cccnc3)ncn1-2] |
| 408 | 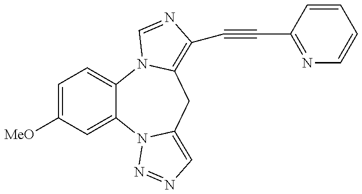 [SMILES: COc1ccc2c(c1)-n1nncc1Cc1c(C#Cc3ccccn3)ncn1-2] |
| 409 | 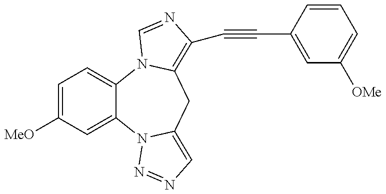 [SMILES: COc1cccc(C#Cc2ncn3c2Cc2cnnn2-c2cc(OC)ccc2-3)c1] |
| 410 | 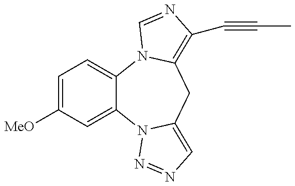 [SMILES: CC#Cc1ncn2c1Cc1cnnn1-c1cc(OC)ccc1-2] |
| 411 | 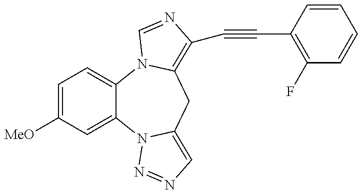 [SMILES: COc1ccc2c(c1)-n1nncc1Cc1c(C#Cc3ccccc3F)ncn1-2] |
| 412 | 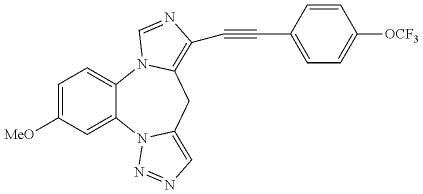 [SMILES: COc1ccc2c(c1)-n1nncc1Cc1c(C#Cc3ccc(OC(F)(F)F)cc3)ncn1-2] |
| 413 | 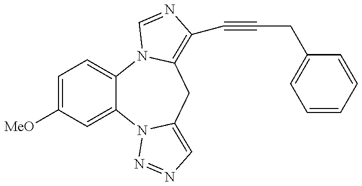 [SMILES: COc1ccc2c(c1)-n1nncc1Cc1c(C#CCc3ccccc3)ncn1-2] |
| 414 | 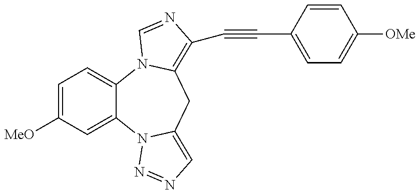 [SMILES: COc1ccc(C#Cc2ncn3c2Cc2cnnn2-c2cc(OC)ccc2-3)cc1] |
| 415 | 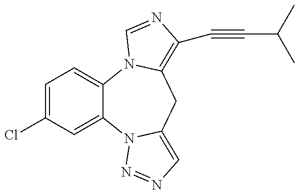 [SMILES: CC(C)C#Cc1ncn2c1Cc1cnnn1-c1cc(Cl)ccc1-2] |
| 416 | 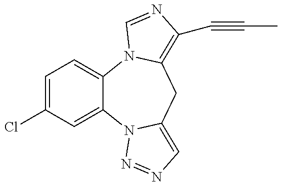 [SMILES: CC#Cc1ncn2c1Cc1cnnn1-c1cc(Cl)ccc1-2] |
| 417 | 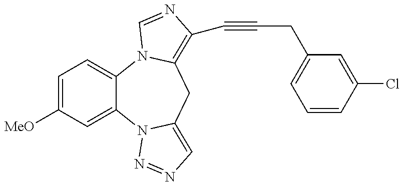 [SMILES: COc1ccc2c(c1)-n1nncc1Cc1c(C#CCc3cccc(Cl)c3)ncn1-2] |
| 418 | 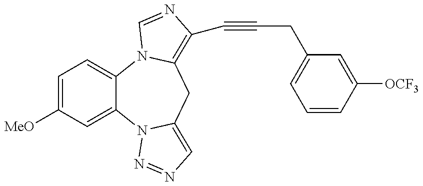 [SMILES: COc1ccc2c(c1)-n1nncc1Cc1c(C#CCc3cccc(OC(F)(F)F)c3)ncn1-2] |
| 419 | 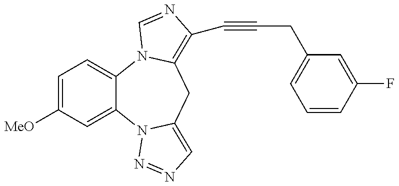 [SMILES: COc1ccc2c(c1)-n1nncc1Cc1c(C#CCc3cccc(F)c3)ncn1-2] |
| 420 | 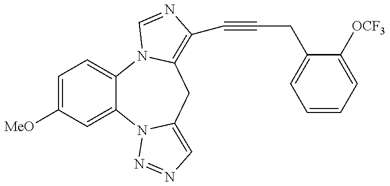 [SMILES: COc1ccc2c(c1)-n1nncc1Cc1c(C#CCc3ccccc3OC(F)(F)F)ncn1-2] |
| 421 | 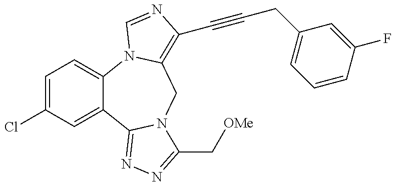 [SMILES: COCc1nnc2n1Cc1c(C#CCc3cccc(F)c3)ncn1-c1ccc(Cl)cc1-2] |
| 422 | 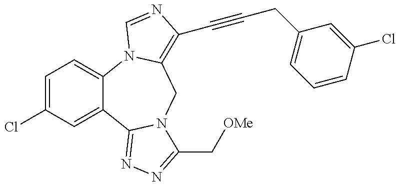 [SMILES: COCc1nnc2n1Cc1c(C#CCc3cccc(Cl)c3)ncn1-c1ccc(Cl)cc1-2] |
| 423 | 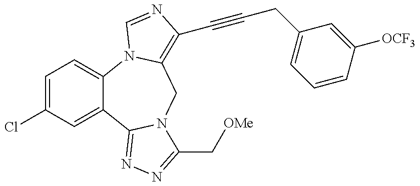 [SMILES: COCc1nnc2n1Cc1c(C#CCc3cccc(OC(F)(F)F)c3)ncn1-c1ccc(Cl)cc1-2] |
| 424 | 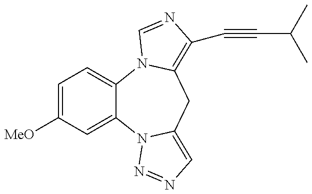 [SMILES: COc1ccc2c(c1)-n1nncc1Cc1c(C#CC(C)C)ncn1-2] |
| 425 | 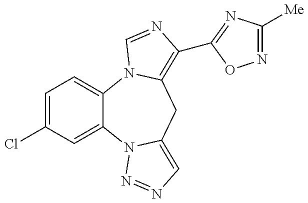 [SMILES: Cc1noc(-c2ncn3c2Cc2cnnn2-c2cc(Cl)ccc2-3)n1] |
| 426 | 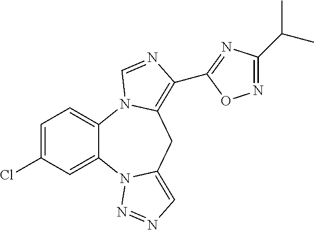 [SMILES: CC(C)c1noc(-c2ncn3c2Cc2cnnn2-c2cc(Cl)ccc2-3)n1] |
| 427 | 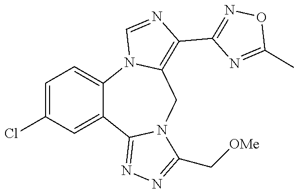 [SMILES: COCc1nnc2n1Cc1c(-c3noc(C)n3)ncn1-c1ccc(Cl)cc1-2] |
| 428 | 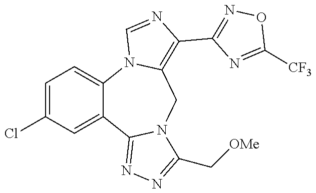 [SMILES: COCc1nnc2n1Cc1c(-c3noc(C(F)(F)F)n3)ncn1-c1ccc(Cl)cc1-2] |
| 429 | 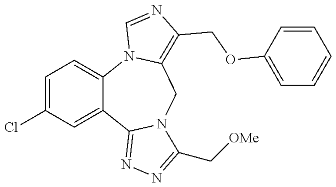 [SMILES: COCc1nnc2n1Cc1c(COc3ccccc3)ncn1-c1ccc(Cl)cc1-2] |
| 430 | 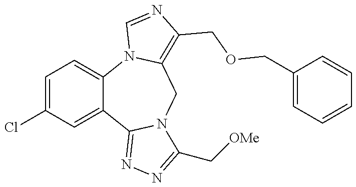 [SMILES: COCc1nnc2n1Cc1c(COCc3ccccc3)ncn1-c1ccc(Cl)cc1-2] |
| 431 | 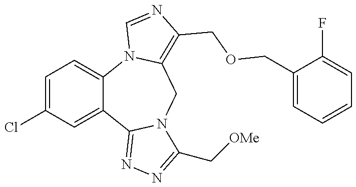 [SMILES: COCc1nnc2n1Cc1c(COCc3ccccc3F)ncn1-c1ccc(Cl)cc1-2] |
| 432 | 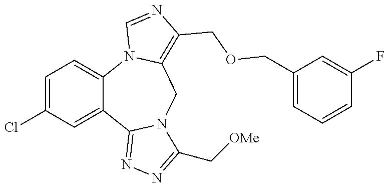 [SMILES: COCc1nnc2n1Cc1c(COCc3cccc(F)c3)ncn1-c1ccc(Cl)cc1-2] |
| 433 | 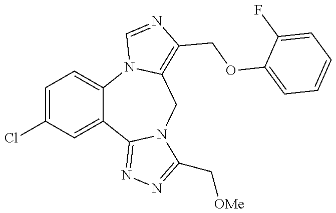 [SMILES: COCc1nnc2n1Cc1c(COc3ccccc3F)ncn1-c1ccc(Cl)cc1-2] |
| 434 | 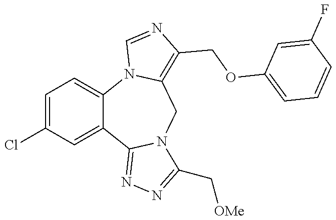 [SMILES: COCc1nnc2n1Cc1c(COc3cccc(F)c3)ncn1-c1ccc(Cl)cc1-2] |
| 435 | 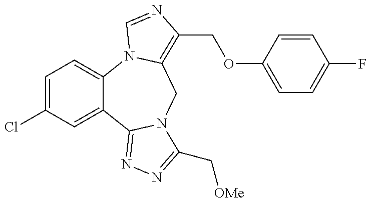 [SMILES: COCc1nnc2n1Cc1c(COc3ccc(F)cc3)ncn1-c1ccc(Cl)cc1-2] |
| 436 | 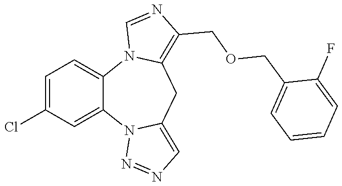 [SMILES: Fc1ccccc1COCc1ncn2c1Cc1cnnn1-c1cc(Cl)ccc1-2] |
| 437 | 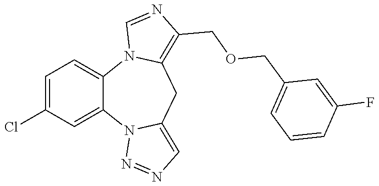 [SMILES: Fc1cccc(COCc2ncn3c2Cc2cnnn2-c2cc(Cl)ccc2-3)c1] |
| 438 | 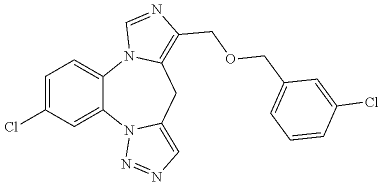 [SMILES: Clc1cccc(COCc2ncn3c2Cc2cnnn2-c2cc(Cl)ccc2-3)c1] |
| 439 | 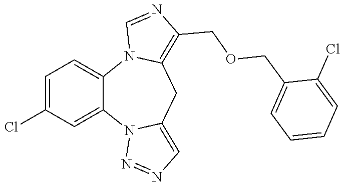 [SMILES: Clc1ccc2c(c1)-n1nncc1Cc1c(COCc3ccccc3Cl)ncn1-2] |
| 440 | 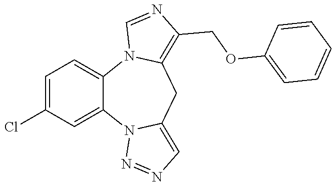 [SMILES: Clc1ccc2c(c1)-n1nncc1Cc1c(COc3ccccc3)ncn1-2] |
| 441 | 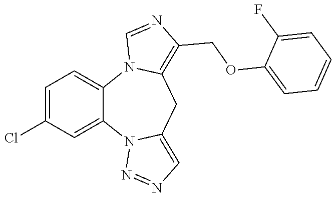 [SMILES: Fc1ccccc1OCc1ncn2c1Cc1cnnn1-c1cc(Cl)ccc1-2] |
| 442 | 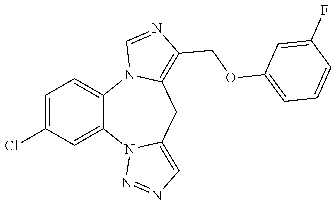 [SMILES: Fc1cccc(OCc2ncn3c2Cc2cnnn2-c2cc(Cl)ccc2-3)c1] |
| 443 | 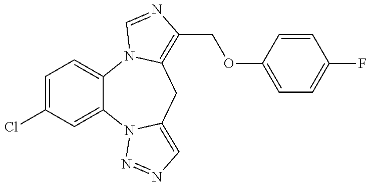 [SMILES: Fc1ccc(OCc2ncn3c2Cc2cnnn2-c2cc(Cl)ccc2-3)cc1] |
| 444 | 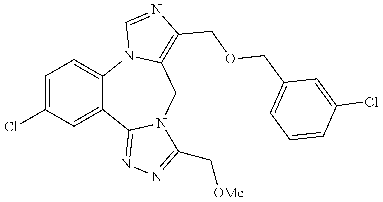 [SMILES: COCc1nnc2n1Cc1c(COCc3cccc(Cl)c3)ncn1-c1ccc(Cl)cc1-2] |
| 445 | 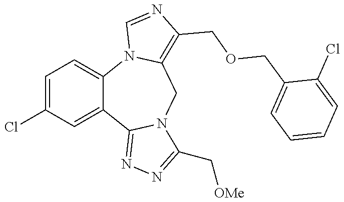 [SMILES: COCc1nnc2n1Cc1c(COCc3ccccc3Cl)ncn1-c1ccc(Cl)cc1-2] |
| 446 | 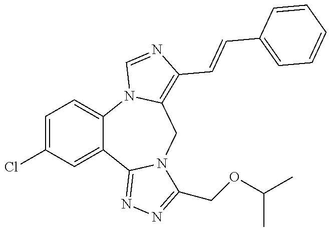 [SMILES: CC(C)OCc1nnc2n1Cc1c(/C=C/c3ccccc3)ncn1-c1ccc(Cl)cc1-2] |
| 447 | 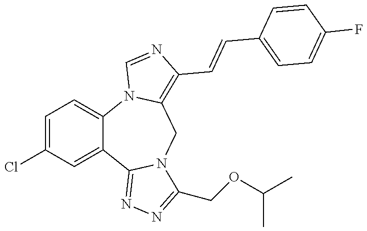 [SMILES: CC(C)OCc1nnc2n1Cc1c(/C=C/c3ccc(F)cc3)ncn1-c1ccc(Cl)cc1-2] |
| 448 | 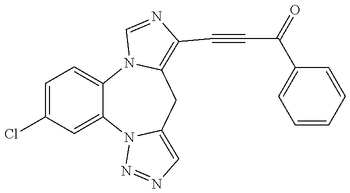 [SMILES: O=C(C#Cc1ncn2c1Cc1cnnn1-c1cc(Cl)ccc1-2)c1ccccc1] |
| 449 | 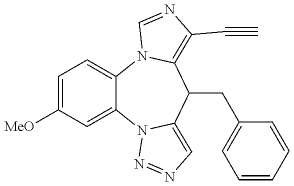 [SMILES: C#Cc1ncn2c1C(Cc1ccccc1)c1cnnn1-c1cc(OC)ccc1-2] |
| 450 | 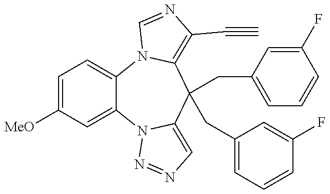 [SMILES: C#Cc1ncn2c1C(Cc1cccc(F)c1)(Cc1cccc(F)c1)c1cnnn1-c1cc(OC)ccc1-2] |
| 451 | 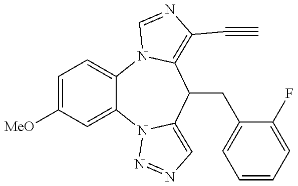 [SMILES: C#Cc1ncn2c1C(Cc1ccccc1F)c1cnnn1-c1cc(OC)ccc1-2] |
| 452 | 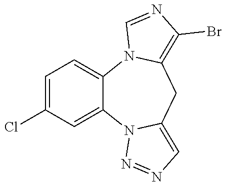 [SMILES: Clc1ccc2c(c1)-n1nncc1Cc1c(Br)ncn1-2] |
| 453 | 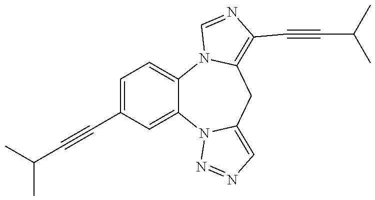 [SMILES: CC(C)C#Cc1ccc2c(c1)-n1nncc1Cc1c(C#CC(C)C)ncn1-2] |
| 454 | 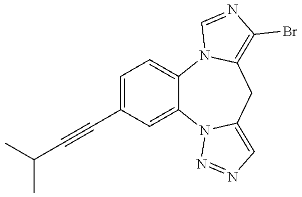 [SMILES: CC(C)C#Cc1ccc2c(c1)-n1nncc1Cc1c(Br)ncn1-2] |
| 455 | 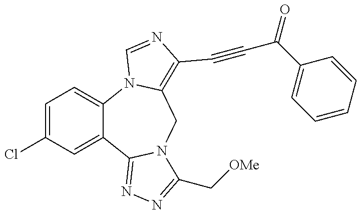 [SMILES: COCc1nnc2n1Cc1c(C#CC(=O)c3ccccc3)ncn1-c1ccc(Cl)cc1-2] |
| 456 | 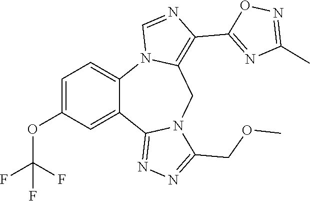 [SMILES: COCc1nnc2n1Cc1c(-c3nc(C)no3)ncn1-c1ccc(OC(F)(F)F)cc1-2] |
| 457 | 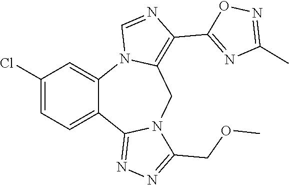 [SMILES: COCc1nnc2n1Cc1c(-c3nc(C)no3)ncn1-c1cc(Cl)ccc1-2] |
| 458 | 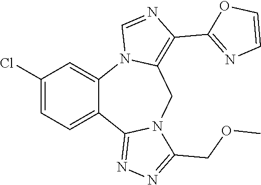 [SMILES: COCc1nnc2n1Cc1c(-c3ncco3)ncn1-c1cc(Cl)ccc1-2] |
| 459 | 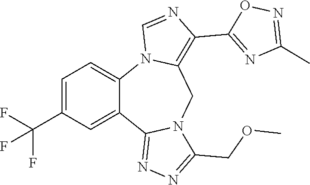 [SMILES: COCc1nnc2n1Cc1c(-c3nc(C)no3)ncn1-c1ccc(C(F)(F)F)cc1-2] |
| 460 | 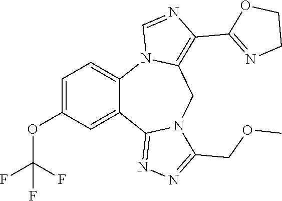 [SMILES: COCc1nnc2n1Cc1c(C3=NCCO3)ncn1-c1ccc(OC(F)(F)F)cc1-2] |
| 461 | 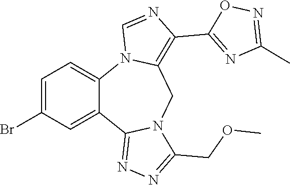 [SMILES: COCc1nnc2n1Cc1c(-c3nc(C)no3)ncn1-c1ccc(Br)cc1-2] |
| 462 | 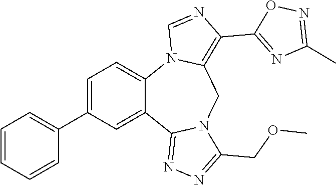 [SMILES: COCc1nnc2n1Cc1c(-c3nc(C)no3)ncn1-c1ccc(-c3ccccc3)cc1-2] |
| 463 | 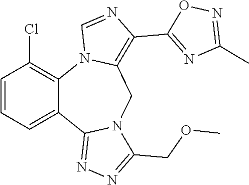 [SMILES: COCc1nnc2n1Cc1c(-c3nc(C)no3)ncn1-c1c(Cl)cccc1-2] |
| 464 | 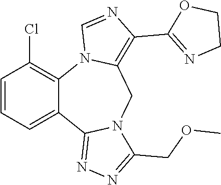 [SMILES: COCc1nnc2n1Cc1c(C3=NCCO3)ncn1-c1c(Cl)cccc1-2] |
| 465 | 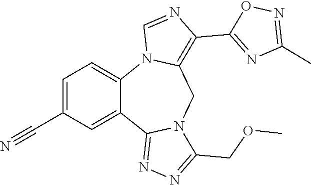 [SMILES: COCc1nnc2n1Cc1c(-c3nc(C)no3)ncn1-c1ccc(C#N)cc1-2] |
| 466 | 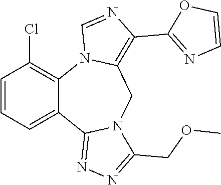 [SMILES: COCc1nnc2n1Cc1c(-c3ncco3)ncn1-c1c(Cl)cccc1-2] |
| 467 | 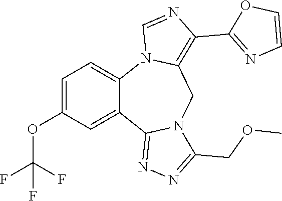 [SMILES: COCc1nnc2n1Cc1c(-c3ncco3)ncn1-c1ccc(OC(F)(F)F)cc1-2] |
| 468 | 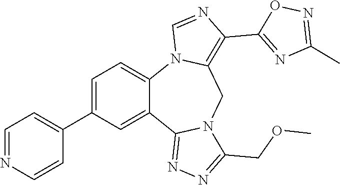 [SMILES: COCc1nnc2n1Cc1c(-c3nc(C)no3)ncn1-c1ccc(-c3ccncc3)cc1-2] |
| 469 | 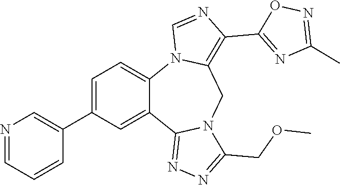 [SMILES: COCc1nnc2n1Cc1c(-c3nc(C)no3)ncn1-c1ccc(-c3cccnc3)cc1-2] |
| 470 | 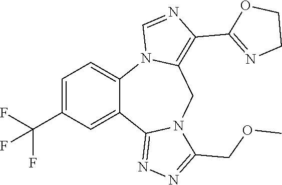 [SMILES: COCc1nnc2n1Cc1c(C3=NCCO3)ncn1-c1ccc(C(F)(F)F)cc1-2] |
| 471 | 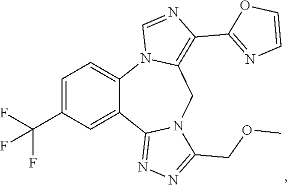 [SMILES: COCc1nnc2n1Cc1c(-c3ncco3)ncn1-c1ccc(C(F)(F)F)cc1-2] |
| 472 | 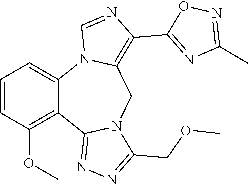 [SMILES: COCc1nnc2n1Cc1c(-c3nc(C)no3)ncn1-c1cccc(OC)c1-2] |
| 473 | 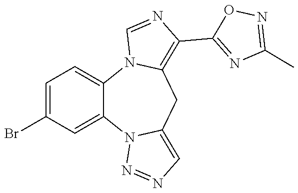 [SMILES: Cc1noc(-c2ncn3c2Cc2cnnn2-c2cc(Br)ccc2-3)n1] |
| 474 | 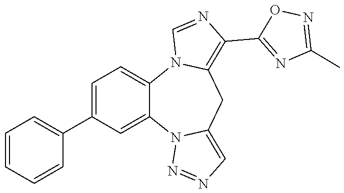 [SMILES: Cc1noc(-c2ncn3c2Cc2cnnn2-c2cc(-c4ccccc4)ccc2-3)n1] |
| 475 | 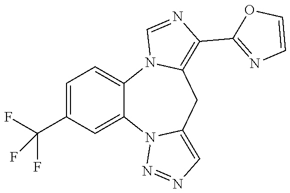 [SMILES: FC(F)(F)c1ccc2c(c1)-n1nncc1Cc1c(-c3ncco3)ncn1-2] |
| 476 | 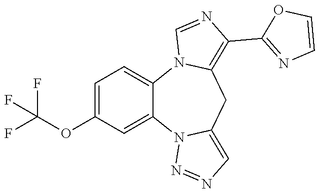 [SMILES: FC(F)(F)Oc1ccc2c(c1)-n1nncc1Cc1c(-c3ncco3)ncn1-2] |
| 477 | 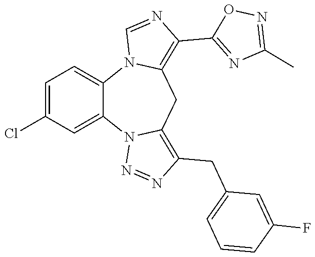 [SMILES: Cc1noc(-c2ncn3c2Cc2c(Cc4cccc(F)c4)nnn2-c2cc(Cl)ccc2-3)n1] |
| 478 | 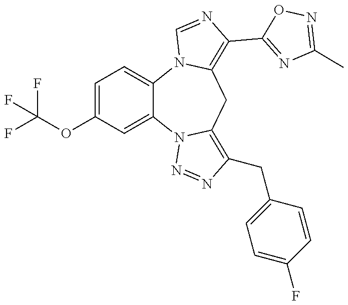 [SMILES: Cc1noc(-c2ncn3c2Cc2c(Cc4ccc(F)cc4)nnn2-c2cc(OC(F)(F)F)ccc2-3)n1] |
| 479 | 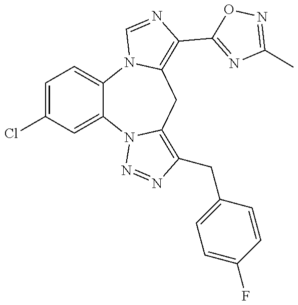 [SMILES: Cc1noc(-c2ncn3c2Cc2c(Cc4ccc(F)cc4)nnn2-c2cc(Cl)ccc2-3)n1] |
| 480 | 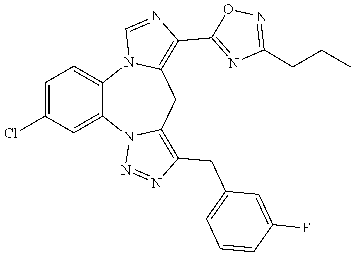 [SMILES: CCCc1noc(-c2ncn3c2Cc2c(Cc4cccc(F)c4)nnn2-c2cc(Cl)ccc2-3)n1] |
| 481 | 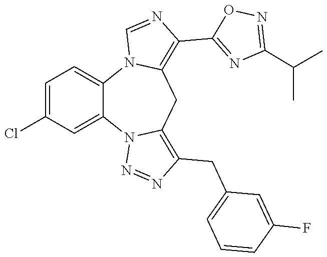 [SMILES: CC(C)c1noc(-c2ncn3c2Cc2c(Cc4cccc(F)c4)nnn2-c2cc(Cl)ccc2-3)n1] |
| 482 | 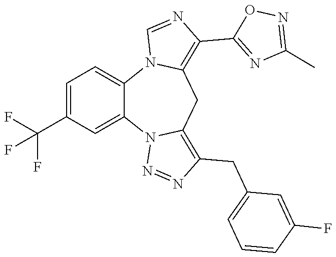 [SMILES: Cc1noc(-c2ncn3c2Cc2c(Cc4cccc(F)c4)nnn2-c2cc(C(F)(F)F)ccc2-3)n1] |
| 483 | 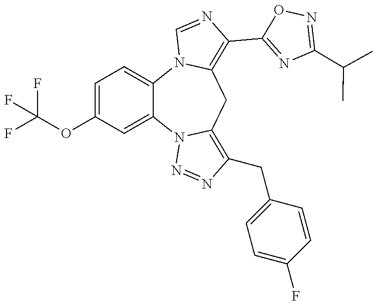 [SMILES: CC(C)c1noc(-c2ncn3c2Cc2c(Cc4ccc(F)cc4)nnn2-c2cc(OC(F)(F)F)ccc2-3)n1] |
| 484 | 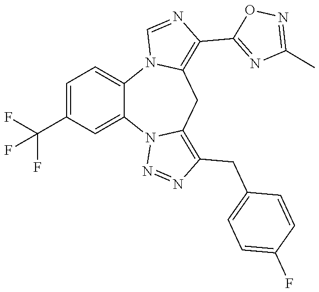 [SMILES: Cc1noc(-c2ncn3c2Cc2c(Cc4ccc(F)cc4)nnn2-c2cc(C(F)(F)F)ccc2-3)n1] |
| 485 | 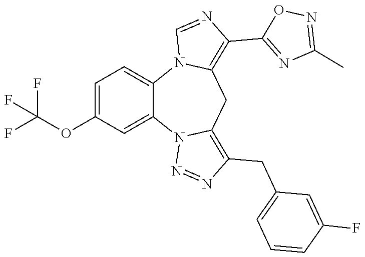 [SMILES: Cc1noc(-c2ncn3c2Cc2c(Cc4cccc(F)c4)nnn2-c2cc(OC(F)(F)F)ccc2-3)n1] |
| 486 | 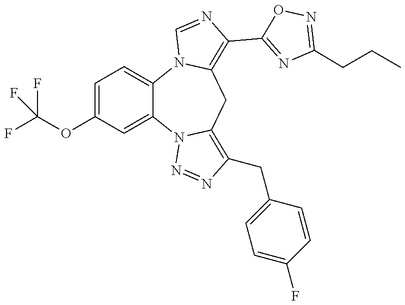 [SMILES: CCCc1noc(-c2ncn3c2Cc2c(Cc4ccc(F)cc4)nnn2-c2cc(OC(F)(F)F)ccc2-3)n1] |
| 487 | 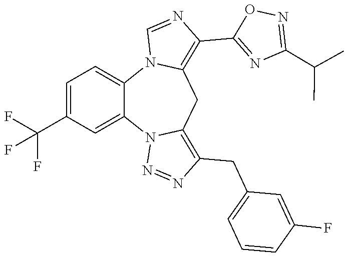 [SMILES: CC(C)c1noc(-c2ncn3c2Cc2c(Cc4cccc(F)c4)nnn2-c2cc(C(F)(F)F)ccc2-3)n1] |
| 488 | 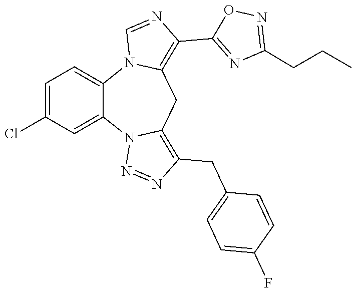 [SMILES: CCCc1noc(-c2ncn3c2Cc2c(Cc4ccc(F)cc4)nnn2-c2cc(Cl)ccc2-3)n1] |
| 489 | 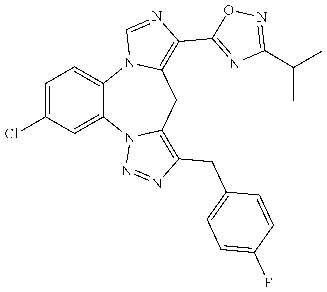 [SMILES: CC(C)c1noc(-c2ncn3c2Cc2c(Cc4ccc(F)cc4)nnn2-c2cc(Cl)ccc2-3)n1] |
| 490 | 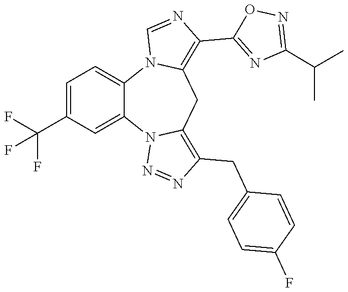 [SMILES: CC(C)c1noc(-c2ncn3c2Cc2c(Cc4ccc(F)cc4)nnn2-c2cc(C(F)(F)F)ccc2-3)n1] |
| 491 | 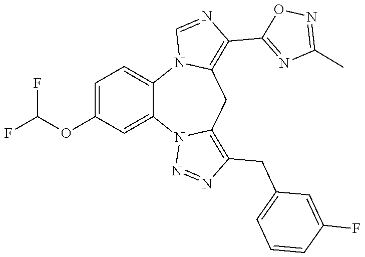 [SMILES: Cc1noc(-c2ncn3c2Cc2c(Cc4cccc(F)c4)nnn2-c2cc(OC(F)F)ccc2-3)n1] |
| 492 | 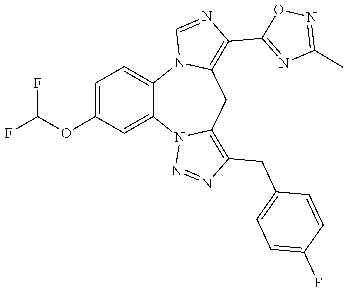 [SMILES: Cc1noc(-c2ncn3c2Cc2c(Cc4ccc(F)cc4)nnn2-c2cc(OC(F)F)ccc2-3)n1] |
| 493 | 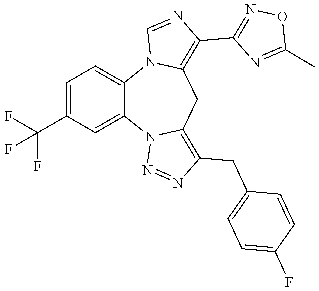 [SMILES: Cc1nc(-c2ncn3c2Cc2c(Cc4ccc(F)cc4)nnn2-c2cc(C(F)(F)F)ccc2-3)no1] |
| 494 | 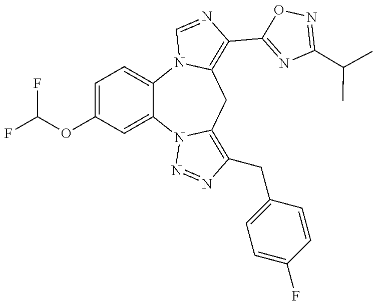 [SMILES: CC(C)c1noc(-c2ncn3c2Cc2c(Cc4ccc(F)cc4)nnn2-c2cc(OC(F)F)ccc2-3)n1] |
| 495 | 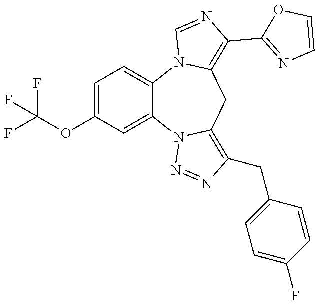 [SMILES: Fc1ccc(Cc2nnn3c2Cc2c(-c4ncco4)ncn2-c2ccc(OC(F)(F)F)cc2-3)cc1] |
| 496 | 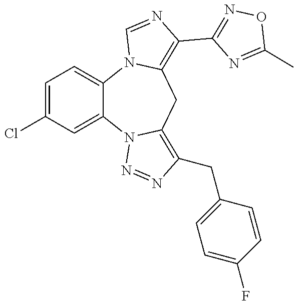 [SMILES: Cc1nc(-c2ncn3c2Cc2c(Cc4ccc(F)cc4)nnn2-c2cc(Cl)ccc2-3)no1] |
| 497 | 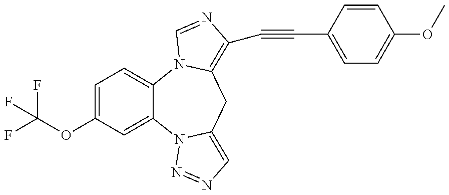 [SMILES: COc1ccc(C#Cc2ncn3c2Cc2cnnn2-c2cc(OC(F)(F)F)ccc2-3)cc1] |
| 498 | 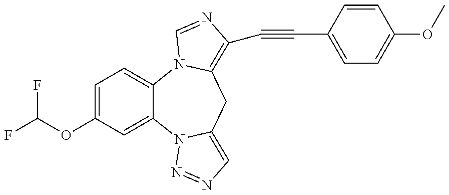 [SMILES: COc1ccc(C#Cc2ncn3c2Cc2cnnn2-c2cc(OC(F)F)ccc2-3)cc1] |
| 499 | 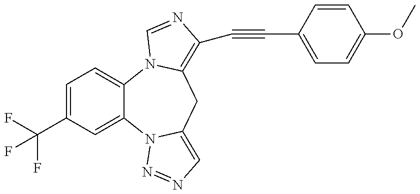 [SMILES: COc1ccc(C#Cc2ncn3c2Cc2cnnn2-c2cc(C(F)(F)F)ccc2-3)cc1] |
| 500 | 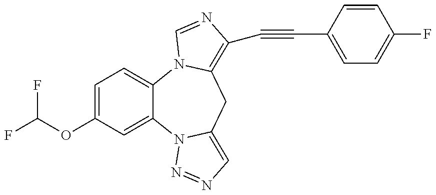 [SMILES: Fc1ccc(C#Cc2ncn3c2Cc2cnnn2-c2cc(OC(F)F)ccc2-3)cc1] |
| 501 | 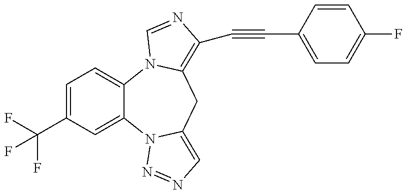 [SMILES: Fc1ccc(C#Cc2ncn3c2Cc2cnnn2-c2cc(C(F)(F)F)ccc2-3)cc1] |
| 502 | 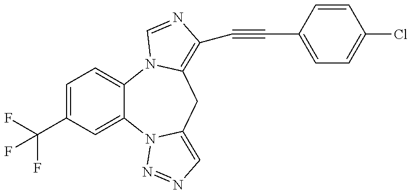 [SMILES: FC(F)(F)c1ccc2c(c1)-n1nncc1Cc1c(C#Cc3ccc(Cl)cc3)ncn1-2] |
| 503 | 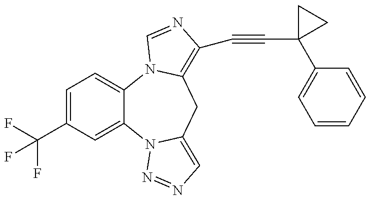 [SMILES: FC(F)(F)c1ccc2c(c1)-n1nncc1Cc1c(C#CC3(c4ccccc4)CC3)ncn1-2] |
| 504 | 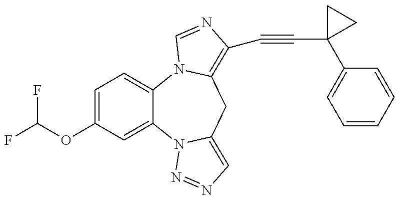 [SMILES: FC(F)Oc1ccc2c(c1)-n1nncc1Cc1c(C#CC3(c4ccccc4)CC3)ncn1-2] |
| 505 | 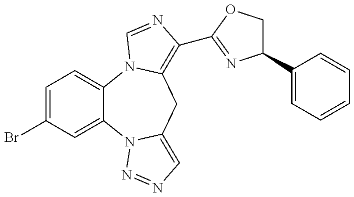 [SMILES: Brc1ccc2c(c1)-n1nncc1Cc1c(C3=N[C@H](c4ccccc4)CO3)ncn1-2] |
| 506 | 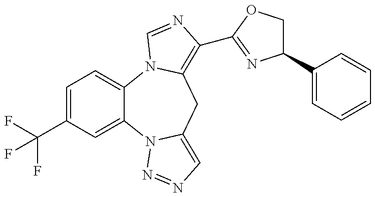 [SMILES: FC(F)(F)c1ccc2c(c1)-n1nncc1Cc1c(C3=N[C@H](c4ccccc4)CO3)ncn1-2] |
| 507 | 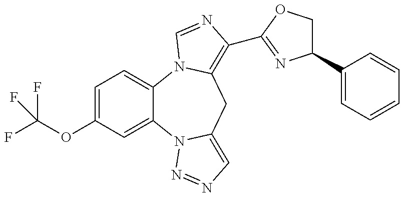 [SMILES: FC(F)(F)Oc1ccc2c(c1)-n1nncc1Cc1c(C3=N[C@H](c4ccccc4)CO3)ncn1-2] |
| 508 | 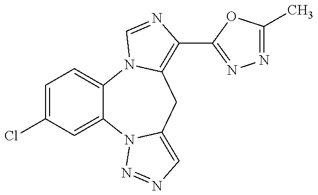 [SMILES: Cc1nnc(-c2ncn3c2Cc2cnnn2-c2cc(Cl)ccc2-3)o1] |
| 509 | 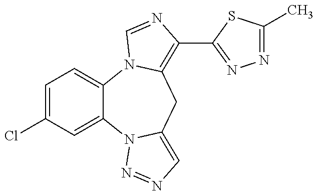 [SMILES: Cc1nnc(-c2ncn3c2Cc2cnnn2-c2cc(Cl)ccc2-3)s1] |
| 510 | 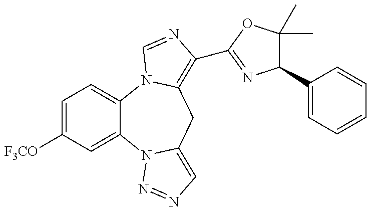 [SMILES: CC1(C)OC(c2ncn3c2Cc2cnnn2-c2cc(OC(F)(F)F)ccc2-3)=N[C@@H]1c1ccccc1] |
| 511 | 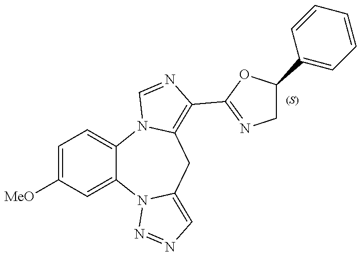 |
| 512 | 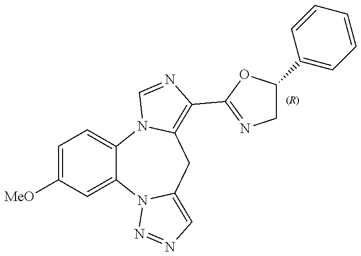 |
| 513 | 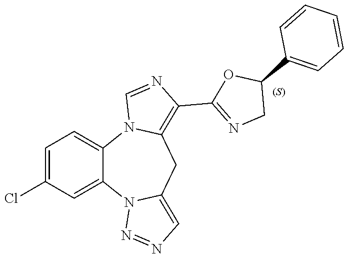 |
| 514 | 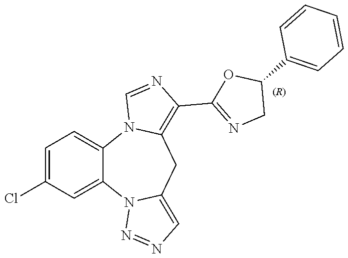 |
| 515 | 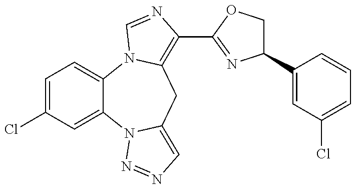 [SMILES: Clc1cccc([C@@H]2COC(c3ncn4c3Cc3cnnn3-c3cc(Cl)ccc3-4)=N2)c1] |
| 516 | 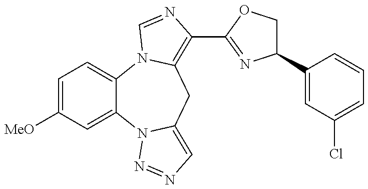 [SMILES: COc1ccc2c(c1)-n1nncc1Cc1c(C3=N[C@H](c4cccc(Cl)c4)CO3)ncn1-2] |
| 517 | 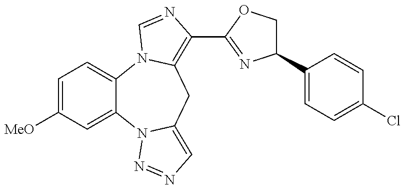 [SMILES: COc1ccc2c(c1)-n1nncc1Cc1c(C3=N[C@H](c4ccc(Cl)cc4)CO3)ncn1-2] |
| 518 | 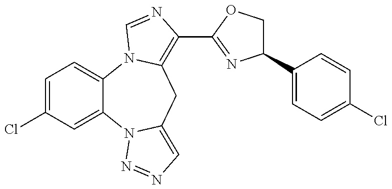 [SMILES: Clc1ccc([C@@H]2COC(c3ncn4c3Cc3cnnn3-c3cc(Cl)ccc3-4)=N2)cc1] |
| 519 | 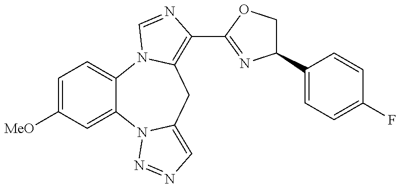 [SMILES: COc1ccc2c(c1)-n1nncc1Cc1c(C3=N[C@H](c4ccc(F)cc4)CO3)ncn1-2] |
| 520 | 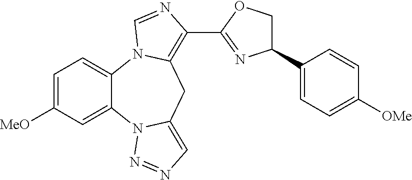 [SMILES: COc1ccc([C@@H]2COC(c3ncn4c3Cc3cnnn3-c3cc(OC)ccc3-4)=N2)cc1] |
| 521 | 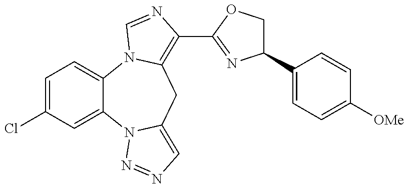 [SMILES: COc1ccc([C@@H]2COC(c3ncn4c3Cc3cnnn3-c3cc(Cl)ccc3-4)=N2)cc1] |
| 522 | 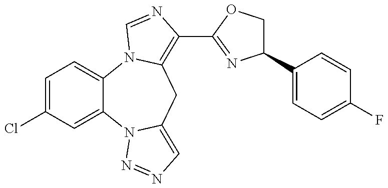 [SMILES: Fc1ccc([C@@H]2COC(c3ncn4c3Cc3cnnn3-c3cc(Cl)ccc3-4)=N2)cc1] |
| 523 | 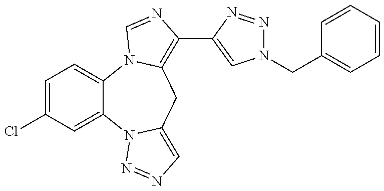 [SMILES: Clc1ccc2c(c1)-n1nncc1Cc1c(-c3cn(Cc4ccccc4)nn3)ncn1-2] |
| 524 | 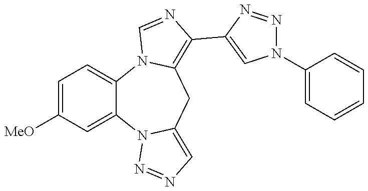 [SMILES: COc1ccc2c(c1)-n1nncc1Cc1c(-c3cn(-c4ccccc4)nn3)ncn1-2] |
| 525 | 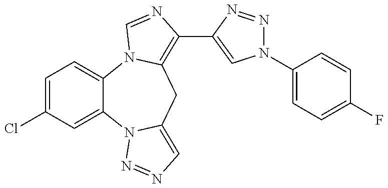 [SMILES: Fc1ccc(-n2cc(-c3ncn4c3Cc3cnnn3-c3cc(Cl)ccc3-4)nn2)cc1] |
| 526 | 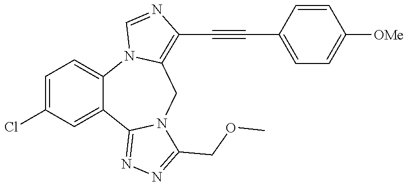 [SMILES: COCc1nnc2n1Cc1c(C#Cc3ccc(OC)cc3)ncn1-c1ccc(Cl)cc1-2] |
| 527 | 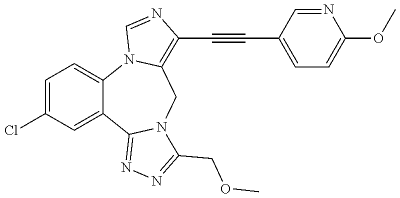 [SMILES: COCc1nnc2n1Cc1c(C#Cc3ccc(OC)nc3)ncn1-c1ccc(Cl)cc1-2] |
| 528 | 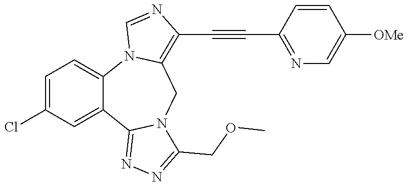 [SMILES: COCc1nnc2n1Cc1c(C#Cc3ccc(OC)cn3)ncn1-c1ccc(Cl)cc1-2] |
| 529 | 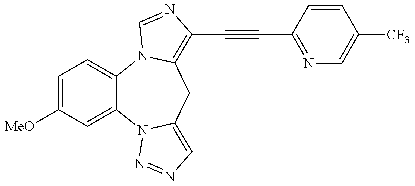 [SMILES: COc1ccc2c(c1)-n1nncc1Cc1c(C#Cc3ccc(C(F)(F)F)cn3)ncn1-2] |
| 530 | 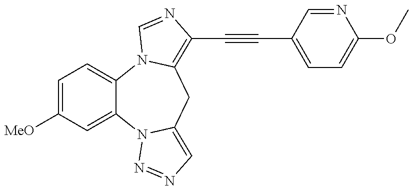 [SMILES: COc1ccc2c(c1)-n1nncc1Cc1c(C#Cc3ccc(OC)nc3)ncn1-2] |
| 531 | 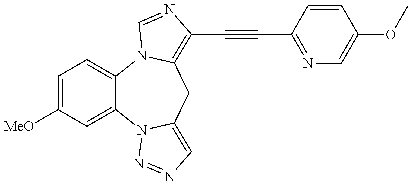 [SMILES: COc1ccc(C#Cc2ncn3c2Cc2cnnn2-c2cc(OC)ccc2-3)nc1] |
| 532 | 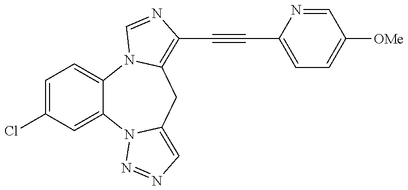 [SMILES: COc1ccc(C#Cc2ncn3c2Cc2cnnn2-c2cc(Cl)ccc2-3)nc1] |
| 533 | 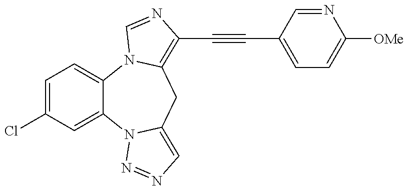 [SMILES: COc1ccc(C#Cc2ncn3c2Cc2cnnn2-c2cc(Cl)ccc2-3)cn1] |
| 534 | 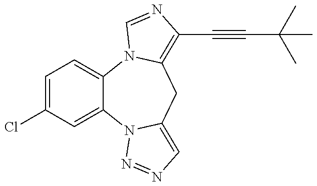 [SMILES: CC(C)(C)C#Cc1ncn2c1Cc1cnnn1-c1cc(Cl)ccc1-2] |
| 535 | 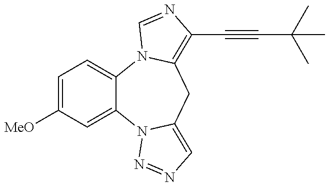 [SMILES: COc1ccc2c(c1)-n1nncc1Cc1c(C#CC(C)(C)C)ncn1-2] |
| 536 | 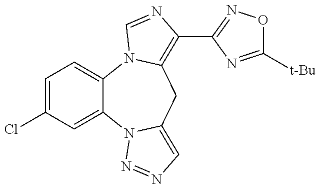 [SMILES: CC(C)(C)c1nc(-c2ncn3c2Cc2cnnn2-c2cc(Cl)ccc2-3)no1] |
| 537 | 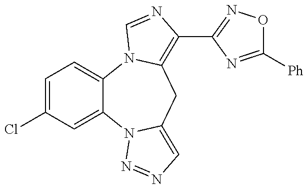 [SMILES: Clc1ccc2c(c1)-n1nncc1Cc1c(-c3noc(-c4ccccc4)n3)ncn1-2] |
| 538 | 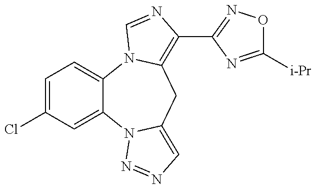 [SMILES: CC(C)c1nc(-c2ncn3c2Cc2cnnn2-c2cc(Cl)ccc2-3)no1] |
| 539 | 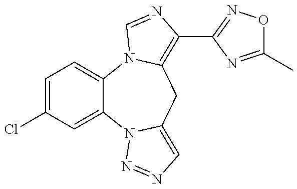 [SMILES: Cc1nc(-c2ncn3c2Cc2cnnn2-c2cc(Cl)ccc2-3)no1] |
| 540 | 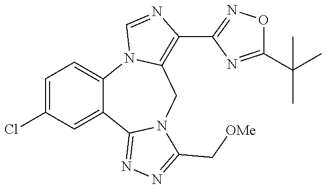 [SMILES: COCc1nnc2n1Cc1c(-c3noc(C(C)(C)C)n3)ncn1-c1ccc(Cl)cc1-2] |
| 541 | 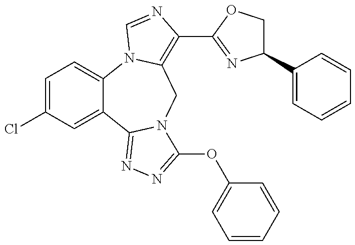 [SMILES: Clc1ccc2c(c1)-c1nnc(Oc3ccccc3)n1Cc1c(C3=N[C@H](c4ccccc4)CO3)ncn1-2] |
| 542 | 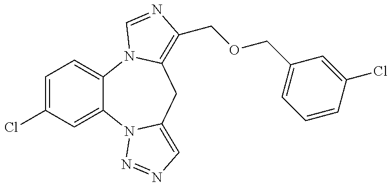 [SMILES: Clc1cccc(COCc2ncn3c2Cc2cnnn2-c2cc(Cl)ccc2-3)c1] |
| 543 | 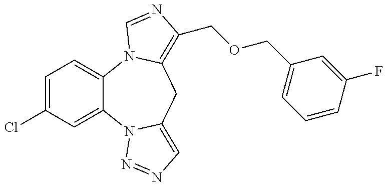 [SMILES: Fc1cccc(COCc2ncn3c2Cc2cnnn2-c2cc(Cl)ccc2-3)c1] |
| 544 | 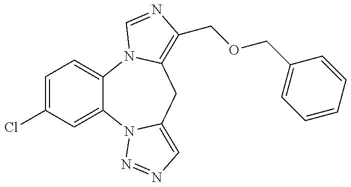 [SMILES: Clc1ccc2c(c1)-n1nncc1Cc1c(COCc3ccccc3)ncn1-2] |
| 545 | 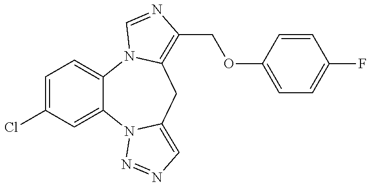 [SMILES: Fc1ccc(OCc2ncn3c2Cc2cnnn2-c2cc(Cl)ccc2-3)cc1] |
| 546 | 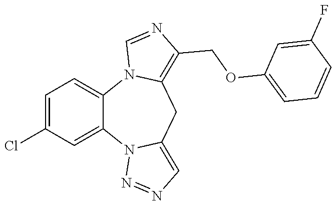 [SMILES: Fc1cccc(OCc2ncn3c2Cc2cnnn2-c2cc(Cl)ccc2-3)c1] |
| 547 | 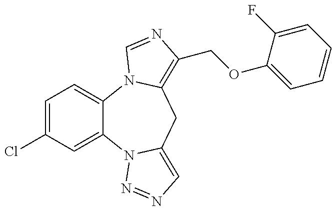 [SMILES: Fc1ccccc1OCc1ncn2c1Cc1cnnn1-c1cc(Cl)ccc1-2] |
| 548 | 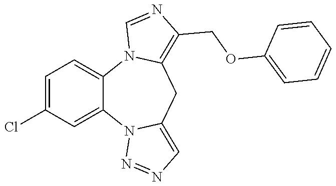 [SMILES: Clc1ccc2c(c1)-n1nncc1Cc1c(COc3ccccc3)ncn1-2] |
| 549 | 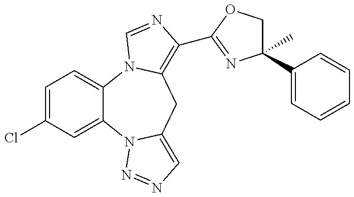 [SMILES: C[C@@]1(c2ccccc2)COC(c2ncn3c2Cc2cnnn2-c2cc(Cl)ccc2-3)=N1] |
| 550 | 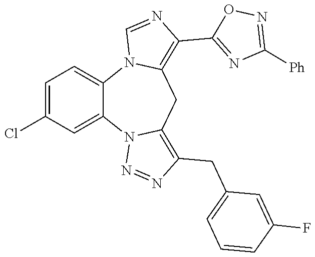 [SMILES: Fc1cccc(Cc2nnn3c2Cc2c(-c4nc(-c5ccccc5)no4)ncn2-c2ccc(Cl)cc2-3)c1] |
| 551 | 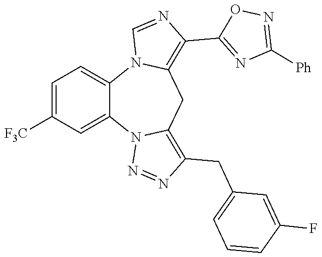 [SMILES: Fc1cccc(Cc2nnn3c2Cc2c(-c4nc(-c5ccccc5)no4)ncn2-c2ccc(C(F)(F)F)cc2-3)c1] |
| 552 | 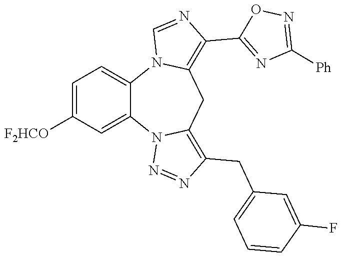 [SMILES: Fc1cccc(Cc2nnn3c2Cc2c(-c4nc(-c5ccccc5)no4)ncn2-c2ccc(OC(F)F)cc2-3)c1] |
| 553 | 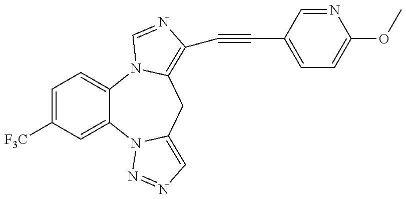 [SMILES: COc1ccc(C#Cc2ncn3c2Cc2cnnn2-c2cc(C(F)(F)F)ccc2-3)cn1] |
| 554 | 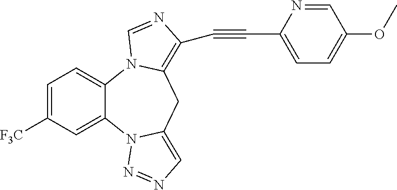 [SMILES: COc1ccc(C#Cc2ncn3c2Cc2cnnn2-c2cc(C(F)(F)F)ccc2-3)nc1] |
| 555 | 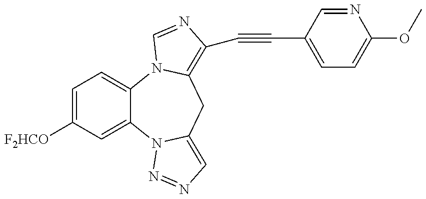 [SMILES: COc1ccc(C#Cc2ncn3c2Cc2cnnn2-c2cc(OC(F)F)ccc2-3)cn1] |
| 556 | 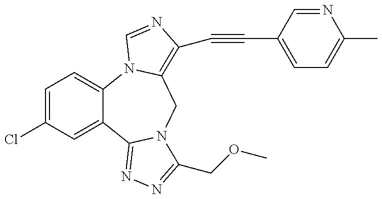 [SMILES: COCc1nnc2n1Cc1c(C#Cc3ccc(C)nc3)ncn1-c1ccc(Cl)cc1-2] |
| 557 | 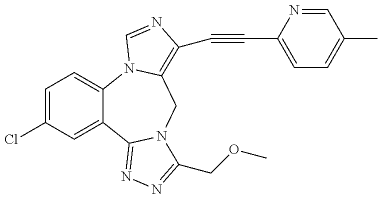 [SMILES: COCc1nnc2n1Cc1c(C#Cc3ccc(C)cn3)ncn1-c1ccc(Cl)cc1-2] |
| 558 | 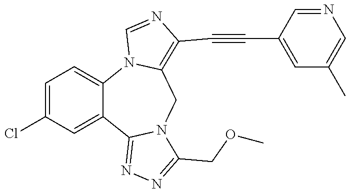 [SMILES: COCc1nnc2n1Cc1c(C#Cc3cncc(C)c3)ncn1-c1ccc(Cl)cc1-2] |
| 559 | 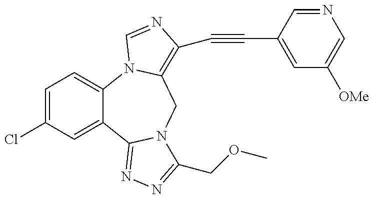 [SMILES: COCc1nnc2n1Cc1c(C#Cc3cncc(OC)c3)ncn1-c1ccc(Cl)cc1-2] |
| 560 | 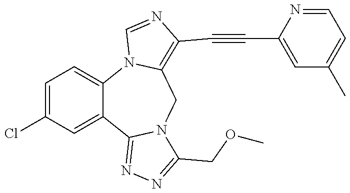 [SMILES: COCc1nnc2n1Cc1c(C#Cc3cc(C)ccn3)ncn1-c1ccc(Cl)cc1-2] |
| 561 | 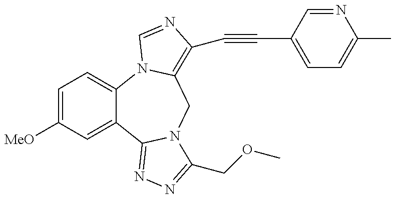 [SMILES: COCc1nnc2n1Cc1c(C#Cc3ccc(C)nc3)ncn1-c1ccc(OC)cc1-2] |
| 562 | 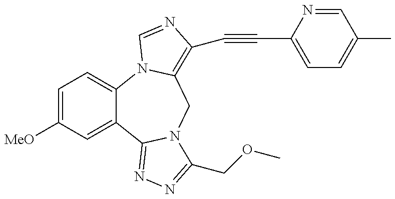 [SMILES: COCc1nnc2n1Cc1c(C#Cc3ccc(C)cn3)ncn1-c1ccc(OC)cc1-2] |
| 563 | 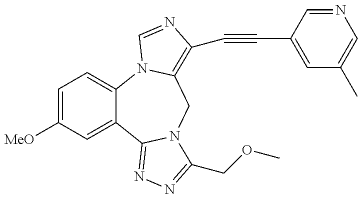 [SMILES: COCc1nnc2n1Cc1c(C#Cc3cncc(C)c3)ncn1-c1ccc(OC)cc1-2] |
| 564 | 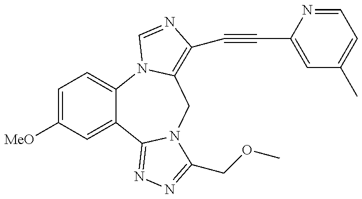 [SMILES: COCc1nnc2n1Cc1c(C#Cc3cc(C)ccn3)ncn1-c1ccc(OC)cc1-2] |
| 565 | 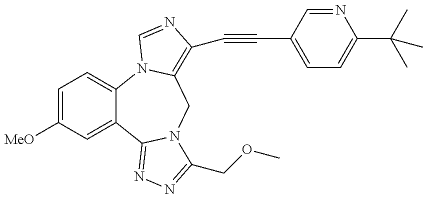 [SMILES: COCc1nnc2n1Cc1c(C#Cc3ccc(C(C)(C)C)nc3)ncn1-c1ccc(OC)cc1-2] |
| 566 | 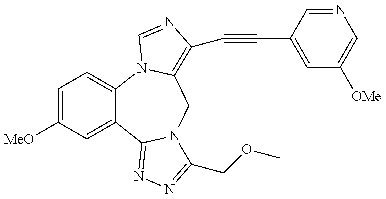 [SMILES: COCc1nnc2n1Cc1c(C#Cc3cncc(OC)c3)ncn1-c1ccc(OC)cc1-2] |
| 567 | 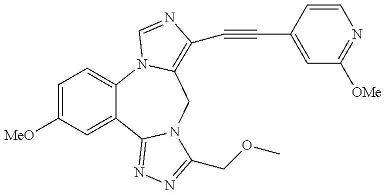 [SMILES: COCc1nnc2n1Cc1c(C#Cc3ccnc(OC)c3)ncn1-c1ccc(OC)cc1-2] |
| 568 | 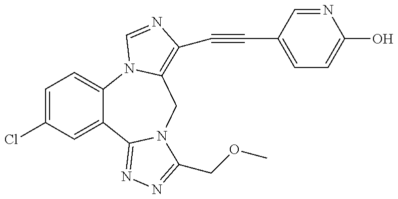 [SMILES: COCc1nnc2n1Cc1c(C#Cc3ccc(O)nc3)ncn1-c1ccc(Cl)cc1-2] |
| 569 | 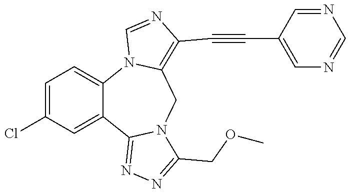 [SMILES: COCc1nnc2n1Cc1c(C#Cc3cncnc3)ncn1-c1ccc(Cl)cc1-2] |
| 570 | 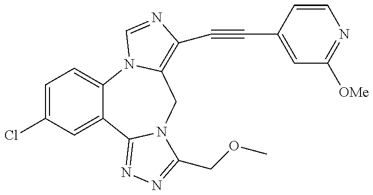 [SMILES: COCc1nnc2n1Cc1c(C#Cc3ccnc(OC)c3)ncn1-c1ccc(Cl)cc1-2] |
| 571 | 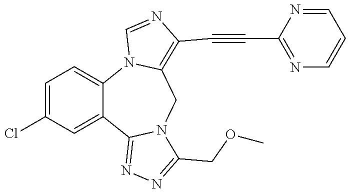 [SMILES: COCc1nnc2n1Cc1c(C#Cc3ncccn3)ncn1-c1ccc(Cl)cc1-2] |
| 572 | 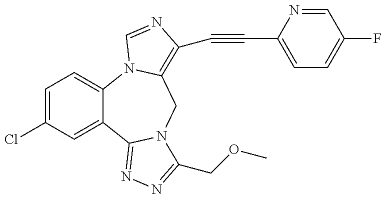 [SMILES: COCc1nnc2n1Cc1c(C#Cc3ccc(F)cn3)ncn1-c1ccc(Cl)cc1-2] |
| 573 | 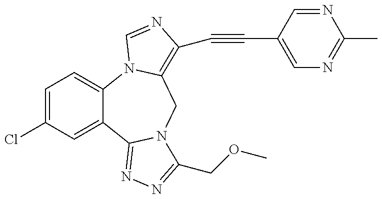 [SMILES: COCc1nnc2n1Cc1c(C#Cc3cnc(C)nc3)ncn1-c1ccc(Cl)cc1-2] |
| 574 | 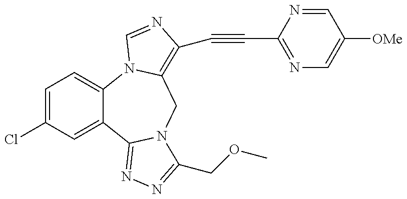 [SMILES: COCc1nnc2n1Cc1c(C#Cc3ncc(OC)cn3)ncn1-c1ccc(Cl)cc1-2] |
| 575 | 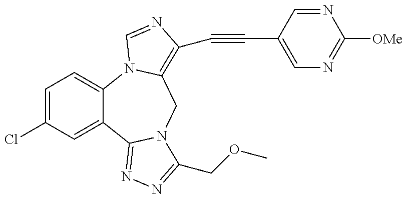 [SMILES: COCc1nnc2n1Cc1c(C#Cc3cnc(OC)nc3)ncn1-c1ccc(Cl)cc1-2] |
| 576 | 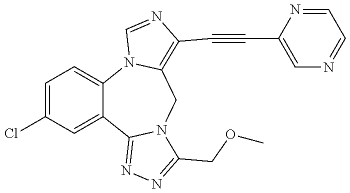 [SMILES: COCc1nnc2n1Cc1c(C#Cc3cnccn3)ncn1-c1ccc(Cl)cc1-2] |
| 577 | 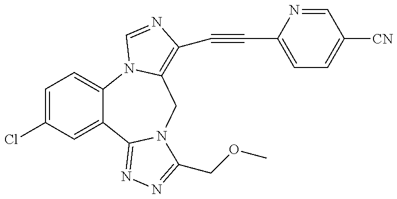 [SMILES: COCc1nnc2n1Cc1c(C#Cc3ccc(C#N)cn3)ncn1-c1ccc(Cl)cc1-2] |
| 578 | 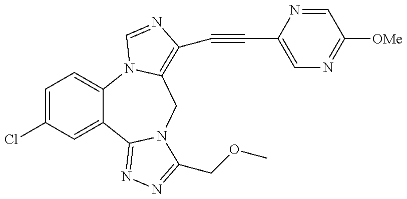 [SMILES: COCc1nnc2n1Cc1c(C#Cc3cnc(OC)cn3)ncn1-c1ccc(Cl)cc1-2] |
| 579 | 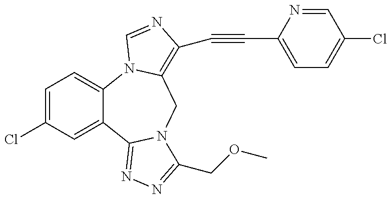 [SMILES: COCc1nnc2n1Cc1c(C#Cc3ccc(Cl)cn3)ncn1-c1ccc(Cl)cc1-2] |
| 580 | 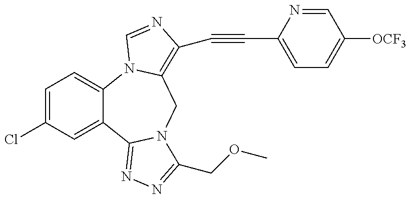 [SMILES: COCc1nnc2n1Cc1c(C#Cc3ccc(OC(F)(F)F)cn3)ncn1-c1ccc(Cl)cc1-2] |
| 581 | 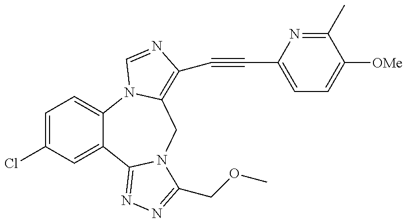 [SMILES: COCc1nnc2n1Cc1c(C#Cc3ccc(OC)c(C)n3)ncn1-c1ccc(Cl)cc1-2] |
| 582 | 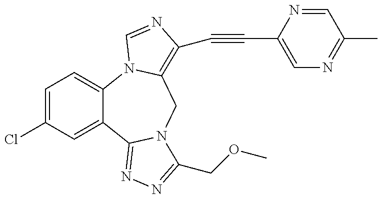 [SMILES: COCc1nnc2n1Cc1c(C#Cc3cnc(C)cn3)ncn1-c1ccc(Cl)cc1-2] |
| 583 | 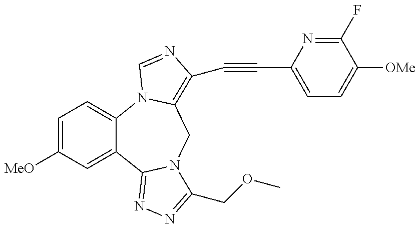 [SMILES: COCc1nnc2n1Cc1c(C#Cc3ccc(OC)c(F)n3)ncn1-c1ccc(OC)cc1-2] |
| 584 | 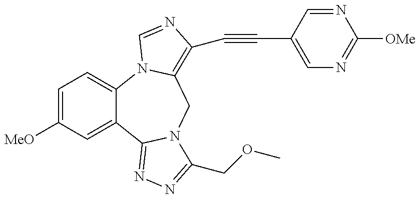 [SMILES: COCc1nnc2n1Cc1c(C#Cc3cnc(OC)nc3)ncn1-c1ccc(OC)cc1-2] |
| 585 | 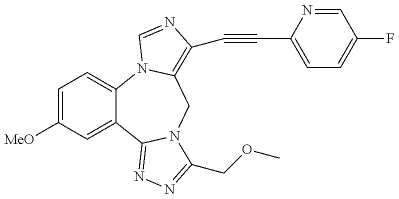 [SMILES: COCc1nnc2n1Cc1c(C#Cc3ccc(F)cn3)ncn1-c1ccc(OC)cc1-2] |
| 586 | 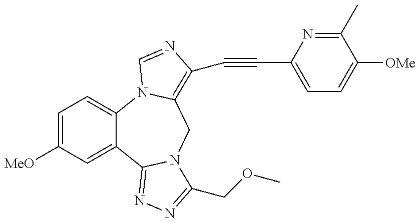 [SMILES: COCc1nnc2n1Cc1c(C#Cc3ccc(OC)c(C)n3)ncn1-c1ccc(OC)cc1-2] |
| 587 | 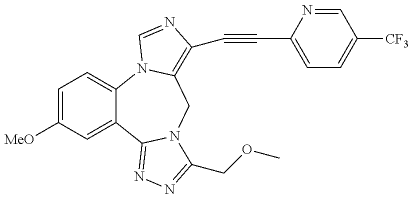 [SMILES: COCc1nnc2n1Cc1c(C#Cc3ccc(C(F)(F)F)cn3)ncn1-c1ccc(OC)cc1-2] |
| 588 | 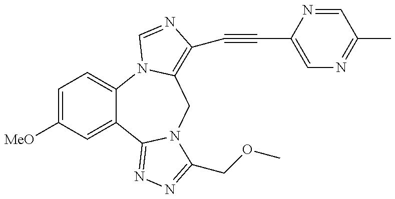 [SMILES: COCc1nnc2n1Cc1c(C#Cc3cnc(C)cn3)ncn1-c1ccc(OC)cc1-2] |
| 589 | 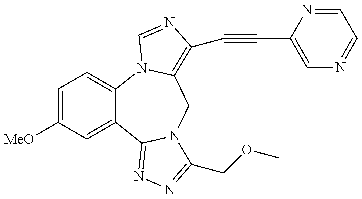 [SMILES: COCc1nnc2n1Cc1c(C#Cc3cnccn3)ncn1-c1ccc(OC)cc1-2] |
| 590 | 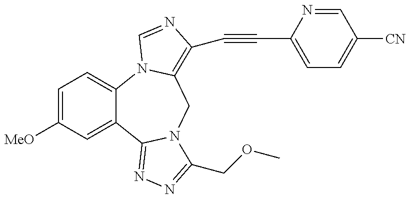 [SMILES: COCc1nnc2n1Cc1c(C#Cc3ccc(C#N)cn3)ncn1-c1ccc(OC)cc1-2] |
| 591 | 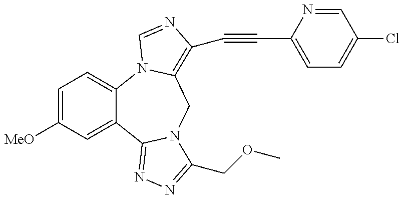 [SMILES: COCc1nnc2n1Cc1c(C#Cc3ccc(Cl)cn3)ncn1-c1ccc(OC)cc1-2] |
| 592 | 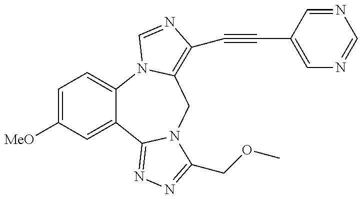 [SMILES: COCc1nnc2n1Cc1c(C#Cc3cncnc3)ncn1-c1ccc(OC)cc1-2] |
| 593 | 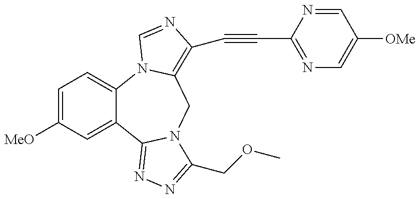 [SMILES: COCc1nnc2n1Cc1c(C#Cc3ncc(OC)cn3)ncn1-c1ccc(OC)cc1-2] |
| 594 | 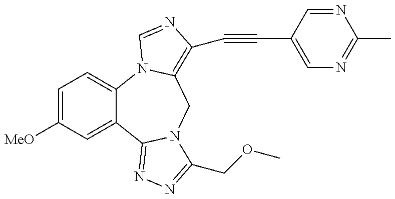 [SMILES: COCc1nnc2n1Cc1c(C#Cc3cnc(C)nc3)ncn1-c1ccc(OC)cc1-2] |
| 595 | 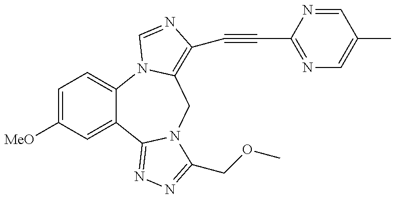 [SMILES: COCc1nnc2n1Cc1c(C#Cc3ncc(C)cn3)ncn1-c1ccc(OC)cc1-2] |
| 596 | 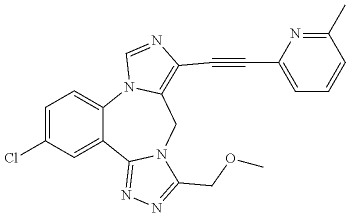 [SMILES: COCc1nnc2n1Cc1c(C#Cc3cccc(C)n3)ncn1-c1ccc(Cl)cc1-2] |
| 597 | 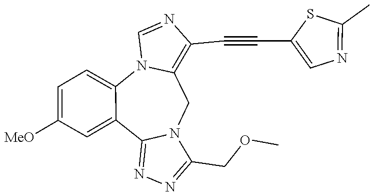 [SMILES: COCc1nnc2n1Cc1c(C#Cc3cnc(C)s3)ncn1-c1ccc(OC)cc1-2] |
| 598 | 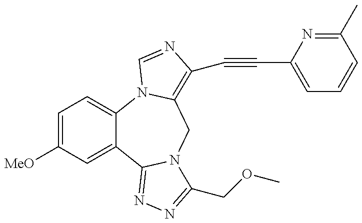 [SMILES: COCc1nnc2n1Cc1c(C#Cc3cccc(C)n3)ncn1-c1ccc(OC)cc1-2] |
| 599 | 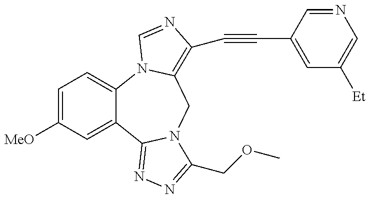 [SMILES: CCc1cncc(C#Cc2ncn3c2Cn2c(COC)nnc2-c2cc(OC)ccc2-3)c1] |
| 600 | 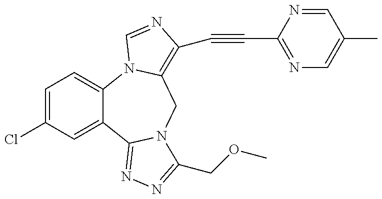 [SMILES: COCc1nnc2n1Cc1c(C#Cc3ncc(C)cn3)ncn1-c1ccc(Cl)cc1-2] |
| 601 | 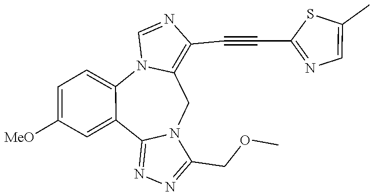 [SMILES: COCc1nnc2n1Cc1c(C#Cc3ncc(C)s3)ncn1-c1ccc(OC)cc1-2] |
| 602 | 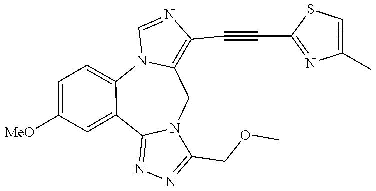 [SMILES: COCc1nnc2n1Cc1c(C#Cc3nc(C)cs3)ncn1-c1ccc(OC)cc1-2] |
| 603 | 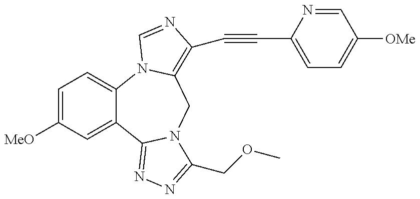 [SMILES: COCc1nnc2n1Cc1c(C#Cc3ccc(OC)cn3)ncn1-c1ccc(OC)cc1-2] |
| 604 | 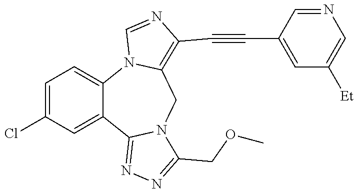 [SMILES: CCc1cncc(C#Cc2ncn3c2Cn2c(COC)nnc2-c2cc(Cl)ccc2-3)c1] |
| 605 | 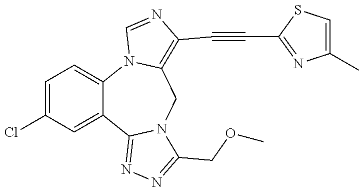 [SMILES: COCc1nnc2n1Cc1c(C#Cc3nc(C)cs3)ncn1-c1ccc(Cl)cc1-2] |
| 606 | 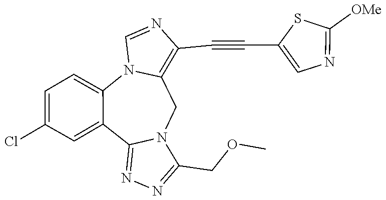 [SMILES: COCc1nnc2n1Cc1c(C#Cc3cnc(OC)s3)ncn1-c1ccc(Cl)cc1-2] |
| 607 | 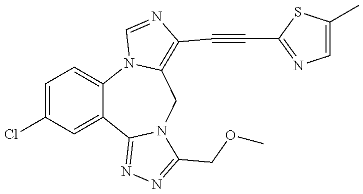 [SMILES: COCc1nnc2n1Cc1c(C#Cc3ncc(C)s3)ncn1-c1ccc(Cl)cc1-2] |
| 608 | 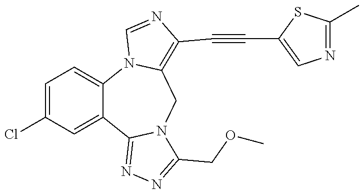 [SMILES: COCc1nnc2n1Cc1c(C#Cc3cnc(C)s3)ncn1-c1ccc(Cl)cc1-2] |
| 609 | 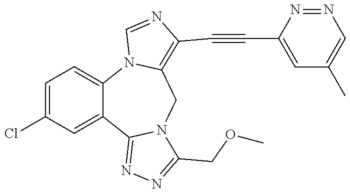 [SMILES: COCc1nnc2n1Cc1c(C#Cc3cc(C)cnn3)ncn1-c1ccc(Cl)cc1-2] |
| 610 | 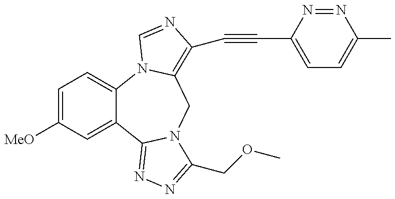 [SMILES: COCc1nnc2n1Cc1c(C#Cc3ccc(C)nn3)ncn1-c1ccc(OC)cc1-2] |
| 611 | 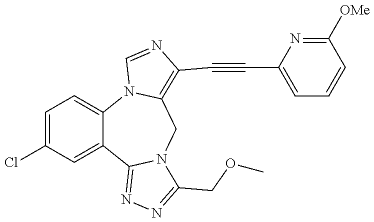 [SMILES: COCc1nnc2n1Cc1c(C#Cc3cccc(OC)n3)ncn1-c1ccc(Cl)cc1-2] |
| 612 | 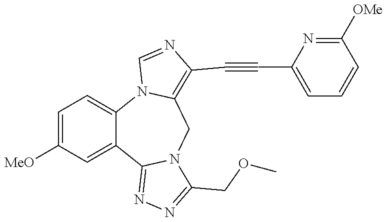 [SMILES: COCc1nnc2n1Cc1c(C#Cc3cccc(OC)n3)ncn1-c1ccc(OC)cc1-2] |
| 613 | 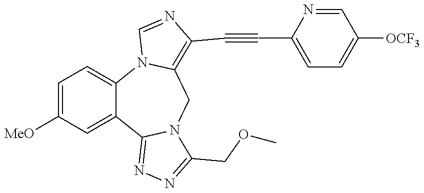 [SMILES: COCc1nnc2n1Cc1c(C#Cc3ccc(OC(F)(F)F)cn3)ncn1-c1ccc(OC)cc1-2] |
| 614 | 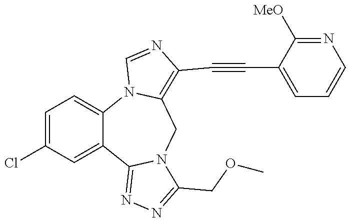 [SMILES: COCc1nnc2n1Cc1c(C#Cc3cccnc3OC)ncn1-c1ccc(Cl)cc1-2] |
| 615 | 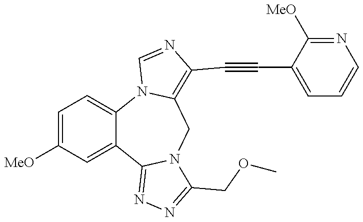 [SMILES: COCc1nnc2n1Cc1c(C#Cc3cccnc3OC)ncn1-c1ccc(OC)cc1-2] |
| 616 | 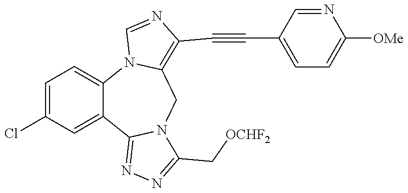 [SMILES: COc1ccc(C#Cc2ncn3c2Cn2c(COC(F)F)nnc2-c2cc(Cl)ccc2-3)cn1] |
| 617 | 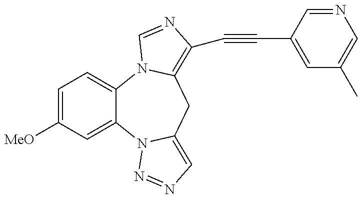 [SMILES: COc1ccc2c(c1)-n1nncc1Cc1c(C#Cc3cncc(C)c3)ncn1-2] |
| 618 | 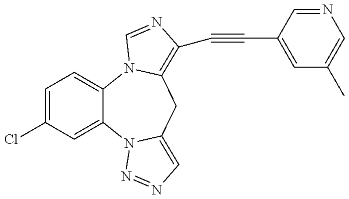 [SMILES: Cc1cncc(C#Cc2ncn3c2Cc2cnnn2-c2cc(Cl)ccc2-3)c1] |
| 619 | 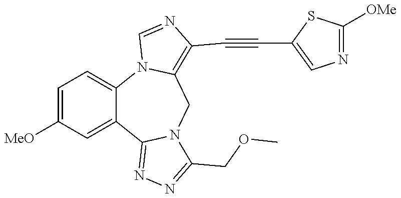 [SMILES: COCc1nnc2n1Cc1c(C#Cc3cnc(OC)s3)ncn1-c1ccc(OC)cc1-2] |
| 620 | 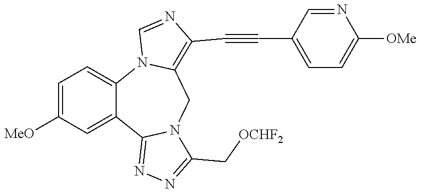 [SMILES: COc1ccc2c(c1)-c1nnc(COC(F)F)n1Cc1c(C#Cc3ccc(OC)nc3)ncn1-2] |
| 621 | 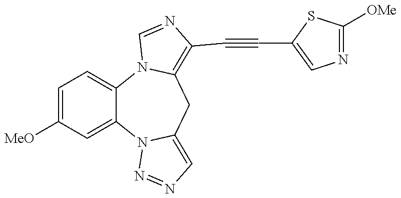 [SMILES: COc1ccc2c(c1)-n1nncc1Cc1c(C#Cc3cnc(OC)s3)ncn1-2] |
| 622 | 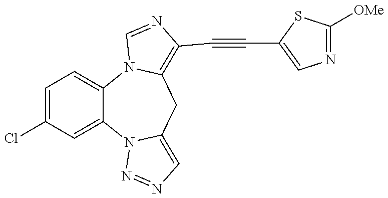 [SMILES: COc1ncc(C#Cc2ncn3c2Cc2cnnn2-c2cc(Cl)ccc2-3)s1] |
| 623 | 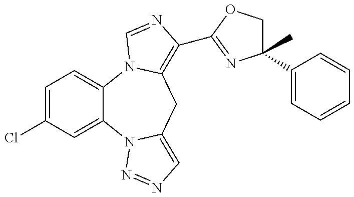 [SMILES: C[C@]1(c2ccccc2)COC(c2ncn3c2Cc2cnnn2-c2cc(Cl)ccc2-3)=N1] |
| 624 | 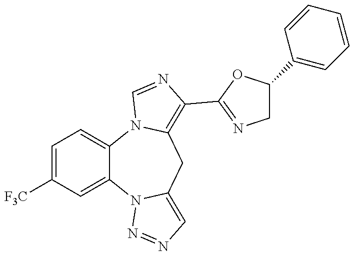 [SMILES: FC(F)(F)c1ccc2c(c1)-n1nncc1Cc1c(C3=NC[C@@H](c4ccccc4)O3)ncn1-2] |
| 625 | 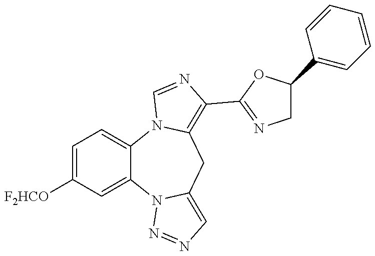 [SMILES: FC(F)Oc1ccc2c(c1)-n1nncc1Cc1c(C3=NC[C@H](c4ccccc4)O3)ncn1-2] |
| 626 | 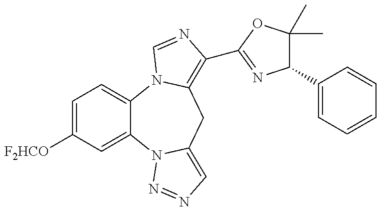 [SMILES: CC1(C)OC(c2ncn3c2Cc2cnnn2-c2cc(OC(F)F)ccc2-3)=N[C@H]1c1ccccc1] |
| 627 | 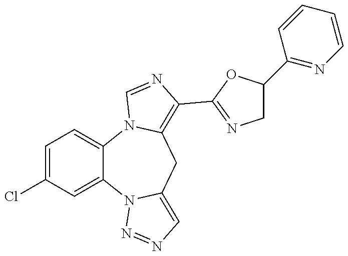 [SMILES: Clc1ccc2c(c1)-n1nncc1Cc1c(C3=NCC(c4ccccn4)O3)ncn1-2] |
| 628 | 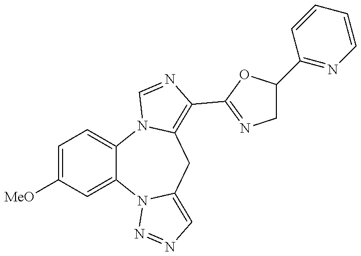 [SMILES: COc1ccc2c(c1)-n1nncc1Cc1c(C3=NCC(c4ccccn4)O3)ncn1-2] |
| 629 | 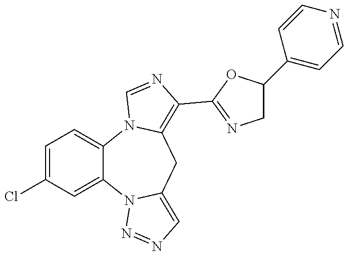 [SMILES: Clc1ccc2c(c1)-n1nncc1Cc1c(C3=NCC(c4ccncc4)O3)ncn1-2] |
| 630 | 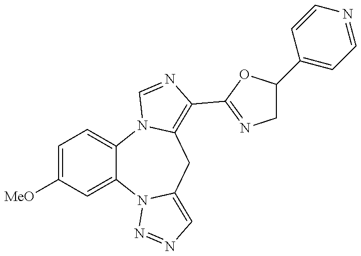 [SMILES: COc1ccc2c(c1)-n1nncc1Cc1c(C3=NCC(c4ccncc4)O3)ncn1-2] |
| 631 | 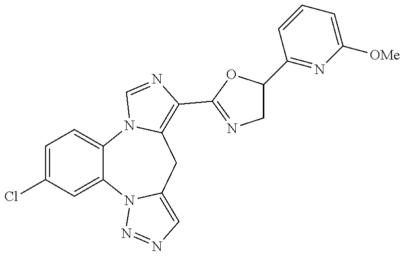 [SMILES: COc1cccc(C2CN=C(c3ncn4c3Cc3cnnn3-c3cc(Cl)ccc3-4)O2)n1] |
| 632 | 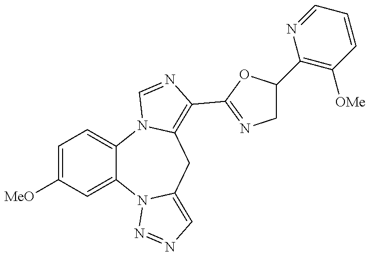 [SMILES: COc1ccc2c(c1)-n1nncc1Cc1c(C3=NCC(c4ncccc4OC)O3)ncn1-2] |
| 633 | 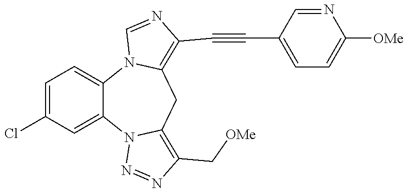 [SMILES: COCc1nnn2c1Cc1c(C#Cc3ccc(OC)nc3)ncn1-c1ccc(Cl)cc1-2] |
| 634 | 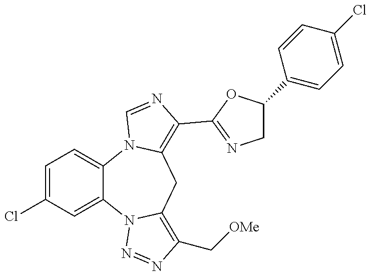 [SMILES: COCc1nnn2c1Cc1c(C3=NC[C@@H](c4ccc(Cl)cc4)O3)ncn1-c1ccc(Cl)cc1-2] |
| 635 | 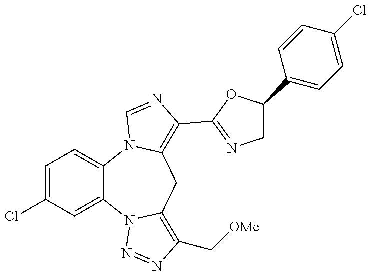 [SMILES: COCc1nnn2c1Cc1c(C3=NC[C@H](c4ccc(Cl)cc4)O3)ncn1-c1ccc(Cl)cc1-2] |
| 636 | 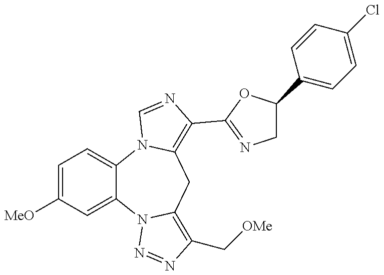 [SMILES: COCc1nnn2c1Cc1c(C3=NC[C@H](c4ccc(Cl)cc4)O3)ncn1-c1ccc(OC)cc1-2] |
| 637 | 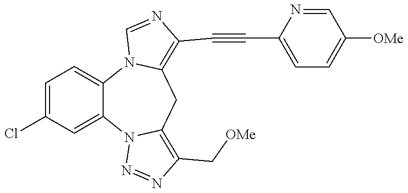 [SMILES: COCc1nnn2c1Cc1c(C#Cc3ccc(OC)cn3)ncn1-c1ccc(Cl)cc1-2] |
| 638 | 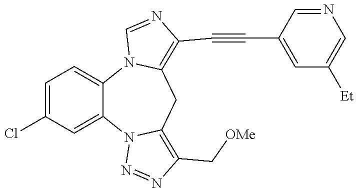 [SMILES: CCc1cncc(C#Cc2ncn3c2Cc2c(COC)nnn2-c2cc(Cl)ccc2-3)c1] |
| 639 | 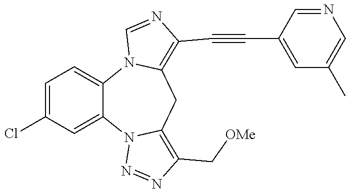 [SMILES: COCc1nnn2c1Cc1c(C#Cc3cncc(C)c3)ncn1-c1ccc(Cl)cc1-2] |
| 640 | 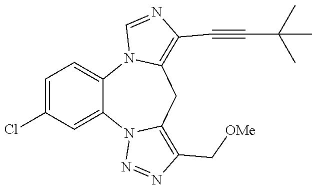 [SMILES: COCc1nnn2c1Cc1c(C#CC(C)(C)C)ncn1-c1ccc(Cl)cc1-2] |
| 641 | 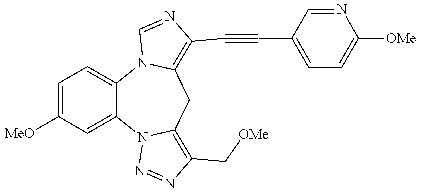 [SMILES: COCc1nnn2c1Cc1c(C#Cc3ccc(OC)nc3)ncn1-c1ccc(OC)cc1-2] |
| 642 | 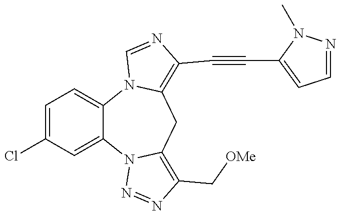 [SMILES: COCc1nnn2c1Cc1c(C#Cc3ccnn3C)ncn1-c1ccc(Cl)cc1-2] |
| 643 | 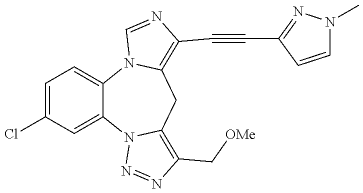 [SMILES: COCc1nnn2c1Cc1c(C#Cc3ccn(C)n3)ncn1-c1ccc(Cl)cc1-2] |
| 644 | 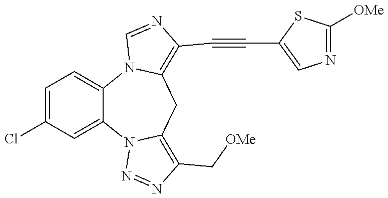 [SMILES: COCc1nnn2c1Cc1c(C#Cc3cnc(OC)s3)ncn1-c1ccc(Cl)cc1-2] |
| 646 | 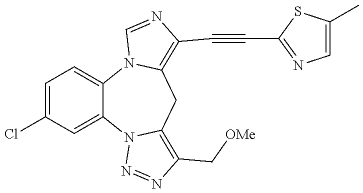 [SMILES: COCc1nnn2c1Cc1c(C#Cc3ncc(C)s3)ncn1-c1ccc(Cl)cc1-2] |
| 647 | 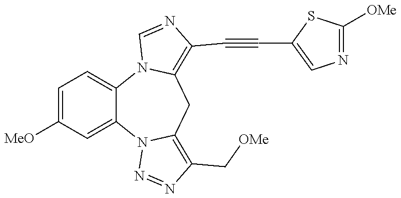 [SMILES: COCc1nnn2c1Cc1c(C#Cc3cnc(OC)s3)ncn1-c1ccc(OC)cc1-2] |
| 648 | 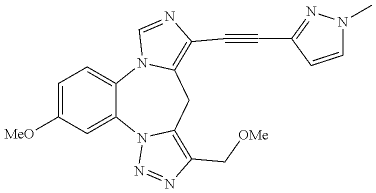 [SMILES: COCc1nnn2c1Cc1c(C#Cc3ccn(C)n3)ncn1-c1ccc(OC)cc1-2] |
| 649 | 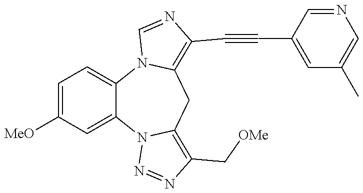 [SMILES: COCc1nnn2c1Cc1c(C#Cc3cncc(C)c3)ncn1-c1ccc(OC)cc1-2] |
| 650 | 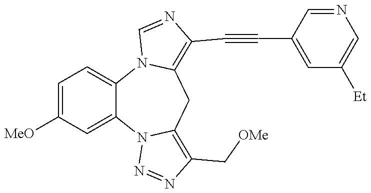 [SMILES: CCc1cncc(C#Cc2ncn3c2Cc2c(COC)nnn2-c2cc(OC)ccc2-3)c1] |
| 651 | 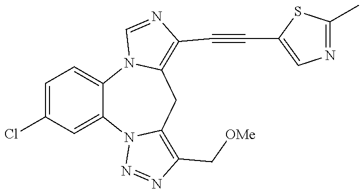 [SMILES: COCc1nnn2c1Cc1c(C#Cc3cnc(C)s3)ncn1-c1ccc(Cl)cc1-2] |
| 652 | 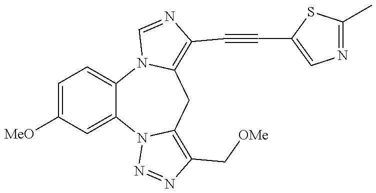 [SMILES: COCc1nnn2c1Cc1c(C#Cc3cnc(C)s3)ncn1-c1ccc(OC)cc1-2] |
| 653 | 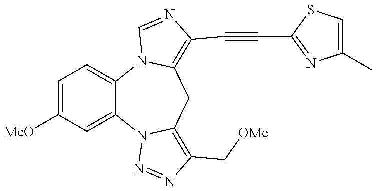 [SMILES: COCc1nnn2c1Cc1c(C#Cc3nc(C)cs3)ncn1-c1ccc(OC)cc1-2] |
| 654 | 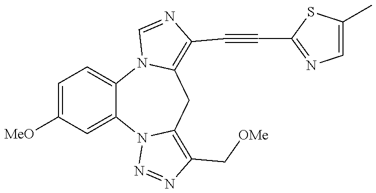 [SMILES: COCc1nnn2c1Cc1c(C#Cc3ncc(C)s3)ncn1-c1ccc(OC)cc1-2] |
| 655 | 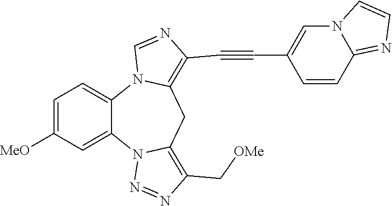 [SMILES: COCc1nnn2c1Cc1c(C#Cc3ccc4nccn4c3)ncn1-c1ccc(OC)cc1-2] |
| 656 | 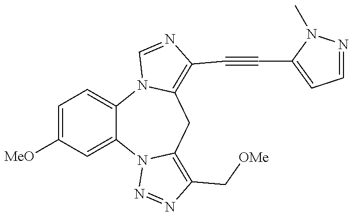 [SMILES: COCc1nnn2c1Cc1c(C#Cc3ccnn3C)ncn1-c1ccc(OC)cc1-2] |
| 657 | 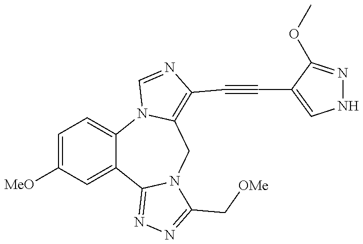 [SMILES: COCc1nnc2n1Cc1c(C#Cc3c[nH]nc3OC)ncn1-c1ccc(OC)cc1-2] |
| 658 | 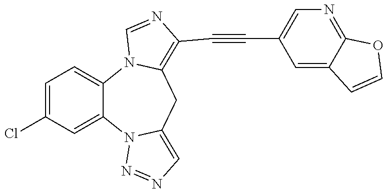 [SMILES: Clc1ccc2c(c1)-n1nncc1Cc1c(C#Cc3cnc4occc4c3)ncn1-2] |
| 659 | 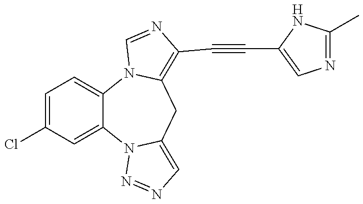 [SMILES: Cc1ncc(C#Cc2ncn3c2Cc2cnnn2-c2cc(Cl)ccc2-3)[nH]1] |
| 660 | 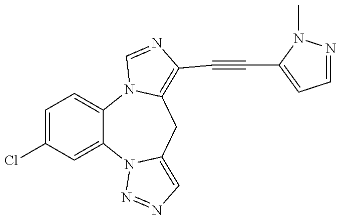 [SMILES: Cn1nccc1C#Cc1ncn2c1Cc1cnnn1-c1cc(Cl)ccc1-2] |
| 661 | 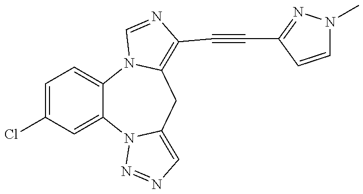 [SMILES: Cn1ccc(C#Cc2ncn3c2Cc2cnnn2-c2cc(Cl)ccc2-3)n1] |
| 662 | 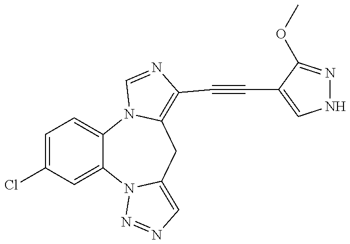 [SMILES: COc1n[nH]cc1C#Cc1ncn2c1Cc1cnnn1-c1cc(Cl)ccc1-2] |
| 663 | 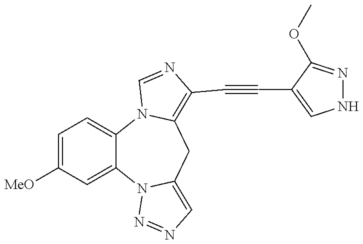 [SMILES: COc1ccc2c(c1)-n1nncc1Cc1c(C#Cc3c[nH]nc3OC)ncn1-2] |
| 664 | 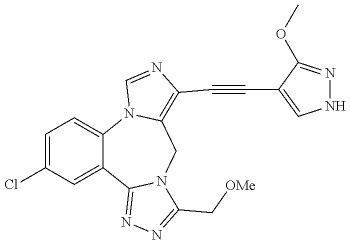 [SMILES: COCc1nnc2n1Cc1c(C#Cc3c[nH]nc3OC)ncn1-c1ccc(Cl)cc1-2] |
| 665 | 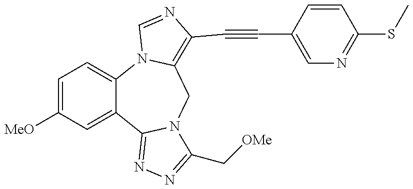 [SMILES: COCc1nnc2n1Cc1c(C#Cc3ccc(SC)nc3)ncn1-c1ccc(OC)cc1-2] |
| 666 | 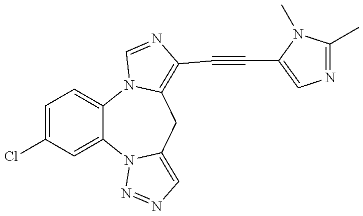 [SMILES: Cc1ncc(C#Cc2ncn3c2Cc2cnnn2-c2cc(Cl)ccc2-3)n1C] |
| 667 | 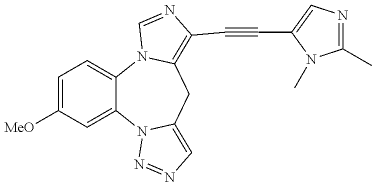 [SMILES: COc1ccc2c(c1)-n1nncc1Cc1c(C#Cc3cnc(C)n3C)ncn1-2] |
| 668 | 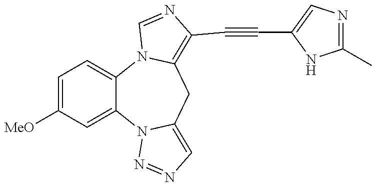 [SMILES: COc1ccc2c(c1)-n1nncc1Cc1c(C#Cc3cnc(C)[nH]3)ncn1-2] |
| 669 | 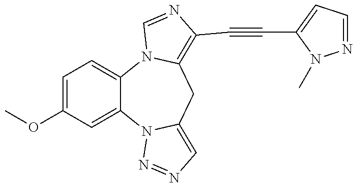 [SMILES: COc1ccc2c(c1)-n1nncc1Cc1c(C#Cc3ccnn3C)ncn1-2] |
| 670 | 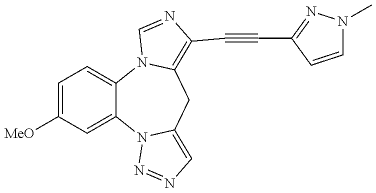 [SMILES: COc1ccc2c(c1)-n1nncc1Cc1c(C#Cc3ccn(C)n3)ncn1-2] |
| 671 | 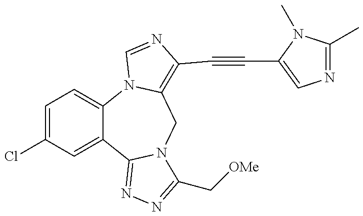 [SMILES: COCc1nnc2n1Cc1c(C#Cc3cnc(C)n3C)ncn1-c1ccc(Cl)cc1-2] |
| 672 | 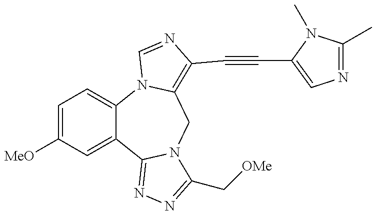 [SMILES: COCc1nnc2n1Cc1c(C#Cc3cnc(C)n3C)ncn1-c1ccc(OC)cc1-2] |
| 673 | 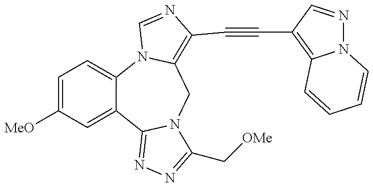 [SMILES: COCc1nnc2n1Cc1c(C#Cc3cnn4ccccc34)ncn1-c1ccc(OC)cc1-2] |
| 674 | 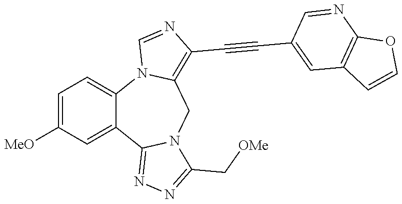 [SMILES: COCc1nnc2n1Cc1c(C#Cc3cnc4occc4c3)ncn1-c1ccc(OC)cc1-2] |
| 675 | 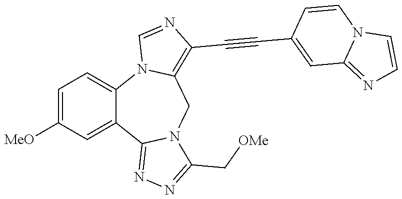 [SMILES: COCc1nnc2n1Cc1c(C#Cc3ccn4ccnc4c3)ncn1-c1ccc(OC)cc1-2] |
| 676 | 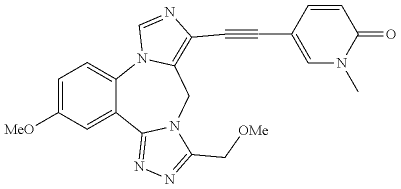 [SMILES: COCc1nnc2n1Cc1c(C#Cc3ccc(=O)n(C)c3)ncn1-c1ccc(OC)cc1-2] |
| 677 | 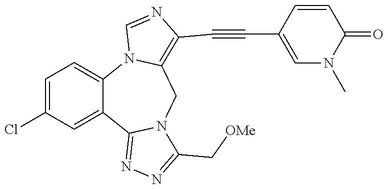 [SMILES: COCc1nnc2n1Cc1c(C#Cc3ccc(=O)n(C)c3)ncn1-c1ccc(Cl)cc1-2] |
| 678 | 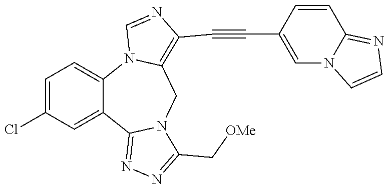 [SMILES: COCc1nnc2n1Cc1c(C#Cc3ccc4nccn4c3)ncn1-c1ccc(Cl)cc1-2] |
| 679 | 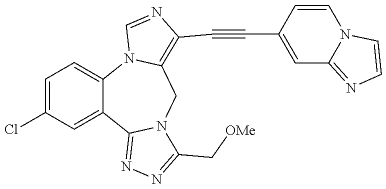 [SMILES: COCc1nnc2n1Cc1c(C#Cc3ccn4ccnc4c3)ncn1-c1ccc(Cl)cc1-2] |
| 680 | 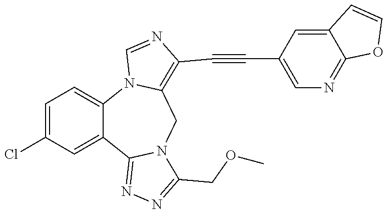 [SMILES: COCc1nnc2n1Cc1c(C#Cc3cnc4occc4c3)ncn1-c1ccc(Cl)cc1-2] |
| 681 | 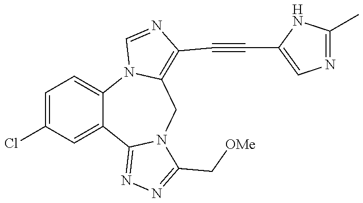 [SMILES: COCc1nnc2n1Cc1c(C#Cc3cnc(C)[nH]3)ncn1-c1ccc(Cl)cc1-2] |
| 682 | 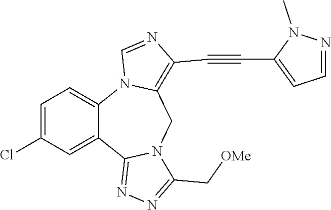 [SMILES: COCc1nnc2n1Cc1c(C#Cc3ccnn3C)ncn1-c1ccc(Cl)cc1-2] |
| 683 | 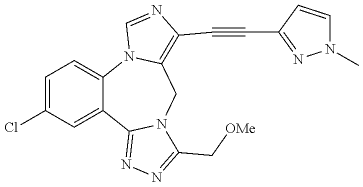 [SMILES: COCc1nnc2n1Cc1c(C#Cc3ccn(C)n3)ncn1-c1ccc(Cl)cc1-2] |
| 684 | 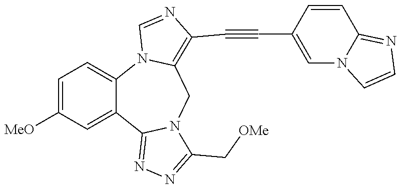 [SMILES: COCc1nnc2n1Cc1c(C#Cc3ccc4nccn4c3)ncn1-c1ccc(OC)cc1-2] |
| 685 | 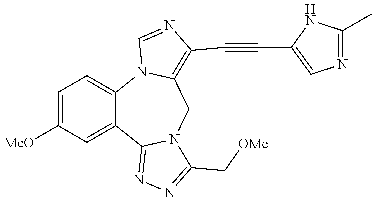 [SMILES: COCc1nnc2n1Cc1c(C#Cc3cnc(C)[nH]3)ncn1-c1ccc(OC)cc1-2] |
| 686 | 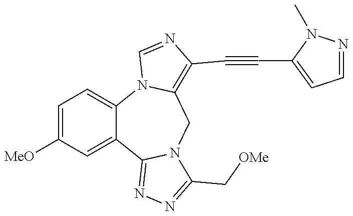 [SMILES: COCc1nnc2n1Cc1c(C#Cc3ccnn3C)ncn1-c1ccc(OC)cc1-2] |
| 687 | 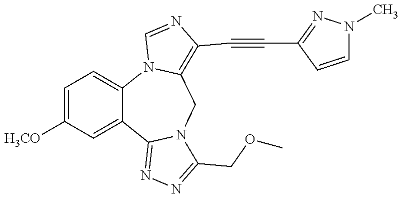 [SMILES: COCc1nnc2n1Cc1c(C#Cc3ccn(C)n3)ncn1-c1ccc(OC)cc1-2] |
| 689 | 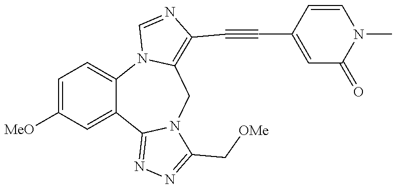 [SMILES: COCc1nnc2n1Cc1c(C#Cc3ccn(C)c(=O)c3)ncn1-c1ccc(OC)cc1-2] |
| 690 | 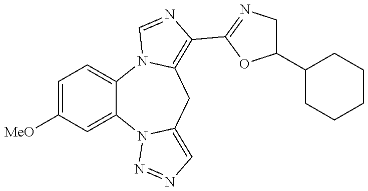 [SMILES: COc1ccc2c(c1)-n1nncc1Cc1c(C3=NCC(C4CCCCC4)O3)ncn1-2] |
| 691 | 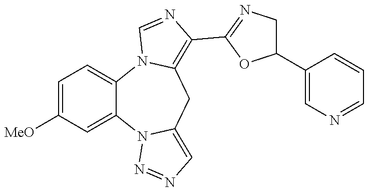 [SMILES: COc1ccc2c(c1)-n1nncc1Cc1c(C3=NCC(c4cccnc4)O3)ncn1-2] |
| 692 | 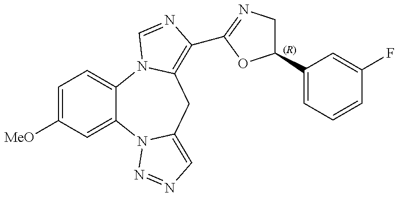 |
| 693 | 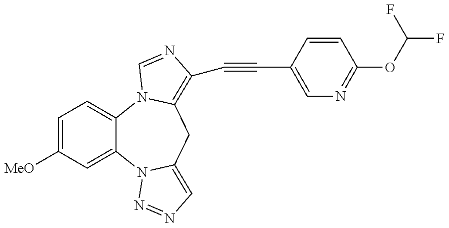 [SMILES: COc1ccc2c(c1)-n1nncc1Cc1c(C#Cc3ccc(OC(F)F)nc3)ncn1-2] |
| 694 | 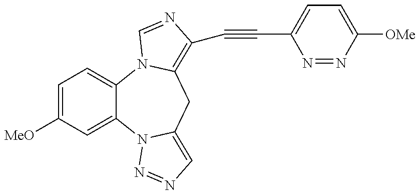 [SMILES: COc1ccc2c(c1)-n1nncc1Cc1c(C#Cc3ccc(OC)nn3)ncn1-2] |
| 695 | 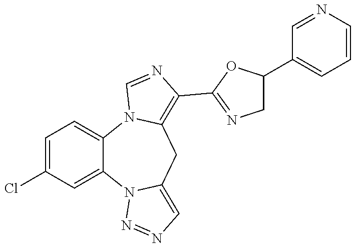 [SMILES: Clc1ccc2c(c1)-n1nncc1Cc1c(C3=NCC(c4cccnc4)O3)ncn1-2] |
| 696 | 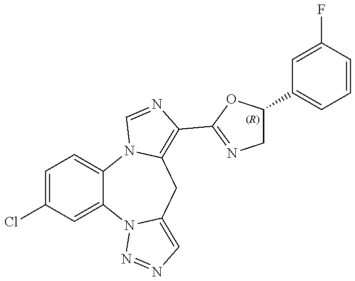 |
| 697 | 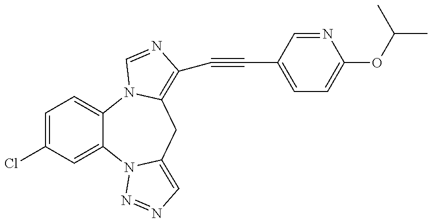 [SMILES: CC(C)Oc1ccc(C#Cc2ncn3c2Cc2cnnn2-c2cc(Cl)ccc2-3)cn1] |
| 698 | 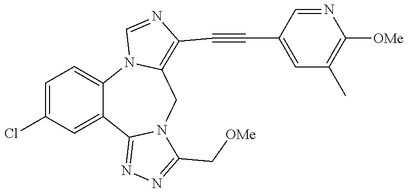 [SMILES: COCc1nnc2n1Cc1c(C#Cc3cnc(OC)c(C)c3)ncn1-c1ccc(Cl)cc1-2] |
| 700 | 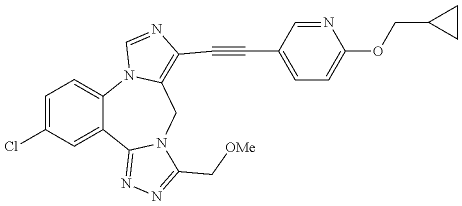 [SMILES: COCc1nnc2n1Cc1c(C#Cc3ccc(OCC4CC4)nc3)ncn1-c1ccc(Cl)cc1-2] |
| 701 | 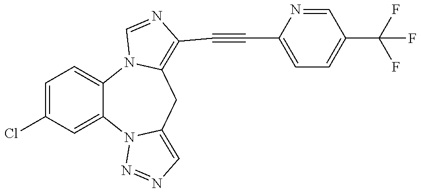 [SMILES: FC(F)(F)c1ccc(C#Cc2ncn3c2Cc2cnnn2-c2cc(Cl)ccc2-3)nc1] |
| 702 | 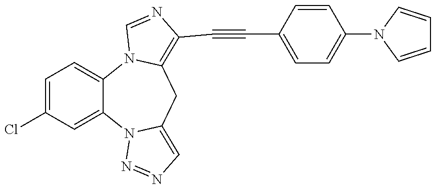 [SMILES: Clc1ccc2c(c1)-n1nncc1Cc1c(C#Cc3ccc(-n4cccc4)cc3)ncn1-2] |
| 703 | 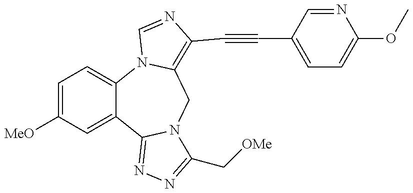 [SMILES: COCc1nnc2n1Cc1c(C#Cc3ccc(OC)nc3)ncn1-c1ccc(OC)cc1-2] |
| 705 | 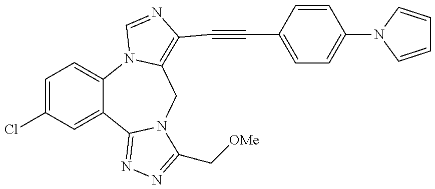 [SMILES: COCc1nnc2n1Cc1c(C#Cc3ccc(-n4cccc4)cc3)ncn1-c1ccc(Cl)cc1-2] |
| 707 | 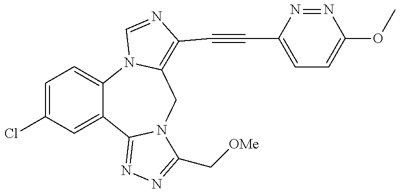 [SMILES: COCc1nnc2n1Cc1c(C#Cc3ccc(OC)nn3)ncn1-c1ccc(Cl)cc1-2] |
| 708 | 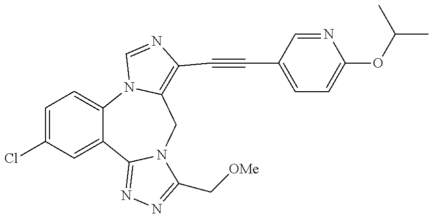 [SMILES: COCc1nnc2n1Cc1c(C#Cc3ccc(OC(C)C)nc3)ncn1-c1ccc(Cl)cc1-2] |
| 709 | 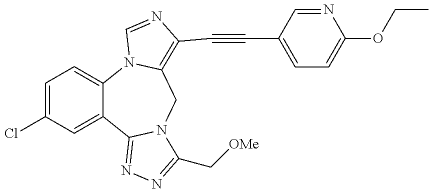 [SMILES: CCOc1ccc(C#Cc2ncn3c2Cn2c(COC)nnc2-c2cc(Cl)ccc2-3)cn1] |
| 710 | 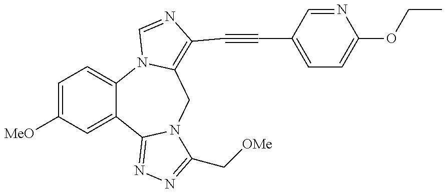 [SMILES: CCOc1ccc(C#Cc2ncn3c2Cn2c(COC)nnc2-c2cc(OC)ccc2-3)cn1] |
| 711 | 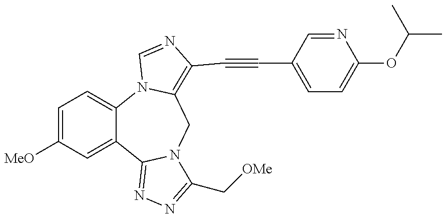 [SMILES: COCc1nnc2n1Cc1c(C#Cc3ccc(OC(C)C)nc3)ncn1-c1ccc(OC)cc1-2] |
| 712 | 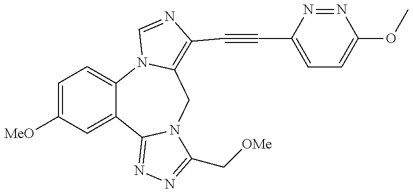 [SMILES: COCc1nnc2n1Cc1c(C#Cc3ccc(OC)nn3)ncn1-c1ccc(OC)cc1-2] |
| 713 | 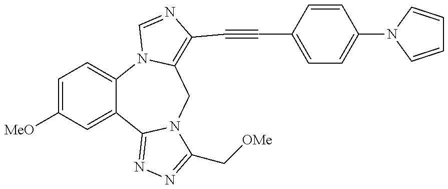 [SMILES: COCc1nnc2n1Cc1c(C#Cc3ccc(-n4cccc4)cc3)ncn1-c1ccc(OC)cc1-2] |
| 714 | 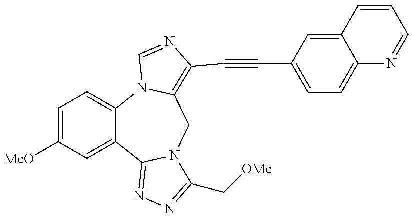 [SMILES: COCc1nnc2n1Cc1c(C#Cc3ccc4ncccc4c3)ncn1-c1ccc(OC)cc1-2] |
| 715 | 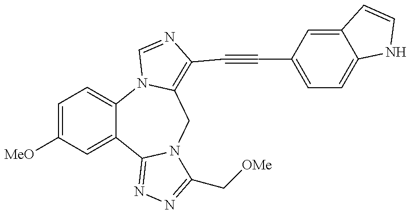 [SMILES: COCc1nnc2n1Cc1c(C#Cc3ccc4[nH]ccc4c3)ncn1-c1ccc(OC)cc1-2] |
| 716 | 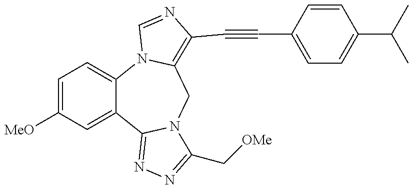 [SMILES: COCc1nnc2n1Cc1c(C#Cc3ccc(C(C)C)cc3)ncn1-c1ccc(OC)cc1-2] |
| 717 | 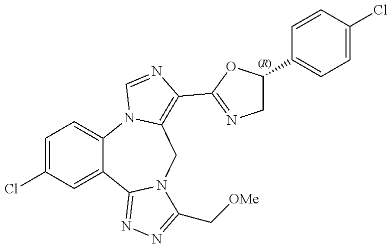 |
| 718 | 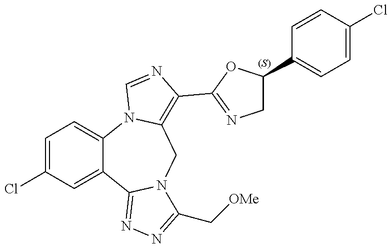 |
| 719 | 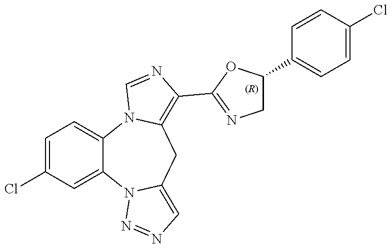 |
| 720 | 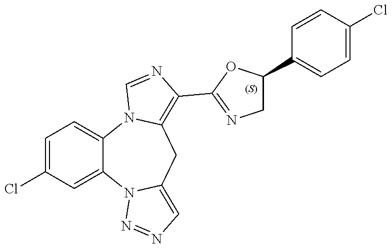 |
| 721 | 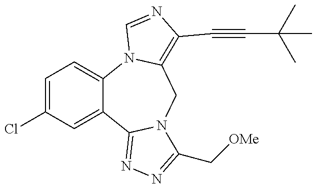 [SMILES: COCc1nnc2n1Cc1c(C#CC(C)(C)C)ncn1-c1ccc(Cl)cc1-2] |
| 723 | 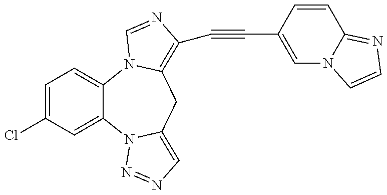 [SMILES: Clc1ccc2c(c1)-n1nncc1Cc1c(C#Cc3ccc4nccn4c3)ncn1-2] |
| 724 | 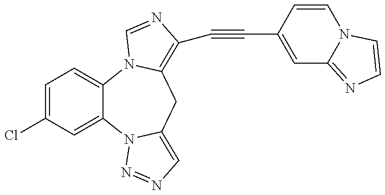 [SMILES: Clc1ccc2c(c1)-n1nncc1Cc1c(C#Cc3ccn4ccnc4c3)ncn1-2] |
| 725 | 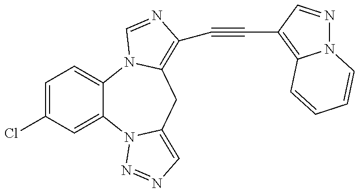 [SMILES: Clc1ccc2c(c1)-n1nncc1Cc1c(C#Cc3cnn4ccccc34)ncn1-2] |
| 726 | 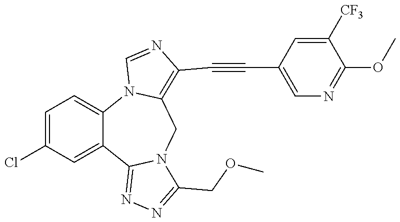 [SMILES: COCc1nnc2n1Cc1c(C#Cc3cnc(OC)c(C(F)(F)F)c3)ncn1-c1ccc(Cl)cc1-2] |
| 727 | 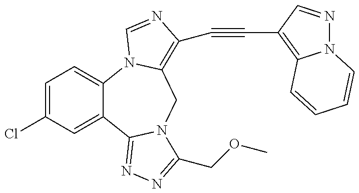 [SMILES: COCc1nnc2n1Cc1c(C#Cc3cnn4ccccc34)ncn1-c1ccc(Cl)cc1-2] |
| 728 | 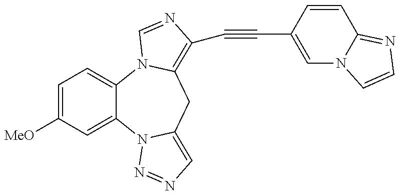 [SMILES: COc1ccc2c(c1)-n1nncc1Cc1c(C#Cc3ccc4nccn4c3)ncn1-2] |
| 729 | 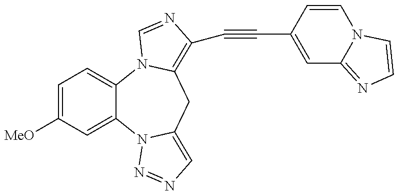 [SMILES: COc1ccc2c(c1)-n1nncc1Cc1c(C#Cc3ccn4ccnc4c3)ncn1-2] |
| 730 | 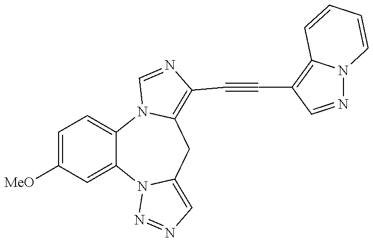 [SMILES: COc1ccc2c(c1)-n1nncc1Cc1c(C#Cc3cnn4ccccc34)ncn1-2] |
| 731 | 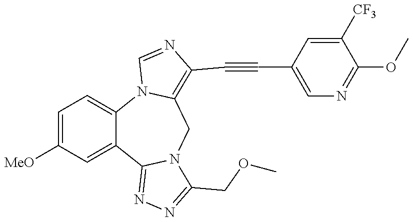 [SMILES: COCc1nnc2n1Cc1c(C#Cc3cnc(OC)c(C(F)(F)F)c3)ncn1-c1ccc(OC)cc1-2] |
| 732 | 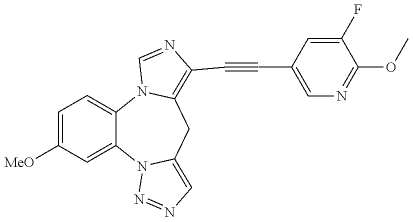 [SMILES: COc1ccc2c(c1)-n1nncc1Cc1c(C#Cc3cnc(OC)c(F)c3)ncn1-2] |
| 733 | 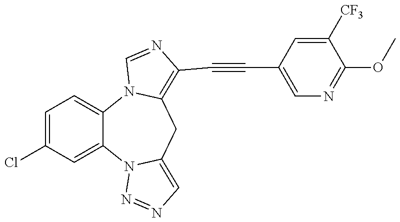 [SMILES: COc1ncc(C#Cc2ncn3c2Cc2cnnn2-c2cc(Cl)ccc2-3)cc1C(F)(F)F] |
| 734 | 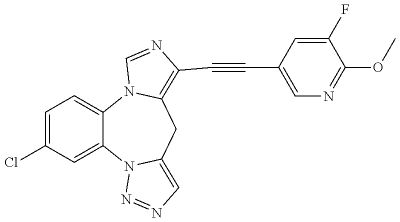 [SMILES: COc1ncc(C#Cc2ncn3c2Cc2cnnn2-c2cc(Cl)ccc2-3)cc1F] |
| 735 | 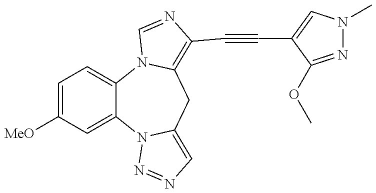 [SMILES: COc1ccc2c(c1)-n1nncc1Cc1c(C#Cc3cn(C)nc3OC)ncn1-2] |
| 736 | 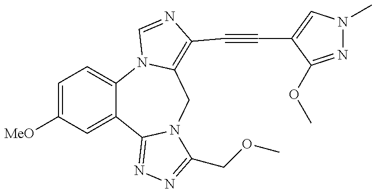 [SMILES: COCc1nnc2n1Cc1c(C#Cc3cn(C)nc3OC)ncn1-c1ccc(OC)cc1-2] |
| 737 | 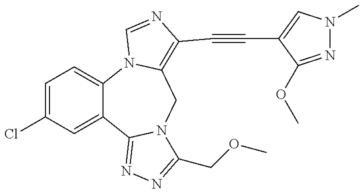 [SMILES: COCc1nnc2n1Cc1c(C#Cc3cn(C)nc3OC)ncn1-c1ccc(Cl)cc1-2] |
| 738 | 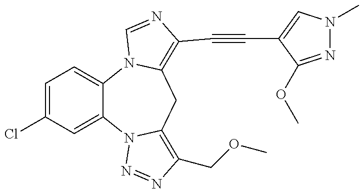 [SMILES: COCc1nnn2c1Cc1c(C#Cc3cn(C)nc3OC)ncn1-c1ccc(Cl)cc1-2] |
| 739 | 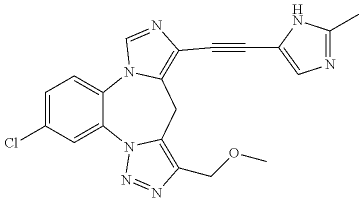 [SMILES: COCc1nnn2c1Cc1c(C#Cc3cnc(C)[nH]3)ncn1-c1ccc(Cl)cc1-2] |
| 740 | 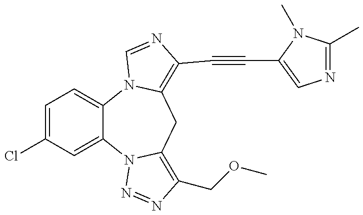 [SMILES: COCc1nnn2c1Cc1c(C#Cc3cnc(C)n3C)ncn1-c1ccc(Cl)cc1-2] |

and their pharmaceutically suitable salts, hydrates, solvates, polymorphs, isomers or combinations.

In some embodiments, the GABA_{A} α5 receptor agonist, or the pharmaceutically acceptable salt, hydrate, solvate, isomer, or polymorph thereof or the GABA_{A} α5 receptor agonist, or the pharmaceutically acceptable salt, hydrate, solvate, isomer, or polymorph thereof of the pharmaceutical composition of this disclosure is the compound having the structure

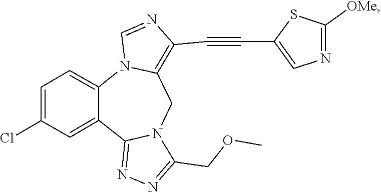 [SMILES: COCc1nnc2n1Cc1c(C#Cc3cnc(OC)s3)ncn1-c1ccc(Cl)cc1-2]

or a pharmaceutically acceptable salt, hydrate, solvate, polymorph, or isomer thereof.

In other embodiments, the GABA_{A} α5 receptor agonist, or the pharmaceutically acceptable salt, hydrate, solvate, isomer, or polymorph thereof or the GABA_{A} α5 receptor agonist, or the pharmaceutically acceptable salt, hydrate, solvate, isomer, or polymorph thereof of the pharmaceutical composition of this disclosure is a polymorph crystalline form of the compound having the structure

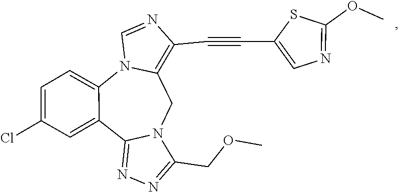 [SMILES: COCc1nnc2n1Cc1c(C#Cc3cnc(OC)s3)ncn1-c1ccc(Cl)cc1-2]

wherein the polymorph crystalline form is Form A and exhibits an XRPD comprising:
a. at least one peak selected from 3.0, and 21.0 degrees 2θ±0.2 degrees 2θ; and
b. at least one additional peak selected from the group consisting of 9.1, 10.7, 13.8, 22.0, 23.1, 23.9, 24.4, and 27.1 degrees 2θ±0.2 degrees 2θ. See, pages 292-295 of WO2022011318, incorporated herein by reference herein in its entirety and in particular in the context of the recited compound, its synthesis and properties.

In some embodiments, the GABA_{A} α5 receptor agonist, or the pharmaceutically acceptable salt, hydrate, solvate, isomer, or polymorph thereof or the GABA_{A} α5 receptor agonist, or the pharmaceutically acceptable salt, hydrate, solvate, isomer, or polymorph thereof of the pharmaceutical composition of this disclosure is a polymorph crystalline form of the compound having the structure

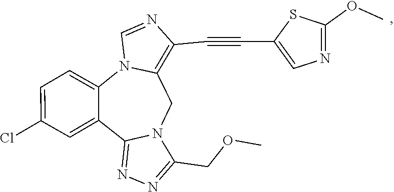 [SMILES: COCc1nnc2n1Cc1c(C#Cc3cnc(OC)s3)ncn1-c1ccc(Cl)cc1-2]

wherein the crystalline form is Form B and exhibits an XRPD comprising:
a. at least one peak selected from 13.0 and 15.3 degrees 2θ±0.2 degrees 2θ; and
b. at least one additional peak selected from the group consisting of 7.0, 9.3, 10.2, 10.4, 12.5, 13.6, 14.0, 22.0, 23.0, 23.6, and 27.3 degrees 2θ±0.2 degrees 2θ. See, pages 295-297 of WO2022011318, incorporated herein by reference herein in its entirety and in particular in the context of the recited compound, its synthesis and properties.

In some embodiments, the GABA_{A} α5 receptor agonist, or the pharmaceutically acceptable salt, hydrate, solvate, isomer, or polymorph thereof or the GABA_{A} α5 receptor agonist, or the pharmaceutically acceptable salt, hydrate, solvate, isomer, or polymorph thereof of the pharmaceutical composition of this disclosure is a solvate crystalline form of the compound having the structure

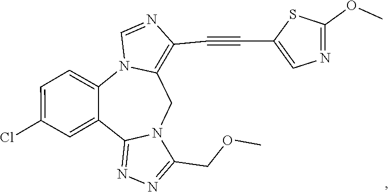 [SMILES: COCc1nnc2n1Cc1c(C#Cc3cnc(OC)s3)ncn1-c1ccc(Cl)cc1-2]

wherein the solvate crystalline form is Form C and exhibits an XRPD comprising:
a. at least one peak selected from 8.5 and 18.9 degrees 2θ±0.2 degrees 2θ; and
b. at least one additional peak selected from the group consisting of 7.1, 9.4, 10.3, 12.3, 12.5, 14.2, 20.7, 22.1, 23.2, 23.7, 24.0, and 26.4 degrees 2θ±0.2 degrees 2θ. See pages 302-305 of WO2022011318, incorporated herein by reference herein in its entirety and in particular in the context of the recited compound, its synthesis and properties.

In some embodiments, the GABA_{A} α5 receptor agonist, or the pharmaceutically acceptable salt, hydrate, solvate, isomer, or polymorph thereof or the GABA_{A} α5 receptor agonist, or the pharmaceutically acceptable salt, hydrate, solvate, isomer, or polymorph thereof of the pharmaceutical composition of this disclosure is a polymorph crystalline form of the compound having the structure

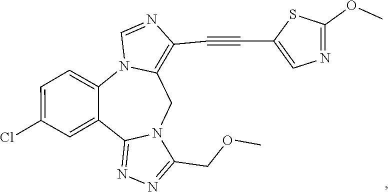 [SMILES: COCc1nnc2n1Cc1c(C#Cc3cnc(OC)s3)ncn1-c1ccc(Cl)cc1-2]

wherein the polymorph crystalline form is Form E and exhibits an XRPD comprising:
a. at least one peak selected from the group consisting of 11.4, 18.1, and 21.6 degrees 2θ±0.2 degrees 2θ; and
b. at least one additional peak selected from the group consisting of 7.2, 22.0, 23.0, 24.2, 25.0, and 26.6 degrees 2θ±0.2 degrees 2θ. See, pages 297-300 of WO2022011318, incorporated herein by reference herein in its entirety and in particular in the context of the recited compound, its synthesis and properties.

In some embodiments, the GABA_{A} α5 receptor agonist, or the pharmaceutically acceptable salt, hydrate, solvate, isomer, or polymorph thereof or the GABA_{A} α5 receptor agonist, or the pharmaceutically acceptable salt, hydrate, solvate, isomer, or polymorph thereof of the pharmaceutical composition of this disclosure is a hydrate crystalline form of the compound having the structure

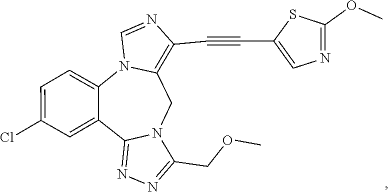 [SMILES: COCc1nnc2n1Cc1c(C#Cc3cnc(OC)s3)ncn1-c1ccc(Cl)cc1-2]

wherein the hydrate crystalline form is Form F and exhibits an XRPD comprising:
a. at least one peak selected from the group consisting of 9.9, 11.9, 17.3, 19.4, and 25.7 degrees 2θ±0.2 degrees 2θ; and
b. at least one additional peak selected from the group consisting of 9.7, 12.1, 20.8, 23.2, 23.7, 24.2, 25.0, and 26.4 degrees 2θ±0.2 degrees 2θ. See, pages 300-302 of WO2022011318, incorporated herein by reference herein in its entirety and in particular in the context of the recited compound, its synthesis and properties.

In some embodiments, the GABA_{A} α5 receptor agonist, or the pharmaceutically acceptable salt, hydrate, solvate, isomer, or polymorph thereof or the GABA_{A} α5 receptor agonist, or the pharmaceutically acceptable salt, hydrate, solvate, isomer, or polymorph thereof of the pharmaceutical composition of this disclosure, is selected from the group consisting of:
i) a compound of formula I-a:

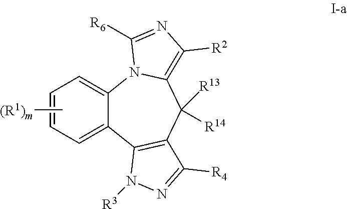

or a pharmaceutically acceptable salt, hydrate, solvate, polymorph, isomer, or combination thereof,
wherein:
m is an integer selected from 0-4;
each R¹ is independently selected from: halogen, —(C6-C10) aryl, —O(C1-C6)alkyl, —CN, —CHF₂, —CF₃, —OCF₃, —OCHF₂, CO(O)R₇, CH₂—OR⁸, —(C1-C6)alkyl-(C6-C10) aryl, -5-10 membered heteroaryl, —(C1-C6)alkyl-5-10 membered heteroaryl and —(C3-C6) cycloalkyl;
each R⁸ is independently selected from —H or —(C1-C6 alkyl)
each R² is selected from CO(O)R₇, C≡C—R⁹, —(C1-C6)alkyl-C≡C—R⁹, -(5-10 membered) heteroaryl, -(3-10 membered) heterocyclyl, (C3-C10)-cycloalkenyl

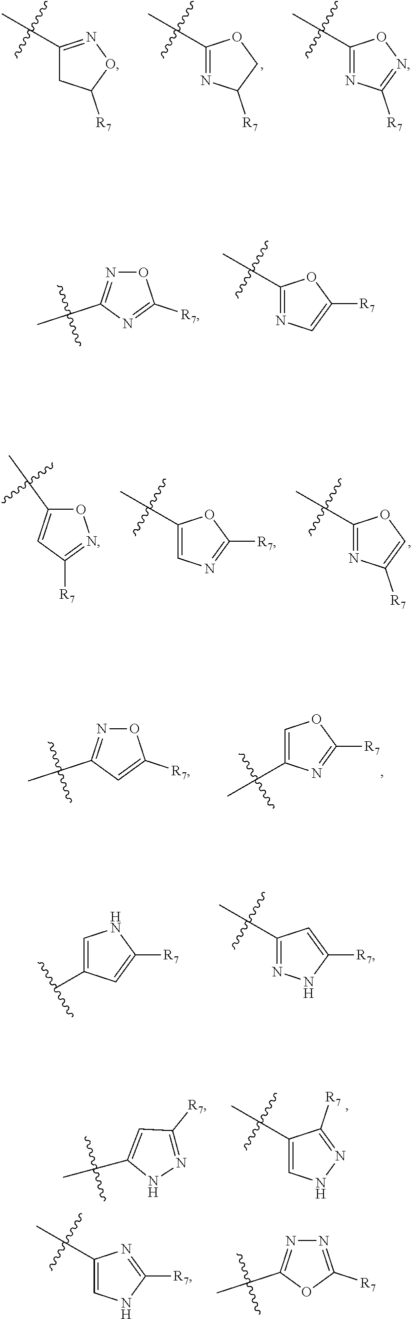

wherein each 5-6-membered heteroaryl and 3-10-membered heterocycle is substituted with 0-4 R₇;
each R⁹ is selected from: —H, —(C1-C6)alkyl, (5- to 10-membered heteroaryl), -(3-10 membered) heterocyclyl, —(C6-C10) aryl, —(C1-C6)alkyl-(C6-C10) aryl

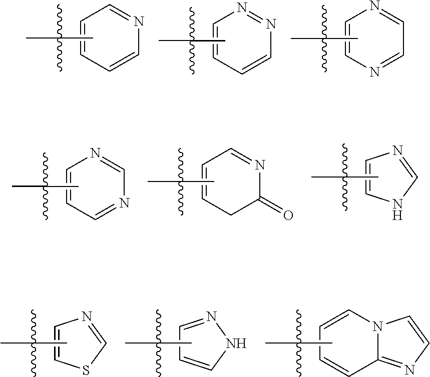

wherein each occurrence of R⁹ is independently substituted by 0-5 R¹¹;
wherein each occurrence of R¹¹ is independently selected from —(C1-C6)alkyl, —O—(C1-C6)alkyl, -halogen, —CF₃, —OCF₃, —(C6-C10) aryl and -5 to 10 membered heteroaryl;
R³ is independently selected from: —H, —(C1-C6)alkyl, -5 to 10 membered heteroaryl, -(3-10 membered) heterocyclyl, —(C1-C6)alkyl-(C3-C6) cycloalkyl and —(C1-C6)alkyl-(C6-C10) aryl, wherein R³ is independently substituted with 0-5 R¹²,
wherein each R¹² is independently selected from: —H, -halogen, —OR^{o}, R^{o}, oxo, —CH₂OR^{o}, —CH₂N(R^{o})₂, —C(O)N(R^{o})₂, —C(O)OR^{o}, —CF₃, —OCF₃ and —N(R^{o})₂, wherein each occurrence of R^{o} is independently selected from: —(C1-C6)-aliphatic, (C3-C6)-cycloalkyl, -(3-10 membered) heterocyclyl, and (C6-C10)-aryl;
wherein each R₇ is selected from —H, —CF₃, —(C1-C6)alkyl, —(C1-C6)alkyloxy, (C1-C6) alkylamino, —(C3-C6) cycloalkyl, -5 to 10 membered heteroaryl, —(C6-C10) aryl, —CH₂—(C6-C10) aryl, -5 to 10 membered heteroaryl-(C1-C6)alkyl, and -3-10 membered heterocyclyl, wherein each R₇ is independently substituted with 0-5 R′;
   or when two R₇ groups bound to the same atom, the two R₇ groups may be taken together with the atom to which they are bound to form a 3-10-membered aromatic or non-aromatic ring having 0-4 heteroatoms independently selected from N, NH, O, S, SO, and SO₂, wherein said ring is optionally substituted with 0-5 R′;
each occurrence of R′ is wherein each occurrence of R′ is independently selected from halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂, wherein R″ is selected from —Cl, —F, —(C1-C6)alkyl, —OMe, and —(C6-C10) aryl;
R″ is independently substituted with 1-3 substituents wherein the substituents are selected from: halogen, —CF₃, —OCF₃, —O—(C1-C6)-aliphatic or —(C1-C6)-aliphatic;
each R⁴ is selected from —H or —(C1-C6)alkyl;
each R⁶ is selected from —H or —(C1-C6)alkyl;
each R¹³ and R¹⁴ is independently selected from H—, (C1-C3)-aliphatic-, or (C3-C6)-cycloalkyl;
ii) a compound of formula I-b:

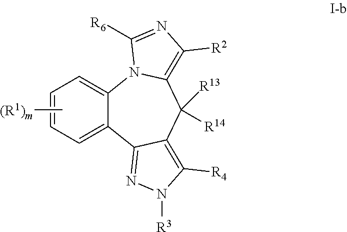

or a pharmaceutically acceptable salt, hydrate, solvate, polymorph, isomer, or combination thereof,
m is an integer selected from 0-4;
wherein:
each R¹ is independently selected from -halogen, —(C6-C10) aryl, —Ome, —CN, —CHF₂, —CF₃, —OCF₃, —OCHF₂, CO(O)R₇, CH₂—OR⁸, —(C1-C6)alkyl-(C6-C10) aryl, -5-10 membered heteroaryl, —(C1-C6)alkyl-5-10 membered heteroaryl and —(C3-C6) cycloalkyl;
each R⁸ is independently selected from —H or —(C1-C6 alkyl);
each R² is selected from CO(O)R₇, C≡C—R⁹, —(C1-C6)alkyl-C≡C—R⁹, -(5-10 membered) heteroaryl, -(3-10 membered) heterocyclyl,

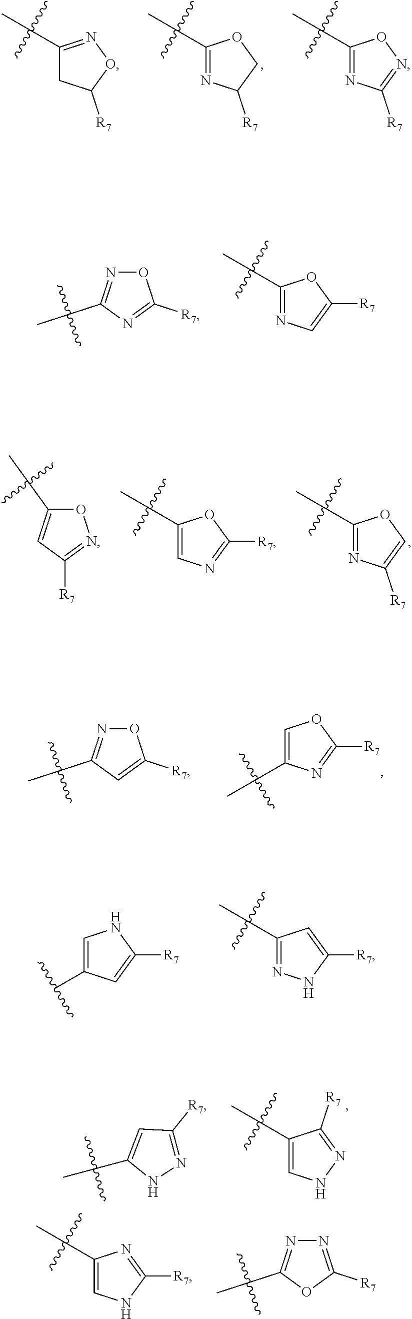

wherein each 5-6 membered heteroaryl or 3-10 membered heterocycle is substituted with 0-4 R₇;
each R⁹ is selected from: —H, —(C1-C6)alkyl, (5- to 10-membered heteroaryl), -(3-10 membered) heterocyclyl, —(C6-C10) aryl, —(C1-C6)alkyl-(C6-C10) aryl

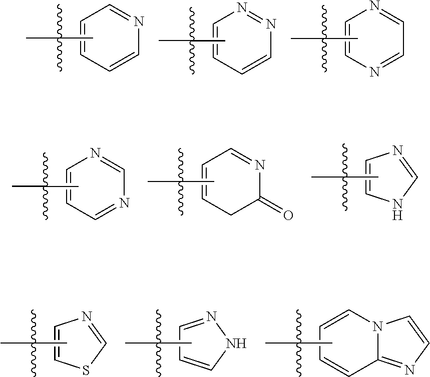

wherein each occurrence of R⁹ is independently substituted by 0-5 R¹¹
wherein each occurrence of R¹¹ is independently selected from —(C1-C6)alkyl, —O—(C1-C6)alkyl, -halogen, —CF₃, —OCF₃, —OMe, —(C6-C10) aryl and -5 to 10 membered heteroaryl;
R³ is independently selected from: —H, —(C1-C6)alkyl, —(C1-C6)alkyl-(C3-C6) cycloalkyl, —(C1-C6)alkyl-OR¹², —(C1-C6)alkyl-N(R¹²)₂, —(C1-C6)alkyl-(C6-C10) aryl, —(C1-C6) alkyl-5-10 membered heteroaryl, -3 to 10 membered heterocyclyl and -5-10 membered heteroaryl, wherein R³ is independently substituted with 0-5 R¹²;
wherein each R¹² is independently selected from: —H, -halogen, —OR^{o}, R^{o}, oxo, —CH₂OR^{o}, —CH₂N(R^{o})₂, —C(O)N(R^{o})₂, —C(O)OR^{o}, —NO₂, —NCS, —CN, —CF₃, —OCF₃ and —N(R^{o})₂,
   wherein each occurrence of R^{o} is independently selected from: —(C1-C6)-aliphatic, (C3-C6)-cycloalkyl, 3- to 6-membered heterocyclyl, 5- to 10-membered heteroaryl-, and (C6-C10)-aryl;
wherein each R₇ is selected from —H, —CF₃, —(C1-C6)alkyl, —(C1-C6)alkyloxy, (C1-C6) alkylamino, —(C3-C6) cycloalkyl, -5 to 10 membered heteroaryl, —(C6-C10) aryl, (C6-C10) aryl-(C1-C6)alkyl-, —(C1-C6)alkyl-5 to 10 membered heteroaryl and -3-10 membered heterocyclyl, wherein each R₇ is independently substituted with 0-5 R′;
   or when two R₇ groups bound to the same atom, the two R₇ groups may be taken together with the atom to which they are bound to form a 3-10-membered aromatic or non-aromatic ring having 0-4 heteroatoms independently selected from N, NH, O, S, SO, and SO₂, wherein said ring is optionally substituted with 0-5 R′;
   each occurrence of R′ is wherein each occurrence of R′ is independently selected from halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂;
wherein R″ is selected from —Cl, —F, —(C1-C6)alkyl, —OMe, —(C1-C6)alkyl-5 to 10 membered heteroaryl, -5 to 10 membered heteroaryl, -3-10 membered heterocyclyl, —(C3-C6) cycloalkyl and —(C6-C10) aryl;
R″ is independently substituted with 1-3 substituents wherein the substituents are selected from: halogen, —CF₃, —OCF₃, —O(C1-C6)-aliphatic, —(C1-C6)-aliphatic and -5 to 10 membered heteroaryl;
each R⁴ and R⁶ is independently selected from —H or —(C1-C6)alkyl;
each R¹³ and R¹⁴ is independently selected from H—, (C1-C3)-aliphatic-, or (C3-C6)-cycloalkyl;
iii) a compound of formula I-c:

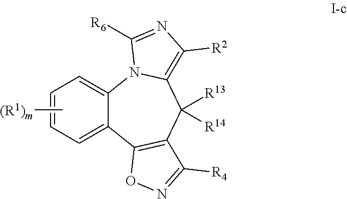

or a pharmaceutically acceptable salt, hydrate, solvate, polymorph, isomer, or combination thereof,
m is an integer selected from 0-4;
each R¹ is independently selected from -halogen, —(C6-C10) aryl, —OMe, —CN, —CHF₂, —CF₃, —OCF₃, —OCHF₂, CO(O)R₇, CH₂—OR⁸, —(C1-C6)alkyl-(C6-C10) aryl, -5-10 membered heteroaryl, —(C1-C6)alkyl-5-10 membered heteroaryl and —(C3-C6) cycloalkyl;
each R⁸ is independently selected from —H or —(C1-C6 alkyl);
each R² is selected from CO(O)R₇, C≡C—R⁹, —(C1-C6)alkyl-C≡C—R⁹, -(5-10 membered) heteroaryl, -(3-10 membered) heterocyclyl,

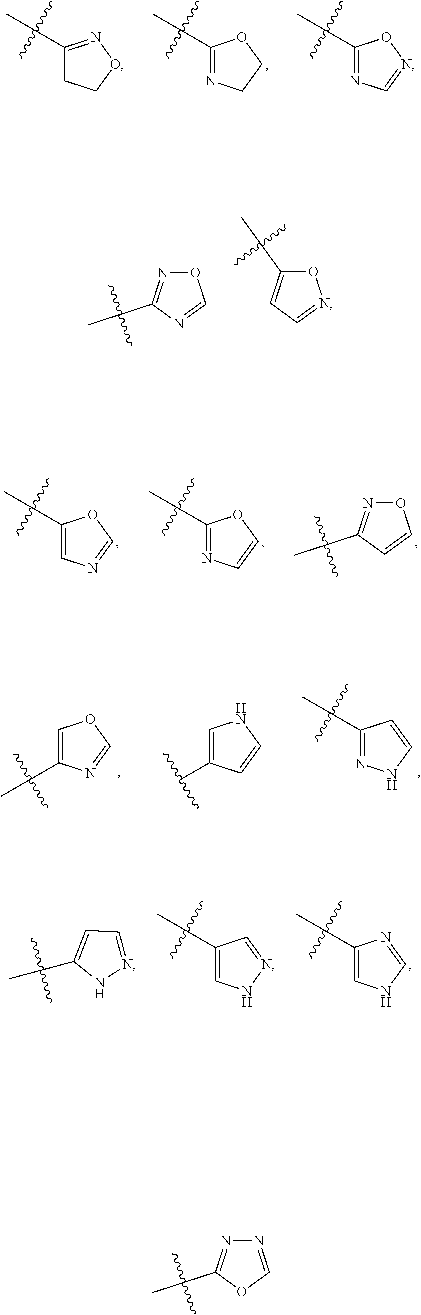

wherein each 5-membered heterocycle or heteroaryl is substituted with 0-4 R₇;
each R⁹ is selected from: —H, —(C1-C6)alkyl, (5- to 10-membered heteroaryl), -(3-10 membered) heterocyclyl

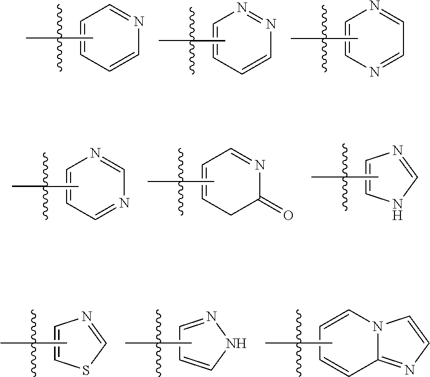

each occurrence of R⁹ is independently substituted by 0-5 R¹¹;
each occurrence of R¹¹ is independently selected from —(C1-C6)alkyl, —O—(C1-C6)alkyl, -halogen, —CF₃, —OCF₃, —OMe, —(C6-C10) aryl and -5 to 10 membered heteroaryl;
each occurrence of R₇ is selected from —CF₃, —(C1-C6)alkyl, —(C3-C6) cycloalkyl, -5 to 10 membered heteroaryl, —(C6-C10) aryl, (C6-C10) aryl-(C1-C6)alkyl-, —(C1-C6)alkyl-5 to 10 membered heteroaryl and -3-10 membered heterocyclyl, wherein each R₇ is independently substituted with 0-5 R′;
each occurrence of R′ is wherein each occurrence of R′ is independently selected from halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂,
wherein each occurrence of R″ is selected from —Cl, —F, —(C1-C6)alkyl, —OMe, —(C1-C6)alkyl-5 to 10 membered heteroaryl, -5 to 10 membered heteroaryl, -3-10 membered heterocyclyl, —(C3-C6) cycloalkyl and —(C6-C10) aryl, and R″ is independently substituted with 1-3 substituents wherein the substituents are selected from: halogen, —CF₃, —OCF₃, —(C1-C6)-aliphatic and -5 to 10 membered heteroaryl;
each R⁴ and R⁶ is independently selected from —H or (C1-C6)alkyl;
each R¹³ and R¹⁴ is independently selected from H—, (C1-C3)-aliphatic-, or (C3-C6)-cycloalkyl;
iv) a compound of formula I-d:

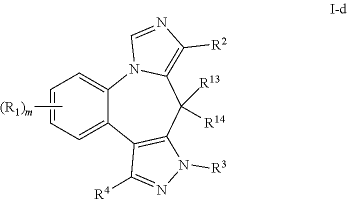

or a pharmaceutically acceptable salt, hydrate, solvate, polymorph, isomer, or combination thereof,
m is an integer selected from 0-4;
wherein:
each R¹ is independently selected from -halogen, —(C6-C10) aryl, —OMe, —CN, —CHF₂, —CF₃, —OCF₃, —OCHF₂, CO(O)R₇, CH₂—OR⁸, —(C1-C6)alkyl-(C6-C10) aryl, -5-10 membered heteroaryl, —(C1-C6)alkyl-5-10 membered heteroaryl and —(C3-C6) cycloalkyl;
each R⁸ is independently selected from —H or —(C1-C6 alkyl);
each R² is selected from CO(O)R₇, C≡C—R⁹, —(C1-C6)alkyl-C≡C—R⁹, -(5-10 membered) heteroaryl, -(3-10 membered) heterocyclyl,

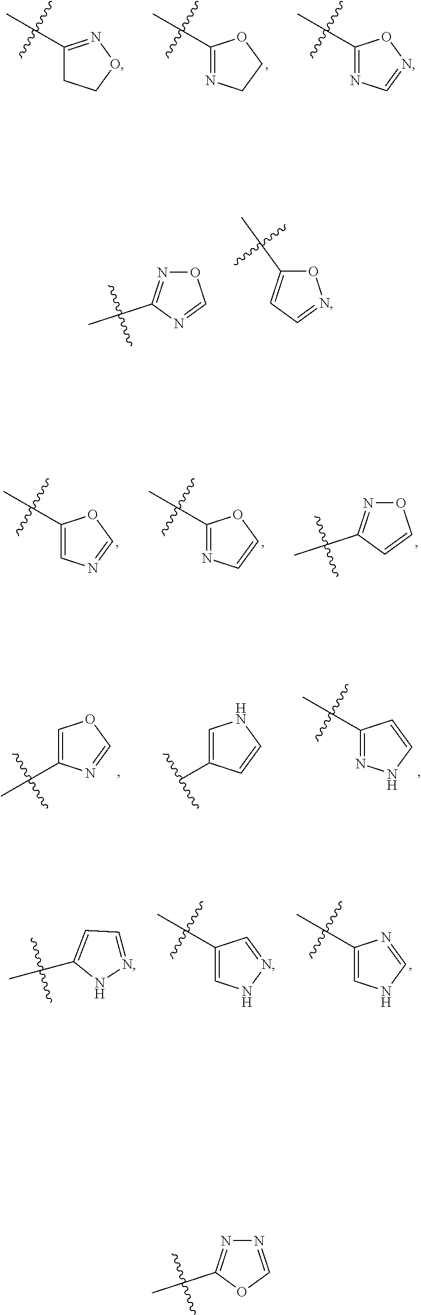

wherein each 5-membered heterocycle or heteroaryl is substituted with 0-4 R₇;
each R⁹ is selected from: —H, —(C1-C6)alkyl, (5- to 10-membered heteroaryl), -(3-10 membered) heterocyclyl, —(C6-C10) aryl, —(C1-C6)alkyl-(C6-C10) aryl

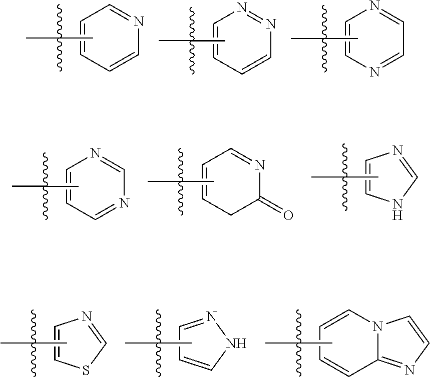

wherein each occurrence of R⁹ is independently substituted by 0-5 R¹¹
wherein each occurrence of R¹¹ is independently selected from —(C1-C6)alkyl, —O—(C1-C6)alkyl, -halogen, —CF₃, —OCF₃, —OMe, —(C6-C10) aryl and -5 to 10 membered heteroaryl;
R³ is independently selected from: —H, —(C1-C6)alkyl, —(C1-C6)alkyl-(C3-C6) cycloalkyl, —(C1-C6)alkyl-OR¹², —(C1-C6)alkyl-N(R¹²)₂, —(C1-C6)alkyl-(C6-C10) aryl, —(C1-C6) alkyl-5-10 membered heteroaryl wherein R³ is independently substituted with 0-5 R¹²;
wherein each R¹² is independently selected from: —H, -halogen, —OR^{o}, R^{o}, oxo, —CH₂OR^{o}, —CH₂N(R^{o})₂, —C(O)N(R^{o})₂, —C(O)OR^{o}, —NO₂, —NCS, —CN, —CF₃, —OCF₃ and —N(R^{o})₂,
   wherein each occurrence of R^{o} is independently selected from: —(C1-C6)-aliphatic, (C3-C6)-cycloalkyl, 3- to 6-membered heterocyclyl, 5- to 10-membered heteroaryl-, and (C6-C10)-aryl;
wherein R₇ is selected from —CF₃, —(C1-C6)alkyl, —(C3-C6) cycloalkyl, -5 to 10 membered heteroaryl, —(C6-C10) aryl, (C6-C10) aryl-(C1-C6)alkyl-, —(C1-C6)alkyl-5 to 10 membered heteroaryl and -3-10 membered heterocyclyl, wherein each R₇ is independently substituted with 0-5 R′;
each occurrence of R′ is wherein each occurrence of R′ is independently selected from halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂;
wherein R″ is selected from —Cl, —F, —(C1-C6)alkyl, —OMe, and —(C6-C10) aryl;
each R⁴ is selected from —H or —(C1-C6)alkyl;
each R⁶ is selected from —H or —(C1-C6)alkyl;
each R¹³ and R¹⁴ is independently selected from H—, (C1-C3)-aliphatic-, or (C3-C6)-cycloalkyl;
v) a compound of formula I-e:

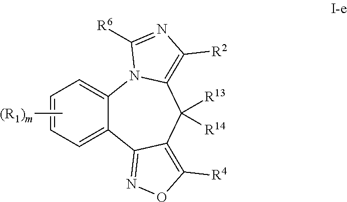

or a pharmaceutically acceptable salt, hydrate, solvate, polymorph, isomer, or combination thereof,
m is an integer selected from 0-4;
each R¹ is independently selected from -halogen, —(C6-C10) aryl, —OMe, —CN, —CHF₂, —CF₃, —OCF₃, —OCHF₂, CO(O)R₇, CH₂—OR⁸, —(C1-C6)alkyl-(C6-C10) aryl, -5-10 membered heteroaryl, —(C1-C6)alkyl-5-10 membered heteroaryl and —(C3-C6) cycloalkyl;
each R⁸ is independently selected from —H or —(C1-C6 alkyl);
each R² is selected from CO(O)R₇, C≡C—R⁹, —(C1-C6)alkyl-C≡C—R⁹, -(5-10 membered) heteroaryl, -(3-10 membered) heterocyclyl,

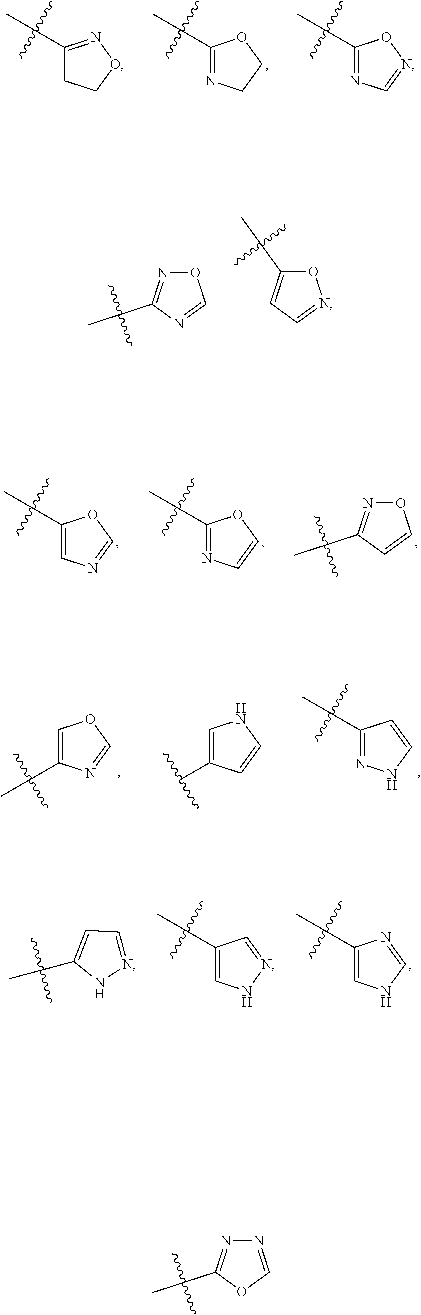

wherein each 5-membered heterocycle or heteroaryl is substituted with 0-4 R₇;
each R⁹ is selected from: —H, —(C1-C6)alkyl, (5- to 10-membered heteroaryl), -(3-10membered) heterocyclyl

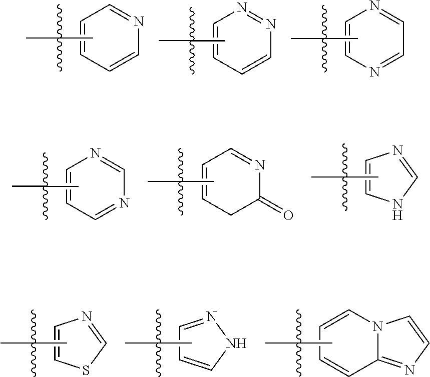

each occurrence of R⁹ is independently substituted by 0-5 R¹¹, wherein each occurrence of R¹¹ is independently selected from —(C1-C6)alkyl, —O—(C1-C6)alkyl, -halogen, —CF₃, —OCF₃, —OMe, —(C6-C10) aryl and -5 to 10 membered heteroaryl;
wherein R₇ is selected from —(C1-C6)alkyl, —(C3-C6) cycloalkyl, -5 to 10 membered heteroaryl, —(C6-C10) aryl, —(C6-C10) aryl-(C1-C6)alkyl, and -5 to 10 membered heteroaryl-(C1-C6)alkyl, and -3-10 membered heterocyclyl, wherein each R₇ is independently substituted with 0-5 R′;
each occurrence of R′ is wherein each occurrence of R′ is independently selected from halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂, wherein each R′ is independently substituted with 0-5 R″;
wherein R″ is selected from —Cl, —F, —(C1-C6)alkyl, —OMe, and —(C6-C10) aryl;
each R⁴ is independently-H or (C1-C6)alkyl;
each R⁶ is independently-H or —(C1-C6)alkyl;
each R¹³ and R¹⁴ is independently selected from H—, (C1-C3)-aliphatic-, or (C3-C6)-cycloalkyl; and
vi) a compound of formula I-f:

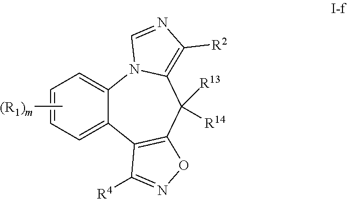

or a pharmaceutically acceptable salt, hydrate, solvate, polymorph, isomer, or combination thereof,
m is an integer selected from 0-4;
each R¹ is independently selected from -halogen, —(C6-C10) aryl, —OMe, —CN, —CHF₂, —CF₃, —OCF₃, —OCHF₂, CO(O)R₇, CH₂—OR⁸, —(C1-C6)alkyl-(C6-C10) aryl, -5-10 membered heteroaryl, —(C1-C6)alkyl-5-10 membered heteroaryl and —(C3-C6) cycloalkyl;
each R⁸ is independently selected from —H or —(C1-C6 alkyl);
each R² is selected from CO(O)R₇, C≡C—R⁹, —(C1-C6)alkyl-C≡C—R⁹, -(5-10 membered) heteroaryl, -(3-10 membered) heterocyclyl,

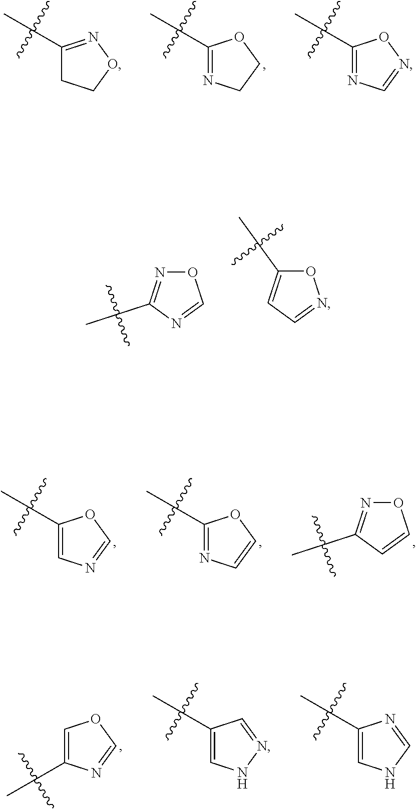

wherein each 5-membered heterocycle or heteroaryl is substituted with 0-4 R₇;
each R⁹ is selected from: —H, —(C1-C6)alkyl, (5- to 10-membered heteroaryl), -(3-10 membered) heterocyclyl

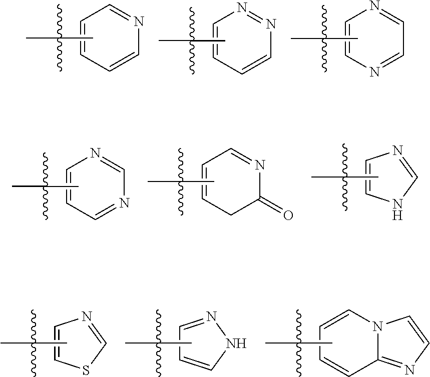

each occurrence of R⁹ is independently substituted by 0-5 R¹¹, wherein each occurrence of R¹¹ is independently selected from —(C1-C6)alkyl, —O—(C1-C6)alkyl, -halogen, —CF₃, —OCF₃, —OMe, —(C6-C10) aryl and -5 to 10 membered heteroaryl;
wherein R₇ is selected from —(C1-C6)alkyl, —(C3-C6) cycloalkyl, -5 to 10 membered heteroaryl, —(C6-C10) aryl, —(C6-C10) aryl-(C1-C6)alkyl, and -5 to 10 membered heteroaryl-(C1-C6)alkyl, and -3-10 membered heterocyclyl, wherein each R₇ is independently substituted with 0-5 R′;
each occurrence of R′ is wherein each occurrence of R′ is independently selected from halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂;
wherein each R′ is independently substituted with 0-5 R″, wherein R″ is selected from —Cl, —F, —(C1-C6)alkyl, —OMe, and —(C6-C10) aryl;
R⁴ is —H or (C1-C6)alkyl;
R⁶ is —H or —(C1-C6)alkyl;
each R¹³ and R¹⁴ is independently selected from H—, (C1-C3)-aliphatic-, or (C3-C6)-cycloalkyl.

In some embodiments, the GABA_{A} α5 receptor agonist or the GABA_{A} α5 receptor agonist of the pharmaceutical composition of this disclosure is selected from the group consisting of:

| Compound | Structure |
|---|---|
| 742 | 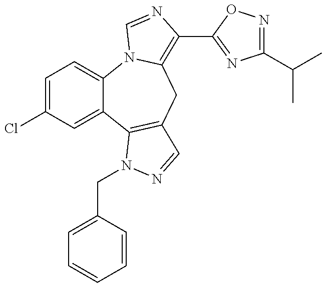 [SMILES: CC(C)c1noc(-c2ncn3c2Cc2cnn(Cc4ccccc4)c2-c2cc(Cl)ccc2-3)n1] |
| 743 | 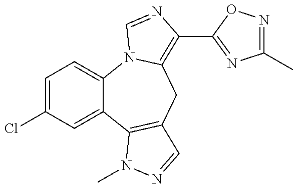 [SMILES: Cc1noc(-c2ncn3c2Cc2cnn(C)c2-c2cc(Cl)ccc2-3)n1] |
| 744 | 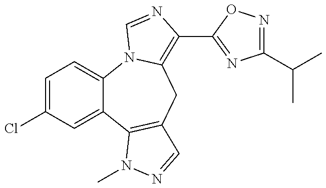 [SMILES: CC(C)c1noc(-c2ncn3c2Cc2cnn(C)c2-c2cc(Cl)ccc2-3)n1] |
| 745 | 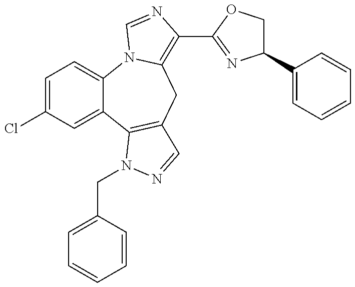 [SMILES: Clc1ccc2c(c1)-c1c(cnn1Cc1ccccc1)Cc1c(C3=N[C@H](c4ccccc4)CO3)ncn1-2] |
| 746 | 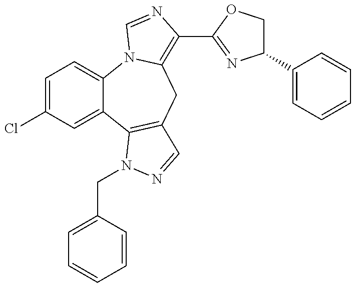 [SMILES: Clc1ccc2c(c1)-c1c(cnn1Cc1ccccc1)Cc1c(C3=N[C@@H](c4ccccc4)CO3)ncn1-2] |
| 747 | 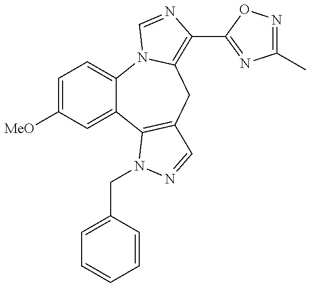 [SMILES: COc1ccc2c(c1)-c1c(cnn1Cc1ccccc1)Cc1c(-c3nc(C)no3)ncn1-2] |
| 748 | 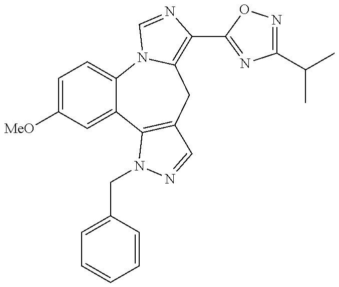 [SMILES: COc1ccc2c(c1)-c1c(cnn1Cc1ccccc1)Cc1c(-c3nc(C(C)C)no3)ncn1-2] |
| 749 | 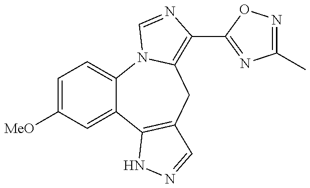 [SMILES: COc1ccc2c(c1)-c1[nH]ncc1Cc1c(-c3nc(C)no3)ncn1-2] |
| 750 | 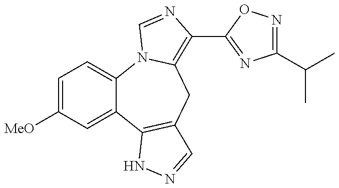 [SMILES: COc1ccc2c(c1)-c1[nH]ncc1Cc1c(-c3nc(C(C)C)no3)ncn1-2] |
| 751 | 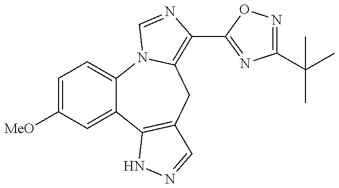 [SMILES: COc1ccc2c(c1)-c1[nH]ncc1Cc1c(-c3nc(C(C)(C)C)no3)ncn1-2] |
| 752 | 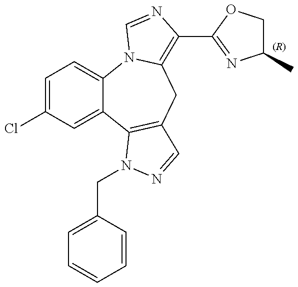 |
| 753 | 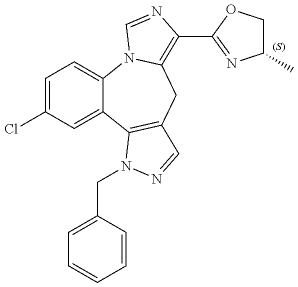 |
| 754 | 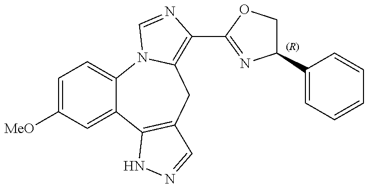 |
| 755 | 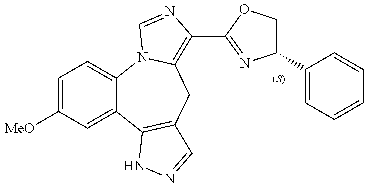 |
| 758 | 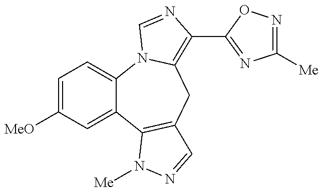 [SMILES: COc1ccc2c(c1)-c1c(cnn1C)Cc1c(-c3nc(C)no3)ncn1-2] |
| 759 | 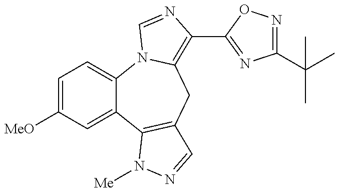 [SMILES: COc1ccc2c(c1)-c1c(cnn1C)Cc1c(-c3nc(C(C)(C)C)no3)ncn1-2] |
| 760 | 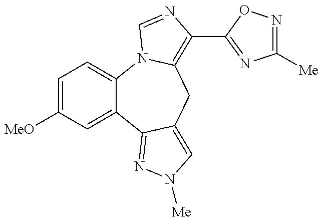 [SMILES: COc1ccc2c(c1)-c1nn(C)cc1Cc1c(-c3nc(C)no3)ncn1-2] |
| 761 | 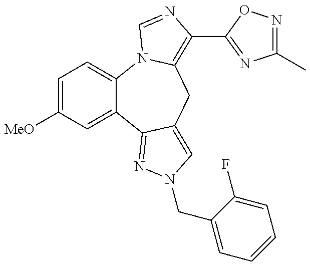 [SMILES: COc1ccc2c(c1)-c1nn(Cc3ccccc3F)cc1Cc1c(-c3nc(C)no3)ncn1-2] |
| 762 | 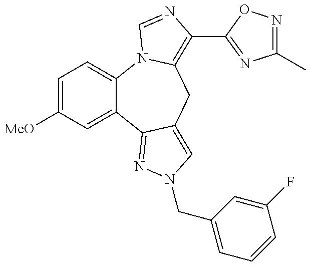 [SMILES: COc1ccc2c(c1)-c1nn(Cc3cccc(F)c3)cc1Cc1c(-c3nc(C)no3)ncn1-2] |
| 763 | 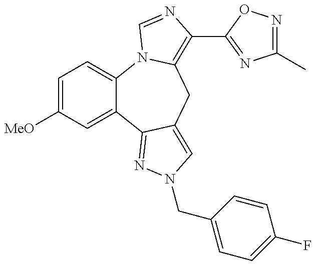 [SMILES: COc1ccc2c(c1)-c1nn(Cc3ccc(F)cc3)cc1Cc1c(-c3nc(C)no3)ncn1-2] |
| 765 | 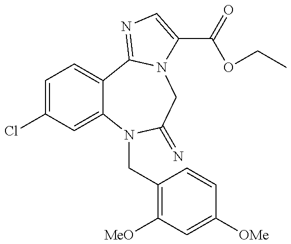 [SMILES: CCOC(=O)c1cnc2n1CC(=N)N(Cc1ccc(OC)cc1OC)c1cc(Cl)ccc1-2] |
| 766 | 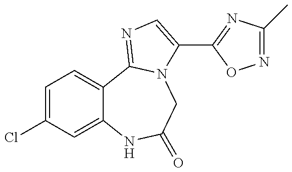 [SMILES: Cc1noc(-c2cnc3n2CC(=O)Nc2cc(Cl)ccc2-3)n1] |
| 767 | 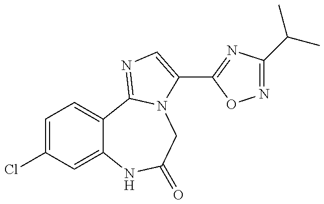 [SMILES: CC(C)c1noc(-c2cnc3n2CC(=O)Nc2cc(Cl)ccc2-3)n1] |
| 768 | 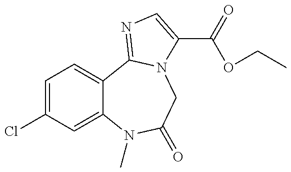 [SMILES: CCOC(=O)c1cnc2n1CC(=O)N(C)c1cc(Cl)ccc1-2] |
| 769 | 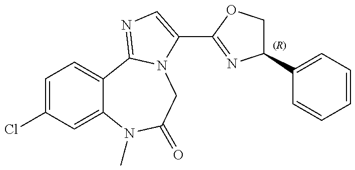 |
| 770 | 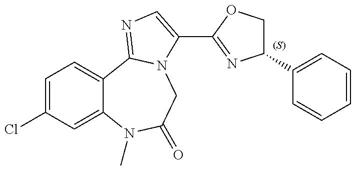 |
| 771 | 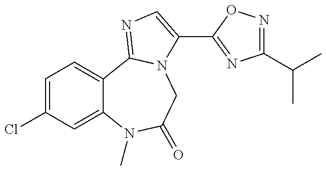 [SMILES: CC(C)c1noc(-c2cnc3n2CC(=O)N(C)c2cc(Cl)ccc2-3)n1] |
| 772 | 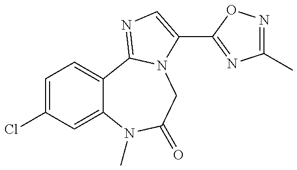 [SMILES: Cc1noc(-c2cnc3n2CC(=O)N(C)c2cc(Cl)ccc2-3)n1] |
| 773 | 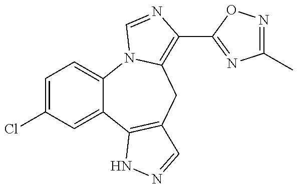 [SMILES: Cc1noc(-c2ncn3c2Cc2cn[nH]c2-c2cc(Cl)ccc2-3)n1] |
| 774 | 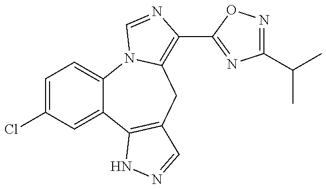 [SMILES: CC(C)c1noc(-c2ncn3c2Cc2cn[nH]c2-c2cc(Cl)ccc2-3)n1] |
| 775 | 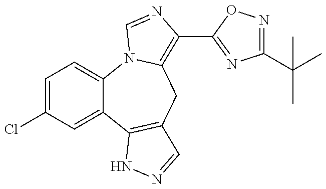 [SMILES: CC(C)(C)c1noc(-c2ncn3c2Cc2cn[nH]c2-c2cc(Cl)ccc2-3)n1] |
| 776 | 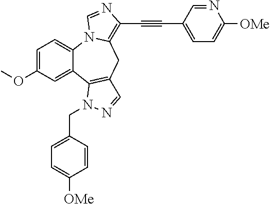 [SMILES: COc1ccc(Cn2ncc3c2-c2cc(OC)ccc2-n2cnc(C#Cc4ccc(OC)nc4)c2C3)cc1] |
| 777 | 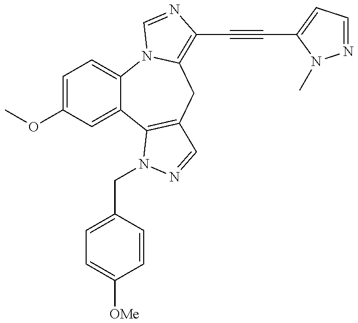 [SMILES: COc1ccc(Cn2ncc3c2-c2cc(OC)ccc2-n2cnc(C#Cc4ccnn4C)c2C3)cc1] |
| 778 | 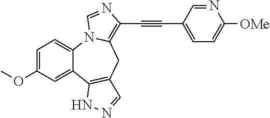 [SMILES: COc1ccc2c(c1)-c1[nH]ncc1Cc1c(C#Cc3ccc(OC)nc3)ncn1-2] |
| 779 | 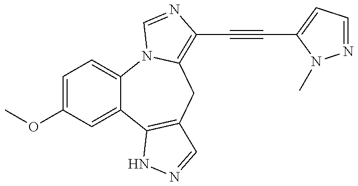 [SMILES: COc1ccc2c(c1)-c1[nH]ncc1Cc1c(C#Cc3ccnn3C)ncn1-2] |
| 781 | 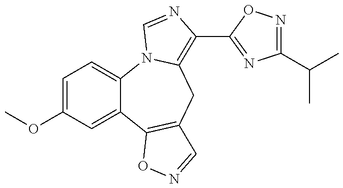 [SMILES: COc1ccc2c(c1)-c1oncc1Cc1c(-c3nc(C(C)C)no3)ncn1-2] |
| 782 | 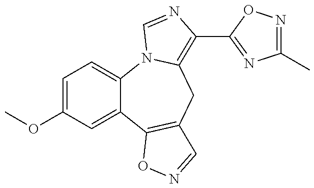 |
| 784 | 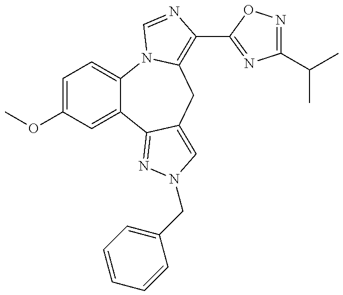 [SMILES: COc1ccc2c(c1)-c1nn(Cc3ccccc3)cc1Cc1c(-c3nc(C(C)C)no3)ncn1-2] |
| 785 | 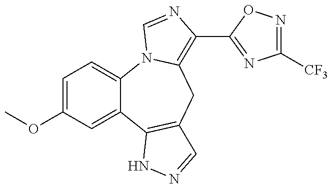 [SMILES: COc1ccc2c(c1)-c1[nH]ncc1Cc1c(-c3nc(C(F)(F)F)no3)ncn1-2] |
| 786 | 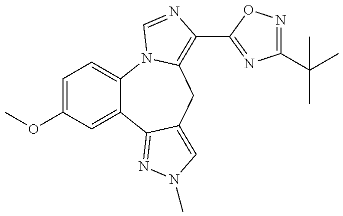 [SMILES: COc1ccc2c(c1)-c1nn(C)cc1Cc1c(-c3nc(C(C)(C)C)no3)ncn1-2] |
| 787 | 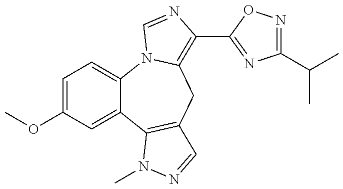 [SMILES: COc1ccc2c(c1)-c1c(cnn1C)Cc1c(-c3nc(C(C)C)no3)ncn1-2] |
| 788 | 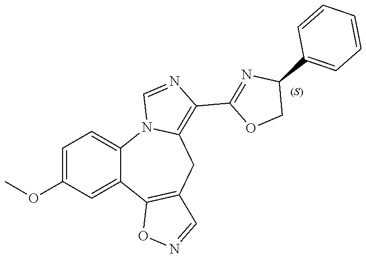 |
| 789 | 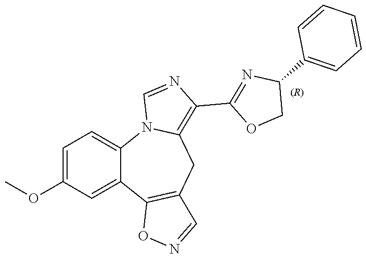 |
| 790 | 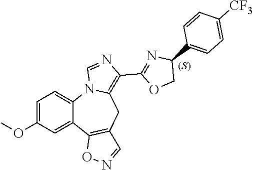 |
| 791 | 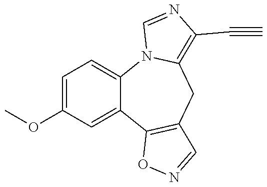 [SMILES: C#Cc1ncn2c1Cc1cnoc1-c1cc(OC)ccc1-2] |
| 792 | 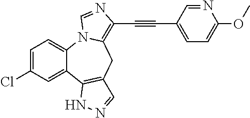 [SMILES: COc1ccc(C#Cc2ncn3c2Cc2cn[nH]c2-c2cc(Cl)ccc2-3)cn1] |
| 793 | 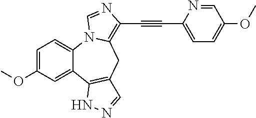 |
| 794 | 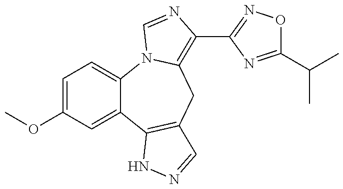 [SMILES: COc1ccc2c(c1)-c1[nH]ncc1Cc1c(-c3noc(C(C)C)n3)ncn1-2] |
| 795 | 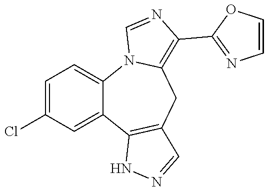 [SMILES: Clc1ccc2c(c1)-c1[nH]ncc1Cc1c(-c3ncco3)ncn1-2] |
| 797 | 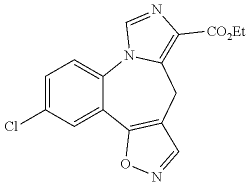 [SMILES: CCOC(=O)c1ncn2c1Cc1cnoc1-c1cc(Cl)ccc1-2] |
| 798 | 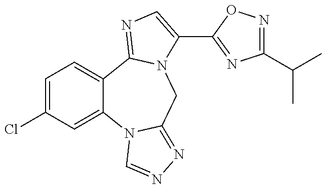 [SMILES: CC(C)c1noc(-c2cnc3n2Cc2nncn2-c2cc(Cl)ccc2-3)n1] |
| 799 | 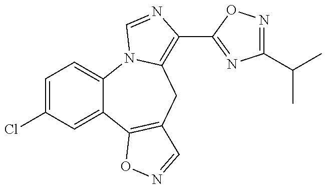 [SMILES: CC(C)c1noc(-c2ncn3c2Cc2cnoc2-c2cc(Cl)ccc2-3)n1] |
| 800 | 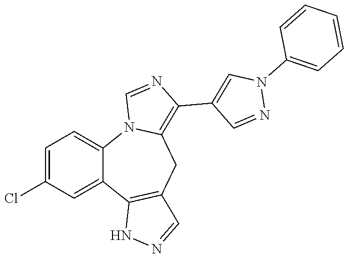 [SMILES: Clc1ccc2c(c1)-c1[nH]ncc1Cc1c(-c3cnn(-c4ccccc4)c3)ncn1-2] |
| 801 | 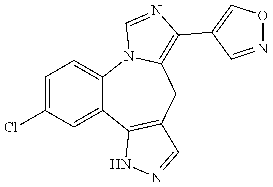 [SMILES: Clc1ccc2c(c1)-c1[nH]ncc1Cc1c(-c3cnoc3)ncn1-2] |
| 802 | 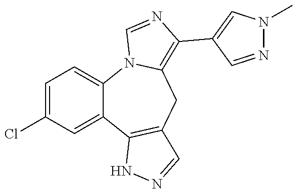 [SMILES: Cn1cc(-c2ncn3c2Cc2cn[nH]c2-c2cc(Cl)ccc2-3)cn1] |
| 803 | 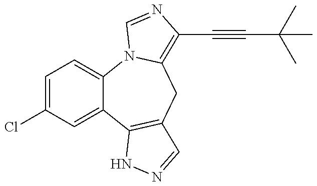 [SMILES: CC(C)(C)C#Cc1ncn2c1Cc1cn[nH]c1-c1cc(Cl)ccc1-2] |
| 804 | 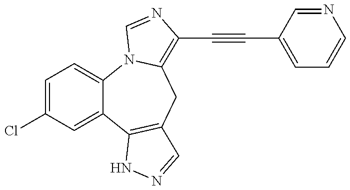 [SMILES: Clc1ccc2c(c1)-c1[nH]ncc1Cc1c(C#Cc3cccnc3)ncn1-2] |
| 805 | 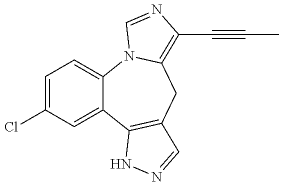 [SMILES: CC#Cc1ncn2c1Cc1cn[nH]c1-c1cc(Cl)ccc1-2] |
| 806 | 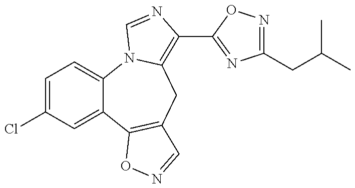 [SMILES: CC(C)Cc1noc(-c2ncn3c2Cc2cnoc2-c2cc(Cl)ccc2-3)n1] |
| 807 | 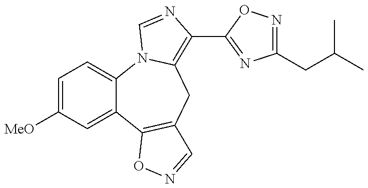 [SMILES: COc1ccc2c(c1)-c1oncc1Cc1c(-c3nc(CC(C)C)no3)ncn1-2] |
| 808 | 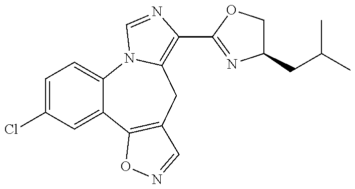 [SMILES: CC(C)C[C@@H]1COC(c2ncn3c2Cc2cnoc2-c2cc(Cl)ccc2-3)=N1] |
| 809 | 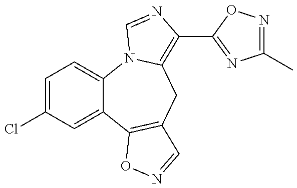 [SMILES: Cc1noc(-c2ncn3c2Cc2cnoc2-c2cc(Cl)ccc2-3)n1] |
| 810 | 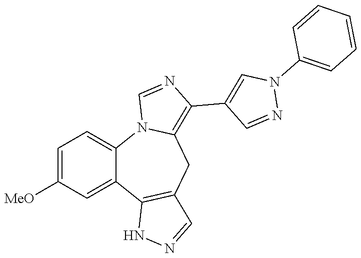 [SMILES: COc1ccc2c(c1)-c1[nH]ncc1Cc1c(-c3cnn(-c4ccccc4)c3)ncn1-2] |
| 813 | 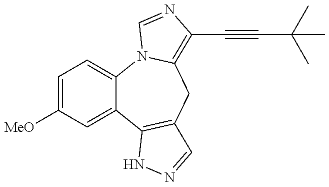 [SMILES: COc1ccc2c(c1)-c1[nH]ncc1Cc1c(C#CC(C)(C)C)ncn1-2] |
| 814 | 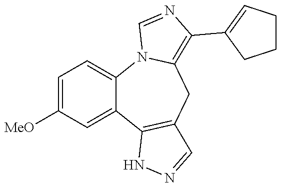 [SMILES: COc1ccc2c(c1)-c1[nH]ncc1Cc1c(C3=CCCC3)ncn1-2] |
| 815 | 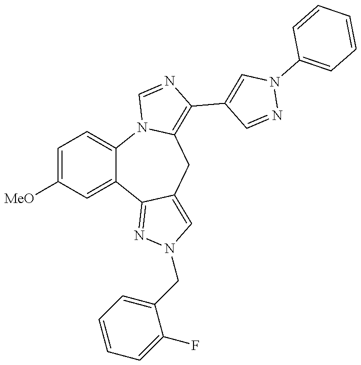 [SMILES: COc1ccc2c(c1)-c1nn(Cc3ccccc3F)cc1Cc1c(-c3cnn(-c4ccccc4)c3)ncn1-2] |
| 816 | 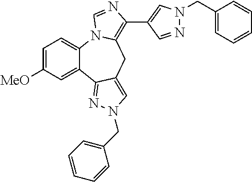 [SMILES: COc1ccc2c(c1)-c1nn(Cc3ccccc3)cc1Cc1c(-c3cnn(Cc4ccccc4)c3)ncn1-2] |
| 817 | 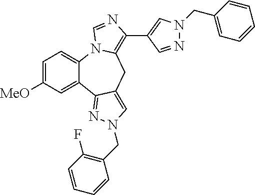 [SMILES: COc1ccc2c(c1)-c1nn(Cc3ccccc3F)cc1Cc1c(-c3cnn(Cc4ccccc4)c3)ncn1-2] |
| 818 | 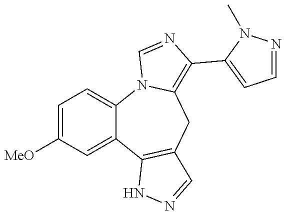 [SMILES: COc1ccc2c(c1)-c1[nH]ncc1Cc1c(-c3ccnn3C)ncn1-2] |
| 819 | 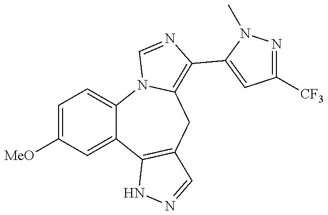 [SMILES: COc1ccc2c(c1)-c1[nH]ncc1Cc1c(-c3cc(C(F)(F)F)nn3C)ncn1-2] |
| 820 | 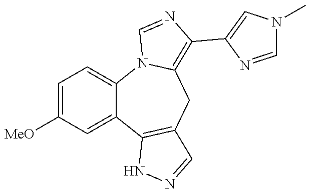 [SMILES: COc1ccc2c(c1)-c1[nH]ncc1Cc1c(-c3cn(C)cn3)ncn1-2] |
| 821 | 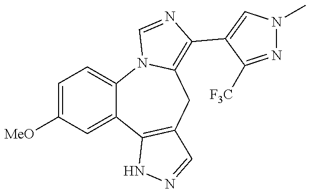 [SMILES: COc1ccc2c(c1)-c1[nH]ncc1Cc1c(-c3cn(C)nc3C(F)(F)F)ncn1-2] |
| 822 | 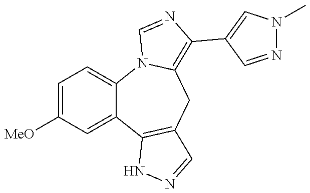 [SMILES: COc1ccc2c(c1)-c1[nH]ncc1Cc1c(-c3cnn(C)c3)ncn1-2] |
| 823 | 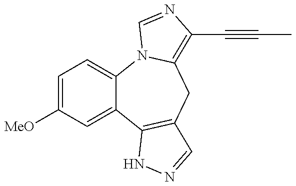 [SMILES: CC#Cc1ncn2c1Cc1cn[nH]c1-c1cc(OC)ccc1-2] |
| 824 | 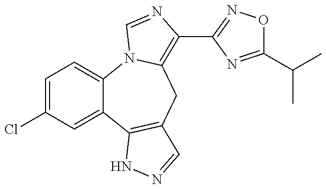 [SMILES: CC(C)c1nc(-c2ncn3c2Cc2cn[nH]c2-c2cc(Cl)ccc2-3)no1] |
| 825 | 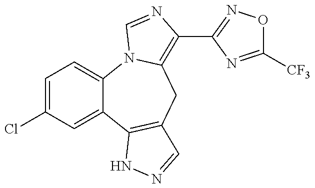 [SMILES: FC(F)(F)c1nc(-c2ncn3c2Cc2cn[nH]c2-c2cc(Cl)ccc2-3)no1] |
| 826 | 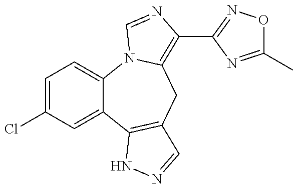 [SMILES: Cc1nc(-c2ncn3c2Cc2cn[nH]c2-c2cc(Cl)ccc2-3)no1] |
| 827 | 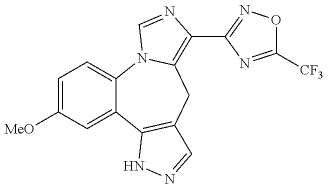 [SMILES: COc1ccc2c(c1)-c1[nH]ncc1Cc1c(-c3noc(C(F)(F)F)n3)ncn1-2] |
| 828 | 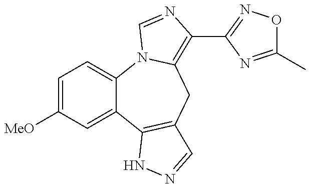 [SMILES: COc1ccc2c(c1)-c1[nH]ncc1Cc1c(-c3noc(C)n3)ncn1-2] |
| 830 | 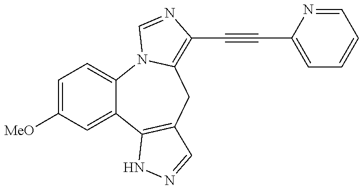 [SMILES: COc1ccc2c(c1)-c1[nH]ncc1Cc1c(C#Cc3ccccn3)ncn1-2] |
| 831 | 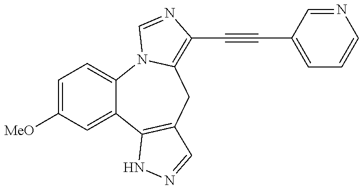 [SMILES: COc1ccc2c(c1)-c1[nH]ncc1Cc1c(C#Cc3cccnc3)ncn1-2] |
| 833 | 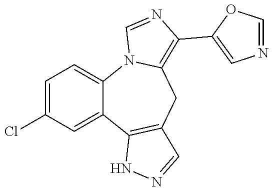 [SMILES: Clc1ccc2c(c1)-c1[nH]ncc1Cc1c(-c3cnco3)ncn1-2] |
| 834 | 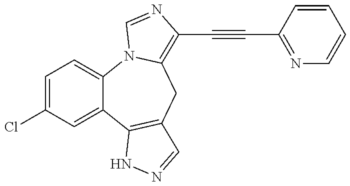 [SMILES: Clc1ccc2c(c1)-c1[nH]ncc1Cc1c(C#Cc3ccccn3)ncn1-2] |
| 835 | 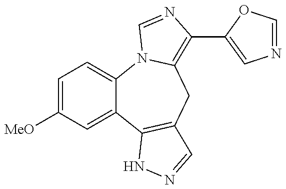 [SMILES: COc1ccc2c(c1)-c1[nH]ncc1Cc1c(-c3cnco3)ncn1-2] |
| 836 | 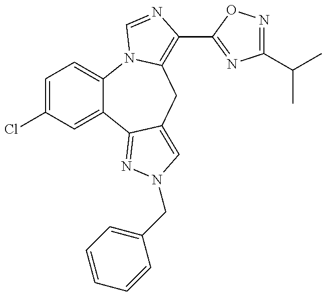 [SMILES: CC(C)c1noc(-c2ncn3c2Cc2cn(Cc4ccccc4)nc2-c2cc(Cl)ccc2-3)n1] |
| 837 | 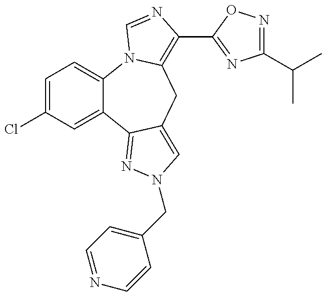 [SMILES: CC(C)c1noc(-c2ncn3c2Cc2cn(Cc4ccncc4)nc2-c2cc(Cl)ccc2-3)n1] |
| 838 | 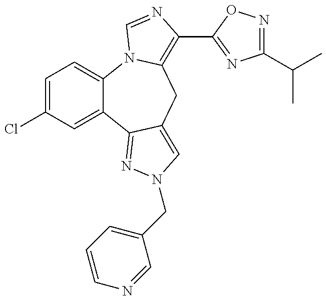 [SMILES: CC(C)c1noc(-c2ncn3c2Cc2cn(Cc4cccnc4)nc2-c2cc(Cl)ccc2-3)n1] |
| 839 | 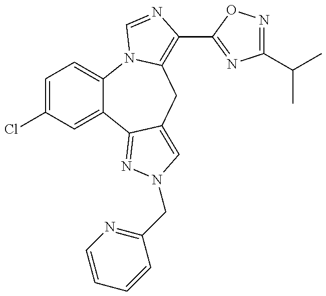 [SMILES: CC(C)c1noc(-c2ncn3c2Cc2cn(Cc4ccccn4)nc2-c2cc(Cl)ccc2-3)n1] |
| 840 | 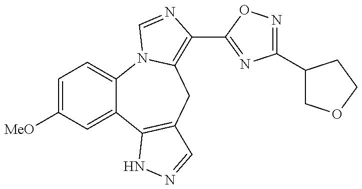 [SMILES: COc1ccc2c(c1)-c1[nH]ncc1Cc1c(-c3nc(C4CCOC4)no3)ncn1-2] |
| 841 | 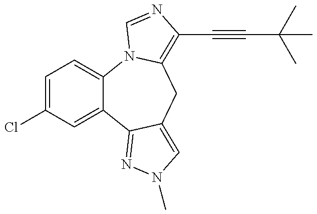 [SMILES: Cn1cc2c(n1)-c1cc(Cl)ccc1-n1cnc(C#CC(C)(C)C)c1C2] |
| 842 | 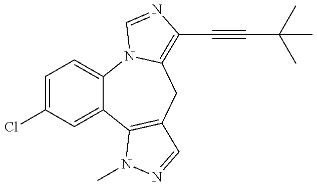 [SMILES: Cn1ncc2c1-c1cc(Cl)ccc1-n1cnc(C#CC(C)(C)C)c1C2] |
| 843 | 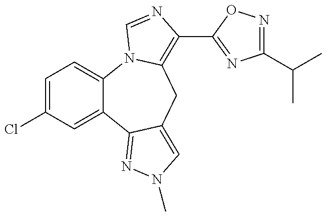 [SMILES: CC(C)c1noc(-c2ncn3c2Cc2cn(C)nc2-c2cc(Cl)ccc2-3)n1] |
| 844 | 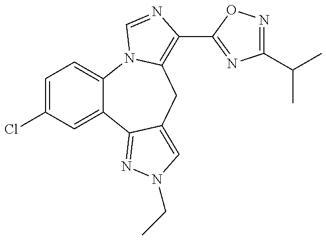 [SMILES: CCn1cc2c(n1)-c1cc(Cl)ccc1-n1cnc(-c3nc(C(C)C)no3)c1C2] |
| 845 | 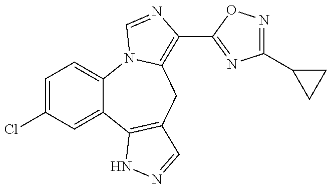 [SMILES: Clc1ccc2c(c1)-c1[nH]ncc1Cc1c(-c3nc(C4CC4)no3)ncn1-2] |
| 846 | 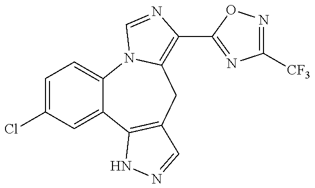 [SMILES: FC(F)(F)c1noc(-c2ncn3c2Cc2cn[nH]c2-c2cc(Cl)ccc2-3)n1] |
| 848 | 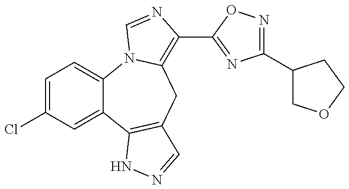 [SMILES: Clc1ccc2c(c1)-c1[nH]ncc1Cc1c(-c3nc(C4CCOC4)no3)ncn1-2] |
| 849 | 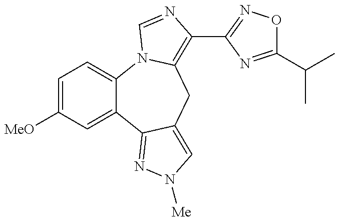 [SMILES: COc1ccc2c(c1)-c1nn(C)cc1Cc1c(-c3noc(C(C)C)n3)ncn1-2] |
| 850 | 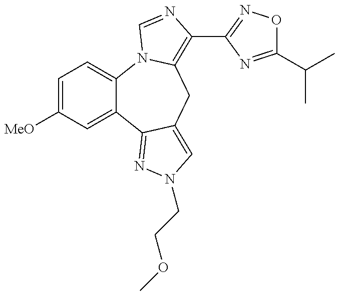 [SMILES: COCCn1cc2c(n1)-c1cc(OC)ccc1-n1cnc(-c3noc(C(C)C)n3)c1C2] |
| 851 | 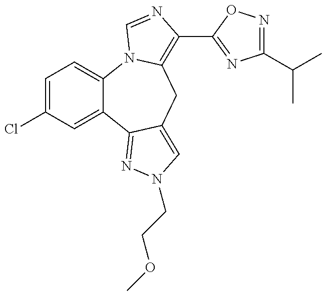 [SMILES: COCCn1cc2c(n1)-c1cc(Cl)ccc1-n1cnc(-c3nc(C(C)C)no3)c1C2] |
| 852 | 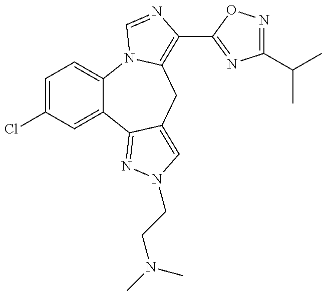 [SMILES: CC(C)c1noc(-c2ncn3c2Cc2cn(CCN(C)C)nc2-c2cc(Cl)ccc2-3)n1] |
| 853 | 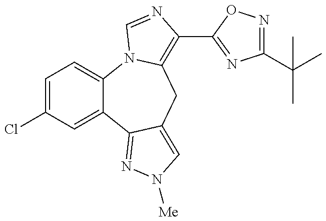 [SMILES: Cn1cc2c(n1)-c1cc(Cl)ccc1-n1cnc(-c3nc(C(C)(C)C)no3)c1C2] |
| 854 | 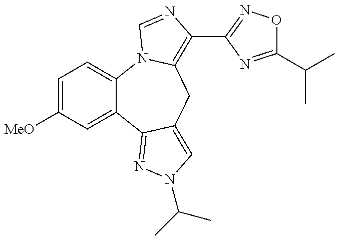 [SMILES: COc1ccc2c(c1)-c1nn(C(C)C)cc1Cc1c(-c3noc(C(C)C)n3)ncn1-2] |
| 855 | 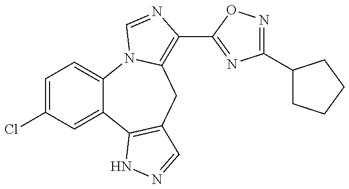 [SMILES: Clc1ccc2c(c1)-c1[nH]ncc1Cc1c(-c3nc(C4CCCC4)no3)ncn1-2] |
| 856 | 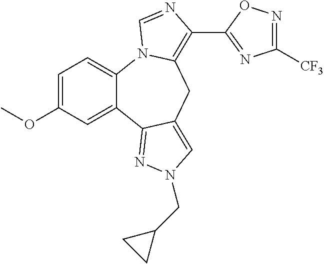 [SMILES: COc1ccc2c(c1)-c1nn(CC3CC3)cc1Cc1c(-c3nc(C(F)(F)F)no3)ncn1-2] |
| 857 | 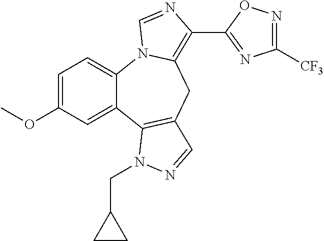 [SMILES: COc1ccc2c(c1)-c1c(cnn1CC1CC1)Cc1c(-c3nc(C(F)(F)F)no3)ncn1-2] |
| 858 | 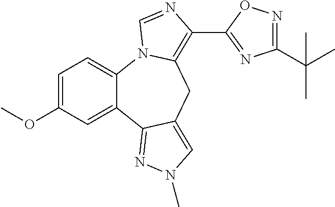 [SMILES: COc1ccc2c(c1)-c1nn(C)cc1Cc1c(-c3nc(C(C)(C)C)no3)ncn1-2] |
| 859 | 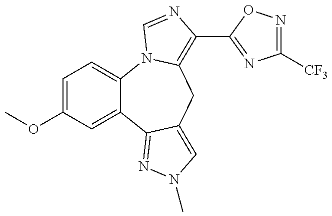 [SMILES: COc1ccc2c(c1)-c1nn(C)cc1Cc1c(-c3nc(C(F)(F)F)no3)ncn1-2] |
| 860 | 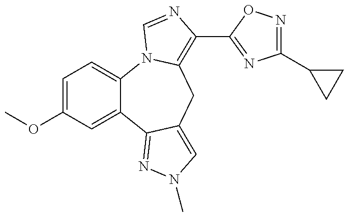 [SMILES: COc1ccc2c(c1)-c1nn(C)cc1Cc1c(-c3nc(C4CC4)no3)ncn1-2] |
| 861 | 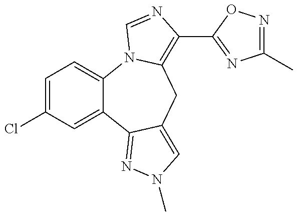 [SMILES: Cc1noc(-c2ncn3c2Cc2cn(C)nc2-c2cc(Cl)ccc2-3)n1] |
| 862 | 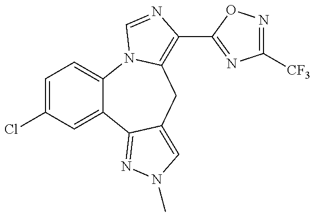 [SMILES: Cn1cc2c(n1)-c1cc(Cl)ccc1-n1cnc(-c3nc(C(F)(F)F)no3)c1C2] |
| 863 | 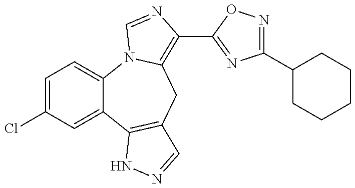 [SMILES: Clc1ccc2c(c1)-c1[nH]ncc1Cc1c(-c3nc(C4CCCCC4)no3)ncn1-2] |
| 864 | 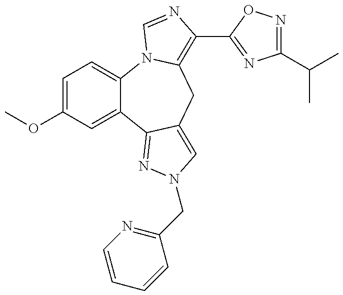 [SMILES: COc1ccc2c(c1)-c1nn(Cc3ccccn3)cc1Cc1c(-c3nc(C(C)C)no3)ncn1-2] |
| 865 | 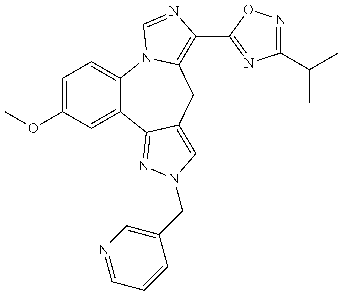 [SMILES: COc1ccc2c(c1)-c1nn(Cc3cccnc3)cc1Cc1c(-c3nc(C(C)C)no3)ncn1-2] |
| 866 | 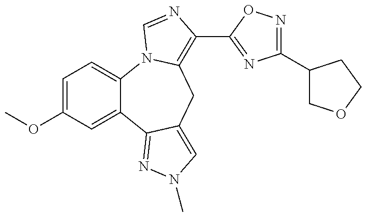 [SMILES: COc1ccc2c(c1)-c1nn(C)cc1Cc1c(-c3nc(C4CCOC4)no3)ncn1-2] |
| 867 | 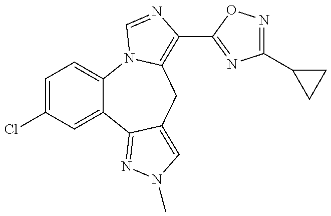 [SMILES: Cn1cc2c(n1)-c1cc(Cl)ccc1-n1cnc(-c3nc(C4CC4)no3)c1C2] |
| 868 | 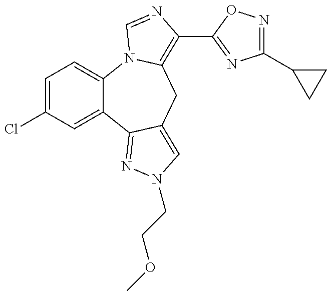 [SMILES: COCCn1cc2c(n1)-c1cc(Cl)ccc1-n1cnc(-c3nc(C4CC4)no3)c1C2] |
| 869 | 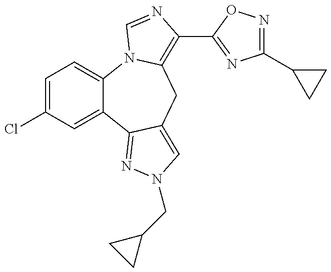 [SMILES: Clc1ccc2c(c1)-c1nn(CC3CC3)cc1Cc1c(-c3nc(C4CC4)no3)ncn1-2] |
| 870 | 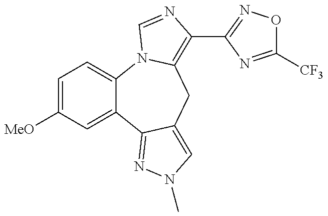 [SMILES: COc1ccc2c(c1)-c1nn(C)cc1Cc1c(-c3noc(C(F)(F)F)n3)ncn1-2] |
| 871 | 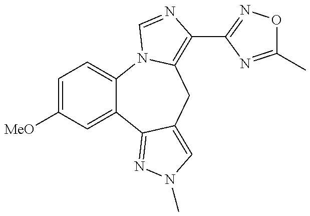 [SMILES: COc1ccc2c(c1)-c1nn(C)cc1Cc1c(-c3noc(C)n3)ncn1-2] |
| 872 | 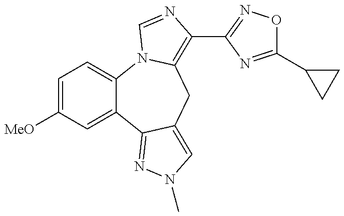 [SMILES: COc1ccc2c(c1)-c1nn(C)cc1Cc1c(-c3noc(C4CC4)n3)ncn1-2] |
| 873 | 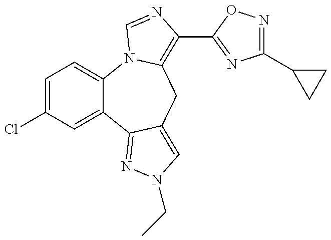 [SMILES: CCn1cc2c(n1)-c1cc(Cl)ccc1-n1cnc(-c3nc(C4CC4)no3)c1C2] |
| 874 | 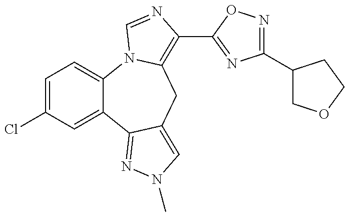 [SMILES: Cn1cc2c(n1)-c1cc(Cl)ccc1-n1cnc(-c3nc(C4CCOC4)no3)c1C2] |
| 875 | 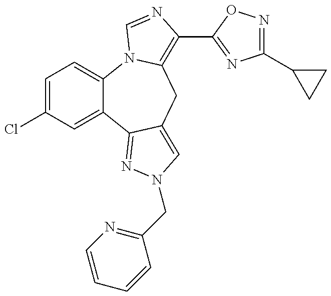 [SMILES: Clc1ccc2c(c1)-c1nn(Cc3ccccn3)cc1Cc1c(-c3nc(C4CC4)no3)ncn1-2] |
| 876 | 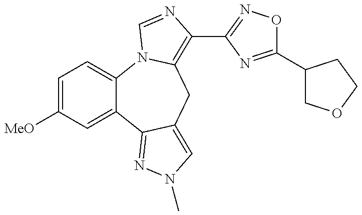 [SMILES: COc1ccc2c(c1)-c1nn(C)cc1Cc1c(-c3noc(C4CCOC4)n3)ncn1-2] |
| 877 | 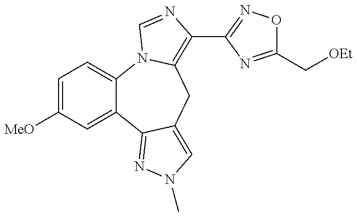 [SMILES: CCOCc1nc(-c2ncn3c2Cc2cn(C)nc2-c2cc(OC)ccc2-3)no1] |
| 878 | 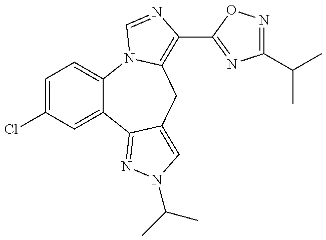 [SMILES: CC(C)c1noc(-c2ncn3c2Cc2cn(C(C)C)nc2-c2cc(Cl)ccc2-3)n1] |
| 879 | 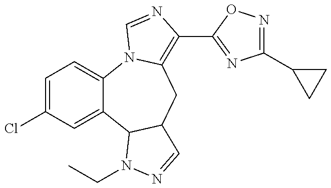 [SMILES: CCN1N=CC2Cc3c(-c4nc(C5CC5)no4)ncn3-c3ccc(Cl)cc3C21] |
| 880 | 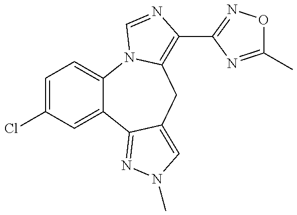 [SMILES: Cc1nc(-c2ncn3c2Cc2cn(C)nc2-c2cc(Cl)ccc2-3)no1] |
| 881 | 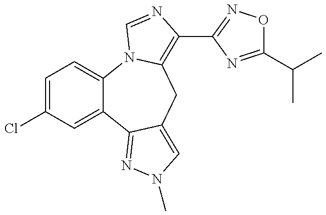 [SMILES: CC(C)c1nc(-c2ncn3c2Cc2cn(C)nc2-c2cc(Cl)ccc2-3)no1] |
| 882 | 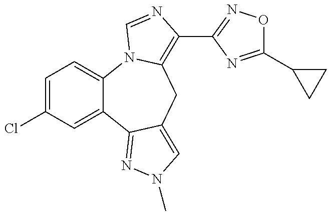 [SMILES: Cn1cc2c(n1)-c1cc(Cl)ccc1-n1cnc(-c3noc(C4CC4)n3)c1C2] |
| 883 | 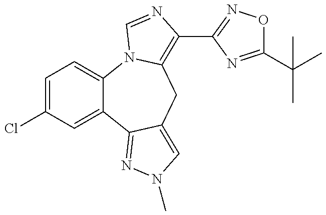 [SMILES: Cn1cc2c(n1)-c1cc(Cl)ccc1-n1cnc(-c3noc(C(C)(C)C)n3)c1C2] |
| 884 | 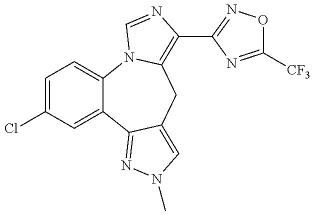 [SMILES: Cn1cc2c(n1)-c1cc(Cl)ccc1-n1cnc(-c3noc(C(F)(F)F)n3)c1C2] |
| 885 | 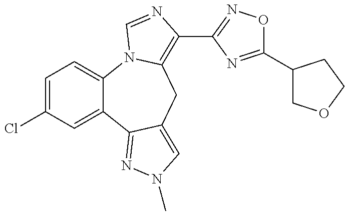 [SMILES: Cn1cc2c(n1)-c1cc(Cl)ccc1-n1cnc(-c3noc(C4CCOC4)n3)c1C2] |
| 886 | 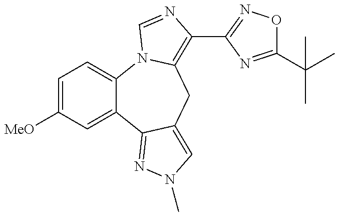 [SMILES: COc1ccc2c(c1)-c1nn(C)cc1Cc1c(-c3noc(C(C)(C)C)n3)ncn1-2] |
| 887 | 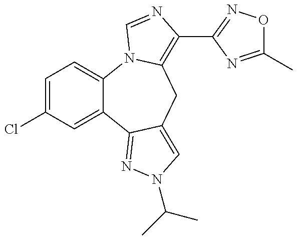 [SMILES: Cc1nc(-c2ncn3c2Cc2cn(C(C)C)nc2-c2cc(Cl)ccc2-3)no1] |
| 888 | 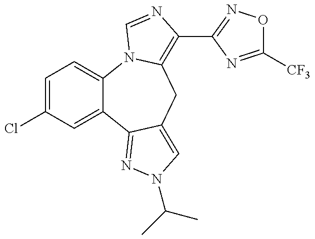 [SMILES: CC(C)n1cc2c(n1)-c1cc(Cl)ccc1-n1cnc(-c3noc(C(F)(F)F)n3)c1C2] |
| 889 | 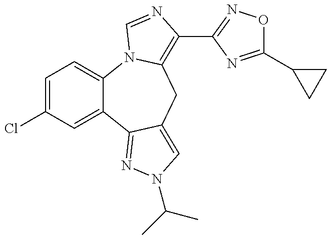 [SMILES: CC(C)n1cc2c(n1)-c1cc(Cl)ccc1-n1cnc(-c3noc(C4CC4)n3)c1C2] |
| 890 | 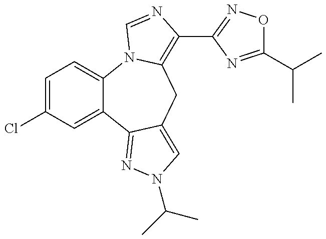 [SMILES: CC(C)c1nc(-c2ncn3c2Cc2cn(C(C)C)nc2-c2cc(Cl)ccc2-3)no1] |
| 891 | 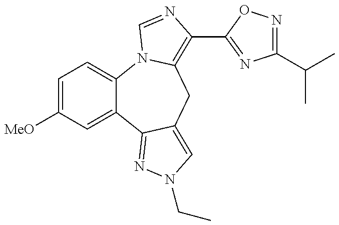 [SMILES: CCn1cc2c(n1)-c1cc(OC)ccc1-n1cnc(-c3nc(C(C)C)no3)c1C2] |
| 893 | 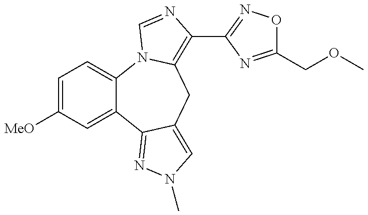 [SMILES: COCc1nc(-c2ncn3c2Cc2cn(C)nc2-c2cc(OC)ccc2-3)no1] |
| 894 | 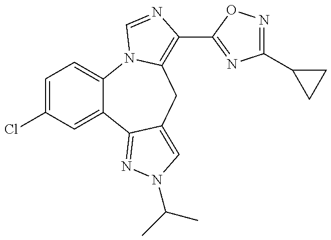 [SMILES: CC(C)n1cc2c(n1)-c1cc(Cl)ccc1-n1cnc(-c3nc(C4CC4)no3)c1C2] |
| 895 | 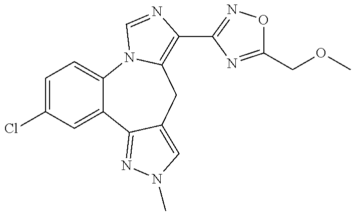 [SMILES: COCc1nc(-c2ncn3c2Cc2cn(C)nc2-c2cc(Cl)ccc2-3)no1] |
| 896 | 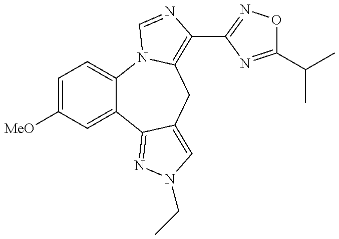 [SMILES: CCn1cc2c(n1)-c1cc(OC)ccc1-n1cnc(-c3noc(C(C)C)n3)c1C2] |
| 897 | 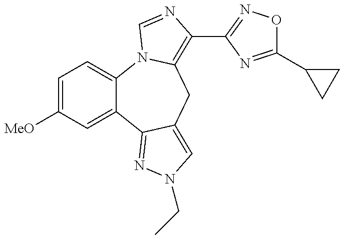 [SMILES: CCn1cc2c(n1)-c1cc(OC)ccc1-n1cnc(-c3noc(C4CC4)n3)c1C2] |
| 898 | 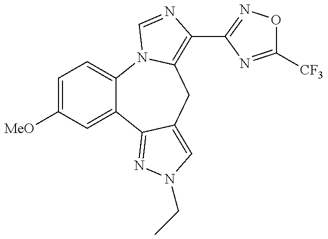 [SMILES: CCn1cc2c(n1)-c1cc(OC)ccc1-n1cnc(-c3noc(C(F)(F)F)n3)c1C2] |
| 899 | 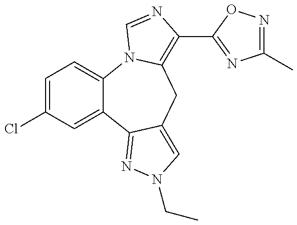 [SMILES: CCn1cc2c(n1)-c1cc(Cl)ccc1-n1cnc(-c3nc(C)no3)c1C2] |
| 900 | 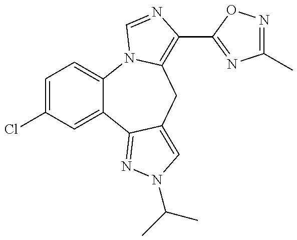 [SMILES: Cc1noc(-c2ncn3c2Cc2cn(C(C)C)nc2-c2cc(Cl)ccc2-3)n1] |
| 901 | 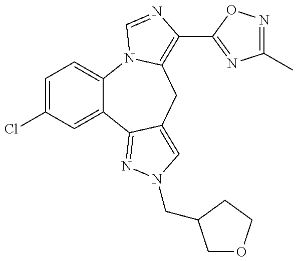 [SMILES: Cc1noc(-c2ncn3c2Cc2cn(CC4CCOC4)nc2-c2cc(Cl)ccc2-3)n1] |
| 902 | 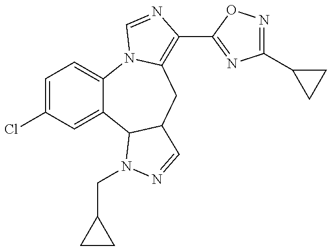 [SMILES: Clc1ccc2c(c1)C1C(C=NN1CC1CC1)Cc1c(-c3nc(C4CC4)no3)ncn1-2] |
| 903 | 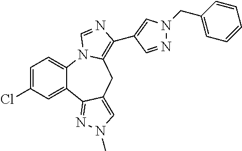 [SMILES: Cn1cc2c(n1)-c1cc(Cl)ccc1-n1cnc(-c3cnn(Cc4ccccc4)c3)c1C2] |
| 905 | 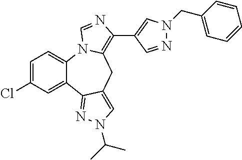 [SMILES: CC(C)n1cc2c(n1)-c1cc(Cl)ccc1-n1cnc(-c3cnn(Cc4ccccc4)c3)c1C2] |
| 907 | 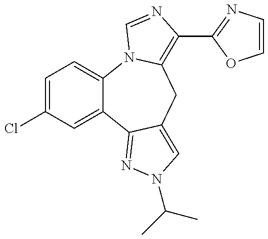 [SMILES: CC(C)n1cc2c(n1)-c1cc(Cl)ccc1-n1cnc(-c3ncco3)c1C2] |
| 908 | 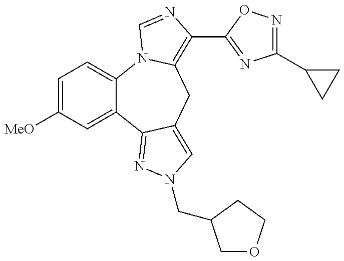 [SMILES: COc1ccc2c(c1)-c1nn(CC3CCOC3)cc1Cc1c(-c3nc(C4CC4)no3)ncn1-2] |
| 909 | 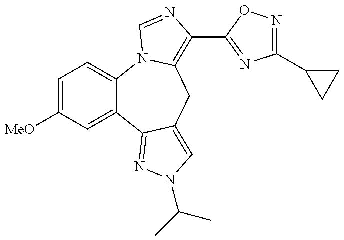 [SMILES: COc1ccc2c(c1)-c1nn(C(C)C)cc1Cc1c(-c3nc(C4CC4)no3)ncn1-2] |
| 910 | 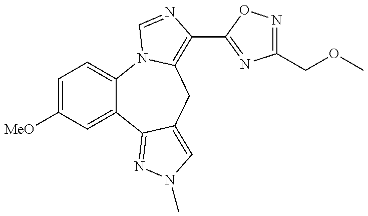 [SMILES: COCc1noc(-c2ncn3c2Cc2cn(C)nc2-c2cc(OC)ccc2-3)n1] |
| 911 | 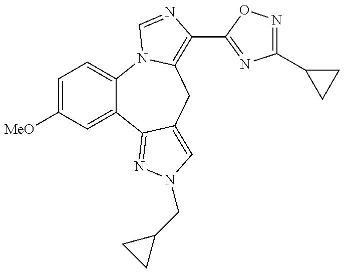 [SMILES: COc1ccc2c(c1)-c1nn(CC3CC3)cc1Cc1c(-c3nc(C4CC4)no3)ncn1-2] |
| 912 | 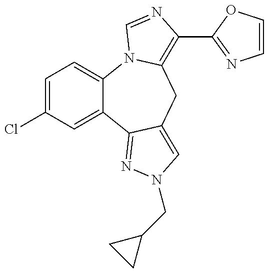 [SMILES: Clc1ccc2c(c1)-c1nn(CC3CC3)cc1Cc1c(-c3ncco3)ncn1-2] |
| 913 | 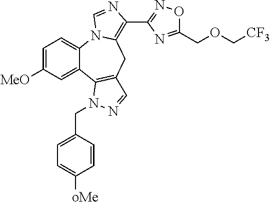 |
| 914 | 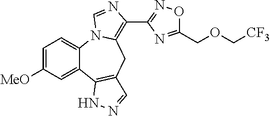 [SMILES: COc1ccc2c(c1)-c1[nH]ncc1Cc1c(-c3noc(COCC(F)(F)F)n3)ncn1-2] |
| 915 | 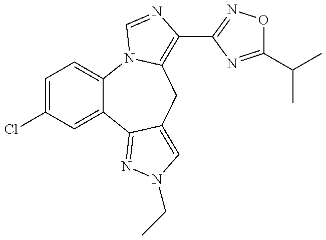 [SMILES: CCn1cc2c(n1)-c1cc(Cl)ccc1-n1cnc(-c3noc(C(C)C)n3)c1C2] |
| 916 | 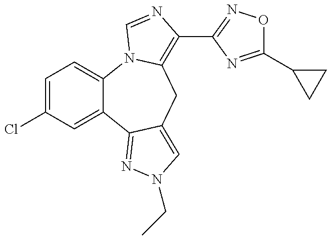 [SMILES: CCn1cc2c(n1)-c1cc(Cl)ccc1-n1cnc(-c3noc(C4CC4)n3)c1C2] |
| 917 | 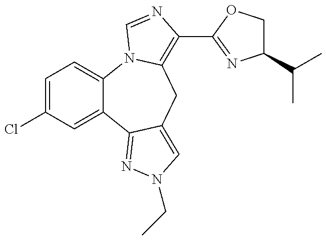 [SMILES: CCn1cc2c(n1)-c1cc(Cl)ccc1-n1cnc(C3=N[C@H](C(C)C)CO3)c1C2] |
| 918 | 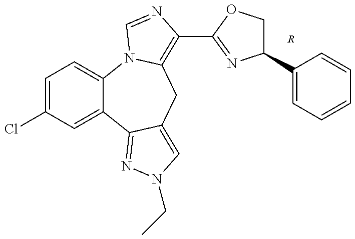 |
| 919 | 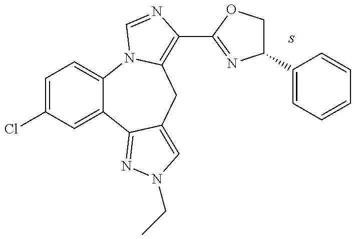 |
| 920 | 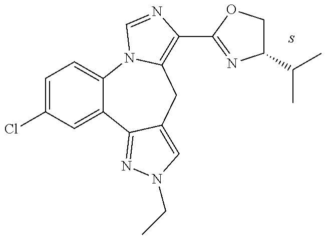 |
| 921 | 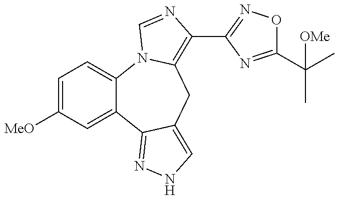 [SMILES: COc1ccc2c(c1)-c1n[nH]cc1Cc1c(-c3noc(C(C)(C)OC)n3)ncn1-2] |
| 922 | 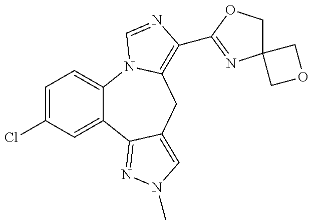 [SMILES: Cn1cc2c(n1)-c1cc(Cl)ccc1-n1cnc(C3=NC4(COC4)CO3)c1C2] |
| 923 | 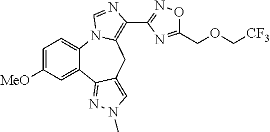 [SMILES: COc1ccc2c(c1)-c1nn(C)cc1Cc1c(-c3noc(COCC(F)(F)F)n3)ncn1-2] |
| 924 | 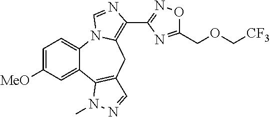 [SMILES: COc1ccc2c(c1)-c1c(cnn1C)Cc1c(-c3noc(COCC(F)(F)F)n3)ncn1-2] |
| 925 | 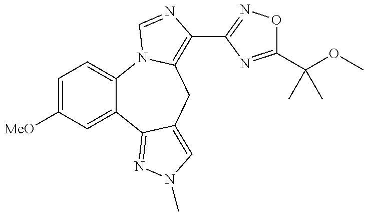 [SMILES: COc1ccc2c(c1)-c1nn(C)cc1Cc1c(-c3noc(C(C)(C)OC)n3)ncn1-2] |
| 926 | 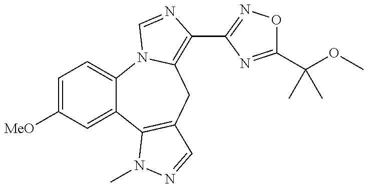 [SMILES: COc1ccc2c(c1)-c1c(cnn1C)Cc1c(-c3noc(C(C)(C)OC)n3)ncn1-2] |
| 927 | 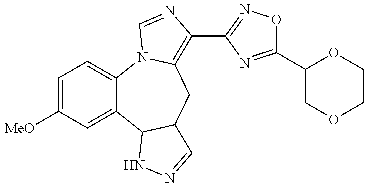 [SMILES: COc1ccc2c(c1)C1NN=CC1Cc1c(-c3noc(C4COCCO4)n3)ncn1-2] |
| 928 | 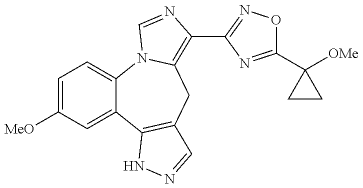 [SMILES: COc1ccc2c(c1)-c1[nH]ncc1Cc1c(-c3noc(C4(OC)CC4)n3)ncn1-2] |
| 929 | 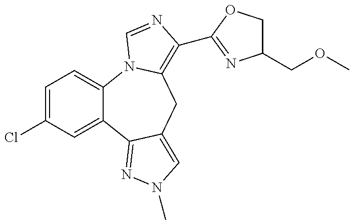 [SMILES: COCC1COC(c2ncn3c2Cc2cn(C)nc2-c2cc(Cl)ccc2-3)=N1] |
| 930 | 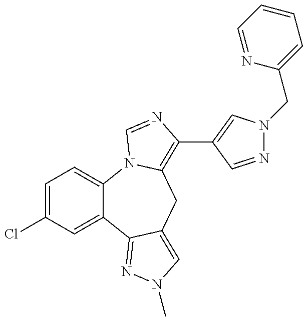 [SMILES: Cn1cc2c(n1)-c1cc(Cl)ccc1-n1cnc(-c3cnn(Cc4ccccn4)c3)c1C2] |
| 931 | 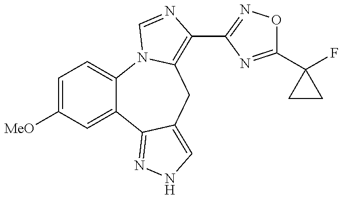 [SMILES: COc1ccc2c(c1)-c1n[nH]cc1Cc1c(-c3noc(C4(F)CC4)n3)ncn1-2] |
| 932 | 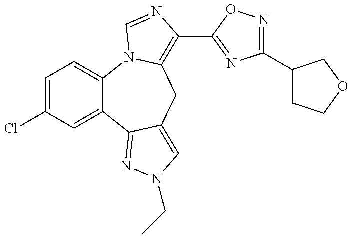 [SMILES: CCn1cc2c(n1)-c1cc(Cl)ccc1-n1cnc(-c3nc(C4CCOC4)no3)c1C2] |
| 933 | 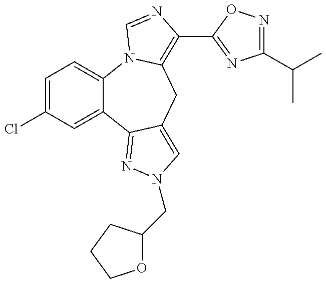 [SMILES: CC(C)c1noc(-c2ncn3c2Cc2cn(CC4CCCO4)nc2-c2cc(Cl)ccc2-3)n1] |
| 934 | 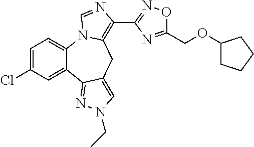 [SMILES: CCn1cc2c(n1)-c1cc(Cl)ccc1-n1cnc(-c3noc(COC4CCCC4)n3)c1C2] |
| 935 | 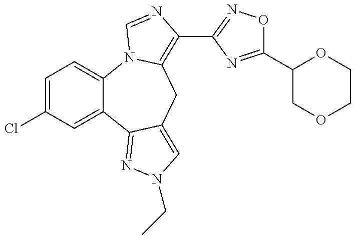 [SMILES: CCn1cc2c(n1)-c1cc(Cl)ccc1-n1cnc(-c3noc(C4COCCO4)n3)c1C2] |
| 936 | 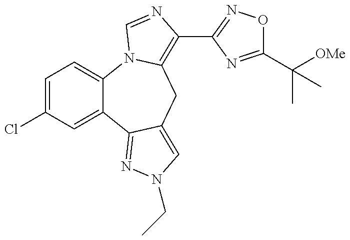 [SMILES: CCn1cc2c(n1)-c1cc(Cl)ccc1-n1cnc(-c3noc(C(C)(C)OC)n3)c1C2] |
| 937 | 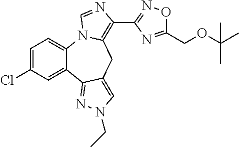 [SMILES: CCn1cc2c(n1)-c1cc(Cl)ccc1-n1cnc(-c3noc(COC(C)(C)C)n3)c1C2] |
| 938 | 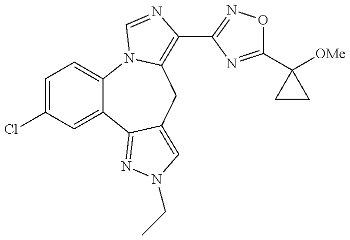 [SMILES: CCn1cc2c(n1)-c1cc(Cl)ccc1-n1cnc(-c3noc(C4(OC)CC4)n3)c1C2] |
| 939 | 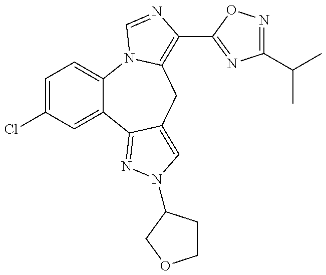 [SMILES: CC(C)c1noc(-c2ncn3c2Cc2cn(C4CCOC4)nc2-c2cc(Cl)ccc2-3)n1] |
| 940 | 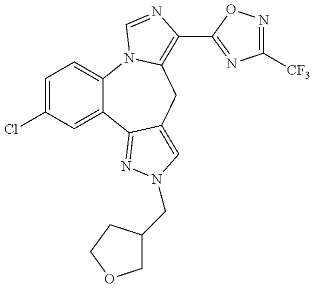 [SMILES: FC(F)(F)c1noc(-c2ncn3c2Cc2cn(CC4CCOC4)nc2-c2cc(Cl)ccc2-3)n1] |
| 941 | 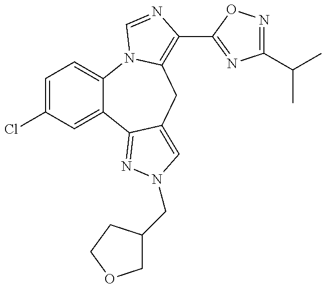 [SMILES: CC(C)c1noc(-c2ncn3c2Cc2cn(CC4CCOC4)nc2-c2cc(Cl)ccc2-3)n1] |
| 942 | 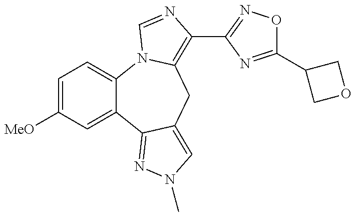 [SMILES: COc1ccc2c(c1)-c1nn(C)cc1Cc1c(-c3noc(C4COC4)n3)ncn1-2] |
| 943 | 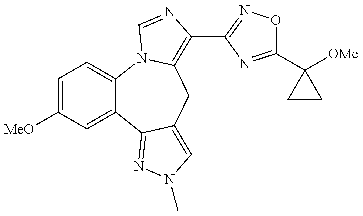 [SMILES: COc1ccc2c(c1)-c1nn(C)cc1Cc1c(-c3noc(C4(OC)CC4)n3)ncn1-2] |
| 944 | 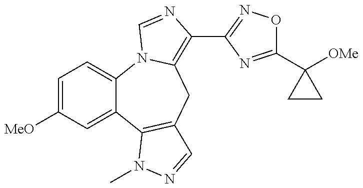 [SMILES: COc1ccc2c(c1)-c1c(cnn1C)Cc1c(-c3noc(C4(OC)CC4)n3)ncn1-2] |
| 945 | 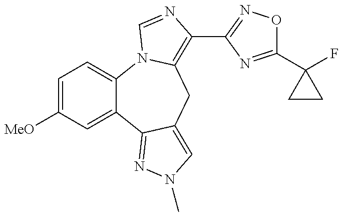 [SMILES: COc1ccc2c(c1)-c1nn(C)cc1Cc1c(-c3noc(C4(F)CC4)n3)ncn1-2] |
| 946 | 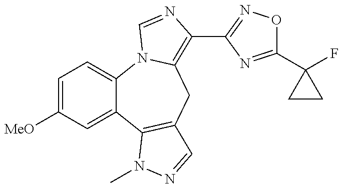 [SMILES: COc1ccc2c(c1)-c1c(cnn1C)Cc1c(-c3noc(C4(F)CC4)n3)ncn1-2] |
| 947 | 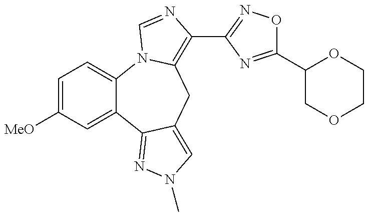 [SMILES: COc1ccc2c(c1)-c1nn(C)cc1Cc1c(-c3noc(C4COCCO4)n3)ncn1-2] |
| 948 | 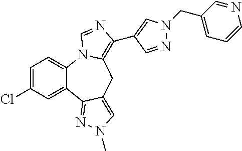 [SMILES: Cn1cc2c(n1)-c1cc(Cl)ccc1-n1cnc(-c3cnn(Cc4cccnc4)c3)c1C2] |
| 949 | 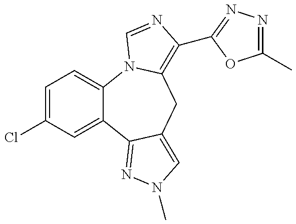 [SMILES: Cc1nnc(-c2ncn3c2Cc2cn(C)nc2-c2cc(Cl)ccc2-3)o1] |
| 950 | 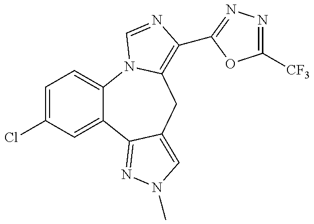 [SMILES: Cn1cc2c(n1)-c1cc(Cl)ccc1-n1cnc(-c3nnc(C(F)(F)F)o3)c1C2] |
| 951 | 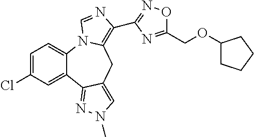 [SMILES: Cn1cc2c(n1)-c1cc(Cl)ccc1-n1cnc(-c3noc(COC4CCCC4)n3)c1C2] |
| 952 | 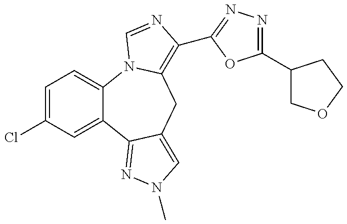 [SMILES: Cn1cc2c(n1)-c1cc(Cl)ccc1-n1cnc(-c3nnc(C4CCOC4)o3)c1C2] |
| 953 | 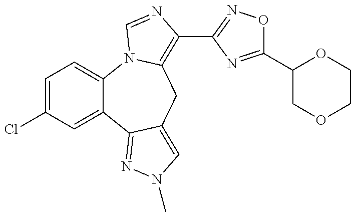 [SMILES: Cn1cc2c(n1)-c1cc(Cl)ccc1-n1cnc(-c3noc(C4COCCO4)n3)c1C2] |
| 954 | 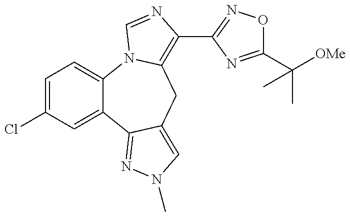 [SMILES: COC(C)(C)c1nc(-c2ncn3c2Cc2cn(C)nc2-c2cc(Cl)ccc2-3)no1] |
| 955 | 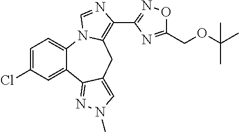 [SMILES: Cn1cc2c(n1)-c1cc(Cl)ccc1-n1cnc(-c3noc(COC(C)(C)C)n3)c1C2] |
| 956 | 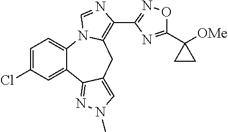 [SMILES: COC1(c2nc(-c3ncn4c3Cc3cn(C)nc3-c3cc(Cl)ccc3-4)no2)CC1] |
| 957 | 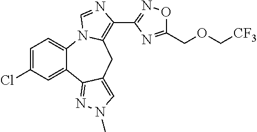 [SMILES: Cn1cc2c(n1)-c1cc(Cl)ccc1-n1cnc(-c3noc(COCC(F)(F)F)n3)c1C2] |
| 958 | 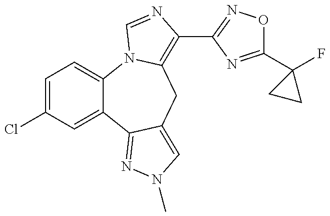 [SMILES: Cn1cc2c(n1)-c1cc(Cl)ccc1-n1cnc(-c3noc(C4(F)CC4)n3)c1C2] |
| 959 | 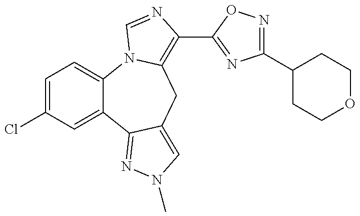 [SMILES: Cn1cc2c(n1)-c1cc(Cl)ccc1-n1cnc(-c3nc(C4CCOCC4)no3)c1C2] |
| 960 | 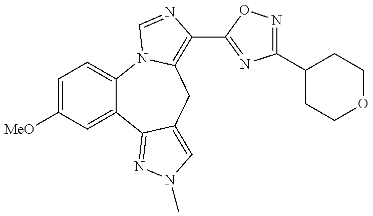 [SMILES: COc1ccc2c(c1)-c1nn(C)cc1Cc1c(-c3nc(C4CCOCC4)no3)ncn1-2] |
| 961 | 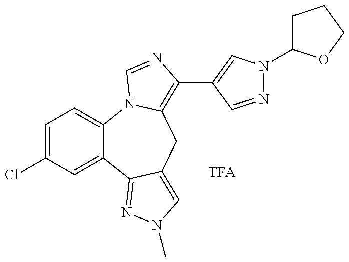 [SMILES: Cn1cc2c(n1)-c1cc(Cl)ccc1-n1cnc(-c3cnn(C4CCCO4)c3)c1C2] |
| 962 | 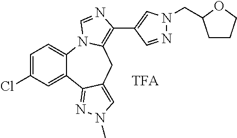 [SMILES: Cn1cc2c(n1)-c1cc(Cl)ccc1-n1cnc(-c3cnn(CC4CCCO4)c3)c1C2] |
| 963 | 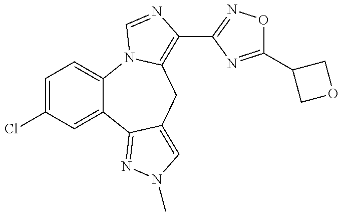 [SMILES: Cn1cc2c(n1)-c1cc(Cl)ccc1-n1cnc(-c3noc(C4COC4)n3)c1C2] |
| 964 | 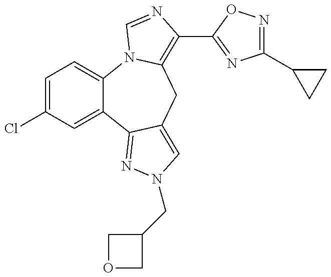 [SMILES: Clc1ccc2c(c1)-c1nn(CC3COC3)cc1Cc1c(-c3nc(C4CC4)no3)ncn1-2] |
| 965 | 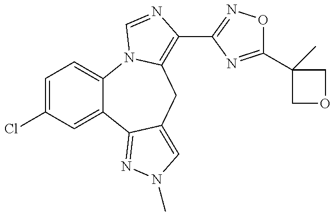 [SMILES: Cn1cc2c(n1)-c1cc(Cl)ccc1-n1cnc(-c3noc(C4(C)COC4)n3)c1C2] |
| 966 | 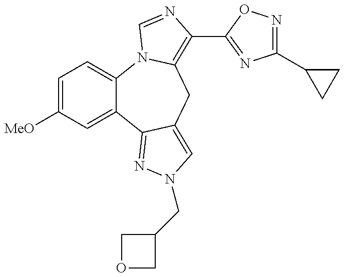 [SMILES: COc1ccc2c(c1)-c1nn(CC3COC3)cc1Cc1c(-c3nc(C4CC4)no3)ncn1-2] |
| 967 | 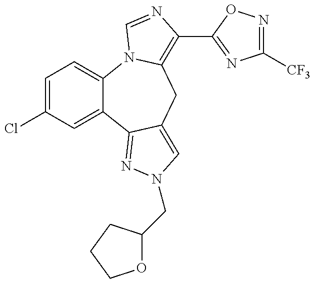 [SMILES: FC(F)(F)c1noc(-c2ncn3c2Cc2cn(CC4CCCO4)nc2-c2cc(Cl)ccc2-3)n1] |
| 968 | 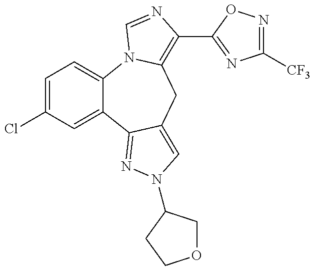 [SMILES: FC(F)(F)c1noc(-c2ncn3c2Cc2cn(C4CCOC4)nc2-c2cc(Cl)ccc2-3)n1] |
| 969 | 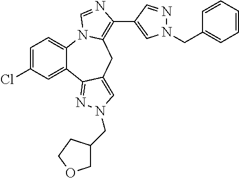 [SMILES: Clc1ccc2c(c1)-c1nn(CC3CCOC3)cc1Cc1c(-c3cnn(Cc4ccccc4)c3)ncn1-2] |
| 970 | 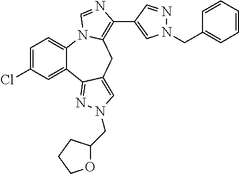 [SMILES: Clc1ccc2c(c1)-c1nn(CC3CCCO3)cc1Cc1c(-c3cnn(Cc4ccccc4)c3)ncn1-2] |
| 971 | 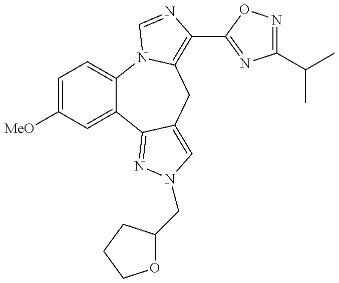 [SMILES: COc1ccc2c(c1)-c1nn(CC3CCCO3)cc1Cc1c(-c3nc(C(C)C)no3)ncn1-2] |
| 972 | 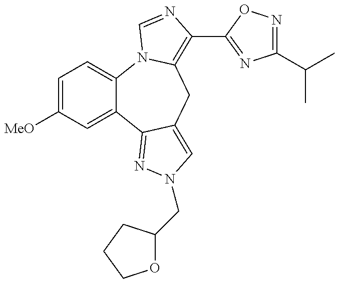 [SMILES: COc1ccc2c(c1)-c1nn(CC3CCCO3)cc1Cc1c(-c3nc(C(C)C)no3)ncn1-2] |
| 973 | 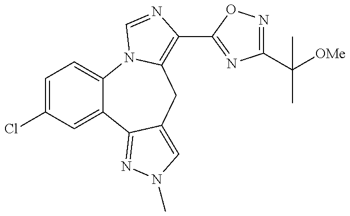 [SMILES: COC(C)(C)c1noc(-c2ncn3c2Cc2cn(C)nc2-c2cc(Cl)ccc2-3)n1] |
| 974 | 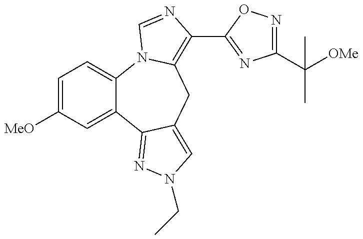 [SMILES: CCn1cc2c(n1)-c1cc(OC)ccc1-n1cnc(-c3nc(C(C)(C)OC)no3)c1C2] |
| 975 | 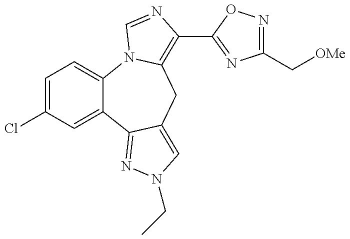 [SMILES: CCn1cc2c(n1)-c1cc(Cl)ccc1-n1cnc(-c3nc(COC)no3)c1C2] |
| 976 | 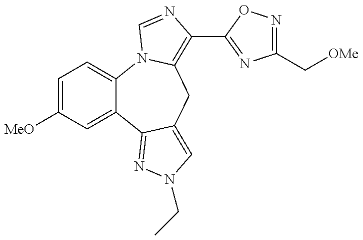 [SMILES: CCn1cc2c(n1)-c1cc(OC)ccc1-n1cnc(-c3nc(COC)no3)c1C2] |
| 977 | 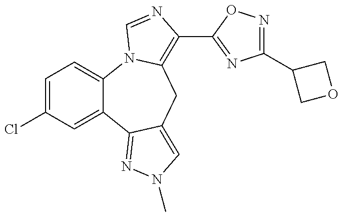 [SMILES: Cn1cc2c(n1)-c1cc(Cl)ccc1-n1cnc(-c3nc(C4COC4)no3)c1C2] |
| 979 | 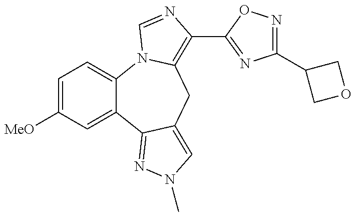 [SMILES: COc1ccc2c(c1)-c1nn(C)cc1Cc1c(-c3nc(C4COC4)no3)ncn1-2] |
| 980 | 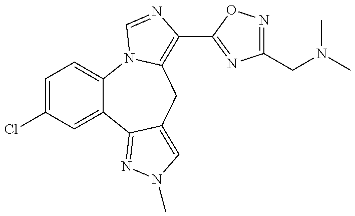 [SMILES: CN(C)Cc1noc(-c2ncn3c2Cc2cn(C)nc2-c2cc(Cl)ccc2-3)n1] |
| 981 | 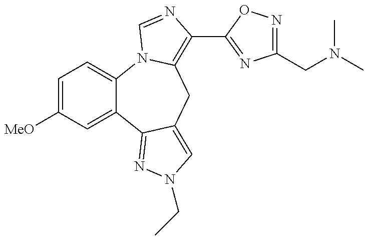 [SMILES: CCn1cc2c(n1)-c1cc(OC)ccc1-n1cnc(-c3nc(CN(C)C)no3)c1C2] |
| 982 | 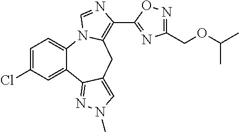 [SMILES: CC(C)OCc1noc(-c2ncn3c2Cc2cn(C)nc2-c2cc(Cl)ccc2-3)n1] |
| 983 | 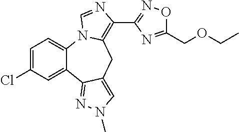 [SMILES: CCOCc1nc(-c2ncn3c2Cc2cn(C)nc2-c2cc(Cl)ccc2-3)no1] |
| 984 | 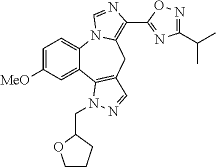 [SMILES: COc1ccc2c(c1)-c1c(cnn1CC1CCCO1)Cc1c(-c3nc(C(C)C)no3)ncn1-2] |
| 985 | 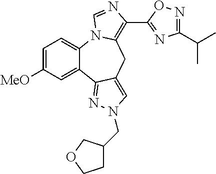 [SMILES: COc1ccc2c(c1)-c1nn(CC3CCOC3)cc1Cc1c(-c3nc(C(C)C)no3)ncn1-2] |
| 986 | 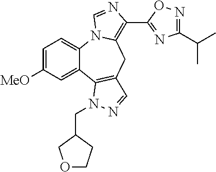 [SMILES: COc1ccc2c(c1)-c1c(cnn1CC1CCOC1)Cc1c(-c3nc(C(C)C)no3)ncn1-2] |
| 987 | 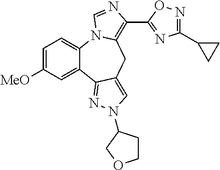 [SMILES: COc1ccc2c(c1)-c1nn(C3CCOC3)cc1Cc1c(-c3nc(C4CC4)no3)ncn1-2] |
| 988 | 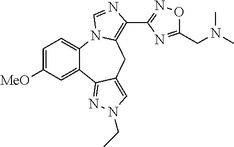 [SMILES: CCn1cc2c(n1)-c1cc(OC)ccc1-n1cnc(-c3noc(CN(C)C)n3)c1C2] |
| 989 | 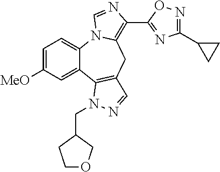 [SMILES: COc1ccc2c(c1)-c1c(cnn1CC1CCOC1)Cc1c(-c3nc(C4CC4)no3)ncn1-2] |
| 990 | 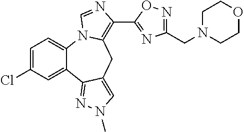 [SMILES: Cn1cc2c(n1)-c1cc(Cl)ccc1-n1cnc(-c3nc(CN4CCOCC4)no3)c1C2] |
| 991 | 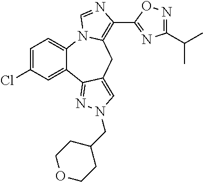 [SMILES: CC(C)c1noc(-c2ncn3c2Cc2cn(CC4CCOCC4)nc2-c2cc(Cl)ccc2-3)n1] |
| 992 | 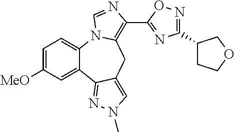 [SMILES: COc1ccc2c(c1)-c1nn(C)cc1Cc1c(-c3nc([C@H]4CCOC4)no3)ncn1-2] |
| 993 | 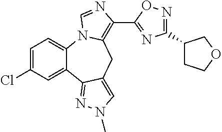 [SMILES: Cn1cc2c(n1)-c1cc(Cl)ccc1-n1cnc(-c3nc([C@H]4CCOC4)no3)c1C2] |
| 994 | 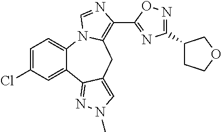 [SMILES: Cn1cc2c(n1)-c1cc(Cl)ccc1-n1cnc(-c3nc([C@H]4CCOC4)no3)c1C2] |
| 995 | 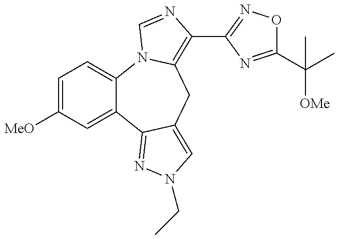 [SMILES: CCn1cc2c(n1)-c1cc(OC)ccc1-n1cnc(-c3noc(C(C)(C)OC)n3)c1C2] |
| 996 | 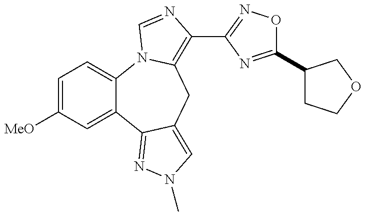 |
| 997 | 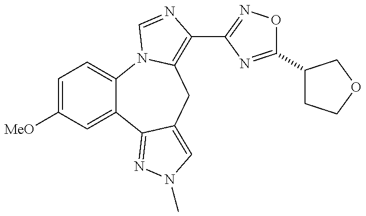 |
| 998 | 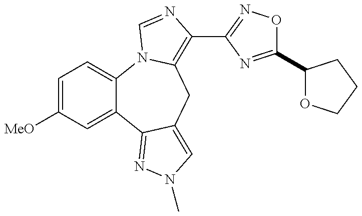 |
| 999 | 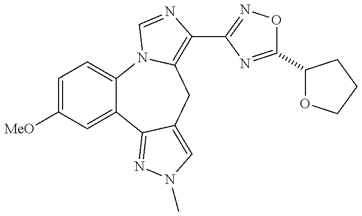 |
| 1000 | 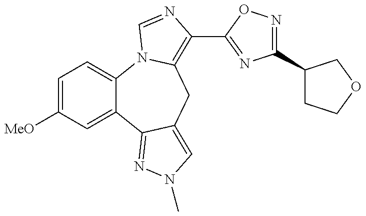 [SMILES: COc1ccc2c(c1)-c1nn(C)cc1Cc1c(-c3nc([C@@H]4CCOC4)no3)ncn1-2] |
| 1001 | 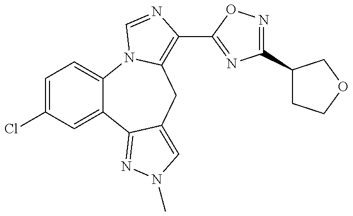 [SMILES: Cn1cc2c(n1)-c1cc(Cl)ccc1-n1cnc(-c3nc([C@@H]4CCOC4)no3)c1C2] |
| 1002 | 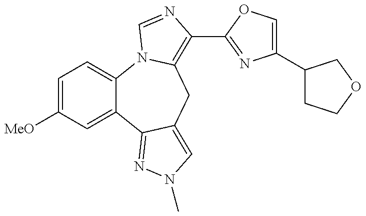 [SMILES: COc1ccc2c(c1)-c1nn(C)cc1Cc1c(-c3nc(C4CCOC4)co3)ncn1-2] |
| 1003 | 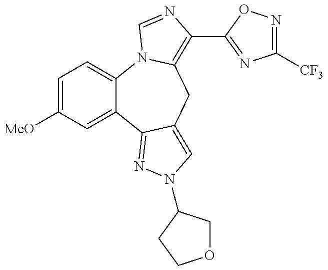 [SMILES: COc1ccc2c(c1)-c1nn(C3CCOC3)cc1Cc1c(-c3nc(C(F)(F)F)no3)ncn1-2] |
| 1004 | 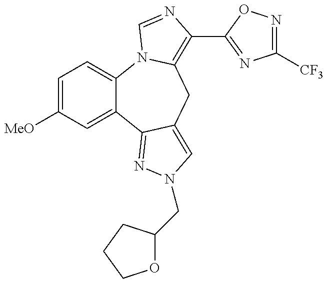 [SMILES: COc1ccc2c(c1)-c1nn(CC3CCCO3)cc1Cc1c(-c3nc(C(F)(F)F)no3)ncn1-2] |
| 1005 | 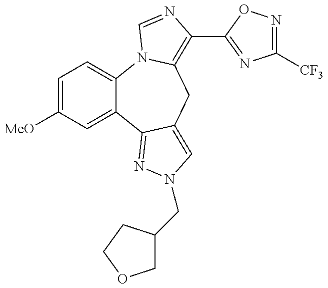 [SMILES: COc1ccc2c(c1)-c1nn(CC3CCOC3)cc1Cc1c(-c3nc(C(F)(F)F)no3)ncn1-2] |
| 1006 | 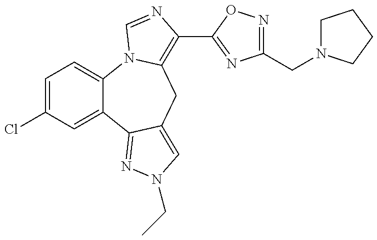 [SMILES: CCn1cc2c(n1)-c1cc(Cl)ccc1-n1cnc(-c3nc(CN4CCCC4)no3)c1C2] |
| 1007 | 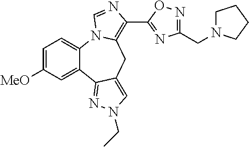 [SMILES: CCn1cc2c(n1)-c1cc(OC)ccc1-n1cnc(-c3nc(CN4CCCC4)no3)c1C2] |
| 1008 | 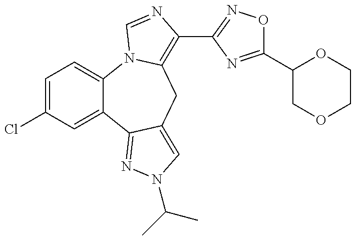 [SMILES: CC(C)n1cc2c(n1)-c1cc(Cl)ccc1-n1cnc(-c3noc(C4COCCO4)n3)c1C2] |
| 1009 | 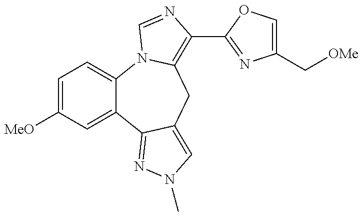 [SMILES: COCc1coc(-c2ncn3c2Cc2cn(C)nc2-c2cc(OC)ccc2-3)n1] |
| 1010 | 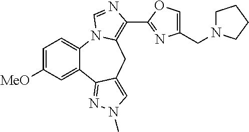 [SMILES: COc1ccc2c(c1)-c1nn(C)cc1Cc1c(-c3nc(CN4CCCC4)co3)ncn1-2] |
| 1011 | 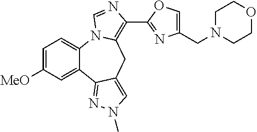 [SMILES: COc1ccc2c(c1)-c1nn(C)cc1Cc1c(-c3nc(CN4CCOCC4)co3)ncn1-2] |
| 1012 | 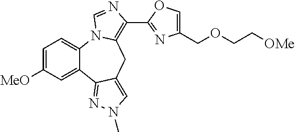 [SMILES: COCCOCc1coc(-c2ncn3c2Cc2cn(C)nc2-c2cc(OC)ccc2-3)n1] |

or a pharmaceutically acceptable salt, hydrate, solvate, polymorph, isomer, or combinations thereof.

In various aspects the present disclosure also provides pharmaceutical compositions that comprise a compound of Formula I-a, I-b, I-c, I-d, I-e, and I-f or a pharmaceutically acceptable salt, hydrate, solvate, polymorph, isomer, or combinations thereof.

In some embodiments, compounds of formula I-a are GABA_{A} α5 receptor positive allosteric modulators. In some embodiments, compounds of formula I-b are GABA_{A} α5 receptor positive allosteric modulators. In some embodiments, compounds of formula I-c are GABA_{A} α5 receptor positive allosteric modulators. In some embodiments, compounds of formula I-d are GABA_{A} α5 receptor positive allosteric modulators. In some embodiments, compounds of formula I-e are GABA_{A} α5 receptor positive allosteric modulators. In some embodiments, compounds of formula I-f are GABA_{A} α5 receptor positive allosteric modulators. In some aspects of this disclosure, one or more of the compounds of formula I-a, I-b, I-c, I-d, I-e or I-f are useful for treating the conditions described herein.

In some embodiments, the GABA_{A} α5 receptor positive allosteric modulators described in this disclosure are used in combination with one or more of the GABA_{A} α5 receptor positive allosteric modulators disclosed in PCT applications WO2015/095783A1, WO2016/205739A1, WO2018/130869A1, WO2019/246300A1, WO2021/127543 and WO2022/011314, including in particular one of more of compounds 1-740, as described individually above. In some embodiments, the GABA_{A} α5 receptor positive allosteric modulators described in this disclosure or the combinations described above may be used in combination with one or more of the SV2a inhibitors, including in some embodiments one of more of levetiracetam, brivaracetam and seletracetam, as disclosed PCT application WO2022/011318 in the treatment of such cognitive impairments and the other conditions described herein.

In some embodiments, the method comprises administering to the subject:
A) levetiracetam, brivaracetam or seletracetam, or a pharmaceutically acceptable salt thereof or a pharmaceutical composition comprising levetiracetam, brivaracetam or seletracetam, or a pharmaceutically acceptable salt thereof and a pharmaceutically acceptable carrier; and
B) a GABA_{A} α5 receptor agonist of this disclosure, or a pharmaceutically acceptable salt, hydrate, solvate, isomer, or polymorph thereof or a pharmaceutical composition comprising a GABA_{A} α5 receptor agonist of this disclosure, or a pharmaceutically acceptable salt, hydrate, solvate, isomer, or polymorph thereof and a pharmaceutically acceptable carrier, or
C) a pharmaceutical composition comprising A and B and a pharmaceutically acceptable carrier.

In some embodiments, the levetiracetam, seletracetam, or brivaracetam, or a pharmaceutically acceptable salt thereof or the pharmaceutical composition comprising the levetiracetam, brivaracetam or seletracetam, or pharmaceutically acceptable salt thereof of A and the GABA_{A} α5 receptor agonist, or pharmaceutically acceptable salt, hydrate, solvate, isomer, or polymorph thereof or the pharmaceutical composition comprising the GABA_{A} α5 receptor agonist, or pharmaceutically acceptable salt, hydrate, solvate, isomer, or polymorph thereof of B, or the pharmaceutical composition of C, is administered orally. In some embodiments, the levetiracetam, seletracetam, or brivaracetam, or a pharmaceutically acceptable salt thereof or the pharmaceutical composition comprising the levetiracetam, brivaracetam or seletracetam, or pharmaceutically acceptable salt thereof of A and the GABA_{A} α5 receptor agonist, or a pharmaceutically acceptable salt, hydrate, solvate, isomer, or polymorph thereof or the pharmaceutical composition comprising the GABA_{A} α5 receptor agonist, or pharmaceutically acceptable salt, hydrate, solvate, isomer, or polymorph thereof of B, or the pharmaceutical composition of C, is administered once daily. In other embodiments, the levetiracetam, seletracetam, or brivaracetam, or a pharmaceutically acceptable salt thereof or the pharmaceutical composition comprising the levetiracetam, brivaracetam or seletracetam, or pharmaceutically acceptable salt thereof of A and the GABA_{A} α5 receptor agonist, or a pharmaceutically acceptable salt, hydrate, solvate, isomer, or polymorph thereof or the pharmaceutical composition comprising the GABA_{A} α5 receptor agonist, or pharmaceutically acceptable salt, hydrate, solvate, isomer, or polymorph thereof of B, or the pharmaceutical composition of C, is administered twice daily.

In some embodiments, the levetiracetam, seletracetam, or brivaracetam, or a pharmaceutically acceptable salt thereof or the pharmaceutical composition comprising the levetiracetam, brivaracetam or seletracetam, or pharmaceutically acceptable salt thereof of A and the GABA_{A} α5 receptor agonist, or a pharmaceutically acceptable salt, hydrate, solvate, isomer, or polymorph thereof or the pharmaceutical composition comprising the GABA_{A} α5 receptor agonist, or pharmaceutically acceptable salt, hydrate, solvate, isomer, or polymorph thereof of B are administered simultaneously. In some embodiments, the levetiracetam, seletracetam, or brivaracetam, or a pharmaceutically acceptable salt thereof or the pharmaceutical composition comprising the levetiracetam, brivaracetam or seletracetam, or pharmaceutically acceptable salt thereof of A and the GABA_{A} α5 receptor agonist, or a pharmaceutically acceptable salt, hydrate, solvate, isomer, or polymorph thereof or the pharmaceutical composition comprising the GABA_{A} α5 receptor agonist, or pharmaceutically acceptable salt, hydrate, solvate, isomer, or polymorph thereof of B are administered sequentially.

In some embodiments, the levetiracetam, brivaracetam or seletracetam, or pharmaceutically acceptable salt thereof of A or Cis administered at a daily dose of 0.7-350 mg. In some embodiments, the levetiracetam or seletracetam, or pharmaceutically acceptable salt thereof of A or C is administered at a daily dose of 125-250 mg. In other embodiments, the levetiracetam or seletracetam, or pharmaceutically acceptable salt thereof of A or C is administered at a daily dose of 220 mg. In some embodiments, the levetiracetam or seletracetam, or pharmaceutically acceptable salt thereof of A or C is administered at a daily dose of 190 mg. In some embodiments, the brivaracetam, or pharmaceutically acceptable salt thereof of A or C is administered at a daily dose of is 0.7-180 mg. In some embodiments, the levetiracetam or seletracetam, or pharmaceutically acceptable salt thereof of A or C is administered at a daily dose of is 7-350 mg. In some embodiments, the pharmaceutical composition of A and the pharmaceutical composition of B, are packaged together. In some embodiments, the pharmaceutical composition of A and the pharmaceutical composition of B, are packaged separately.

In some embodiments, one or more of the pharmaceutical composition of A and the pharmaceutical composition of B, or the pharmaceutical composition of C, is formulated in an oral form, an extended release form or a single-unit-dosage-form or for once-a-day administration. In some embodiments, the extended release form is a controlled release form, a prolonged release form, a sustained release form, a delayed release form, or a slow release form.

In some embodiments of the methods of this disclosure, the pharmaceutical composition of A comprises levetiracetam or pharmaceutically acceptable salt in an amount of 220 mg and further comprises 280 mg-350 mg of hydroxypropyl methylcellulose, 1.2 mg-1.4 mg of colloidal silicon dioxide, 92.8 mg-119.2 mg of silicified microcrystalline cellulose, and 6.0 mg-6.7 mg of magnesium stearate. In some embodiments, the pharmaceutical composition comprises levetiracetam or pharmaceutically acceptable salt in an amount of 220 mg and further comprises 280 mg or 347.5 mg of hydroxypropyl methylcellulose, 1.2 mg of colloidal silicon dioxide, 92.8 mg or 119.2 mg of silicified microcrystalline cellulose, and 6.0 mg or 6.7 mg of magnesium stearate. In some embodiments, the pharmaceutical composition comprises levetiracetam or pharmaceutically acceptable salt in an amount of 190 mg and further comprises 300 mg of hydroxypropyl methylcellulose, 1.2 mg of colloidal silicon dioxide, 102.8 mg of silicified microcrystalline cellulose or anhydrous dicalcium phosphate, and 6 mg of magnesium stearate. In some embodiments, the hydroxypropyl methylcellulose is hypromellose 2208. In some embodiments, the silicified microcrystalline cellulose is silicified microcrystalline cellulose SMCC 90.

In some embodiments of the methods of this disclosure, the pharmaceutical composition comprising the daily dose of the levetiracetam or pharmaceutically acceptable salt thereof of A or C is in extended release form and provides a steady state plasma concentration of levetiracetam in a subject of between 1.9 μg/mL and 4.4 μg/mL within 3 hours after administration and extending for at least 8 hours of a 24-hour period after said administration. In some embodiments, the pharmaceutical composition comprising the daily dose of the levetiracetam or pharmaceutically acceptable salt thereof of A or Cis in extended release form and provides a steady state plasma concentration of levetiracetam in a subject of between 1.9 μg/mL and 4.4 μg/mL within 2 hours after said administration and extending for at least 13 hours of a 24-hour period after said administration. In some embodiments, the pharmaceutical composition comprising the daily dose of the levetiracetam or pharmaceutically acceptable salt thereof of A or C is in extended release form and provides a steady state plasma concentration of levetiracetam in a subject of between 1.9 μg/mL and 4.4 μg/mL within 1 hour after said administration and extending for at least 13 hours of a 24-hour period after said administration. In other embodiments, the pharmaceutical composition comprising the daily dose of the levetiracetam or pharmaceutically acceptable salt thereof of A or C provides a steady state plasma concentration of levetiracetam in a subject of between 1.9 μg/mL and 4.4 μg/mL within 1 hour after administration and extending for at least 13 to 16 hours of a 24-hour period after said administration. (See, e.g. FIG. 2 and WO2016191288, which is incorporated by reference herein in its entirety).

In some embodiments of the methods of this disclosure, one of more of the pharmaceutical compositions comprising the daily dose of the levetiracetam or pharmaceutically acceptable salt thereof of A or C is formulated in an oral form, an extended release form or a single-unit-dosage-form or for once-a-day administration. In other embodiments, the extended release form is a controlled release form, a prolonged release form, a sustained release form, a delayed release form, or a slow release form.

In some embodiments of the methods of this disclosure, the subject to be treated is at risk of developing cognitive decline or cognitive impairment, wherein the risk is associated with the presence of altered hippocampal functional connectivity in the subject.

In some embodiments of the methods of this disclosure, the subject to be treated is at risk of developing cognitive decline or cognitive impairment, wherein the risk is associated with aging.

In some embodiments of the methods of this disclosure, the subject to be treated is at risk of developing cognitive decline or cognitive impairment, wherein the risk is a genetic risk associated with the presence of one or more genomic variants, mutations, or polymorphs associated with a change in the expression of the genes selected from the group consisting of ABCA7, CLU, CR1, PICALM, PLD3, TREM2, and SORL1 in the genome of the subject. In some embodiments, the subject is at risk of developing cognitive decline or cognitive impairment, wherein the risk is associated with the presence of at least one allele of the APOE4 gene in the genome of the subject. In some embodiments, the subject is at risk of developing cognitive decline or cognitive impairment, wherein the risk is associated with the presence of one of more biofluid biomarkers selected from the group consisting of p-tau, t-tau, and amyloid β42 in the subject. In some embodiments, the subject is a human.

In some embodiments of the methods of this disclosure, the GABA_{A} α5 receptor agonist, or the pharmaceutically acceptable salt, hydrate, solvate, polymorph, or isomer thereof in the pharmaceutical composition of B or C, is a compound of Formula II as recited in paragraph 19 or is a compound of Formula II as recited in paragraph 20, or a pharmaceutically acceptable salt, hydrate, solvate, polymorph, or isomer thereof. In some embodiments, the GABA_{A} α5 receptor agonist, or the pharmaceutically acceptable salt, hydrate, solvate, polymorph, or isomer thereof, in the pharmaceutical composition of B or C is a compound of Formula IV as recited in paragraph 19 or is a compound of Formula IV as recited in paragraph 20, or a pharmaceutically acceptable salt, hydrate, solvate, polymorph, or isomer thereof. In some embodiments of the methods of this disclosure, the GABA_{A} α5 receptor agonist, or the pharmaceutically acceptable salt, hydrate, solvate, polymorph, or isomer thereof in the pharmaceutical composition of B or C, is selected from the group consisting of compounds 1-12, 44-56, 101-268, 270-644, 646-687, 689-698, 700-703, 705, 707-721, and 723-740, or their pharmaceutically acceptable salts, hydrates, solvates, polymorphs, or isomers.

In some embodiments of the methods of this disclosure, the GABA_{A} α5 receptor agonist, or the pharmaceutically acceptable salt, hydrate, solvate, polymorph, or isomer thereof in the pharmaceutical composition of B or C, is a compound of Formula I-a to I-f as recited in paragraph 30, or a pharmaceutically acceptable salt, hydrate, solvate, polymorph, or isomer thereof. In some embodiments of the methods of this disclosure, the GABA_{A} α5 receptor agonist, or the pharmaceutically acceptable salt, hydrate, solvate, polymorph, or isomer thereof in the pharmaceutical composition of B or C, is a compound of Formula I-a as recited in paragraph 30, or a pharmaceutically acceptable salt, hydrate, solvate, polymorph, or isomer thereof. In some embodiments, the GABA_{A} α5 receptor agonist, or the pharmaceutically acceptable salt, hydrate, solvate, polymorph, or isomer thereof, in the pharmaceutical composition of B or C is a compound of Formula I-b as recited in paragraph 30, or a pharmaceutically acceptable salt, hydrate, solvate, polymorph, or isomer thereof. In some embodiments of the methods of this disclosure, the GABA_{A} α5 receptor agonist, or the pharmaceutically acceptable salt, hydrate, solvate, polymorph, or isomer thereof in the pharmaceutical composition of B or C, is a compound of Formula I-c as recited in paragraph 30, or a pharmaceutically acceptable salt, hydrate, solvate, polymorph, or isomer thereof. In some embodiments, the GABA_{A} α5 receptor agonist, or the pharmaceutically acceptable salt, hydrate, solvate, polymorph, or isomer thereof, in the pharmaceutical composition of B or C is a compound of Formula I-d as recited in paragraph 30, or a pharmaceutically acceptable salt, hydrate, solvate, polymorph, or isomer thereof. In some embodiments of the methods of this disclosure, the GABA_{A} α5 receptor agonist, or the pharmaceutically acceptable salt, hydrate, solvate, polymorph, or isomer thereof in the pharmaceutical composition of B or C, is a compound of Formula I-e as recited in paragraph 30, or a pharmaceutically acceptable salt, hydrate, solvate, polymorph, or isomer thereof. In some embodiments, the GABA_{A} α5 receptor agonist, or the pharmaceutically acceptable salt, hydrate, solvate, polymorph, or isomer thereof, in the pharmaceutical composition of B or C is a compound of Formula I-f as recited in paragraph 30, or a pharmaceutically acceptable salt, hydrate, solvate, polymorph, or isomer thereof. In some embodiments of the methods of this disclosure, the GABA_{A} α5 receptor agonist, or the pharmaceutically acceptable salt, hydrate, solvate, polymorph, or isomer thereof in the pharmaceutical composition of B or C, is selected from the group consisting of compounds 742-755, 758-763, 765-779. 781-795, 797-810, 813-828, 830, 831, 833-846, 848-891, 893-903, 905, 907-977, 979-1012 or their pharmaceutically acceptable salts, hydrates, solvates, polymorphs, or isomers.

In some embodiments of the methods of this disclosure, the compound of Formula IV as recited in paragraph 19 or is a compound of Formula IV as recited in paragraph 20, or the pharmaceutically acceptable salt, hydrate, solvate, polymorph, or isomer thereof, in the composition of B or C is a crystalline form of Compound 606, wherein the polymorph crystalline form is Form A, Form B, Form C, Form E, or Form F. In some embodiments, the pharmaceutical composition of B or C comprises one or more crystalline forms of Compound 606, wherein the one or more crystalline forms are selected from the group consisting of Form A, Form B, Form C, Form E, and Form F. See, WO2022011318, incorporated herein by reference herein in its entirety and in particular in the context of the recited compound, its synthesis and properties.

In some embodiments of the pharmaceutical compositions of B or C, the GABA_{A} α5 receptor agonist, or the pharmaceutically acceptable salt, hydrate, solvate, isomer, or polymorph thereof, is present in an amount between 5 mg and 1000 mg. In some embodiments, the pharmaceutical composition further comprises a pharmaceutically acceptable carrier. In some embodiments, the pharmaceutical composition is formulated as a tablet, capsule, pill, lozenge, powder, granule, solution, or suspension.

In some embodiments, one or more of the GABA_{A} α5 receptor agonist, or the pharmaceutically acceptable salt, hydrate, solvate, isomer, or polymorph thereof in the compositions of B or C, is in an extended release form, a non-extended release form, or an immediate release form. In some embodiments, the GABA_{A} α5 receptor agonist, or the pharmaceutically acceptable salt, hydrate, solvate, isomer, or polymorph thereof, in the compositions of B or C is in an extended release form. In some embodiments, the extended release form is a controlled release form, a prolonged release form, a sustained release form, a delayed release form, or a slow release form. In some embodiments, the GABA_{A} α5 receptor agonist, or the pharmaceutically acceptable salt, hydrate, solvate, isomer, or polymorph thereof in the compositions of B or C, is in a non-extended release form.

One aspect of the disclosure relates to use of one or more of a pharmaceutical composition or combination of this disclosure in the manufacture of a medicament for preventing or slowing the progression of cognitive impairment or preventing the development or reducing the rate of cognitive decline in a subject displaying or presenting with cognitive performance within the normal range for the subject's age. In some embodiments, as described above, the subject is a risk for developing cognitive impairment or decline or the progression of the impairment or decline. In other embodiments, this disclosure relates to the use of one or more of a compound, a pharmaceutical composition or combination of this disclosure for preventing or slowing the progression of cognitive impairment or preventing the development or reducing the rate of cognitive decline in a subject displaying or presenting with cognitive performance within the normal range for the subject's age. In some embodiments, as described above, the subject is a risk for developing cognitive impairment or decline or the progression of the impairment or decline.

In some embodiments of the methods and uses of this disclosure, the compound, pharmaceutical composition, combination or medicament is administered subcutaneously, intravenously, orally, sublingually, buccally, transdermally, arterially, intradermally, intramuscularly, intraperitoneally, ocularly, intranasally, intraspinally or intracerebrally. In some embodiments, the compound, pharmaceutical composition, combination or medicament is administered orally. In some embodiments the subject is a human. In some embodiments the compound, pharmaceutical composition, combination or medicament is administered once daily. In some embodiments, the compound, pharmaceutical composition, combination or medicament is administered twice daily.

### BRIEF DESCRIPTION OF THE DRAWINGS

FIG. 1 depicts the plasma concentrations of levetiracetam effective in treating cognitive impairment based on aged-impaired rat studies and a phase II study in aMCI patients. In one aspect of this disclosure, the effective plasma concentration is between 1.9 and 4.4 μg/mL. In another aspect, the effective plasma concentration is between 2.9 and 4.4 μg/mL. In another aspect, the effective plasma concentration is between 1.9 and 3.9 μg/mL.

FIG. 2 shows the steady state modeling of the PK profile of the 190 mg Tablet A of Table 1, indicating that this tablet affords a plasma concentration of levetiracetam of between 1.9 and 4.4 μg/mL.

FIG. 3 shows the steady state modeling of the PK profile of the 220 mg Tablet D of Table 2, indicating that this tablet affords a plasma concentration of levetiracetam of between 2.9 and 4.4 μg/mL.

FIG. 4 is a flow diagram of one embodiment of a process for manufacturing extended release compositions of levetiracetam (e.g., the 190 mg and 220 mg tablets listed in Tables 1 and 2).

FIG. 5 demonstrates a flow diagram of a phase II, randomized, double-blind, placebo-controlled crossover clinical trial to test the effects of levetiracetam on cognitively normal (CN) subjects who have abnormal hippocampus functional connectivity.

FIG. 6 demonstrates changes in hippocampal functional connectivity between placebo treated and levetiracetam-treated subjects. The * symbol denotes the significant differences between groups.

### DETAILED DESCRIPTION OF THE DISCLOSURE

Unless otherwise defined herein, scientific and technical terms used in this application shall have the meanings that are commonly understood by those of ordinary skill in the art. Generally, nomenclature used in connection with, and techniques of, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art. See, e.g., “Principles of Neural Science,” McGraw-Hill Medical, New York, N. Y. (2000); Motulsky, “Intuitive Biostatistics,” Oxford University Press, Inc. (1995); Lodish et al., “Molecular Cell Biology, 4^{th} ed.,” W.H. Freeman & Co., New York (2000); Griffiths et al., “Introduction to Genetic Analysis, 7^{th} ed.,” W.H. Freeman & Co., N.Y. (1999); Gilbert et al., “Developmental Biology, 6^{th} ed.,” Sinauer Associates, Inc., Sunderland, MA (2000).

Chemistry terms used herein are used according to conventional usage in the art, as exemplified by “The McGraw-Hill Dictionary of Chemical Terms,” Parker S., Ed., McGraw-Hill, San Francisco, CA (1985).

All publications, patents and published patent applications referred to in this application are specifically incorporated by reference herein. In case of conflict, the present specification, including its specific definitions, will control.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The singular forms “a,” “an,” and “the” include the plurals unless the context clearly dictates otherwise.

“Including” is used to mean “including but not limited to.” “Including” and “including but not limited to” are used interchangeably.

“Patient,” “subject”, or “individual” are used interchangeably and refers to either a human or a non-human animal. Patient, subject, or individual may include mammals, such as humans, primates, livestock animals (including bovines, porcines, etc.), companion animals (e.g., canines, felines, etc.) and rodents (e.g., mice and rats). In some embodiments, the patient, subject, or individual is a human.

“Preventing” the development or progression of cognitive decline or impairment refers to affecting normal or unimpaired cognitive performance such that it does not decline or does not fall below that observed in the subject upon first presentation or diagnosis or delays such decline.

“Slowing” the development or progression of cognitive decline refers to delaying the progression of cognitive decline in a subject. This may be determined by a physician or by comparison with normal population.

“Cognitive impairment” or “cognitive decline” refers to cognitive performance in subjects that is not as robust as the normal range expected in a subject of similar age. In some cases, cognitive performance is reduced by about 5%, about 10%, about 30%, or more, compared to normal range of cognitive performance expected in a subject of similar age. In some cases, “cognitive impairment” in subjects affected by aged-related cognitive impairment may refer to cognitive performance in subjects that is not as robust as the normal range that is expected in an aged-matched subject, or the performance of a young adult subject (e.g., subjects with mean scores for a given age in a test of cognitive performance).

As used herein, “Genetic risk” or “genetic predisposition” refers to “genomic polymorph”, “genomic variants”, or “genomic mutations” associated with the development of a disease or disorder that is associated with cognitive impairment of cognitive decline. The genetic risks may be identified using GWASs or preclinical models. Genetic risk can also be associated with changes in expression, including increases, decreases, or aberrant protein expression. Genetic risk factors may be used as predictive indicators but does not absolutely indicate the progression or development of said disease or disorder.

“Cognitive performance” refers to measurable cognitive behavior or cognitive ability in a subject. There are various art-recognized tests for assessing cognitive performance in humans, for example and without limitation, the clinical global impression of change scale (CIBIC-plus scale); the Mini Mental State Exam (MMSE); the Neuropsychiatric Inventory (NPI); the Clinical Dementia Rating Scale (CDR); the Cambridge Neuropsychological Test Automated Battery (CANTAB); the Sandoz Clinical Assessment-Geriatric (SCAG), the Buschke Selective Reminding Test (Buschke and Fuld, 1974); the Verbal Paired Associates subtest; the Logical Memory subtest; the Visual Reproduction subtest of the Wechsler Memory Scale-Revised (WMS-R) (Wechsler, 1997); the Benton Visual Retention Test, or MATRICS consensus neuropsychological test battery which includes tests of working memory, speed of processing, attention, verbal learning, visual learning, reasoning and problem solving and social cognition. See Folstein et al., J Psychiatric Res 12:189-98, (1975); Robbins et al., Dementia 5:266-81, (1994); Rey, L'examen clinique en psychologie, (1964); Kluger et al., J Geriatr Psychiatry Neurol 12:168-79, (1999); Marquis et al., 2002 and Masur et al., 1994. Also see Buchanan, R. W., Keefe, R. S. E., Umbricht, D., Green, M. F., Laughren, T., and Marder, S. R. (2011), The FDA-NIMH-MATRICS guidelines for clinical trial design of cognitive-enhancing drugs: what do we know 5 years later? Schizophr. Bull. 37, 1209-1217. Another example of a cognitive test in humans is the explicit 3-alternative forced choice task. In this test, subjects are presented with color photographs of common objects consisting of a mix of three types of image pairs: similar pairs, identical pairs and unrelated foils. The second of the pair of similar objects is referred to as the “lure.” These image pairs are fully randomized and presented individually as a series of images. Subjects are instructed to make a judgment as to whether the objects seen are new, old or similar. A “similar” response to the presentation of a lure stimulus indicates successful memory retrieval by the subject. By contrast, calling the lure stimulus “old” or “new” indicates that correct memory retrieval did not occur.

In addition to assessing cognitive performance, the progression of cognitive impairment and dementia may be monitored by assessing surrogate changes in the brain of the subject. Surrogate changes include, without limitation, changes in regional brain volumes, perforant path degradation, and changes seen in brain function through resting state fMRI (R-fMRI), positron emission tomography (PET), single photon emission computed Tomography (SPECT), fluorodeoxyglucose positron emission tomography (FDG-PET), or any other imaging technique that allows one to measure brain function. Examples of regional brain volumes useful in monitoring the progression of age-related cognitive impairment and dementia include reduction of hippocampal volume and reduction in volume or thickness of entorhinal cortex. These volumes may be measured in a subject by, for example, MRI. Aisen et al., Alzheimer's & Dementia 6:239-246 (2010). Perforant path degradation has been shown to be linked to age, as well as reduced cognitive performance. For example, older adults with more perforant path degradation tend to perform worse in hippocampus-dependent memory tests. Perforant path degradation may be monitored in subjects through ultrahigh-resolution diffusion tensor imaging (DTI). Yassa et al., PNAS 107:12687-12691 (2010). Resting-state fMRI (R-fMRI) involves imaging the brain during rest and recording large-amplitude spontaneous low-frequency (<0.1 Hz) fluctuations in the fMRI signal that are temporally correlated across functionally related areas. Seed-based functional connectivity, independent component analyses, and/or frequency-domain analyses of the signals are used to reveal functional connectivity between brain areas, particularly those areas whose connectivity increase or decrease with age, as well as the extent of cognitive impairment and/or dementia. FDG-PET uses the uptake of FDG as a measure of regional metabolic activity in the brain. Decline of FDG uptake in regions such as the posterior cingulated cortex, temporoparietal cortex, and prefrontal association cortex has been shown to relate to the extent of cognitive decline and dementia. Aisen et al., Alzheimer's & Dementia 6:239-246 (2010), Herholz et al., NeuroImage 17:302-316 (2002).

“Pharmaceutically acceptable salts” include, but are not limited to, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, sethionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

“Pharmaceutically acceptable salt” includes both acid and base addition salts. “Pharmaceutically acceptable acid addition salt” may refer to those salts which retain the biological effectiveness and properties of the free bases, which are not biologically or otherwise undesirable, and which are formed with inorganic acids such as, but are not limited to, hydrohalic acids, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, hydroxyacetic acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refer to those salts which retain the biological effectiveness and properties of the free acids, which are not biologically or otherwise undesirable. These salts may be prepared from addition of an inorganic base or an organic base to the free acid. Salts derived from inorganic bases include, but are not limited to, alkali and earth alkaline metal salts, sodium, potassium, lithium, ammonium, calcium, magnesium, iron, zinc, copper, manganese, aluminum salts, and the like. Salts derived from organic bases include, but are not limited to, salts of N-methyl-D-glucamine; primary, secondary, and tertiary amines; substituted amines including naturally occurring substituted amines, cyclic amines; basic ion exchange resins; isopropylamine; trimethylamine; diethylamine; triethylamine; tripropylamine; diethanolamine; ethanolamine; deanol; 2-dimethylaminoethanol; 2-diethylaminoethanol; dicyclohexylamine; amino acids; lysine; arginine; histidine; caffeine; procaine; hydrabamine; choline; betaine; benethamine; benzathine; ethylenediamine; glucosamine; methylglucamine; theobromine; triethanolamine; tromethamine; purines; piperazine; piperidine; N-ethylpiperidine; polyamine resins; and the like.

Conversely, said salt forms can be converted into the free forms by treatment with an appropriate base or acid.

“Hydrate” refers to a combination of water with a compound wherein the water retains its molecular state as water and is either absorbed, adsorbed or contained within a crystal lattice of the compound.

“Polymorph” refers to different crystalline forms of the same compound and other solid state molecular forms including pseudo-polymorphs, such as hydrates (e.g., bound water present in the crystalline structure) and solvates (e.g., bound solvents other than water) of the same compound. Different crystalline polymorphs have different crystal structures due to a different packing of the molecules in the lattice. This results in a different crystal symmetry and/or unit cell parameters, which directly influence physical properties such the X-ray diffraction characteristics of crystals or powders. A different polymorph, for example, will in general diffract at a different set of angles and will give different values for the intensities. Therefore, X-ray powder diffraction can be used to identify different polymorphs, or a solid form that comprises more than one polymorph, in a reproducible and reliable way. Certain polymorphic forms may exhibit enhanced thermodynamic stability or may be more readily manufactured in high purity in large quantities, and thus are more suitable for inclusion in pharmaceutical formulations. Certain polymorphs may display other advantageous physical properties such as lack of hygroscopic tendencies, improved solubility, and enhanced rates of dissolution due to different lattice energies.

This application contemplates all the isomers of the compounds of the disclosure. “Isomer,” as used herein, includes optical isomers (such as stereoisomers, e.g., enantiomers and diastereoisomers), Z (zusammen) or E (entgegen) isomers, and tautomers. Many of the compounds useful in the methods, uses, combinations, pharmaceutical compositions, medicaments. Combinations for use, or pharmaceutical compositions for use of the disclosure have at least one stereogenic center in their structure. This stereogenic center may be present in a R or a S configuration, said R and S notation is used in correspondence with the rules described in Pure Appl. Chem. (1976), 45, 11-30. The disclosure also relates to all stereoisomeric forms such as enantiomeric and diastereoisomeric forms of the compounds or mixtures thereof (including all possible mixtures of stereoisomers). See, e.g., WO 01/062726. Furthermore, certain compounds which contain alkenyl groups may exist as Z (zusammen) or E (entgegen) isomers. In each instance, the disclosure includes both mixture and separate individual isomers. Multiple substituents on a piperidinyl or the azepanyl ring can also stand in either cis or trans relationship to each other with respect to the plane of the piperidinyl or the azepanyl ring. Some of the compounds may also exist in tautomeric forms. Such forms, although not explicitly indicated, are intended to be included within the scope of the present disclosure. With respect to the methods, uses, combinations, pharmaceutical compositions, combinations for use, or pharmaceutical compositions for use of the present disclosure, reference to a compound or compounds is intended to encompass that compound in each of its possible isomeric forms and mixtures thereof unless the particular isomeric form is referred to specifically. See, e.g., WO 01/062726.

“Aliphatic” as used herein refers to a straight chained or branched alkyl, alkenyl or alkynyl. It is understood that alkenyl or alkynyl embodiments need at least two carbon atoms in the aliphatic chain. Aliphatic groups typically contain from 1 (or 2) to 12 carbons, such as from 1 (or 2) to 4 carbons.

“Aryl” as used herein refer to a monocyclic or bicyclic carbocyclic aromatic ring system. Aryl as used herein includes a (C6-C12)-aryl-. For example, aryl as used herein can be a C6-C10 monocyclic or C8-C12 bicyclic carbocyclic aromatic ring system. In some embodiments, aryl as used herein can be a (C6-C10)-aryl-. Phenyl (or Ph) is an example of a monocyclic aromatic ring system. Bicyclic aromatic ring systems include systems wherein both rings are aromatic, e.g., naphthyl, and systems wherein only one of the two rings is aromatic, e.g., tetralin.

“Heterocyclic” as used herein refer to a monocyclic or bicyclic non-aromatic ring system having 1 to 4 heteroatom or heteroatom groups selected from O, N, NH, S, SO, or SO₂ in a chemically stable arrangement. Heterocyclic as used herein includes a 3- to 12-membered heterocyclyl-having 1-4 heteroatoms independently selected from O, N, NH, S, SO, or SO₂. For example, heterocyclic as used herein can be a 3- to 10-membered monocyclic or 8- to 12-membered bicyclic non-aromatic ring system having 1 to 4 heteroatom or heteroatom groups selected from O, N, NH, S, SO, or SO₂ in a chemically stable arrangement. In some embodiments, heterocyclic as used herein can be a 3- to 10-membered heterocyclyl-having 1-4 heteroatoms independently selected from O, N, NH, S, SO, or SO₂. In a bicyclic non-aromatic ring system embodiment of “heterocyclyl,” one or both rings may contain said heteroatom or heteroatom groups. In another bicyclic “heterocyclyl” embodiment, one of the two rings may be aromatic. In yet another heterocyclic ring system embodiment, a non-aromatic heterocyclic ring may optionally be fused to an aromatic carbocycle.

Examples of heterocyclic rings include 3-1H-benzimidazol-2-one, 3-(1-alkyl)-benzimidazol-2-one, 2-tetrahydrofuranyl, 3-tetrahydrofuranyl, 2-tetrahydrothiophenyl, 3-tetrahydrothiophenyl, 2-morpholino, 3-morpholino, 4-morpholino, 2-thiomorpholino, 3-thiomorpholino, 4-thiomorpholino, 1-pyrrolidinyl, 2-pyrrolidinyl, 3-pyrrolidinyl, 1-tetrahydropiperazinyl, 2-tetrahydropiperazinyl, 3-tetrahydropiperazinyl, 1-piperidinyl, 2-piperidinyl, 3-piperidinyl, 1-pyrazolinyl, 3-pyrazolinyl, 4-pyrazolinyl, 5-pyrazolinyl, 1-piperidinyl, 2-piperidinyl, 3-piperidinyl, 4-piperidinyl, 2-thiazolidinyl, 3-thiazolidinyl, 4-thiazolidinyl, 1-imidazolidinyl, 2-imidazolidinyl, 4-imidazolidinyl, 5-imidazolidinyl, indolinyl, tetrahydroquinolinyl, tetrahydroisoquinolinyl, benzothiolane, benzodithiane, and 1,3-dihydro-imidazol-2-one.

“Heteroaryl” as used herein refer to a monocyclic or bicyclic aromatic ring system having 1 to 4 heteroatom or heteroatom groups selected from O, N, NH or S in a chemically stable arrangement. Heteroaryl as used herein includes a 5- to 12-membered heteroaryl having 1-4 heteroatoms independently selected from O, N, NH or S. In some embodiments, heteroaryl as used herein can be a 5- to 10-membered heteroaryl having 1-4 heteroatoms independently selected from O, N, NH or S. For example, heteroaryl as used herein can be a 5- to 10-membered monocyclic or 8- to 12-membered bicyclic aromatic ring system having 1 to 4 heteroatom or heteroatom groups selected from O, N, NH or S in one or both rings in a chemically stable arrangement. In such a bicyclic aromatic ring system embodiment of “heteroaryl”:
both rings are aromatic; and
one or both rings may contain said heteroatom or heteroatom groups.

Examples of heteroaryl rings include 2-furanyl, 3-furanyl, N-imidazolyl, 2-imidazolyl, 4-imidazolyl, 5-imidazolyl, benzimidazolyl, 3-isoxazolyl, 4-isoxazolyl, 5-isoxazolyl, 2-oxazolyl, 4-oxazolyl, 5-oxazolyl, N-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, pyridazinyl (e.g., 3-pyridazinyl), 2-thiazolyl, 4-thiazolyl, 5-thiazolyl, tetrazolyl (e.g., 5-tetrazolyl), triazolyl (e.g., 2-triazolyl and 5-triazolyl), 2-thienyl, 3-thienyl, benzofuryl, benzothiophenyl, indolyl (e.g., 2-indolyl), pyrazolyl (e.g., 2-pyrazolyl), isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, purinyl, pyrazinyl, 1,3,5-triazinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl).

“Cycloalkyl or cycloalkenyl” refers to a monocyclic or fused or bridged bicyclic carbocyclic ring system that is not aromatic. For example, cycloalkyl or cycloalkenyl as used herein can be a C3-C10 monocyclic or fused or bridged C8-C12 bicyclic carbocyclic ring system that is not aromatic. Cycloalkenyl rings have one or more units of unsaturation. Preferred cycloalkyl or cycloalkenyl groups include cyclopropyl, cyclobutyl, cyclopentyl, cyclohexyl, cyclohexenyl, cycloheptyl, cycloheptenyl, norbornyl, adamantyl and decalinyl.

“Heteroaralkyl” refers to an alkyl in which a heteroaryl group is substituted for an alkyl H atom. For example, the alkyl group may be any straight chain hydrocarbon, and can include from 1 to 12 carbon atoms (e.g., methyl, ethyl, propyl, butyl, pentyl, hexyl, heptyl, octyl, nonyl, decyl, undecyl and dodecyl), wherein said alkyl group can be substituted with any heteroaryl group, including but not limited to, 2-furanyl, 3-furanyl, N-imidazolyl, 2-imidazolyl, 4-imidazolyl, 5-imidazolyl, benzimidazolyl, 3-isoxazolyl, 4-isoxazolyl, 5-isoxazolyl, 2-oxazolyl, 4-oxazolyl, 5-oxazolyl, N-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, pyridazinyl (e.g., 3-pyridazinyl), 2-thiazolyl, 4-thiazolyl, 5-thiazolyl, tetrazolyl (e.g., 5-tetrazolyl), triazolyl (e.g., 2-triazolyl and 5-triazolyl), 2-thienyl, 3-thienyl, benzofuryl, benzothiophenyl, indolyl (e.g., 2-indolyl), pyrazolyl (e.g., 2-pyrazolyl), isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, purinyl, pyrazinyl, 1,3,5-triazinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl.

When a substituted moiety is described without indicating the atom via which such moiety is bonded to a substituent, then the substituent may be bonded via any appropriate atom in such moiety. For example, for a substituted 5- to 10-membered heteroaryl, a substituent on the heteroaryl can be bonded to any of the ring-forming atoms of the heteroaryl ring that are substitutable (i.e., atoms bound to one or more hydrogen atoms).

When a bond to a substituent is shown to cross a bond connecting two atoms in a ring, then such substituent may be bonded to any of the ring-forming atoms in that ring that are substitutable (i.e., atoms bound to one or more hydrogen atoms), unless otherwise specified or otherwise implicit from the context. For example, when a R group is defined as a pyridine, and said pyridine is depicted as follows:

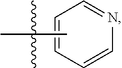

the pyridine ring may be bound to the benzodiazepine derivative through any one of the ring carbon atoms in the pyridine ring. As another example, when a R group is defined as a pyrazole, and said pyrazole is depicted as follows:

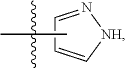

the pyrazole ring may be bound to the benzodiazepine derivative through any one of the ring carbon atoms of the pyrazole ring, or to the sp³ N-atom.

As used herein, the carbon atom designations may have the indicated integer and any intervening integer. For example, the number of carbon atoms in a (C1-C4)-alkyl group is 1, 2, 3, or 4. It should be understood that these designations refer to the total number of atoms in the appropriate group. For example, in a (C3-C10)-heterocyclyl the total number of carbon atoms and heteroatoms is 3 (as in aziridine), 4, 5, 6 (as in morpholine), 7, 8, 9, or 10.

### Compounds, Compositions, Combinations and Medicaments Useful in the Methods and of the Disclosure

Compounds, compositions, combinations and medicaments useful in the methods and uses of the disclosure are characterized by one or more of levetiracetam, brivaracetam or seletracetam, or pharmaceutically acceptable salts thereof, and GABA_{A} α5 receptor agonists, or pharmaceutically acceptable salts, hydrates, solvates, polymorphs, or isomers thereof.

### Levetiracetam, Brivaracetam and Seletracetam

Levetiracetam refers to the compound (2S)-2-(2-oxopyrrolidin-1-yl) butanamide (International Union of Pure and Applied Chemistry (IUPAC) name). Levetiracetam is a widely used antiepileptic drug. Levetiracetam binds to a specific site in the CNS: the synaptic vesicle protein 2A (SV2A) (See, e.g., Noyer et al. 1995; Fuks et al. 2003; Lynch et al. 2004; Gillard et al. 2006) and has further been shown to directly inhibit synaptic activity and neurotransmission by inhibiting presynaptic neurotransmitter release (Yang et al., 2007). Levetiracetam is sold as the FDA approved antiepileptic drug Keppra®. Typically, the therapeutically effective dose of levetiracetam (Keppra®) is in a range of 1000-3000 mg/day.

Levetiracetam is rapidly and almost completely absorbed after oral administration, and its bioavailability is not affected by food. Plasma half-life of levetiracetam is approximately 7±1 hour (expected to be 9-10 hours in elderly due to decreased renal function). Absorption is rapid, with peak plasma concentrations occurring about 1 hour following oral administration. Steady state can be achieved after 2 days of multiple twice-daily dosing.

A typical starting dose of levetiracetam in treating epilepsy in humans is 500 mg twice a day. The dosage is then increased until optimal efficacy, up to 3000 mg per day.

Brivaracetam refers to the compound (2S)-2-[(4R)-2-oxo-4-propylpyrrolidin-1-yl]butanamide (IUPAC name). It has anticonvulsant activity and binds to SV2A in the brain. It is approved under the name Briviact®. The typical starting dose is 50 mg orally twice per day, with a maintenance dose of 25-100 mg orally twice a day.

Seletracetam refers to the compound (2S)-2-[(4S)-4-(2,2-difluoroethenyl)-2-oxopyrrolidin-1-yl]butanamide (IUPAC name). It is an antiepileptic agent and binds to SV2A in the brain.

In the methods and uses of this disclosure, levetiracetam, brivaracetam, or seletracetam, or the pharmaceutically acceptable salt thereof, may be administered at doses as disclosed, for example, in U.S. patent application Ser. No. 12/580,464 (Pub. No. US-2010-0099735), U.S. patent application Ser. No. 13/287,531 (Pub. No. US-2012-0046336), U.S. patent application Ser. No. 13/370,253 (Pub. No. US-2012-0214859), WO2010044878, WO2012109491, WO2014144663, and WO2022011318. Each of these published documents is incorporated by reference herein in its entirety.

In some embodiments, the interval of administration of the levetiracetam, brivaracetam, or seletracetam, or the pharmaceutically acceptable salt thereof, or a pharmaceutical composition comprising any of the foregoing, is once every 12 hours (twice daily) or 24 hours (once daily). Administration at less frequent intervals, such as once every 6 hours, may also be used.

In some embodiments, the levetiracetam, or a pharmaceutically acceptable salt thereof, is administered at a daily dose of 70 mg to 140 mg, or 7 mg to 180 mg, or 25 mg to 180 mg, or 40 mg to 130 mg, or 140 to 300 mg, or 200 to 300 mg, or 140 to 200 mg, or 7 mg to 350 mg, 70 mg to 350 mg, 100 mg to 300 mg, or 125 mg to 250 mg. In some embodiments, the levetiracetam, or a pharmaceutically acceptable salt thereof, is administered at a daily dose of 190 mg to 220 mg. In some embodiments, the levetiracetam, or a pharmaceutically acceptable salt thereof, is administered at a daily dose of 190 mg to 240 mg. In some embodiments, the levetiracetam, or a pharmaceutically acceptable salt thereof, is administered at a daily dose of 220 mg. In some embodiments, the levetiracetam, or a pharmaceutically acceptable salt thereof, is administered at a daily dose of 190 mg.

In some embodiments of the methods of this disclosure, the levetiracetam, or a pharmaceutically acceptable salt thereof, or a pharmaceutical composition comprising levetiracetam, or a pharmaceutically acceptable salt thereof, is administered is in an oral form, extended release form (e.g., a controlled release form, a prolonged release form, a sustained release form, a delayed release form, or a slow release form), or a single-unit-dosage form or for once-a-day administration. In some embodiments, the levetiracetam, or the pharmaceutically acceptable salt thereof, or a pharmaceutical composition comprising levetiracetam, or a pharmaceutically acceptable salt thereof, is administered once or twice daily.

In certain embodiments of the disclosure, the brivaracetam, or the pharmaceutically acceptable salt thereof, is administered at a daily dose of 7 to 15 mg, or 0.7 to 180 mg, or 2.5 to 180 mg, or 4.0 to 130 mg, or 14 to 30 mg. In other embodiments. The brivaracetam, or the pharmaceutically acceptable salt of 0.7-50 mg, 0.7-75 mg, 0.7-100 mg, 0.7-150 mg, 0.7-180 mg, 1.8-50 mg, 1.8-75 mg, 1.8-100 mg, 1.8-150 mg, 1.8-180 mg, 3.5-50 mg, 3.5-75 mg, 3.5-100 mg, 3.5-150 mg, 3.5-180 mg, 5-50 mg, 5-75 mg, 5-100 mg, 5-150 mg, 5-180 mg, 7-50 mg, 7-75 mg, 7-100 mg, 7-150 mg, 7-180 mg, 15-50 mg, 15-75 mg, 15-100 mg, 15-150 mg, 15-180 mg, 35-50 mg, 35-75 mg, 35-100 mg, 35-150 mg, 35-180 mg.

In some embodiments, the brivaracetam, or a pharmaceutically acceptable salt thereof, or a pharmaceutical composition comprising brivaracetam, or a pharmaceutically acceptable salt thereof, is administered is in an oral form, extended release form (e.g., a controlled release form, a prolonged release form, a sustained release form, a delayed release form, or a slow release form), or a single-unit-dosage form or for once-a-day administration. In some embodiments, the brivaracetam, or the pharmaceutically acceptable salt thereof, or a pharmaceutical composition comprising brivaracetam, or a pharmaceutically acceptable salt thereof, is administered once or twice daily.

In some embodiments, the seletracetam, or a pharmaceutically acceptable salt thereof, is administered at a daily dose of 70 mg to 140 mg, or 7 mg to 180 mg, or 25 mg to 180 mg, or 40 mg to 130 mg, or 140 to 300 mg, or 200 to 300 mg, or 140 to 200 mg, or 7 mg to 350 mg, 70 mg to 350 mg, 100 mg to 300 mg, or 125 mg to 250 mg. In some embodiments, the seletracetam, or a pharmaceutically acceptable salt thereof, is administered at a daily dose of 190 mg to 220 mg. In some embodiments, the seletracetam, or a pharmaceutically acceptable salt thereof, is administered at a daily dose of 190 mg to 240 mg. In some embodiments, the seletracetam, or a pharmaceutically acceptable salt thereof, is administered at a daily dose of 220 mg. In some embodiments, the seletracetam, or a pharmaceutically acceptable salt thereof, is administered at a daily dose of 190 mg.

In some embodiments of the methods of this disclosure, the seletracetam, or a pharmaceutically acceptable salt thereof, or a pharmaceutical composition comprising seletracetam, or a pharmaceutically acceptable salt thereof, is administered is in an oral form, extended release form (e.g., a controlled release form, a prolonged release form, a sustained release form, a delayed release form, or a slow release form), or a single-unit-dosage form or for once-a-day administration. In some embodiments, the seletracetam, or the pharmaceutically acceptable salt thereof, or a pharmaceutical composition comprising seletracetam, or a pharmaceutically acceptable salt thereof, is administered once or twice daily.

In certain embodiments of the methods and uses of this disclosure, the daily dose of the levetiracetam or pharmaceutically acceptable salt thereof in the pharmaceutical composition is 220 mg and the pharmaceutical composition further comprises 280 mg-350 mg of hydroxypropyl methylcellulose, 1.2 mg-1.4 mg of colloidal silicon dioxide, 92.8 mg-119.2 mg of silicified microcrystalline cellulose, and 6.0 mg-6.7 mg of magnesium stearate. In other embodiments, the daily dose of the levetiracetam or pharmaceutically acceptable salt thereof in the pharmaceutical composition is 220 mg and the pharmaceutical composition further comprises 280 mg of hydroxypropyl methylcellulose, 1.2 mg of colloidal silicon dioxide, 92.8 mg of silicified microcrystalline cellulose, and 6.0 mg of magnesium stearate. In other embodiments, the daily dose of the levetiracetam or pharmaceutically acceptable salt thereof in the pharmaceutical composition is 220 mg and the pharmaceutical composition further comprises 347.5 mg of hydroxypropyl methylcellulose, 1.4 mg of colloidal silicon dioxide, 119.2 mg of silicified microcrystalline cellulose, and 6.7 mg of magnesium stearate. In some embodiments, the hydroxypropyl methylcellulose is hypromellose 2208. In some embodiments, the silicified microcrystalline cellulose is silicified microcrystalline cellulose SMCC 90.

In some embodiments of the methods and uses of this disclosure the daily dose of the levetiracetam or pharmaceutically acceptable salt thereof in the pharmaceutical composition is 190 mg and the pharmaceutical composition further comprises 300 mg of hydroxypropyl methylcellulose, 1.2 mg of colloidal silicon dioxide, 102.8 mg of silicified microcrystalline cellulose or anhydrous dicalcium phosphate, and 6 mg of magnesium stearate. In some embodiments, the hydroxypropyl methylcellulose is hypromellose 2208. In some embodiments, the silicified microcrystalline cellulose is silicified microcrystalline cellulose SMCC 90. In some embodiments, the extended release pharmaceutical composition of levetiracetam or a pharmaceutically acceptable salt thereof is in a solid form. In some embodiments, the extended release pharmaceutical composition is in the form of a tablet or capsule.

In some embodiments, the pharmaceutical composition comprising the daily dose of the levetiracetam or pharmaceutically acceptable salt thereof is in extended release form and provides a steady state plasma concentration of levetiracetam in a subject of between 1.9 μg/mL and 4.4 μg/mL within 3 hours after administration and extending for at least 8 hours of a 24-hour period after said administration. In some embodiments, the pharmaceutical composition comprising the daily dose of the levetiracetam or pharmaceutically acceptable salt thereof is in extended release form and provides a steady state plasma concentration of levetiracetam within 2 hours after said administration and extending for at least 13 hours of a 24-hour period after said administration. In some embodiments, the pharmaceutical composition comprising the daily dose of the levetiracetam or pharmaceutically acceptable salt thereof is in extended release form and provides a steady state plasma concentration of levetiracetam within 1 hour after said administration and extending for at least 13 hours of a 24-hour period after said administration. In other embodiments, the pharmaceutical composition provides said steady state plasma concentration of levetiracetam within 1 hour after administration and extending for at least 13 to 16 hours of a 24-hour period after said administration. See, e.g. WO2016191288.

In some embodiments, the pharmaceutical composition comprising the daily dose of the levetiracetam or pharmaceutically acceptable salt thereof is formulated in one or more of an oral form, an extended release form or a single-unit-dosage-form or for once-a-day administration. In other embodiments, the extended release form is a controlled release form, a prolonged release form, a sustained release form, a delayed release form, or a slow release form. In some embodiments, the extended release pharmaceutical composition of levetiracetam or a pharmaceutically acceptable salt thereof is in a solid form. In some embodiments, the extended release pharmaceutical composition of levetiracetam or a pharmaceutically acceptable salt thereof is in the form of a tablet or capsule.

Table 1 provides a description of three formulations of levetiracetam (190 mg Tablets A, B, and C). In some embodiments, the pharmaceutical composition useful in the methods and uses of this disclosure is the formulations in Table 1. In one embodiment of the methods and uses of this disclosure, the pharmaceutical composition is the 190 mg Tablet A formulation.

TABLE 1
A process for making extended release compositions
comprising 190 mg of levetiracetam

| | | Tablet A | Tablet B | Tablet C |
|---|---|---|---|---|
| Ingredient | Functionality | (Mg/Tablet) | (Mg/Tablet) | (Mg/Tablet) |
| Levetiracetam Base | API | 190.0 | 190.0 | 190.0 |
| Hypromellose | Matrix Former | 300.0 | — | — |
| (Methocel ™ K15M CR) | | | | |
| Hypromellose | Matrix Former | — | 300.0 | 300.0 |
| (Methocel ™ K100M | | | | |
| Premium CR) | | | | |
| Colloidal | Glidant | 1.2 | 1.2 | 1.2 |
| Silicon Dioxide | | | | |
| Silicified | Diluent | 102.8 | 102.8 | — |
| Microcrystalline | | | | |
| Cellulose | | | | |
| ProSolv ™ HD90 | | | | |
| Encompress, Anhydrous | Diluent | — | — | 102.8 |
| dicalcium phosphate | | | | |
| Magnesium Stearate | Lubricant | 6.0 | 6.0 | 6.0 |

| | | | |
|---|---|---|---|
| Total | 600 | 600 | 600 |

Table 2 provides a description of two formulations of levetiracetam (220 mg Tablets D and E). In some embodiments of the methods and uses of this disclosure, the pharmaceutical composition is the formulations in Table 2. In one embodiment, the pharmaceutical composition is the 220 mg Tablet D formulation.

TABLE 2
A process for making extended release compositions
comprising 220 mg of levetiracetam

| | | Tablet D | Tablet E |
|---|---|---|---|
| Ingredient | Functionality | (Mg/Tablet) | (Mg/Tablet) |
| Levetiracetam | API | 220.0 | 220.0 |
| Hypromellose | Matrix Former | 280.0 | 347.5 |
| (Methocel ™ | | | |
| K15M CR) | | | |
| Colloidal | Glidant | 1.2 | 1.4 |
| Silicon Dioxide | | | |
| Silicified | Diluent | 92.8 | 119.2 |
| Microcrystalline | | | |
| Cellulose | | | |
| ProSolv ™ HD90 | | | |
| Magnesium Stearate | Lubricant | 6.0 | 6.7 |

| | | |
|---|---|---|
| Total | 600 | 695 |

### GABA_{A} α5 Receptor Agonists

GABA_{A} receptors (GABA_{A} R) are pentameric assemblies from a pool of different subunits (α1-6, β1-3, γ1-3, δ, ε, π, θ) that form a Cl— permeable channel that is gated by the neurotransmitter γ-aminobutyric acid (GABA). Various pharmacological effects, including anxiety disorders, epilepsy, insomnia, pre-anesthetic sedation, and muscle relaxation, are mediated by different GABA_{A} subtypes.

Various studies have demonstrated that reduced GABA signaling is linked to various CNS disorders with cognitive impairment. For example, some studies have demonstrated a reduction of hippocampal expression of the α5 subunit of the GABA_{A} receptor in rats with age-related cognitive decline (See, e.g., WO 2007/019312). Other studies have shown that positive allosteric modulators of α5-containing GABA_{A} R, GABA_{A} α5 receptor agonists are useful for the treatment of cognitive impairment associated with said CNS disorders, cognitive impairment associated with a brain cancer, a brain cancer, or Parkinson's disease psychosis. See, e.g., WO 2015/095783, WO 2018/130868, WO 2016/205739, WO 2018/130869, WO 2019/246300, and U.S. 62/950,886, all of which are specifically incorporated herein by reference.

In some embodiments of the methods and uses of this disclosure, the GABA_{A} α5 receptor agonist, or the pharmaceutically acceptable salt, hydrate, solvate, isomer, or polymorph thereof or the GABA_{A} α5 receptor agonist, or the pharmaceutically acceptable salt, hydrate, solvate, isomer, or polymorph thereof included as a part of a pharmaceutical composition, is selected from the group consisting of:
i) a compound of formula II:

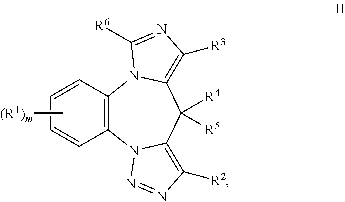

or a pharmaceutically acceptable salt, hydrate, solvate, polymorph, or isomer thereof,
wherein:
m is 0-3;
each occurrence of R¹, R², R⁴, and R⁵ are each independently selected from:
   halogen, —R, —OR, —NO₂, —NCS, —CN, —CF₃, —OCF₃, —SiR₃, —N(R)₂, —SR, —SOR, —SO₂R, —SO₂N(R)₂, —SO₃R, —(CR₂)₁₋₃R, —(CR₂)₁₋₃—OR, —(CR₂)₀₋₃—C(O)NR(CR₂)₀₋₃R, —(CR₂)₀₋₃—C(O)NR(CR₂)₀₋₃OR, —C(O)R, —C(O)C(O)R, —C(O)CH₂C(O)R, —C(S)R, —C(S)OR, —C(O)OR, —C(O)C(O)OR, —C(O)C(O)N(R)₂, —OC(O)R, —C(O)N(R)₂, —OC(O)N(R)₂, —C(S)N(R)₂, —(CR₂)₀₋₃NHC(O)R, —N(R)N(R)COR, —N(R)N(R)C(O)OR, —N(R)N(R)CON(R)₂, —N(R)SO₂R, —N(R)SO₂N(R)₂, —N(R)C(O)OR, —N(R)C(O)R, —N(R)C(S)R, —N(R)C(O)N(R)₂, —N(R)C(S)N(R)₂, —N(COR)COR, —N(OR)R, —C(═NH)N(R)₂, —C(O)N(OR)R, —C(═NOR)R, —OP(O)(OR)₂, —P(O)(R)₂, —P(O)(OR)₂, and —P(O)(H)(OR);
R³ is selected from the group consisting of:
halogen, —R, —OR, —NO₂, —NCS, —CN, —CF₃, —OCF₃, —SiR₃, —N(R)₂, —SR, —SOR, —SO₂R, —SO₂N(R)₂, —SO₃R, —(CR₂)₁₋₃R, —(CR₂)₁₋₃—OR, —(CR₂)₀₋₃—C(O)NR(CR₂)₀₋₃R, —(CR₂)₀₋₃—C(O)NR(CR₂)₀₋₃OR, —C(O)R, —C(O)C(O)R, —C(O)CH₂C(O)R, —C(S)R, —C(S)OR, —C(O)OR, —C(O)C(O)OR, —C(O)C(O)N(R)₂, —OC(O)R, —C(O)N(R)₂, —OC(O)N(R)₂, —C(S)N(R)₂, —(CR₂)₀₋₃NHC(O)R, —N(R)N(R)COR, —N(R)N(R)C(O)OR, —N(R)N(R)CON(R)₂, —N(R)SO₂R, —N(R)SO₂N(R)₂, —N(R)C(O)OR, —N(R)C(O)R, —N(R)C(S)R, —N(R)C(O)N(R)₂, —N(R)C(S)N(R)₂, —N(COR)COR, —N(OR)R, —C(═NH)N(R)₂, —C(O)N(OR)R, —C(═NOR)R, —OP(O)(OR)₂, —P(O)(R)₂, —P(O)(OR)₂, and —P(O)(H)(OR); —C≡CH, —C≡CR⁹, —(C1-C6)alkyl-C≡C—R¹⁰, —CH₂—O—R¹⁰, —CH₂—O—CH₂-R¹⁰

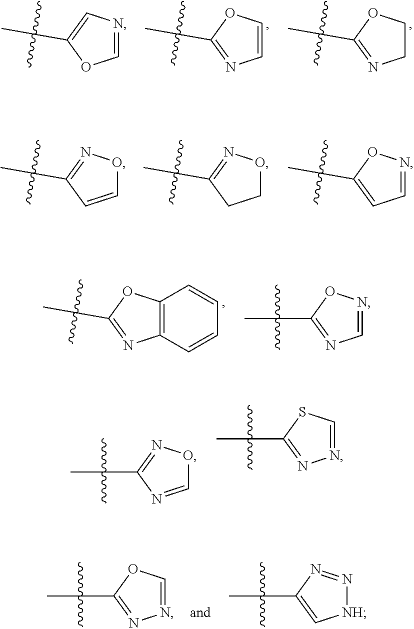

wherein each 5-member heterocycle or heteroaryl is substituted with 0-4 R₇;
wherein R³ is independently substituted with 0-5 R′;
R⁶ is selected from the group consisting of —H and —(C1-C6)alkyl;
wherein R₇ is selected from the group consisting of —(C1-C6)alkyl, —(C3-C6) cycloalkyl, -5 to 10 membered heteroaryl, —(C6-C10) aryl, (C6-C10) aryl-(C1-C6)alkyl-, -5 to 10 membered heteroaryl-(C1-C6)alkyl, and -5-10 membered heteroaryl; wherein each R₇ is independently substituted with 0-5 R′;
wherein each R⁸ is independently selected from the group consisting of —H, —(C1-C6)alkyl, —(C3-C6) cycloalkyl, —(C1-C6)alkyl-(C3-C6) cycloalkyl, —(C1-C6)alkyl-(C6-C10) aryl, —(C6-C10) aryl, -5-10 membered heteroaryl, and —(C1-C6)alkyl-5-10 membered heteroaryl;
   wherein each R⁸ excluding-H and —(C1-C6)alkyl is independently substituted by 0-5 of -halogen, —(C1-C6)alkyl, —CF₃, —OCF₃, or O—(C1-C6)alkyl;
wherein R⁹ is selected from the group consisting of —H, —(C1-C6)alkyl, —(C6-C10) aryl, -5-10 membered heteroaryl, —(C1-C6)alkyl-(C6-C10) aryl, —(C1-C6)alkyl-5-10 membered heteroaryl, —(C3-C6) cycloalkyl, —(C1-C6)alkyl-(C3-C6) cycloalkyl, —C(O)—(C6-C10) aryl, 5-10 membered heterocycle,

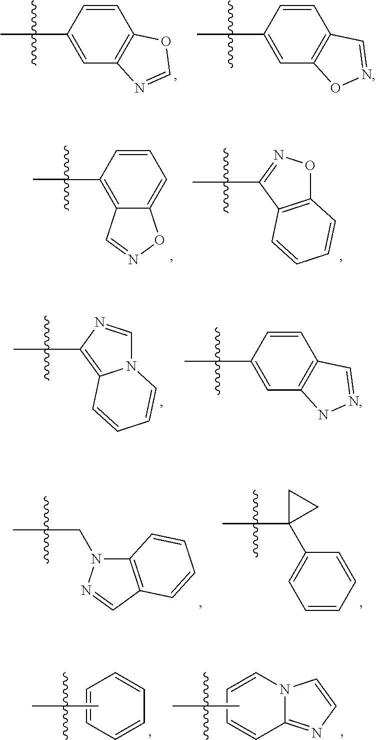

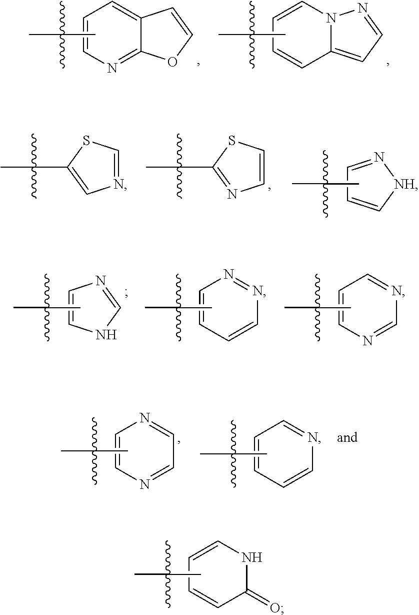

wherein each R⁹ is independently substituted with 0-5 R¹¹;
wherein R¹⁰ is selected from the group consisting of —H, halogen, —(C1-C6)alkyl, —(C6-C10) aryl, -5-10 membered heteroaryl, —(C3-C6) cycloalkyl, —CH₂—(C3-C6) cycloalkyl, —CH₂—(C6-C10) aryl, and —CH₂-5-10-membered heteroaryl,
   wherein each R¹⁰ is substituted with 0-5 R′;
wherein each occurrence of R¹¹ is independently selected from the group consisting of -halogen, —CN, SCH₃, —CF₃, —OH, —OCF₃, OCHF₂, —O(C1-C6)alkyl, —(C6-C10) aryl, —(C1-C6)alkyl, and -5 to 10 membered heteroaryl;
   each R is independently selected from the group consisting of:
      H—,
      (C1-C12)-aliphatic-,
      (C3-C10)-cycloalkyl-,
      (C3-C10)-cycloalkenyl-,
      [(C3-C10)-cycloalkyl]-(C1-C12)-aliphatic-,
      [(C3-C10)-cycloalkenyl]-(C1-C12)-aliphatic-,
      [(C3-C10)-cycloalkyl]-O—(C1-C12)-aliphatic-,
      [(C3-C10)-cycloalkenyl]-O—(C1-C12)-aliphatic-,
      (C6-C10)-aryl-,
      (C6-C10)-aryl-(C1-C12) aliphatic-,
      (C6-C10)-aryl-O—(C1-C12) aliphatic-,
      (C6-C10)-aryl-N(R″)—(C1-C12) aliphatic-,
      3- to 10-membered heterocyclyl-,
      (3- to 10-membered heterocyclyl)-(C1-C12) aliphatic-,
      (3- to 10-membered heterocyclyl)-O—(C1-C12) aliphatic-,
      (3- to 10-membered heterocyclyl)-N(R″)—(C1-C12) aliphatic-,
      5- to 10-membered heteroaryl-,
      (5- to 10-membered heteroaryl)-(C1-C12)-aliphatic-,
      (5- to 10-membered heteroaryl)-O—(C1-C12)-aliphatic-; and
      (5- to 10-membered heteroaryl)-N(R″)—(C1-C12)-aliphatic-;
wherein said heterocyclyl has 1-4 heteroatoms independently selected from the group consisting of N, NH, O, S, SO, and SO₂, and said heteroaryl has 1-4 heteroatoms independently selected from the group consisting of N, NH, O, and S;
   wherein each occurrence of R is independently substituted with 0-5 R′;
or when two R groups bound to the same atom, the two R groups may be taken together with the atom to which they are bound to form a 3- to 10-membered aromatic or non-aromatic ring having 0-4 heteroatoms independently selected from the group consisting of N, NH, O, S, SO, and SO₂, wherein said ring is optionally substituted with 0-5 R′, and wherein said ring is optionally fused to a (C6-C10) aryl, 5- to 10-membered heteroaryl, (C3-C10) cycloalkyl, or a 3- to 10-membered heterocyclyl;
wherein each occurrence of R′ is independently selected from the group consisting of halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂;
wherein each occurrence of R″ is independently selected from the group consisting of H, —(C1-C6)-alkyl, —(C1-C6)-aliphatic, (C3-C6)-cycloalkyl, 3- to 6-membered heterocyclyl, 5- to 10-membered heteroaryl-, (C6-C10)-aryl-, (5- to 10-membered heteroaryl)-(C1-C6)-alkyl-, (C6-C10)-aryl-(C1-C6)-alkyl-, (5- to 10-membered heteroaryl)-O—(C1-C6)-alkyl-, (C6-C10)-aryl-O—(C1-C6)-alkyl-, and (C6-C10)-aryl-O—(C1-C6)-alkyl-,
wherein each occurrence of R″ is independently substituted with 0-5 substituents selected from the group consisting of: halogen, —R^{o}, —OR^{o}, oxo, —CH₂OR^{o}, —CH₂N(R^{o})₂, —C(O)N(R^{o})₂, —C(O)OR^{o}, —NO₂, —NCS, —CN, —CF₃, —OCF₃ and —N(R^{o})₂, wherein each occurrence of R^{o} is independently selected from the group consisting of: —(C1-C6)-aliphatic, (C3-C6)-cycloalkyl, 3- to 6-membered heterocyclyl, 5- to 10-membered heteroaryl-, and (C6-C10)-aryl; and
   ii) a compound of formula IV:

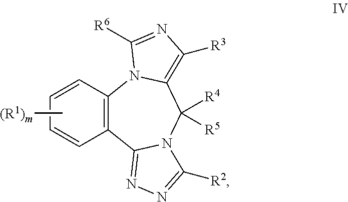

or a pharmaceutically acceptable salt, hydrate, solvate, polymorph, or isomer thereof,
wherein:
m is 0-3;
each R¹, R⁴ and R⁵ is independently selected from: each occurrence of R¹, R⁴, and R⁵ are each independently selected from:
   halogen, —R, —OR, —NO₂, —NCS, —CN, —CF₃, —OCF₂H—OCF₃, —SiR₃, —N(R)₂, —SR, —SOR, —SO₂R, —SO₂N(R)₂, —SO₃R, —(CR₂)₁₋₃R, —(CR₂)₁₋₃—OR, —(CR₂)₁₋₃—O(CR₂)₁₋₃—R, —(CR₂)₀₋₃—C(O)NR(CR₂)₀₋₃R, —(CR₂)₀₋₃—C(O)NR(CR₂)₀₋₃OR, —C(O)R, —C(O)C(O)R, —C(O)CH₂C(O)R, —C(S)R, —C(S)OR, —C(O)OR, —C(O)C(O)OR, —C(O)C(O)N(R)₂, —OC(O)R, —C(O)N(R)₂, —OC(O)N(R)₂, —C(S)N(R)₂, (CR₂)₀₋₃NHC(O)R, —N(R)N(R)COR, —N(R)N(R)C(O)OR, —N(R)N(R)CON(R)₂, —N(R)SO₂R, —N(R)SO₂N(R)₂, —N(R)C(O)OR, —N(R)C(O)R, —N(R)C(S)R, —N(R)C(O)N(R)₂, —N(R)C(S)N(R)₂, —N(COR)COR, —N(OR)R, —C(═NH)N(R)₂, —C(O)N(OR)R, —C(═NOR)R, —OP(O)(OR)₂, —P(O)(R)₂, —P(O)(OR)₂, —P(O)(H)(OR), C≡C—R⁸, CH₂CF₃, or CHF₂;
R² is selected from —OR⁸, —SR⁸, —(CH₂)ₙOR8, —(CH₂)ₙO(CH₂)ₙR⁸, —(CH₂)ₚR⁸ or —(CH₂)ₙN(R″)R¹⁰, wherein n is an integer selected from 0-4; p is an integer selected from 2-4;
   wherein R² is independently substituted with 0-5 R′;
each R³ is independently selected from:
   —H, —CN, halogen, —(C1-C6) aliphatic, —CH═CR⁹, —C≡CR⁹, —SO₂((C1-C6)alkyl), —C(O)N((C1-C6)alkyl)₂), —C(O)NH((C1-C6) aliphatic), (C6-C10)-aryl-(C1-C12) aliphatic-, —C(O)((C1-C6)alkyl), —C(O)O((C1-C6)alkyl), 5- or 6-membered heterocyclyl, 5- or 6-membered heteroaryl, —(C1-C6)alkyl-C≡C—R¹⁰, —CH₂—O—R¹⁰, —CH₂—O—CH₂—R¹⁰

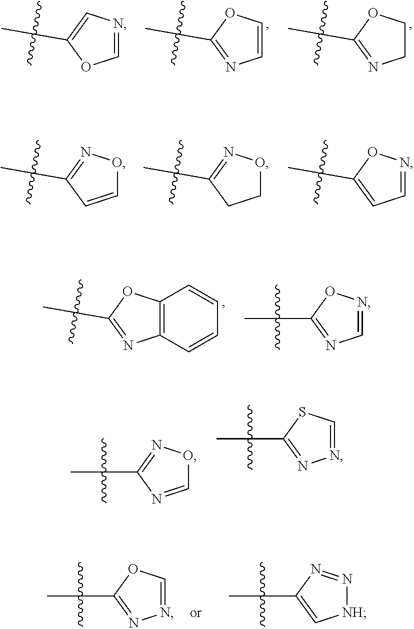

wherein each 5-10-membered heterocycle or heteroaryl are substituted with 0-3 R₇;
   wherein R³ is independently substituted with 0-5 R′;
R⁶ is selected from the group consisting of —H and —(C1-C6)alkyl;
R₇ is selected from the group consisting of —(C1-C6)alkyl, —(C3-C6) cycloalkyl, -5 to 10 membered heteroaryl, —(C6-C10) aryl, —(C6-C10) aryl-(C1-C6)alkyl, and -5 to 10 membered heteroaryl-(C1-C6)alkyl, and -5-10 membered heteroaryl;
   wherein each R₇ is independently substituted with 0-5 R′;
R⁸ is independently selected from the group consisting of —H, —(C1-C6)alkyl, —(C3-C10)-cycloalkyl, (C6-C10)-aryl, or 5- to 10-membered heteroaryl, 5-10 membered heteroaryl-(C1-C6)alkyl-, —(C1-C6)alkyl-(C6-C10) aryl, and —(C1-C6)alkyl-(C3-C6) cycloalkyl;
   wherein each occurrence of R⁸ is independently substituted with 0-5 R′;
wherein R⁹ is selected from the group consisting of —H, —(C1-C6)alkyl, —(C3-C6) cycloalkyl, —(C1-C6)alkyl-(C3-C6) cycloalkyl, —(C1-C6)alkyl-(C6-C10) aryl, —(C6-C10) aryl, -5-10 membered heteroaryl, —(C1-C6)alkyl-5-10 membered heteroaryl, 5-10 membered heterocycle, —C(O)—(C6-C10) aryl,

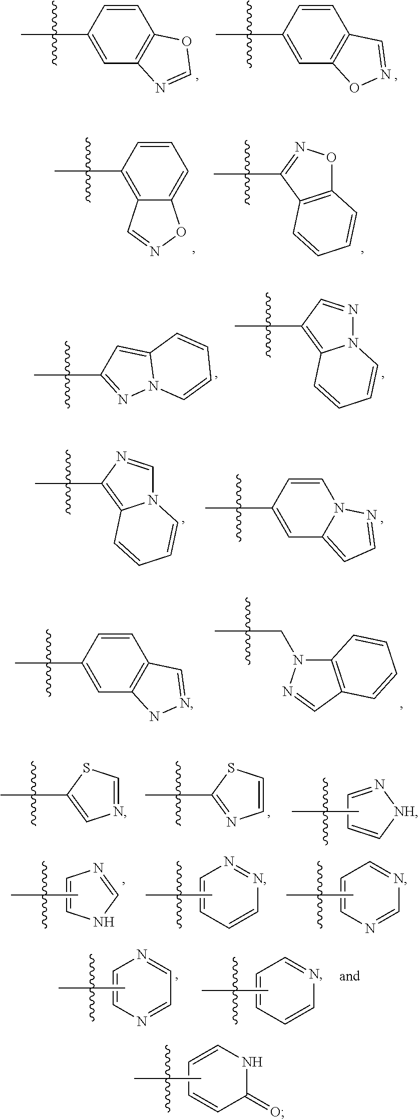

wherein each wherein each R⁹ is independently substituted with 0-5 R¹¹;
R¹⁰ is selected from the group consisting of —H, —(C1-C6)alkyl, —(C3-C10)-cycloalkyl, 3- to 10-membered heterocyclyl-, (C6-C10)-aryl, 5- to 10-membered heteroaryl, —CH₂—(C3-C6) cycloalkyl, —CH₂—(C6-C10) aryl, and —CH₂-5-10-membered heteroaryl,
   wherein each occurrence of R¹⁰ is independently substituted with 0-5 R′;
wherein each occurrence of R¹¹ is independently selected from the group consisting of -halogen, —CF₃, —OCF₃, —OH, OCF₂H, —O—(C1-C6)alkyl, —(C6-C10) aryl, —(C1-C6)alkyl, —O—CH₂—(C3-C6) cycloalkyl, —CN, and -5 to 10 membered heteroaryl;
wherein each occurrence of R′ is independently selected from the group consisting of halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂;
wherein each occurrence of R″ is independently selected from the group consisting of H, —(C1-C6)-aliphatic, —(C1-C6)-alkyl, (C3-C6)-cycloalkyl, 3- to 6-membered heterocyclyl, 5- to 10-membered heteroaryl-, (C6-C10)-aryl-, (5- to 10-membered heteroaryl)-(C1-C6)-alkyl-, (C6-C10)-aryl-(C1-C6)-alkyl-, (5- to 10-membered heteroaryl)-O—(C1-C6)-alkyl-, and (C6-C10)-aryl-O—(C1-C6)-alkyl-;
wherein each occurrence of R″ is independently substituted with 0-5 R^{t} independently selected from the group consisting of: halogen, —R^{o}, —OR^{o}, oxo, —CH₂OR^{o}, —CH₂N(R^{o})₂, —C(O)N(R^{o})₂, —C(O)OR^{o}, —NO₂, —NCS, —CN, —CF₃, —OCF₃ and —N(R^{o})₂, wherein
   each occurrence of R^{o} is independently selected from: —(C1-C6)-aliphatic, (C3-C6)-cycloalkyl, 3- to 6-membered heterocyclyl, 5- to 10-membered heteroaryl-, and (C6-C10)-aryl-.

In some embodiments, the GABA_{A} α5 receptor agonist, or the pharmaceutically acceptable salt, hydrate, solvate, isomer, or polymorph thereof or the GABA_{A} α5 receptor agonist, or pharmaceutically acceptable salt, hydrate, solvate, isomer, or polymorph thereof included as part of a pharmaceutical composition, is selected from the group consisting of:
i) a compound of formula II:

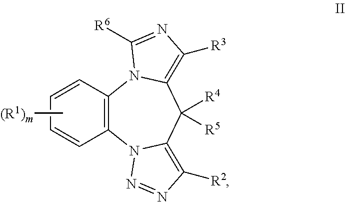

or a pharmaceutically acceptable salt, hydrate, solvate, polymorph, or isomer thereof,
wherein:
m is 0-3;
each R¹ is independently selected from the group consisting of: halogen, —H, —(C1-C6)alkyl, —OH, —O((C1-C6)alkyl), —NO₂, —CN, —CF₃, —OCF₃, —OCHF₂, —OMe, —C≡C—R⁸, —CHF₂, —CH₂CF₃, —(C6-C10) aryl, —(C1-C6)alkyl-(C6-C10) aryl, -5-10 membered heteroaryl, —(C1-C6)alkyl-5-10 membered heteroaryl, and —(C3-C6) cycloalkyl; wherein R¹ is independently substituted with 0-5 R′;
R² is selected from the group consisting of:
   —H, halogen, —OH, —(C1-C6) aliphatic, —O((C1-C6)alkyl), —C(O)O((C1-C6)alkyl), —C(O)NR₂, —(CR₂)₁₋₃—OR, —(CR₂)₁₋₃—O(CR₂)₁₋₃—R, —OR⁹, —C(O)R⁸, —CH₂R⁸, —CH₃, —CH₂—OR⁸, (C6-C10)-aryl-,
      (C6-C10)-aryl-(C1-C12) aliphatic-,
      (C6-C10)-aryl-O—(C1-C12) aliphatic-,
      (C6-C10)-aryl-N(R″)—(C1-C12) aliphatic-,
      (5- to 10-membered heteroaryl)-(C1-C12) aliphatic-,
      (5- to 10-membered heteroaryl)-O—(C1-C12) aliphatic-,
      (5- to 10-membered heteroaryl)-N(R″)—(C1-C12) aliphatic-,
      (3- to 10-membered heterocyclyl)-(C1-C12) aliphatic-,
      (3- to 10-membered heterocyclyl)-O—(C1-C12) aliphatic-, and
      (3- to 10-membered heterocyclyl)-N(R″)—(C1-C12) aliphatic-,
      wherein R² is independently substituted with 0-5 R′;
R³ is selected from the group consisting of:
   —(C1-C6)alkyl, —(C2-C6) alkenyl, —C≡CH, —C≡CR⁹, —CN, halogen, —SO₂((C6-C10)-aryl), —SO₂((C1-C6)alkyl), —C(O)N((C1-C6)alkyl)₂, —C(O)NH₂, —C(O)O((C1-C6)alkyl), —C(O)((C1-C6)alkyl), —(C6-C10) aryl, 5- to 10-membered heteroaryl, 5- to 10-membered heterocyclyl, —(C1-C6)alkyl-C≡C—R¹⁰, —CH₂—O—R¹⁰, —CH₂—O—CH₂-R¹⁰

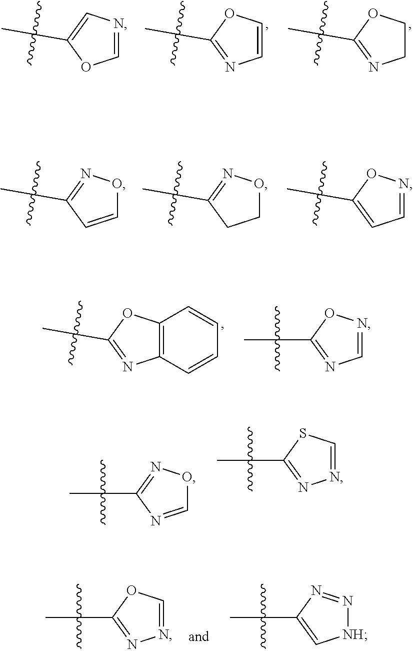

wherein each 5-member heterocycle or heteroaryl is substituted with 0-4 R₇;
wherein R³ is independently substituted with 0-5 R′;
R⁴ and R⁵ are each independently selected from the group consisting of —H, halogen, —(C1-C6)alkyl, or —(C1-C6)alkyl-(C6-C10) aryl; the (C6-C10) aryl being independently substituted with 0-5 halogen;
   R⁶ is selected from the group consisting of —H and —(C1-C6)alkyl;
wherein R₇ is selected from the group consisting of (C1-C6)alkyl, —(C3-C6) cycloalkyl, -5 to 10 membered heteroaryl, —(C6-C10) aryl, (C6-C10) aryl-(C1-C6)alkyl-, -5 to 10 membered heteroaryl-(C1-C6)alkyl, and -5-10 membered heteroaryl; wherein each R₇ is independently substituted with 0-5 R′;
wherein each R⁸ is independently selected from the group consisting of —H, —(C1-C6)alkyl, —(C3-C6) cycloalkyl, —(C1-C6)alkyl-(C3-C6) cycloalkyl, —(C1-C6)alkyl-(C6-C10) aryl, —(C6-C10) aryl, -5-10 membered heteroaryl, and —(C1-C6)alkyl-5-10 membered heteroaryl;
wherein each R⁸ excluding-H and —(C1-C6)alkyl is independently substituted by 0-5 of -halogen, —(C1-C6)alkyl, —CF₃, —OCF₃, or O—(C1-C6)alkyl;
wherein R⁹ is selected from the group consisting of —H, —(C1-C6)alkyl, —(C6-C10) aryl, -5-10 membered heteroaryl, —(C1-C6)alkyl-(C6-C10) aryl, —(C1-C6)alkyl-5-10 membered heteroaryl, —(C3-C6) cycloalkyl, —(C1-C6)alkyl-(C3-C6) cycloalkyl, —C(O)—(C6-C10) aryl, 5-10 membered heterocycle,

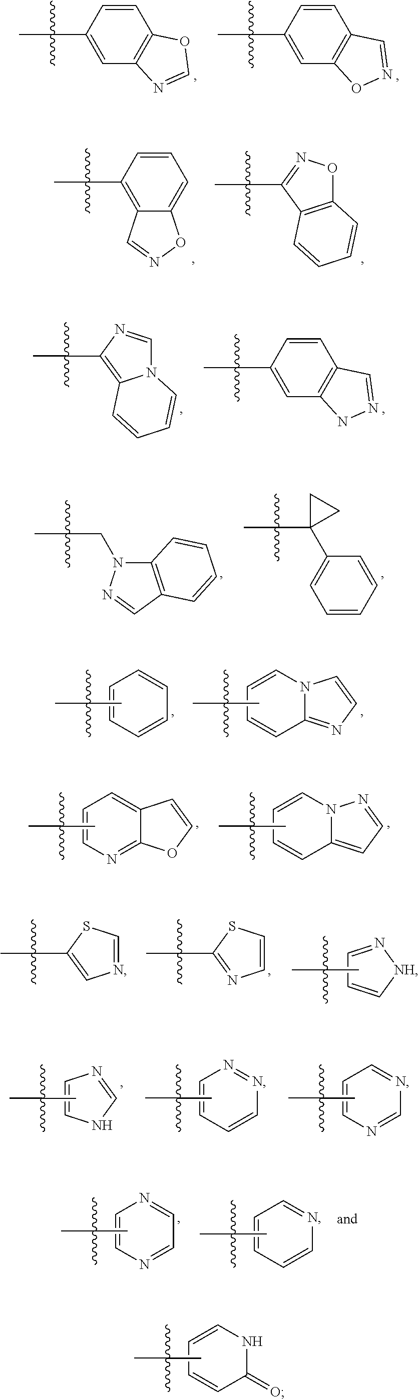

wherein each R′ is independently substituted with 0-5 R¹¹;
wherein R¹⁰ is selected from the group consisting of —H, halogen, —(C1-C6)alkyl, —(C6-C10) aryl, -5-10 membered heteroaryl, —(C3-C6) cycloalkyl, —CH₂—(C3-C6) cycloalkyl, —CH₂—(C6-C10) aryl, and —CH₂-5-10-membered heteroaryl,
   wherein each R¹⁰ is substituted with 0-5 R′;
wherein each occurrence of R¹¹ is independently selected from the group consisting of -halogen, —CN, SCH₃, —CF₃, —OH, —OCF₃, OCHF₂, —O(C1-C6)alkyl, —(C6-C10) aryl, —(C1-C6)alkyl, and -5 to 10 membered heteroaryl;
   each R is independently selected from the group consisting of:
      H—,
      (C1-C12)-aliphatic-,
      (C3-C10)-cycloalkyl-,
      (C3-C10)-cycloalkenyl-,
      [(C3-C10)-cycloalkyl]-(C1-C12)-aliphatic-,
      [(C3-C10)-cycloalkenyl]-(C1-C12)-aliphatic-,
      [(C3-C10)-cycloalkyl]-O—(C1-C12)-aliphatic-,
      [(C3-C10)-cycloalkenyl]-O—(C1-C12)-aliphatic-,
      (C6-C10)-aryl-,
      (C6-C10)-aryl-(C1-C12) aliphatic-,
      (C6-C10)-aryl-O—(C1-C12) aliphatic-,
      (C6-C10)-aryl-N(R″)—(C1-C12) aliphatic-,
      3- to 10-membered heterocyclyl-,
      (3- to 10-membered heterocyclyl)-(C1-C12) aliphatic-,
      (3- to 10-membered heterocyclyl)-O—(C1-C12) aliphatic-,
      (3- to 10-membered heterocyclyl)-N(R″)—(C1-C12) aliphatic-,
      5- to 10-membered heteroaryl-,
      (5- to 10-membered heteroaryl)-(C1-C12)-aliphatic-,
      (5- to 10-membered heteroaryl)-O—(C1-C12)-aliphatic-; and
      (5- to 10-membered heteroaryl)-N(R″)—(C1-C12)-aliphatic-;
wherein said heterocyclyl has 1-4 heteroatoms independently selected from the group consisting of N, NH, O, S, SO, and SO₂, and said heteroaryl has 1-4 heteroatoms independently selected from the group consisting of N, NH, O, and S;
   wherein each occurrence of R is independently substituted with 0-5 R′;
   or when two R groups bound to the same atom, the two R groups may be taken together with the atom to which they are bound to form a 3- to 10-membered aromatic or non-aromatic ring having 0-4 heteroatoms independently selected from the group consisting of N, NH, O, S, SO, and SO₂, wherein said ring is optionally substituted with 0-5 R′, and
   wherein said ring is optionally fused to a (C6-C10) aryl, 5- to 10-membered heteroaryl, (C3-C10) cycloalkyl, or a 3- to 10-membered heterocyclyl;
wherein each occurrence of R′ is independently selected from the group consisting of halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂;
wherein each occurrence of R″ is independently selected from the group consisting of H, —(C1-C6)-alkyl, —(C1-C6)-aliphatic, (C3-C6)-cycloalkyl, 3- to 6-membered heterocyclyl, 5- to 10-membered heteroaryl-, (C6-C10)-aryl-, (5- to 10-membered heteroaryl)-(C1-C6)-alkyl-, (C6-C10)-aryl-(C1-C6)-alkyl-, (5- to 10-membered heteroaryl)-O—(C1-C6)-alkyl-, (C6-C10)-aryl-O—(C1-C6)-alkyl-, and (C6-C10)-aryl-O—(C1-C6)-alkyl-,
wherein each occurrence of R″ is independently substituted with 0-5 substituents selected from the group consisting of: halogen, —R^{o}, —OR^{o}, oxo, —CH₂OR^{o}, —CH₂N(R^{o})₂, —C(O)N(R^{o})₂, —C(O)OR^{o}, —NO₂, —NCS, —CN, —CF₃, —OCF₃ and —N(R^{o})₂, wherein each occurrence of R^{o} is independently selected from the group consisting of: —(C1-C6)-aliphatic, (C3-C6)-cycloalkyl, 3- to 6-membered heterocyclyl, 5- to 10-membered heteroaryl-, and (C6-C10)-aryl; and
   ii) a compound of formula IV:

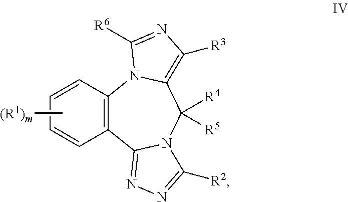

or a pharmaceutically acceptable salt, hydrate, solvate, polymorph, or isomer thereof,
wherein:
m is 0-3;
each R¹ is independently selected from the group consisting of: halogen, —H, —(C1-C6)alkyl, —C≡C—R⁹, —OH, —O((C1-C6)alkyl), —NO₂, —CN, —CF₃, —OCF₃, —CHF₂, —CH₂CF₃, —(C6-C10) aryl, —(C1-C6)alkyl-(C6-C10) aryl, -5-10 membered heteroaryl, —(C1-C6)alkyl-5-10 membered heteroaryl, and —(C3-C6) cycloalkyl;
   wherein R¹ is independently substituted with 0-5 R′;
R² is selected from the group consisting of —OR⁸, —SR⁸, —(CH₂)ₙOR8, —(CH₂)ₙO(CH₂)ₙR⁸, —(CH₂)ₚR⁸ and —(CH₂)ₙN(R″)R¹⁰, wherein n is an integer selected from 0-4; p is an integer selected from 2-4;
   wherein R² is independently substituted with 0-5 R′;
each R³ is independently selected from the group consisting of:
   —H, —CN, halogen, —(C1-C6) aliphatic, —CH═CR⁹, —C≡CR⁹, —SO₂((C1-C6)alkyl), —C(O)N((C1-C6)alkyl)₂), —C(O)NH((C1-C6) aliphatic), (C6-C10)-aryl-(C1-C12) aliphatic-, —C(O)((C1-C6)alkyl), —C(O)O((C1-C6)alkyl), 5- or 6-membered heterocyclyl, 5- or 6-membered heteroaryl, —CH₂—O—R¹⁰, —CH₂—O—CH₂—R¹⁰

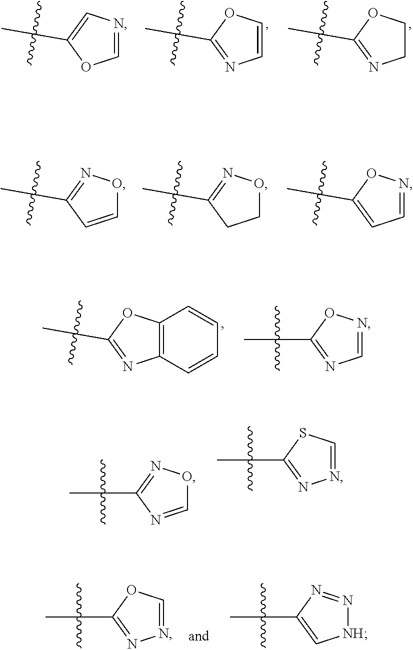

wherein each 5-10-membered heterocycle or heteroaryl are substituted with 0-3 R₇;
wherein R³ is independently substituted with 0-5 R′;
R⁴ and R⁵ are each independently selected from the group consisting of —H, halogen and —(C1-C6)alkyl;
R⁶ is selected from the group consisting of —H and —(C1-C6)alkyl;
R₇ is selected from the group consisting of —(C1-C6)alkyl, —(C3-C6) cycloalkyl, -5 to 10 membered heteroaryl, —(C6-C10) aryl, —(C6-C10) aryl-(C1-C6)alkyl, and -5 to 10 membered heteroaryl-(C1-C6)alkyl, and -5-10 membered heteroaryl;
   wherein each R₇ is independently substituted with 0-5 R′;
R⁸ is independently selected from the group consisting of —H, —(C1-C6)alkyl, —(C3-C10)-cycloalkyl, (C6-C10)-aryl, or 5- to 10-membered heteroaryl, 5-10 membered heteroaryl-(C1-C6)alkyl-, —(C1-C6)alkyl-(C6-C10) aryl, and —(C1-C6)alkyl-(C3-C6) cycloalkyl;
   wherein each occurrence of R⁸ is independently substituted with 0-5 R′;
wherein R⁹ is selected from the group consisting of —H, —(C1-C6)alkyl, —(C3-C6) cycloalkyl, —(C1-C6)alkyl-(C3-C6) cycloalkyl, —(C1-C6)alkyl-(C6-C10) aryl, —(C6-C10) aryl, -5-10 membered heteroaryl, —(C1-C6)alkyl-5-10 membered heteroaryl, 5-10 membered heterocycle, —C(O)—(C6-C10) aryl,

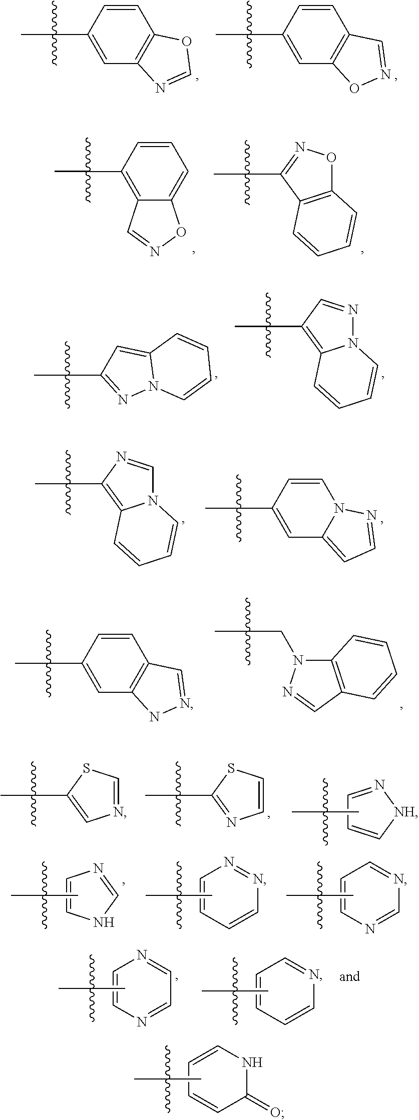

wherein each wherein each R⁹ is independently substituted with 0-5 R¹¹;
R¹⁰ is selected from the group consisting of —H, —(C1-C6)alkyl, —(C3-C10)-cycloalkyl, 3- to 10-membered heterocyclyl-, (C6-C10)-aryl, 5- to 10-membered heteroaryl, —CH₂—(C3-C6) cycloalkyl, —CH₂—(C6-C10) aryl, and —CH₂-5-10-membered heteroaryl,
   wherein each occurrence of R¹⁰ is independently substituted with 0-5 R′;
wherein each occurrence of R¹¹ is independently selected from the group consisting of -halogen, —CF₃, —OCF₃, OCF₂H, —O—(C1-C6)alkyl, —(C6-C10) aryl, —(C1-C6)alkyl, —O—CH₂—(C3-C6) cycloalkyl, and -5 to 10 membered heteroaryl;
wherein each occurrence of R′ is independently selected from the group consisting of halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂;
wherein each occurrence of R″ is independently selected from the group consisting of H, —(C1-C6)-aliphatic, —(C1-C6)-alkyl, (C3-C6)-cycloalkyl, 3- to 6-membered heterocyclyl, 5- to 10-membered heteroaryl-, (C6-C10)-aryl-, (5- to 10-membered heteroaryl)-(C1-C6)-alkyl-, (C6-C10)-aryl-(C1-C6)-alkyl-, (5- to 10-membered heteroaryl)-O—(C1-C6)-alkyl-, and (C6-C10)-aryl-O—(C1-C6)-alkyl-;
wherein each occurrence of R″ is independently substituted with 0-5 R^{t} independently selected from the group consisting of: halogen, —R^{o}, —OR^{o}, oxo, —CH₂OR^{o}, —CH₂N(R^{o})₂, —C(O)N(R^{o})₂, —C(O)OR^{o}, —NO₂, —NCS, —CN, —CF₃, —OCF₃ and —N(R^{o})₂, wherein
   each occurrence of R^{o} is independently selected from: —(C1-C6)-aliphatic, (C3-C6)-cycloalkyl, 3- to 6-membered heterocyclyl, 5- to 10-membered heteroaryl-, and (C6-C10)-aryl-.

In some embodiments, the compound has a structure of formula I-a:

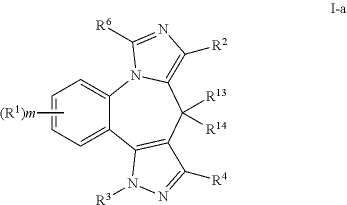

or a pharmaceutically acceptable salt, hydrate, solvate, polymorph, isomer, or combination thereof, wherein m is an integer selected from 0-4;
each R¹ is independently selected from halogen, —(C6-C10) aryl, —O(C1-C6)alkyl, —CN, —NCS, —NO₂, —CHF₂, —CF₃, —OCF₃, —OCHF₂, CO(O)R₇, CH₂—OR⁸, —(C1-C6)alkyl-(C6-C10) aryl, -5-10 membered heteroaryl, —(C1-C6)alkyl-5-10 membered heteroaryl and —(C3-C6) cycloalkyl;
   each R² is selected from CO(O)R₇, C≡C—R⁹, —(C1-C6)alkyl-C≡C—R⁹, -(5-10 membered) heteroaryl, -(3-10 membered) heterocyclyl, and (C3-C10)-cycloalkenyl; wherein each 5-6-membered heteroaryl and 3-10-membered heterocycle is substituted with 0-4 R₇;
   wherein each R₇ is selected from —H, —CF₃, —(C1-C6)alkyl, —(C3-C6) cycloalkyl, -5 to 10 membered heteroaryl, —(C6-C10) aryl, —(C6-C10) aryl-(C1-C6)alkyl, and -5 to 10 membered heteroaryl-(C1-C6)alkyl, and -3-10 membered heterocyclyl, wherein each R₇ is independently substituted with 0-5 R′;
   or when two R₇ groups bound to the same atom, the two R₇ groups may be taken together with the atom to which they are bound to form a 3-10-membered aromatic or non-aromatic ring having 0-4 heteroatoms independently selected from N, NH, O, S, SO, and SO₂, wherein said ring is optionally substituted with 0-5 R′;
each R⁸ is selected from —H, —(C1-C6 alkyl), -(5-10-membered heteroaryl), -(3-10 membered) heterocyclyl, (C3-C10)-cycloalkenyl, —(C6-C10) aryl, —(C3-C6) cycloalkyl, —CH₂—(C3-C6) cycloalkyl, —CH₂—(C6-C10) aryl and —CH₂-5-10-membered heteroaryl, wherein each occurrence of R⁸ is independently substituted by 0-5 R′.
each R⁹ is selected from: —H, —(C1-C6)alkyl, (5- to 10-membered heteroaryl), -(3-10 membered) heterocyclyl, (C3-C10)-cycloalkenyl, —(C6-C10) aryl, —(C3-C6) cycloalkyl, —(C1-C6)alkyl-(C6-C10) aryl, —(C1-C6)alkyl-5-10 membered heteroaryl, —(C1-C6)alkyl-(C3-C6) cycloalkyl, —C(O)—(C6-C10) aryl, wherein each occurrence of R⁹ is independently substituted by 0-5 R¹¹;
wherein each occurrence of R¹¹ is independently selected from —(C1-C6)alkyl, —O—(C1—C6)alkyl, -halogen, —CF₃, —OCF₃, —Ome, —(C6-C10) aryl and -5 to 10 membered heteroaryl; wherein said heterocyclyl has 1-4 heteroatoms independently selected from N, NH, O, S, SO, and SO₂, and said heteroaryl has 1-4 heteroatoms independently selected from N, NH, O, and S.
R³ is independently selected from: —H, —(C1-C6)alkyl, —(C1-C6)alkyl-(C3-C6) cycloalkyl, —(C1-C6)alkyl-OR¹², —(C1-C6)alkyl-N(R¹²)₂, —(C1-C6)alkyl-(C6-C10) aryl, —(C1-C6) alkyl-5-10 membered heteroaryl, -(3-10 membered) heterocyclyl, -5-10 membered heteroaryl, —C(O)—(C6-C10) aryl, —C(O)—(C1-C6)alkyl, —C(O)—(C3-C6) wherein R³ is independently substituted with 0-5 R¹²;
wherein each R¹² is independently selected from: —H, -halogen, —OR^{o}, R^{o}, oxo, —CH₂OR^{o}, —CH₂N(R^{o})₂, —C(O)N(R^{o})₂, —C(O)OR^{o}, —NO₂, —NCS, —CN, —CF₃, —OCF₃ and —N(R^{o})₂,
   wherein each occurrence of R^{o} is independently selected from: —(C1-C6)-aliphatic, (C3-C6)-cycloalkyl, 3- to 6-membered heterocyclyl, 5- to 10-membered heteroaryl-, and (C6-C10)-aryl wherein said heterocyclyl has 1-4 heteroatoms independently selected from N, NH, O, S, SO, and SO₂, and said heteroaryl has 1-4 heteroatoms independently selected from N, NH, O, and S;
R⁴ is selected from —H or —(C1-C6)alkyl;
R⁶ is selected from —H or —(C1-C6)alkyl
each R¹³ and R¹⁴ is independently selected from H—, (C1-C3)-aliphatic-, or (C3-C6)-cycloalkyl;
wherein each occurrence of R′ is independently selected from halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂;
wherein each occurrence of R″ is independently selected from —H, —(C1-C6)-aliphatic, (C3-C6)-cycloalkyl, 3- to 6-membered heterocyclyl, 5- to 10-membered heteroaryl-, (C6-C10)-aryl-, (5- to 10-membered heteroaryl)-(C1-C6)-alkyl-, (C6-C10)-aryl-(C1-C6)-alkyl-, (5- to 10-membered heteroaryl)-O—(C1-C6)-alkyl-, and (C6-C10)-aryl-O—(C1-C6)-alkyl-, wherein each occurrence of R″ is independently substituted with 0-3 substituents, and in particular, in some aspects of the disclosure, R″ is independently substituted with 1-3 substituents, wherein the substituents are selected from: halogen, —R^{o}, —OR^{o}, oxo, —CH₂OR^{o}, —CH₂N(R^{o})₂, —C(O)N(R^{o})₂, —C(O)OR^{o}, —NO₂, —NCS, —CN, —CF₃, —OCF₃ and —N(R^{o})₂, wherein each occurrence of R^{o} is independently selected from: —(C1-C6)-aliphatic, —O(C1-C6)-aliphatic, (C3-C6)-cycloalkyl, 3- to 6-membered heterocyclyl, 5- to 10-membered heteroaryl-, and (C6-C10)-aryl- wherein said heterocyclyl has 1-4 heteroatoms independently selected from N, NH, O, S, SO, and SO₂, and said heteroaryl has 1-4 heteroatoms independently selected from N, NH, O, and S.

In some embodiments, the compound has a structure of formula I-b:

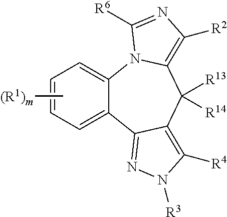

or a pharmaceutically acceptable salt, hydrate, solvate, polymorph, isomer, or combination thereof, wherein m, R¹, R², R³, R⁴, R⁶, R¹³ and R¹⁴ are as defined in formula I-a.

In some embodiments, the compound has a structure of formula I-c:

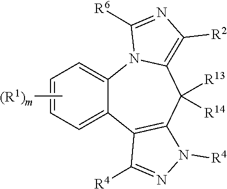

or a pharmaceutically acceptable salt, hydrate, solvate, polymorph, isomer, or combination thereof, wherein m, R¹, R², R³, R⁴, R⁶, R¹³ and R¹⁴ are as defined in formula I-a.

In some embodiments, the compound has a structure of formula I-d:

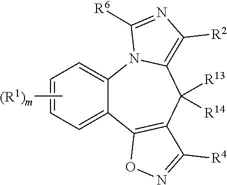

or a pharmaceutically acceptable salt, hydrate, solvate, polymorph, isomer, or combination thereof, wherein m, R¹, R², R⁴, R⁶, R¹³ and R¹⁴ are as defined in formula I-a.

In some embodiments, the compound has a structure of formula I-e:

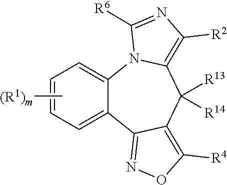

or a pharmaceutically acceptable salt, hydrate, solvate, polymorph, isomer, or combination thereof, wherein m, R¹, R², R⁴, R⁶, R¹³ and R¹⁴ are as defined in formula I-a.

In some embodiments the compound has a structure of formula I-f:

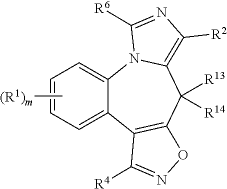

or a pharmaceutically acceptable salt, hydrate, solvate, polymorph, isomer, or combination thereof, wherein m, R¹, R², R⁴, R⁶, R¹³ and R¹⁴ are as defined in formula I-a.

In some embodiments, the present disclosure provides a compound of formula I-a:

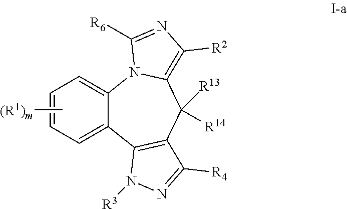

or a pharmaceutically acceptable salt, hydrate, solvate, polymorph, isomer, or combination thereof,
wherein:
m is an integer selected from 0-4;
each R¹ is independently selected from: halogen, —(C6-C10) aryl, —O(C1-C6)alkyl, —CN, —CHF₂, —CF₃, —OCF₃, —OCHF₂, CO(O)R₇, CH₂—OR⁸, —(C1-C6)alkyl-(C6-C10) aryl, -5-10 membered heteroaryl, —(C1-C6)alkyl-5-10 membered heteroaryl and —(C3-C6) cycloalkyl;
each R⁸ is independently selected from —H or —(C1-C6 alkyl)
each R² is selected from CO(O)R₇, C≡C—R⁹, —(C1-C6)alkyl-C≡C—R⁹, -(5-10 membered) heteroaryl, -(3-10 membered) heterocyclyl, (C3-C10)-cycloalkenyl

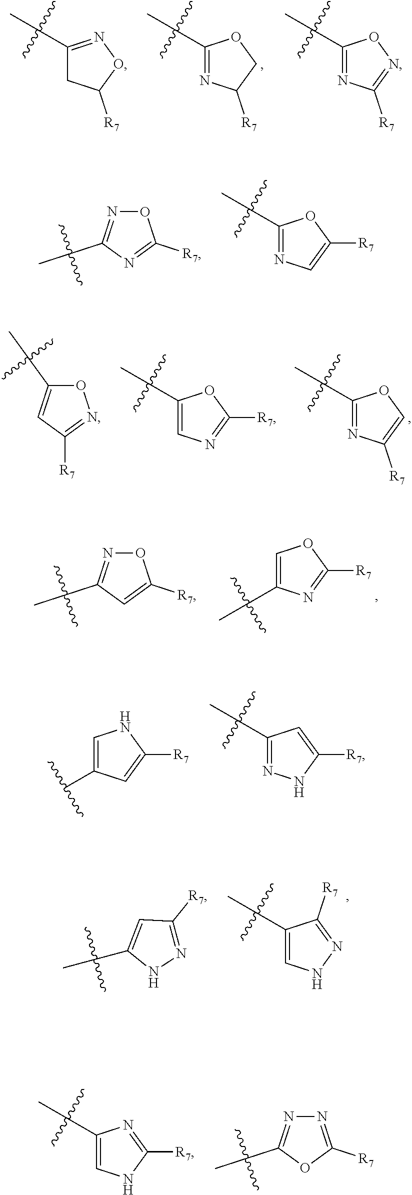

wherein each 5-6-membered heteroaryl and 3-10-membered heterocycle is substituted with 0-4 R₇ each R⁹ is selected from: —H, —(C1-C6)alkyl, (5- to 10-membered heteroaryl), -(3-10 membered) heterocyclyl, —(C6-C10) aryl, —(C1-C6)alkyl-(C6-C10) aryl

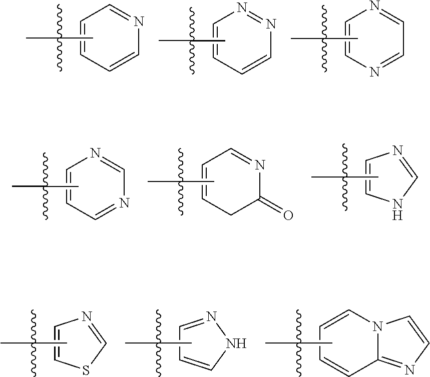

wherein each occurrence of R⁹ is independently substituted by 0-5 R¹¹;
wherein each occurrence of R¹¹ is independently selected from —(C1-C6)alkyl, —O—(C1-C6)alkyl, -halogen, —CF₃, —OCF₃, —(C6-C10) aryl and -5 to 10 membered heteroaryl;
R³ is independently selected from: —H, —(C1-C6)alkyl, -5 to 10 membered heteroaryl, -(3-10 membered) heterocyclyl, —(C1-C6)alkyl-(C3-C6) cycloalkyl and —(C1-C6)alkyl-(C6-C10) aryl, wherein R³ is independently substituted with 0-5 R¹²;
wherein each R¹² is independently selected from: —H, -halogen, —OR^{o}, R^{o}, oxo, —CH₂OR^{o}, —CH₂N(R^{o})₂, —C(O)N(R^{o})₂, —C(O)OR^{o}, —CF₃, —OCF₃ and —N(R^{o})₂, wherein each occurrence of R^{o} is independently selected from: —(C1-C6)-aliphatic, (C3-C6)-cycloalkyl, -(3-10 membered) heterocyclyl, and (C6-C10)-aryl;
   wherein each R₇ is selected from —CF₃, —(C1-C6)alkyl, —(C3-C6) cycloalkyl, -5 to 10 membered heteroaryl, —(C6-C10) aryl, —CH₂—(C6-C10) aryl, -5 to 10 membered heteroaryl-(C1—C6)alkyl, and -3-10 membered heterocyclyl, wherein each R₇ is independently substituted with 0-5 R′;
   or when two R₇ groups bound to the same atom, the two R₇ groups may be taken together with the atom to which they are bound to form a 3-10-membered aromatic or non-aromatic ring having 0-4 heteroatoms independently selected from N, NH, O, S, SO, and SO₂, wherein said ring is optionally substituted with 0-5 R′;
each occurrence of R′ is wherein each occurrence of R′ is independently selected from halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂, wherein R″ is selected from —Cl, —F, —(C1-C6)alkyl, —OMe, and —(C6-C10) aryl;
R″ is independently substituted with 1-3 substituents wherein the substituents are selected from: halogen, —CF₃, —OCF₃, —O(C1-C6)-aliphatic or —(C1-C6)-aliphatic;
each R⁴ is selected from —H or —(C1-C6)alkyl;
each R⁶ is selected from —H or —(C1-C6)alkyl;
each R¹³ and R¹⁴ is independently selected from H—, (C1-C3)-aliphatic-, or (C3-C6)-cycloalkyl;

In some embodiments of a compound of formula I-a, or a pharmaceutically acceptable salt, hydrate, solvate, polymorph, isomer, or combination thereof,
wherein: m is 0, 1 or 2;
when m is 1 or 2, at least one occurrence of R¹ is halogen or —O((C1-C6)alkyl);
each R¹ is independently selected from: -halogen and —O(C1-C6)alkyl;
R² is selected from: C≡C—R⁹, (C3-C10)-cycloalkenyl

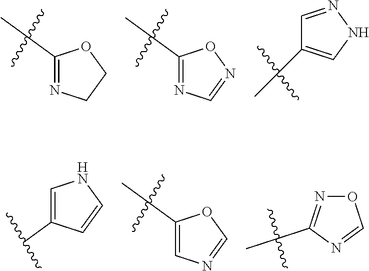

wherein each 5-membered heterocycle or heteroaryl is substituted with 0-4 R₇;
each R⁹ is selected from: —(C1-C6)alkyl,

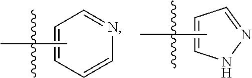

wherein each occurrence of R⁹ is independently substituted by 0-5 R¹¹
wherein each occurrence of R¹¹ is independently selected from —(C1-C6)alkyl and —O—(C1-C6)alkyl;
R³ is independently selected from: —H, —(C1-C6)alkyl, -(3-10 membered) heterocyclyl, —(C1-C6)alkyl-(C3-C6) cycloalkyl and —(C1-C6)alkyl-(C6-C10) aryl, wherein R³ is independently substituted with 0-5 R¹²;
wherein R₇ is selected from —CF₃, —(C1-C6)alkyl, —(C6-C10) aryl or —CH₂—(C6-C10) aryl,
   wherein each R₇ is independently substituted with 0-5 R′;
   each occurrence of R⁴ and R⁶ is —H.

In some embodiments, the present disclosure provides a compound of formula I-b:

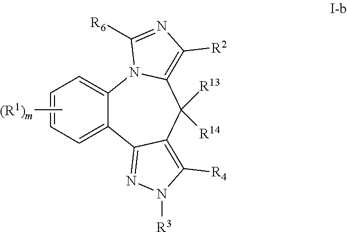

or a pharmaceutically acceptable salt, hydrate, solvate, polymorph, isomer, or combination thereof,
m is an integer selected from 0-4;
wherein:
each R¹ is independently selected from -halogen, —(C6-C10) aryl, —Ome, —CN, —CHF₂, —CF₃, —OCF₃, —OCHF₂, CO(O)R₇, CH₂—OR⁸, —(C1-C6)alkyl-(C6-C10) aryl, -5-10 membered heteroaryl, —(C1-C6)alkyl-5-10 membered heteroaryl and —(C3-C6) cycloalkyl;
each R⁸ is independently selected from —H or —(C1-C6 alkyl);
each R² is selected from CO(O)R₇, C≡C—R⁹, —(C1-C6)alkyl-C≡C—R⁹, -(5-10 membered) heteroaryl, -(3-10 membered) heterocyclyl,

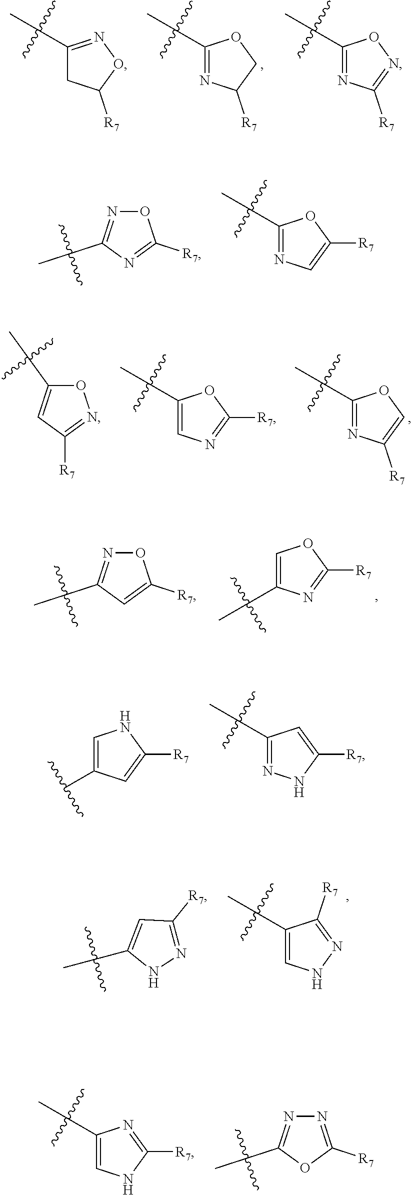

wherein each 5-6 membered heteroaryl or 3-10 membered heterocycle is substituted with 0-4 R₇;
each R⁹ is selected from: —H, —(C1-C6)alkyl, (5- to 10-membered heteroaryl), -(3-10 membered) heterocyclyl, —(C6-C10) aryl, —(C1-C6)alkyl-(C6-C10) aryl

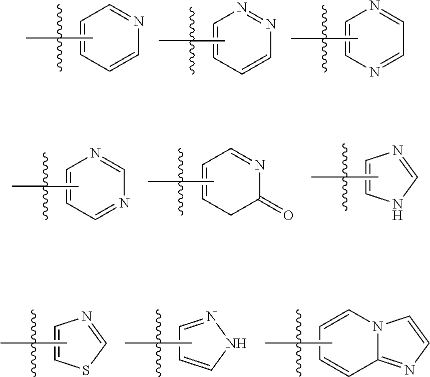

wherein each occurrence of R⁹ is independently substituted by 0-5 R¹¹
wherein each occurrence of R¹¹ is independently selected from —(C1-C6)alkyl, —O—(C1-C6)alkyl, -halogen, —CF₃, —OCF₃, —Ome, —(C6-C10) aryl and -5 to 10 membered heteroaryl;
R³ is independently selected from: —H, —(C1-C6)alkyl, —(C1-C6)alkyl-(C3-C6) cycloalkyl, —(C1-C6)alkyl-OR¹², —(C1-C6)alkyl-N(R¹²)₂, —(C1-C6)alkyl-(C6-C10) aryl, —(C1-C6) alkyl-5-10 membered heteroaryl, -3 to 10 membered heterocyclyl and -5-10 membered heteroaryl, wherein R³ is independently substituted with 0-5 R¹²;
wherein each R¹² is independently selected from: —H, -halogen, —OR^{o}, R^{o}, oxo, —CH₂OR^{o}, —CH₂N(R^{o})₂, —C(O)N(R^{o})₂, —C(O)OR^{o}, —NO₂, —NCS, —CN, —CF₃, —OCF₃ and —N(R^{o})₂,
   wherein each occurrence of R^{o} is independently selected from: —(C1-C6)-aliphatic, (C3-C6)-cycloalkyl, 3- to 6-membered heterocyclyl, 5- to 10-membered heteroaryl-, and (C6-C10)-aryl.
wherein each R₇ is selected from —H, —CF₃, —(C1-C6)alkyl, —(C3-C6) cycloalkyl, -5 to 10 membered heteroaryl, —(C6-C10) aryl, (C6-C10) aryl-(C1-C6)alkyl-, —(C1-C6)alkyl-5 to 10 membered heteroaryl and -3-10 membered heterocyclyl, wherein each R₇ is independently substituted with 0-5 R′;
   or when two R₇ groups bound to the same atom, the two R₇ groups may be taken together with the atom to which they are bound to form a 3-10-membered aromatic or non-aromatic ring having 0-4 heteroatoms independently selected from N, NH, O, S, SO, and SO₂, wherein said ring is optionally substituted with 0-5 R′;
   each occurrence of R′ is wherein each occurrence of R′ is independently selected from halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂;
wherein R″ is selected from —Cl, —F, —(C1-C6)alkyl, —OMe, —(C1-C6)alkyl-5 to 10 membered heteroaryl, -5 to 10 membered heteroaryl, -3-10 membered heterocyclyl, —(C3-C6) cycloalkyl and —(C6-C10) aryl;
R″ is independently substituted with 1-3 substituents wherein the substituents are selected from: halogen, —CF₃, —OCF₃, —O—(C1-C6) aliphatic, —(C1-C6)-aliphatic and -5 to 10 membered heteroaryl;
each R⁴ and R⁶ is independently selected from —H or —(C1-C6)alkyl;
each R¹³ and R¹⁴ is independently selected from H—, (C1-C3)-aliphatic-, or (C3-C6)-cycloalkyl;

In some embodiments of a compound of formula I-b or a pharmaceutically acceptable salt, hydrate, solvate, polymorph, isomer, or combination thereof,
wherein: m is 0, 1 or 2;
when m is 1 or 2, at least one occurrence of R¹ is halogen or —O((C1-C6)alkyl);
each R¹ is independently selected from: -halogen and —O(C1-C6)alkyl;
R² is selected from: C≡C—R⁹,

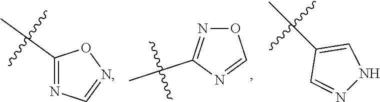 [SMILES: *c1ncno1 ; *c1ncon1 ; *c1cn[nH]c1]

wherein each 5-membered heterocycle or heteroaryl is substituted with 0-4 R₇;
each R⁹ is selected from: —(C1-C6)alkyl, wherein each occurrence of R⁹ is independently substituted by 0-5 R¹¹
wherein each occurrence of R¹¹ is selected from —(C1-C6)alkyl;
R³ is independently selected from: —H, —(C1-C6)alkyl or —CH₂—(C6-C10) aryl, and -3 to 10 membered heterocyclyl, wherein R³ is independently substituted with 0-5 R¹²;
wherein R₇ is selected from —(C1-C6)alkyl, —CH₂—(C6-C10) aryl, —(C3-C6) cycloalkyl, and -(3-10) membered heterocyclyl, wherein each R₇ is independently substituted with 0-5 R′;
each occurrence of R⁴ and R⁶ is —H.

In some embodiments, the present disclosure provides a compound of formula I-c:

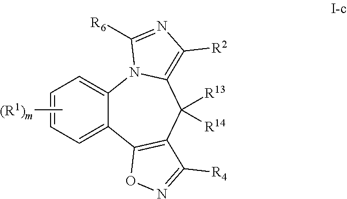

or a pharmaceutically acceptable salt, hydrate, solvate, polymorph, isomer, or combination thereof,
m is an integer selected from 0-4;
each R¹ is independently selected from -halogen, —(C6-C10) aryl, —OMe, —CN, —CHF₂, —CF₃, —OCF₃, —OCHF₂, CO(O)R₇, CH₂—OR⁸, —(C1-C6)alkyl-(C6-C10) aryl, -5-10 membered heteroaryl, —(C1-C6)alkyl-5-10 membered heteroaryl and —(C3-C6) cycloalkyl;
each R⁸ is independently selected from —H or —(C1-C6 alkyl);
each R² is selected from CO(O)R₇, C≡C—R⁹, —(C1-C6)alkyl-C≡C—R⁹, -(5-10 membered) heteroaryl, -(3-10 membered) heterocyclyl,

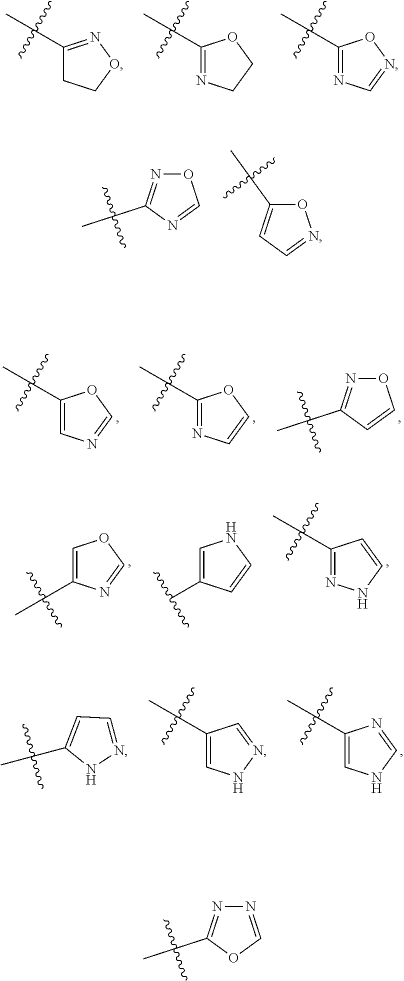

wherein each 5-membered heterocycle or heteroaryl is substituted with 0-4 R₇;
each R⁹ is selected from: —H, —(C1-C6)alkyl, (5- to 10-membered heteroaryl), -(3-10 membered) heterocyclyl

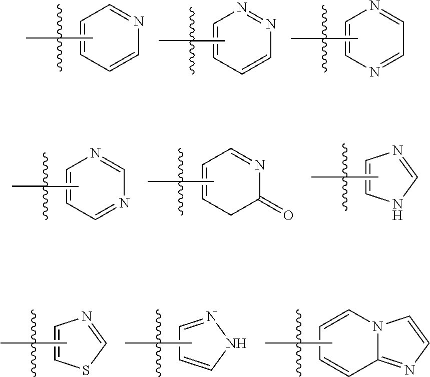

each occurrence of R⁹ is independently substituted by 0-5 R¹¹;
each occurrence of R¹¹ is independently selected from —(C1-C6)alkyl, —O—(C1-C6)alkyl, -halogen, —CF₃, —OCF₃, —OMe, —(C6-C10) aryl and -5 to 10 membered heteroaryl;
each occurrence of R₇ is selected from —CF₃, —(C1-C6)alkyl, —(C3-C6) cycloalkyl, -5 to 10 membered heteroaryl, —(C6-C10) aryl, (C6-C10) aryl-(C1-C6)alkyl-, —(C1-C6)alkyl-5 to 10 membered heteroaryl and -3-10 membered heterocyclyl, wherein each R₇ is independently substituted with 0-5 R′;
each occurrence of R′ is wherein each occurrence of R′ is independently selected from halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂,
wherein each occurrence of R″ is selected from —Cl, —F, —(C1-C6)alkyl, —Ome, —(C1-C6)alkyl-5 to 10 membered heteroaryl, -5 to 10 membered heteroaryl, -3-10 membered heterocyclyl, —(C3-C6) cycloalkyl and —(C6-C10) aryl, and R″ is independently substituted with 1-3 substituents wherein the substituents are selected from: halogen, —CF₃, —OCF₃, —(C1-C6)-aliphatic and -5 to 10 membered heteroaryl;
each R⁴ and R⁶ is independently selected from —H or (C1-C6)alkyl;
each R¹³ and R¹⁴ is independently selected from H—, (C1-C3)-aliphatic-, or (C3-C6)-cycloalkyl.

In some embodiments of a compound of formula I-c or a pharmaceutically acceptable salt, hydrate, solvate, polymorph, isomer, or combination thereof,
wherein: m is 0, 1 or 2;
when m is 1 or 2, at least one occurrence of R¹ is halogen or —O((C1-C6)alkyl);
each R¹ is independently selected from: -halogen and —O(C1-C6)alkyl;
R² is selected from: CO(O)R₇, C≡C—R⁹,

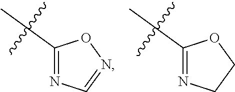 [SMILES: *c1ncno1 ; *C1=NCCO1]

wherein each 5-membered heterocycle or heteroaryl is substituted with 0-4 R₇;
each R⁹ is selected from: H or —(C1-C6)alkyl, wherein each occurrence of R⁹ is independently substituted by 0-5 R¹¹
each occurrence of R¹¹ is selected from —(C1-C6)alkyl;
each occurrence of R⁴ and R⁶ is —H.

In some embodiments, the present disclosure provides a compound of formula I-d:

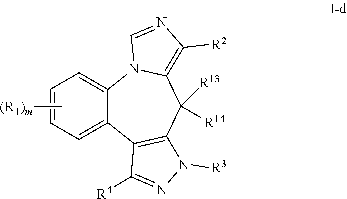

or a pharmaceutically acceptable salt, hydrate, solvate, polymorph, isomer, or combination thereof,
m is an integer selected from 0-4;
wherein:
each R¹ is independently selected from -halogen, —(C6-C10) aryl, —Ome, —CN, —CHF₂, —CF₃, —OCF₃, —OCHF₂, CO(O)R₇, CH₂—OR⁸, —(C1-C6)alkyl-(C6-C10) aryl, -5-10 membered heteroaryl, —(C1-C6)alkyl-5-10 membered heteroaryl and —(C3-C6) cycloalkyl;
each R⁸ is independently selected from —H or —(C1-C6 alkyl);
each R² is selected from CO(O)R₇, C≡C—R⁹, —(C1-C6)alkyl-C≡C—R⁹, -(5-10 membered) heteroaryl, -(3-10 membered) heterocyclyl,

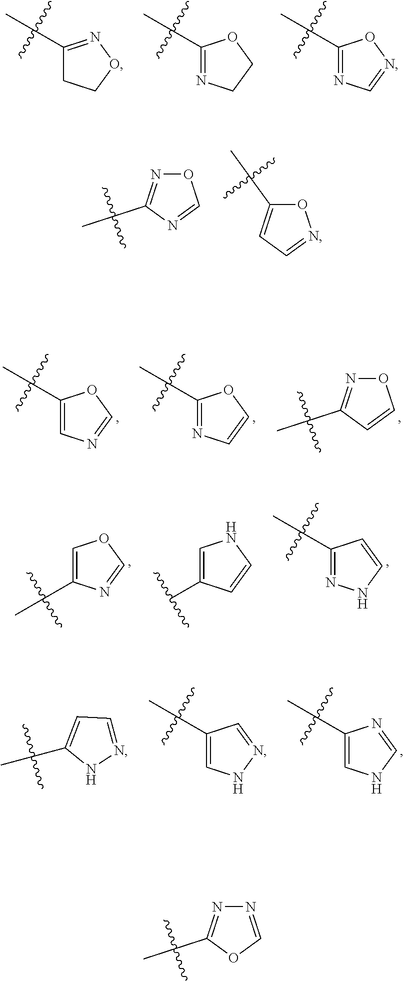

wherein each 5-membered heterocycle or heteroaryl is substituted with 0-4 R₇;
each R⁹ is selected from: —H, —(C1-C6)alkyl, (5- to 10-membered heteroaryl), -(3-10 membered) heterocyclyl, —(C6-C10) aryl, —(C1-C6)alkyl-(C6-C10) aryl

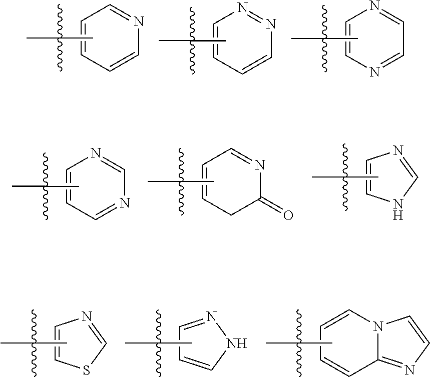

wherein each occurrence of R⁹ is independently substituted by 0-5 R¹¹
wherein each occurrence of R¹¹ is independently selected from —(C1-C6)alkyl, —O—(C1-C6)alkyl, -halogen, —CF₃, —OCF₃, —Ome, —(C6-C10) aryl and -5 to 10 membered heteroaryl;
R³ is independently selected from: —H, —(C1-C6)alkyl, —(C1-C6)alkyl-(C3-C6) cycloalkyl, —(C1-C6)alkyl-OR¹², —(C1-C6)alkyl-N(R¹²)₂, —(C1-C6)alkyl-(C6-C10) aryl, —(C1-C6) alkyl-5-10 membered heteroaryl wherein R³ is independently substituted with 0-5 R¹²;
wherein each R¹² is independently selected from: —H, -halogen, —OR^{o}, R^{o}, oxo, —CH₂OR^{o}, —CH₂N(R^{o})₂, —C(O)N(R^{o})₂, —C(O)OR^{o}, —NO₂, —NCS, —CN, —CF₃, —OCF₃ and —N(R^{o})₂,
   wherein each occurrence of R^{o} is independently selected from: —(C1-C6)-aliphatic, (C3-C6)-cycloalkyl, 3- to 6-membered heterocyclyl, 5- to 10-membered heteroaryl-, and (C6-C10)-aryl.

Wherein R₇ is selected from —CF₃, —(C1-C6)alkyl, —(C3-C6) cycloalkyl, -5 to 10 membered heteroaryl, —(C6-C10) aryl, (C6-C10) aryl-(C1-C6)alkyl-, —(C1-C6)alkyl-5 to 10 membered heteroaryl and -3-10 membered heterocyclyl, wherein each R₇ is independently substituted with 0-5 R′;
each occurrence of R′ is wherein each occurrence of R′ is independently selected from halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂;
wherein R″ is selected from —Cl, —F, —(C1-C6)alkyl, —OMe, and —(C6-C10) aryl;
each R⁴ is selected from —H or —(C1-C6)alkyl;
each R⁶ is selected from —H or —(C1-C6)alkyl;
each R¹³ and R¹⁴ is independently selected from H—, (C1-C3)-aliphatic-, or (C3-C6)-cycloalkyl.

In some embodiments of a compound of formula I-d or a pharmaceutically acceptable salt, hydrate, solvate, polymorph, isomer, or combination thereof,
wherein: m is 0, 1 or 2;
when m is 1 or 2, at least one occurrence of R¹ is halogen or —O((C1-C6)alkyl);
each R¹ is independently selected from: -halogen and —O(C1-C6)alkyl;
R² is selected from: C≡C—R⁹,

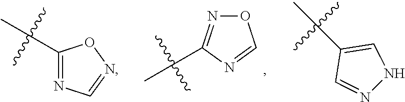 [SMILES: *c1ncno1 ; *c1ncon1 ; *c1cn[nH]c1]

wherein each 5-membered heterocycle or heteroaryl is substituted with 0-4 R₇;
each R⁹ is selected from: —(C1-C6)alkyl, wherein each occurrence of R⁹ is independently substituted by 0-5 R¹¹, wherein each occurrence of R¹¹ is selected from —(C1-C6)alkyl;
R³ is independently selected from: —H, —(C1-C6)alkyl or —CH₂—(C6-C10) aryl, wherein R³ is independently substituted with 0-5 R¹², wherein each R¹² is independently selected from: —H or —F,
wherein R₇ is selected from —(C1-C6)alkyl, —CH₂—(C6-C10) aryl, —(C3-C6) cycloalkyl, and -(3-10) membered heterocyclyl, wherein each R₇ is independently substituted with 0-5 R′;
each occurrence of R⁴ and R⁶ is —H.

In some embodiments, the present disclosure provides a compound of formula I-e:

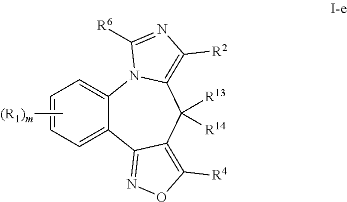

or a pharmaceutically acceptable salt, hydrate, solvate, polymorph, isomer, or combination thereof,
m is an integer selected from 0-4;
each R¹ is independently selected from -halogen, —(C6-C10) aryl, —OMe, —CN, —CHF₂, —CF₃, —OCF₃, —OCHF₂, CO(O)R₇, CH₂—OR⁸, —(C1-C6)alkyl-(C6-C10) aryl, -5-10 membered heteroaryl, —(C1-C6)alkyl-5-10 membered heteroaryl and —(C3-C6) cycloalkyl;
each R⁸ is independently selected from —H or —(C1-C6 alkyl);
each R² is selected from CO(O)R₇, C≡C—R⁹, —(C1-C6)alkyl-C≡C—R⁹, -(5-10 membered) heteroaryl, -(3-10 membered) heterocyclyl,

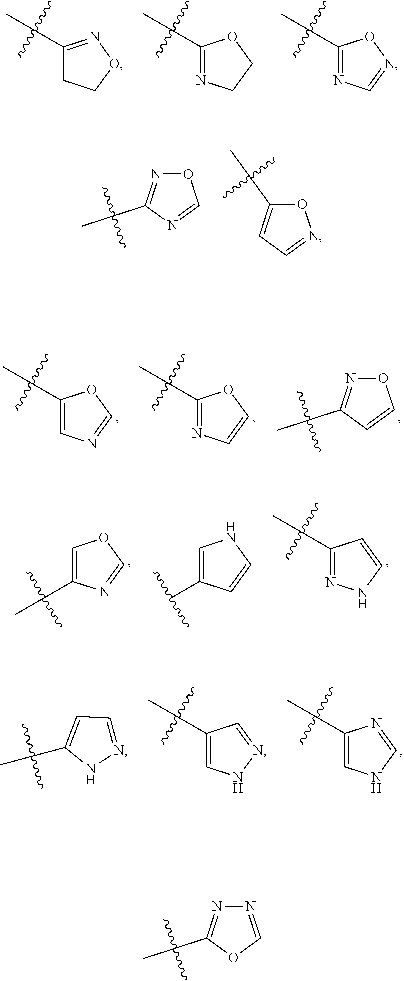

wherein each 5-membered heterocycle or heteroaryl is substituted with 0-4 R₇;
each R⁹ is selected from: —H, —(C1-C6)alkyl, (5- to 10-membered heteroaryl), -(3-10 membered) heterocyclyl

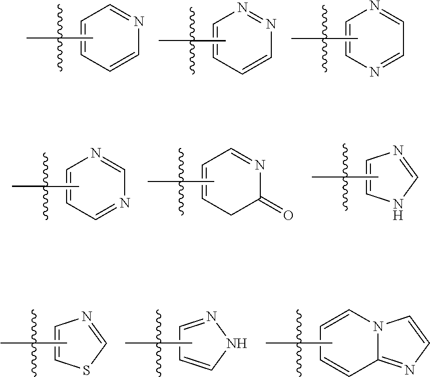

each occurrence of R⁹ is independently substituted by 0-5 R¹¹, wherein each occurrence of R¹¹ is independently selected from —(C1-C6)alkyl, —O—(C1-C6)alkyl, -halogen, —CF₃, —OCF₃, —OMe, —(C6-C10) aryl and -5 to 10 membered heteroaryl;
wherein R₇ is selected from —(C1-C6)alkyl, —(C3-C6) cycloalkyl, -5 to 10 membered heteroaryl, —(C6-C10) aryl, —(C6-C10) aryl-(C1-C6)alkyl, and -5 to 10 membered heteroaryl-(C1-C6)alkyl, and -3-10 membered heterocyclyl, wherein each R₇ is independently substituted with 0-5 R′;
each occurrence of R′ is wherein each occurrence of R′ is independently selected from halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂, wherein each R′ is independently substituted with 0-5 R″;
wherein R″ is selected from —Cl, —F, —(C1-C6)alkyl, —OMe, and —(C6-C10) aryl;
each R⁴ is independently-H or (C1-C6)alkyl;
each R⁶ is independently-H or —(C1-C6)alkyl.
Each R¹³ and R¹⁴ is independently selected from H—, (C1-C3)-aliphatic-, or (C3-C6)-cycloalkyl.

In some embodiments of a compound of formula I-e or a pharmaceutically acceptable salt, hydrate, solvate, polymorph, isomer, or combination thereof,
wherein: m is 0, 1 or 2;
when m is 1 or 2, at least one occurrence of R¹ is halogen or —O((C1-C6)alkyl);
each R¹ is independently selected from: -halogen and —O(C1-C6)alkyl;
R² is selected from: CO(O)R₇, C≡C—R⁹,

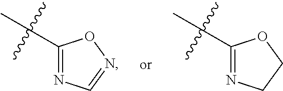 [SMILES: *c1ncno1 ; *C1=NCCO1]

wherein each 5-membered heterocycle or heteroaryl is substituted with 0-4 R₇;
each R⁹ is selected from: H or —(C1-C6)alkyl, wherein each occurrence of R⁹ is independently substituted by 0-5 R¹¹
wherein each occurrence of R¹¹ is selected from —(C1-C6)alkyl;
R⁴ and R⁶ are both-H.

In some embodiments, the present disclosure provides a compound of formula I-f:

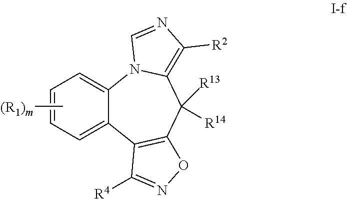

or a pharmaceutically acceptable salt, hydrate, solvate, polymorph, isomer, or combination thereof,
m is an integer selected from 0-4;
each R¹ is independently selected from -halogen, —(C6-C10) aryl, —OMe, —CN, —CHF₂, —CF₃, —OCF₃, —OCHF₂, CO(O)R₇, CH₂—OR⁸, —(C1-C6)alkyl-(C6-C10) aryl, -5-10 membered heteroaryl, —(C1-C6)alkyl-5-10 membered heteroaryl and —(C3-C6) cycloalkyl;
each R⁸ is independently selected from —H or —(C1-C6 alkyl);
each R² is selected from CO(O)R₇, C≡C—R⁹, —(C1-C6)alkyl-C≡C—R⁹, -(5-10 membered) heteroaryl, -(3-10 membered) heterocyclyl,

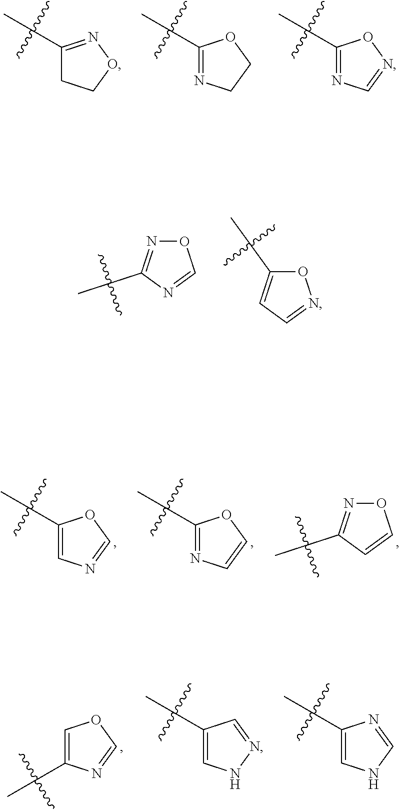

wherein each 5-membered heterocycle or heteroaryl is substituted with 0-4 R₇;
each R⁹ is selected from: —H, —(C1-C6)alkyl, (5- to 10-membered heteroaryl), -(3-10 membered) heterocyclyl

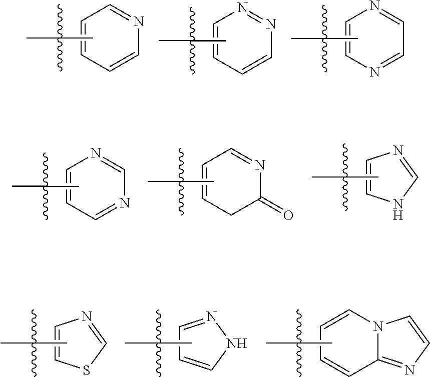

each occurrence of R⁹ is independently substituted by 0-5 R¹¹, wherein each occurrence of R¹¹ is independently selected from —(C1-C6)alkyl, —O—(C1-C6)alkyl, -halogen, —CF₃, —OCF₃, —OMe, —(C6-C10) aryl and -5 to 10 membered heteroaryl;
wherein R₇ is selected from —(C1-C6)alkyl, —(C3-C6) cycloalkyl, -5 to 10 membered heteroaryl, —(C6-C10) aryl, —(C6-C10) aryl-(C1-C6)alkyl, and -5 to 10 membered heteroaryl-(C1-C6)alkyl, and -3-10 membered heterocyclyl, wherein each R₇ is independently substituted with 0-5 R′;
each occurrence of R′ is wherein each occurrence of R′ is independently selected from halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂;
wherein each R′ is independently substituted with 0-5 R″, wherein R″ is selected from —Cl, —F, —(C1-C6)alkyl, —OMe, and —(C6-C10) aryl;
R⁴ is —H or (C1-C6)alkyl;
R⁶ is —H or —(C1-C6)alkyl;
each R¹³ and R¹⁴ is independently selected from H—, (C1-C3)-aliphatic-, or (C3-C6)-cycloalkyl.

In some embodiments of a compound of formula I-f or a pharmaceutically acceptable salt, hydrate, solvate, polymorph, isomer, or combination thereof, wherein: m is 0, 1 or 2;
when m is 1 or 2, at least one occurrence of R¹ is halogen or —O((C1-C6)alkyl);
each R¹ is independently selected from: -halogen and —O(C1-C6)alkyl;
R² is selected from: CO(O)R₇, C≡C—R⁹,

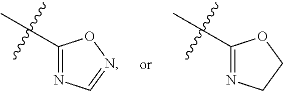 [SMILES: *c1ncno1 ; *C1=NCCO1]

wherein each 5-membered heterocycle or heteroaryl is substituted with 0-4 R₇;
each R⁹ is selected from: H or —(C1-C6)alkyl, wherein each occurrence of R⁹ is independently substituted by 0-5 R¹¹, and each occurrence of R¹¹ is selected from —(C1-C6)alkyl;
R⁴ and R⁶ are both-H.

In some embodiments, the GABA_{A} α5 receptor agonist is a compound of Formula II as recited in paragraph 19 or is a compound of Formula II as recited in paragraph 20, or is a compound of Formula IV as recited in paragraph 19 or is a compound of Formula IV as recited in paragraph 20, or is a compound of Formula I-a as recited in paragraph 30; or is a compound of Formula I-b as recited in paragraph 30, or is a compound of Formula I-c as recited in paragraph 30; or is a compound of Formula I-d as recited in paragraph 30; or is a compound of Formula I-e as recited in paragraph 30; or is a compound of Formula I-f as recited in paragraph 30; or is a Compound 1-12, 44-56, 101-268, 270-644, 646-687, 689-698, 700-703, 705, 707-721, 723-740, 742-755, 758-763, 765-779. 781-795, 797-810, 813-828, 830, 831, 833-846, 848-891, 893-903, 905, 907-977, 979-1012 as described above, or is Compound 606, Form A, Compound 606, Form B, Compound 606, Form C, Compound 606, Form E, or Compound 606, Form F, or a pharmaceutically acceptable salt, hydrate, solvate, polymorph, or isomer thereof. See, pages 284-305 of WO2022011318, incorporated by reference herein in its entirety and in particular in the context of the recited compounds, their synthesis and properties.

In some embodiments, the GABA_{A} α5 receptor agonist, or the pharmaceutically acceptable salt, hydrate, solvate, polymorph, or isomer thereof or the GABA_{A} α5 receptor agonist of this disclosure, or the pharmaceutically acceptable salt, hydrate, solvate, isomer, or polymorph thereof included as part of a pharmaceutical composition of this disclosure, is a compound of Formula II as recited in paragraph 19 or is a compound of Formula II as recited in paragraph 20, or a pharmaceutically acceptable salt, hydrate, solvate, polymorph, or isomer thereof. In other embodiments, the GABA_{A} α5 receptor agonist, or the pharmaceutically acceptable salt, hydrate, solvate, polymorph, or isomer thereof or the GABA_{A} α5 receptor agonist of this disclosure, or the pharmaceutically acceptable salt, hydrate, solvate, isomer, or polymorph thereof included as part of a pharmaceutical composition of this disclosure, is a compound of Formula IV as recited in paragraph 19 or is a compound of Formula IV as recited in paragraph 20, or a pharmaceutically acceptable salt, hydrate, solvate, polymorph, or isomer thereof. In some embodiments, the GABA_{A} α5 receptor agonist, or the pharmaceutically acceptable salt, hydrate, solvate, polymorph, or isomer thereof of this disclosure or the GABA_{A} α5 receptor agonist, or the pharmaceutically acceptable salt, hydrate, solvate, isomer, or polymorph thereof included as part of a pharmaceutical composition of this disclosure, a compound of Formula I-a, I-b, I-c, I-d, I-e, and I-f as recited in paragraph 30.

In other aspects of the methods and uses of this disclosure, the compounds are crystalline forms of the GABA_{A} α5 receptor agonists, or pharmaceutically acceptable salts, hydrates, solvates, or isomers thereof. Such crystalline forms may, for example, be selected from the group consisting of Compound 606, Form A (polymorph crystalline form), Compound 606, Form B (polymorph crystalline form), Compound 606, Form C (solvate crystalline form), Compound 606, Form E (polymorph crystalline form), and Compound 606, Form F (hydrate crystalline form). In other embodiments of the methods and uses of this disclosure, the pharmaceutical compositions comprise one of more of the crystalline forms of the GABA_{A} α5 receptor agonists, or pharmaceutically acceptable salts, hydrates, solvates, or isomers thereof, including, for example those selected from the group consisting of Compound 606, Form A (polymorph crystalline form), Compound 606, Form B (polymorph crystalline form), Compound 606, Form C (solvate crystalline form), Compound 606, Form E (polymorph crystalline form), and Compound 606, Form F (hydrate crystalline form). In some embodiments, the crystalline form is Compound 606, Form A. In some embodiments, the crystalline form is Compound 606, Form B. In some embodiments, the crystalline form is Compound 606, Form C. In some embodiments, the crystalline form is Compound 606, Form E. In some embodiments, the crystalline form is Compound 606, Form F. See WO2022011318, which is incorporated by reference herein in its entirety and in particular in the context of the recited compounds, their synthesis and properties.

In some embodiments, the crystalline form is an anhydrous crystalline form of Compound 606, wherein the crystalline form corresponds to Form A, Form B, or Form E. In some embodiments, the crystalline form is a solvated crystalline form of Compound 606, wherein the crystalline form corresponds to Form C or Form F. In certain such embodiments, the solvated crystalline form of Compound 606 is a methanolate or a hydrate.

In some embodiments of the methods and uses of this disclosure, the crystalline form is Compound 606, Form A characterized by an x-ray powder diffraction (XRPD) pattern substantially as set forth in FIG. 29 of WO2022011318 having at least one of 3.0 and/or 21.0 degrees 2θ±0.2 degrees 2θ and further comprising one or more of additional peaks selected from 9.1, 10.7, 13.8, 22.0, 23.1, 23.9, 24.4, and 27.1 degrees 2θ=0.2 degrees 2θ. In some embodiments, the crystalline form is Compound 606, Form A characterized by a C2/c single crystal x-ray diffraction space group. In some embodiments, the crystalline form is Compound 606, Form A characterized by a single crystal x-ray diffraction unit cell having the parameters: a=58.1415 (14) Å, b=4.03974 (8) Å, c=17.1204 (3) Å, α=90°, β=90.261 (2)°, γ=90°, V=4021.15 (14) Å³. In some embodiments, the crystalline form is Compound 606, Form A characterized by a differential scanning calorimetry (DSC) curve substantially as set forth in FIG. 27B of WO2022011318. In some embodiments, the crystalline form is Compound 606, Form A characterized by a differential scanning calorimetry (DSC) curve having an exotherm with an onset at about 207° C. In some embodiments, the crystalline form is Compound 606, Form A characterized by two or more of: (a) an x-ray powder diffraction (XRPD) pattern substantially as set forth in FIG. 29 of WO2022011318; (b) a C2/c single crystal x-ray diffraction space group; (c) a single crystal x-ray diffraction unit cell having the parameters: a=58.1415 (14) Å, b=4.03974 (8) Å, c=17.1204 (3) Å, α=90°, β=90.261 (2)°, γ=90°, V=4021.15 (14) Å³; (d) a differential scanning calorimetry (DSC) curve substantially as set forth in FIG. 27B of WO2022011318; and (e) a differential scanning calorimetry (DSC) curve having an exotherm with an onset at about 207° C.

In some embodiments of the methods and uses of this disclosure, the crystalline form is Compound 606, Form B characterized by an x-ray powder diffraction (XRPD) pattern substantially as set forth in FIG. 34 of WO2022011318, having at least one peak selected from 13.0 and/or 15.3 degrees 2θ±0.2 degrees 2θ and further comprising one or more of additional peaks selected from 7.0, 9.3, 10.2, 10.4, 12.5, 13.6, 14.0, 22.0, 23.0, 23.6, and 27.3 degrees 2θ±0.2 degrees 2θ. In some embodiments, the crystalline form is Compound 606, Form B characterized by a monoclinic single crystal x-ray diffraction unit cell. In some embodiments, the crystalline form is Compound 606, Form B characterized by a single crystal x-ray diffraction formula unit volume of about 497 Å3. In some embodiments, the crystalline form is Compound 606, Form B characterized by a differential scanning calorimetry (DSC) curve having an exotherm with an onset at about 190° C. In some embodiments, the crystalline form is Compound 606, Form B characterized by two or more of: (a) an x-ray powder diffraction (XRPD) pattern substantially as set forth in FIG. 34 of WO2022011318; (b) a single crystal x-ray diffraction formula unit volume of about 497 Å3; and (c) a differential scanning calorimetry (DSC) curve having an exotherm with an onset at about 190° C.

In some embodiments of the methods and uses of this disclosure, the crystalline form is Compound 606, Form C characterized by an x-ray powder diffraction (XRPD) pattern substantially as set forth in FIG. 41 of WO2022011318, having at least one peak selected from 8.5, and/or 18.9 degrees 2θ±0.2 degrees 2θ and further comprising one or more of additional peaks selected from 7.1, 9.4, 10.3, 12.3, 12.5, 14.2, 20.7, 22.1, 23.2, 23.7, 24.0, and 26.4 degrees 2θ±0.2 degrees 2θ. In some embodiments, the crystalline form is Compound 606, Form C characterized by a monoclinic single crystal x-ray diffraction unit cell. In some embodiments, the crystalline form is Compound 606, Form C characterized by a single crystal x-ray diffraction formula unit volume of about 544 Å3. In some embodiments, the crystalline form is Compound 606, Form C characterized by a differential scanning calorimetry (DSC) curve substantially as set forth in FIG. 42B of WO2022011318. In some embodiments, the crystalline form is Compound 606, Form C characterized by a differential scanning calorimetry (DSC) curve having an exotherm with an onset at about 190° C. In some embodiments, the crystalline form is Compound 606, Form C characterized by two or more of: (a) an x-ray powder diffraction (XRPD) pattern substantially as set forth in FIG. 41 of WO2022011318; (b) a monoclinic single crystal x-ray diffraction unit cell; (c) a single crystal x-ray diffraction formula unit volume of about 544 Å3; (d) a differential scanning calorimetry (DSC) curve substantially as set forth in FIG. 42B of WO2022011318; and (e) a differential scanning calorimetry (DSC) curve having an exotherm with an onset at about 190° C.

In some embodiments of the methods and uses of this disclosure, the crystalline form is Compound 606, Form E characterized by an x-ray powder diffraction (XRPD) pattern substantially as set forth in FIG. 45 of WO2022011318, having at least one peak selected from 11.4, 18.1, and/or 21.6 degrees 2θ±0.2 degrees 2θ and further comprising one or more of additional peaks selected from 7.2, 22.0, 23.0, 24.2, 25.0, and 26.6 degrees 2θ±0.2 degrees 2θ. In some embodiments, the crystalline form is Compound 606, Form E characterized by a P2₁/n single crystal x-ray diffraction space group. In some embodiments, the crystalline form is Compound 606, Form E characterized by a single crystal x-ray diffraction unit cell having the parameters: a=11.83974 (13) Å, b=23.5195 (2) Å, c=14.48807 (17) Å, α=90°, β=101.5333 (11)°, γ=90°, V=3952.96 (7) Å³. In some embodiments, the crystalline form is Compound 606, Form E characterized by a differential scanning calorimetry (DSC) curve substantially as set forth in FIG. 36B of WO2022011318. In some embodiments, the crystalline form is Compound 606, Form E characterized by a differential scanning calorimetry (DSC) curve having an exotherm with an onset at about 201° C. In some embodiments, the crystalline form is Compound 606, Form E characterized by two or more of (a) an x-ray powder diffraction (XRPD) pattern substantially as set forth in FIG. 35 of WO2022011318; (b) a P2₁/n single crystal x-ray diffraction space group; (c) a single crystal x-ray diffraction unit cell having the parameters: a=11.83974 (13) Å, b=23.5195 (2) Å, c=14.48807 (17) Å, a=90°, β=101.5333 (11)°, Y=90°, V=3952.96 (7) Å³; (d) a differential scanning calorimetry (DSC) curve substantially as set forth in FIG. 36B of WO2022011318; and (e) a differential scanning calorimetry (DSC) curve having an exotherm with an onset at about 201° C.

In some embodiments of the methods and uses of this disclosure, the crystalline form is Compound 606, Form F characterized by an x-ray powder diffraction (XRPD) pattern substantially as set forth in FIG. 37 of WO2022011318, having at least one peak selected from 9.9, 11.9, 17.3, 19.4, and/or 25.7 degrees 2θ±0.2 degrees 2θ and further comprising one or more of additional peaks selected from 9.7, 12.1, 20.8, 23.2, 23.7, 24.2, 25.0, and 26.4 degrees 2θ±0.2 degrees 2θ. In some embodiments, the crystalline form is Compound 606, Form F characterized by a triclinic single crystal x-ray diffraction unit cell. In some embodiments, the crystalline form is Compound 606, Form F characterized by a single crystal x-ray diffraction formula unit volume of about 511 Å3. In some embodiments, the crystalline form is Compound 606, Form F characterized by a differential scanning calorimetry (DSC) curve having an exotherm at above about 120° C. In some embodiments, the crystalline form is Compound 606, Form F characterized by two or more of: (a) an x-ray powder diffraction (XRPD) pattern substantially as set forth in FIG. 37 of WO2022011318; (b) a triclinic single crystal x-ray diffraction unit cell; (c) a single crystal x-ray diffraction formula unit volume of about 511 Å3; and (d) a differential scanning calorimetry (DSC) curve having an exotherm at above about 120° C.

Pharmaceutical compositions or combinations (or components thereof) of the methods and uses of this disclosure may be administered by intraoral and oral delivery (including sublingual and buccal administration, e.g. Danckwerts (2003). Intraoral Drug Delivery: A Comparative Review. American Journal of Drug Delivery. 1. 171-186). Such delivery may be in the form of bioadhesive polymers, tablets, patches, thin films, liquids and semisolids (see e.g., Smart JD. Buccal drug delivery. Expert Opin Drug Deliv. 2005 May; 2 (3): 507-17).

A person of ordinary skill in the art, such as a physician, is readily able to determine the required amount of GABA_{A} α5 receptor agonist to treat the subject using the compositions and methods of the disclosure. It is understood that the dosage regimen will be determined for an individual, taking into consideration, for example, various factors that modify the action of the selected GABA_{A} α5 receptor agonist, the severity or stage of the disease, route of administration, and characteristics unique to the individual, such as age, weight, size, and extent of cognitive impairment.

In certain embodiments of the disclosure, the daily dose of the GABA_{A} α5 receptor agonist or the pharmaceutically acceptable salt, hydrate, solvate, polymorph, or isomer thereof, is in an amount of 0.0015 mg to 5000 mg or 5 mg to 1000 mg. In some embodiments of the disclosure, the dose of the GABA_{A} α5 receptor agonist or the pharmaceutically acceptable salt, hydrate, solvate, polymorph, or isomer thereof, is about 0.1-500 mg/day. In some embodiments of the disclosure, the dose of the GABA_{A} α5 receptor agonist or the pharmaceutically acceptable salt, hydrate, solvate, polymorph, or isomer thereof, is about 1-250 mg/day. In some embodiments of the disclosure, the dose of the GABA_{A} α5 receptor agonist or the pharmaceutically acceptable salt, hydrate, solvate, polymorph, or isomer thereof, is about 10-100 mg/day. In some embodiments of the disclosure, the dose of the GABA_{A} α5 receptor agonist or the pharmaceutically acceptable salt, hydrate, solvate, polymorph, or isomer thereof, is about 1-10 mg/day. Daily doses that may be used include, but are not limited to, 0.0015 mg/kg, 0.002 mg/kg, 0.0025 mg/kg, 0.005 mg/kg, 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.2 mg/kg, 1.4 mg/kg, 1.5 mg/kg, 1.6 mg/kg, 1.8 mg/kg, 2.0 mg/kg, 2.2 mg/kg, 2.4 mg/kg, 2.5 mg/kg, 2.6 mg/kg, 2.8 mg/kg, 3.0 mg/kg, 3.5 mg/kg, 4.0 mg/kg, 4.5 mg/kg, 5.0 mg/kg, 6.0 mg/kg, 7.0 mg/kg, 10 mg/kg, 14 mg/kg, 18 mg/kg, 36 mg/kg, 50 mg/kg, 70 mg/kg; or 0.1 mg, 0.15 mg, 0.18 mg, 0.35 mg, 0.7 mg, 1.5 mg, 2.0 mg, 2.5 mg, 2.8 mg, 3.0 mg, 3.5 mg, 4.2 mg, 5 mg, 5.5 mg, 6.0 mg, 7 mg, 8 mg, 9 mg, 10 mg, 12 mg, 15 mg, 20 mg, 25 mg, 28 mg, 30 mg, 35 mg, 40 mg, 45 mg, 50 mg, 55 mg, 60 mg, 70 mg, 75 mg, 80 mg, 85 mg, 90 mg, 95 mg, 100 mg, 110 mg, 120 mg, 125 mg, 140 mg, 150 mg, 170 mg, 175 mg, 180 mg, 190 mg, 200 mg, 210 mg, 225 mg, 250 mg, 280 mg, 300 mg, 350 mg, 400 mg, 500 mg, 750 mg, 1000 mg, 1250 mg, 2500 mg, 3500 mg, or 5000 mg. In some embodiments, the dose of the GABA_{A} α5 receptor agonist or the pharmaceutically acceptable salt, hydrate, solvate, polymorph, or isomer thereof is in an amount of about 0.5 mg, about 5 mg, about 20 mg, about 50 mg, about 75 mg, about 100 mg, about 150 mg, about 250 mg, about 500 mg, about 750 mg, about 1000 mg, about 1250 mg, about 2500 mg, about 3500 mg, or 5000 mg of the GABA_{A} α5 receptor agonist. Other doses higher than, intermediate to, or less than these doses may also be used and may be determined by one skilled in the art following the methods of this disclosure.

In certain embodiments, the dose of the GABA_{A} α5 receptor agonist is between 0.0001 and 100 mg/kg/day in rodents (which, given a typical human subject of 70 kg, is between 0.007 and 7000 mg/day).

In certain embodiments of the disclosure, the frequency of administration of the above daily doses is 12 or 24 hours. In some embodiments, once daily administration is used. In some embodiments, Administration at more frequent intervals, such as once every 6 hours, may also be used. For repeated administrations over several days or weeks or longer, depending on the condition, the treatment is sustained until a sufficient level of cognitive function is achieved.

Dosing at less frequent intervals may also be used. For example, if administered by an implant, a device or a slow or extended-release formulation, the GABA_{A} α5 receptor agonist can be administered one time, or one or more times periodically throughout the lifetime of the patient as necessary. Other administration intervals intermediate to or shorter than these dosage intervals may also be used and may be determined by one skilled in the art following the methods of this invention.

Desired time of administration can be determined by routine experimentation by one skilled in the art. For example, the GABA_{A} α5 receptor agonist may be administered for a period of 1-4 weeks, 1-3 months, 3-6 months, 6-12 months, 1-2 years, or more, up to the lifetime of the patient.

In some embodiments of the methods and uses of this disclosure, the compounds, pharmaceutical compositions, combinations and medicaments comprising levetiracetam, brivaracetam, or seletracetam, or a pharmaceutically acceptable salt thereof, and/or a GABA_{A} α5 receptor agonist (e.g., a compound of Formula II as recited in paragraph 19 or 20 or Formula IV as recited in paragraph 19 or 20; a compound of Formula I-a, I-b, I-c, I-d, I-e, and I-f as recited in paragraph 30; Compounds 1-12, 44-56, 101-268, 270-644, 646-687, 689-698, 700-703, 705, 707-721, 723-740, 742-755, 758-763, 765-779. 781-795, 797-810, 813-828, 830, 831, 833-846, 848-891, 893-903, 905, 907-977, 979-1012 as described above; or Compound 606, Form A; Compound 606, Form B; Compound 606, Form C; Compound 606, Form E; or Compound 606, Form F), or a pharmaceutically acceptable salt, hydrate, solvate, polymorph, or isomer thereof) may be in a solid dosage form such as a capsule, tablet, medicinal dragée, pill, lozenge, cachet, powder, troche, wafer, or granule. In such forms, one or more compounds, pharmaceutical compositions, medicaments of combinations useful in the methods and uses of this disclosure (e.g., comprising levetiracetam, brivaracetam, or seletracetam), or a pharmaceutically acceptable salt thereof, and/or a GABA_{A} α5 receptor agonist (e.g., a compound of Formula II as recited in paragraph 19 or 20 or Formula IV as recited in paragraph 19 or 20; a compound of Formula I-a, I-b, I-c, I-d, I-e, and I-f as recited in paragraph 30; Compounds 1-12, 44-56, 101-268, 270-644, 646-687, 689-698, 700-703, 705, 707-721, 723-740, 742-755, 758-763, 765-779. 781-795, 797-810, 813-828, 830, 831, 833-846, 848-891, 893-903, 905, 907-977, 979-1012 as described above, or Compound 606, Form A; Compound 606, Form B; Compound 606, Form C; Compound 606, Form E; or Compound 606, Form F), or a pharmaceutically acceptable salt, hydrate, solvate, polymorph, or isomer thereof) may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions or combinations (or components thereof) may also comprise buffering agents. Solid pharmaceutical compositions or combinations (or components thereof) of a similar type may also be employed as fillers in soft and hard-filled gelatin capsules using such excipients as lactose or milk sugars, as well as high molecular weight polyethylene glycols and the like.

The pharmaceutical composition or combination (or a component thereof) useful in the methods and uses of this disclosure (e.g., a pharmaceutical composition or combination (or a component thereof) comprising levetiracetam, brivaracetam, or seletracetam, or a pharmaceutically acceptable salt thereof, and/or a GABA_{A} α5 receptor agonist (e.g., a compound of Formula II as recited in paragraph 19 or 20 or Formula IV as recited in paragraph 19 or 20; a compound of Formula I-a, I-b, I-c, I-d, I-e, and I-f as recited in paragraph 30; Compounds 1-12, 44-56, 101-268, 270-644, 646-687, 689-698, 700-703, 705, 707-721, 723-740, 742-755, 758-763, 765-779. 781-795, 797-810, 813-828, 830, 831, 833-846, 848-891, 893-903, 905, 907-977, 979-1012 as described above; or Compound 606, Form A; Compound 606, Form B; Compound 606, Form C; Compound 606, Form E; or Compound 606, Form F), or a pharmaceutically acceptable salt, hydrate, solvate, polymorph, or isomer thereof) may be in an aqueous or non-aqueous liquid dosage form, including solution, emulsion, microemulsion, suspension, syrup, pastille, or elixir. In some embodiments, the pharmaceutical composition or combination (or a component thereof) is in an aqueous solution. In some embodiments, the pharmaceutical composition or combination (or a component thereof) is in a suspension form.

Where appropriate, the pharmaceutical composition or combination (or a component thereof) may be prepared with coatings such as enteric coatings or they may be formulated so as to provide extended release (e.g., a controlled release, a prolonged release, a sustained release, a delayed release, or a slow release) of one or more compound useful in the methods and uses of this disclosure (e.g., levetiracetam, brivaracetam, or seletracetam), or a pharmaceutically acceptable salt thereof, and/or a GABA_{A} α5 receptor agonist (e.g., a compound of Formula II as recited in paragraph 19 or 20 or Formula IV as recited in paragraph 19 or 20; a compound of Formula I-a, I-b, I-c, I-d, I-e, and I-f as recited in paragraph 30; Compounds 1-12, 44-56, 101-268, 270-644, 646-687, 689-698, 700-703, 705, 707-721, 723-740, 742-755, 758-763, 765-779. 781-795, 797-810, 813-828, 830, 831, 833-846, 848-891, 893-903, 905, 907-977, 979-1012 as described above, or Compound 606, Form A; Compound 606, Form B; Compound 606, Form C; Compound 606, Form E; or Compound 606, Form F), or a pharmaceutically acceptable salt, hydrate, solvate, polymorph, or isomer thereof) according to methods well known in the art. Liquid dosage forms may also comprise inert diluents commonly used in the art, such as water or other solvents, solubilizing agents and emulsifiers, such as ethyl alcohol (ethanol), isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, oral pharmaceutical compositions or combinations (or components thereof) useful in the methods and uses of this disclosure can also include adjuvants such as wetting agents, emulsifying and suspending agents, sweetening, flavoring, coloring, perfuming, and preservative agents. Suspensions, in addition to the compounds (e.g., levetiracetam, brivaracetam, or seletracetam), or a pharmaceutically acceptable salt thereof, and/or a GABA_{A} α5 receptor agonist (e.g., a compound of Formula II as recited in paragraph 19 or 20 or Formula IV as recited in paragraph 19 or 20; a compound of Formula I-a, I-b, I-c, I-d, I-e, and I-f as recited in paragraph 30; Compounds 1-12, 44-56, 101-268, 270-644, 646-687, 689-698, 700-703, 705, 707-721, 723-740, 742-755, 758-763, 765-779. 781-795, 797-810, 813-828, 830, 831, 833-846, 848-891, 893-903, 905, 907-977, 979-1012 as described above; or Compound 606, Form A; Compound 606, Form B; Compound 606, Form C; Compound 606, Form E; or Compound 606, Form F), or a pharmaceutically acceptable salt, hydrate, solvate, polymorph, or isomer thereof) useful in the methods and uses of this disclosure, may comprise suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

Pharmaceutical compositions or combinations (or components thereof) useful in the methods and uses of this disclosure may be formulated for respiratory delivery (pulmonary and nasal delivery). In such form, they may be delivered by devices and in forms that include but are not limited to a variety of pressurized metered dose inhalers, dry powder inhalers, nebulizers, aqueous mist inhalers, drops, solutions, suspensions, sprays, powders, gels, ointments, and specialized systems such as liposomes and microspheres (see e.g. Owens DR, Zinman B, Bolli G. Alternative routes of insulin delivery. Diabet Med. 2003 November; 20 (11): 886-98 and Martini G, Ciani L. Electron spin resonance spectroscopy in drug delivery. Phys Chem Phys. 2009).

Pharmaceutical compositions or combinations (or components thereof) useful in the methods and uses of this disclosure may also be formulated for transdermal delivery using formats, including but are not limited to colloids, patches, and microemulsions.

In some embodiments of the methods and uses of this disclosure, one or more of the compounds, pharmaceutical compositions or medicaments maybe packaged together. In other embodiments, one or more of the compounds, pharmaceutical compositions or medicaments may be packaged separately. Combinations useful in the methods and uses of this disclosure also encompass formulations of the levetiracetam, brivaracetam, or seletracetam, or the pharmaceutically acceptable salt thereof, and the GABA_{A} α5 receptor agonist (e.g., a compound of Formula II as recited in paragraph 19 or 20 or Formula IV as recited in paragraph 19 or 20; a compound of Formula I-a, I-b, I-c, I-d, I-e, and I-f as recited in paragraph 30; Compounds 1-12, 44-56, 101-268, 270-644, 646-687, 689-698, 700-703, 705, 707-721, 723-740, 742-755, 758-763, 765-779. 781-795, 797-810, 813-828, 830, 831, 833-846, 848-891, 893-903, 905, 907-977, 979-1012 as described above; or Compound 606, Form A; Compound 606, Form B; Compound 606, Form C; Compound 606, Form E; or Compound 606, Form F), or the pharmaceutically acceptable salt, hydrate, solvate, polymorph, or isomer thereof, or pharmaceutical compositions containing each of them together in one formulation or in separate formulations.

Other suitable administration forms for the pharmaceutical compositions or combinations (or components thereof) useful in the methods and uses of the disclosure include depot injectable formulations, suppositories, sprays, ointments, cremes, gels, inhalants, dermal patches, implants, devices, and formulations for ocular administration.

The pharmaceutical compositions or combinations (or components thereof) useful in the methods and uses of this disclosure may also comprise adjuvants, such as preservatives, wetting agents, emulsifying agents and dispersing agents. Prevention of the action of microorganisms may be afforded by the inclusion of various antibacterial and antifungal agents, for example, paraben, chlorobutanol, phenol sorbic acid, and the like. It may also be desirable to include isotonic agents, such as sugars, sodium chloride, and the like into the pharmaceutical compositions or combinations (or components thereof). In addition, prolonged absorption of the injectable pharmaceutical form may be brought about by the inclusion of agents which delay absorption, such as aluminum monostearate and gelatin.

The pharmaceutical compositions, medicaments or combinations (or components thereof) useful in the methods and uses of this disclosure can be prepared by methods well known in the art of pharmacy, see, e.g., Goodman and Gilman's The Pharmacological Basis of Therapeutics. 10th Edition Edited by J. G. Hardman, L. E. Limbird, and A. G. Gilman. McGraw Hill, New York. 2001; Ansel et al., Pharmaceutical Calculations, The Pharmacist's Handbook 2004, Lippincott Williams & Wilkins; Stoklosa et al., 2001 Pharmaceutical Calculations 11th edition (9780781731720)—Textbooks.com; and Bustamante et al., “A Modification of the Extended Hildebrand Approach to Predict the Solubility of Structurally Related Drugs in Solvent Mixtures,” Journal of Pharmacy and Pharmacology, 45:253-257 (1993).

In some embodiments, the methods of this disclosure comprise administering to the subject levetiracetam, brivaracetam or seletracetam, or a pharmaceutically acceptable salt. In some embodiments, the method comprises administering to the subject a pharmaceutical composition comprising the daily dose of the levetiracetam, brivaracetam or seletracetam or pharmaceutical salt thereof and a pharmaceutically acceptable carrier. In some embodiments, the method comprises administering to the subject levetiracetam, or a pharmaceutically acceptable salt. In some embodiments, the method comprises administering to the subject brivaracetam, or a pharmaceutically acceptable salt. In some embodiments, the method comprises administering to the subject seletracetam, or a pharmaceutically acceptable salt. In some embodiments, the method comprises administering to the subject a pharmaceutical composition comprising the daily dose of the levetiracetam or pharmaceutical salt thereof and a pharmaceutically acceptable carrier. In some embodiments, the method comprises administering to the subject a pharmaceutical composition comprising the daily dose of the brivaracetam or pharmaceutical salt thereof and a pharmaceutically acceptable carrier. In some embodiments, the method comprises administering to the subject a pharmaceutical composition comprising the daily dose of the seletracetam or pharmaceutical salt thereof and a pharmaceutically acceptable carrier.

### General Synthetic Methodology

The compounds of this disclosure may be prepared in general by methods known to those skilled in the art. Schemes 1-4 below provide general synthetic routes for the preparation of compounds of Formula I-a to I-f. Other equivalent schemes, which will be readily apparent to the ordinary skilled organic chemist, may alternatively be used to synthesize various portions (or the entirety) of the molecules as illustrated by the general schemes below.

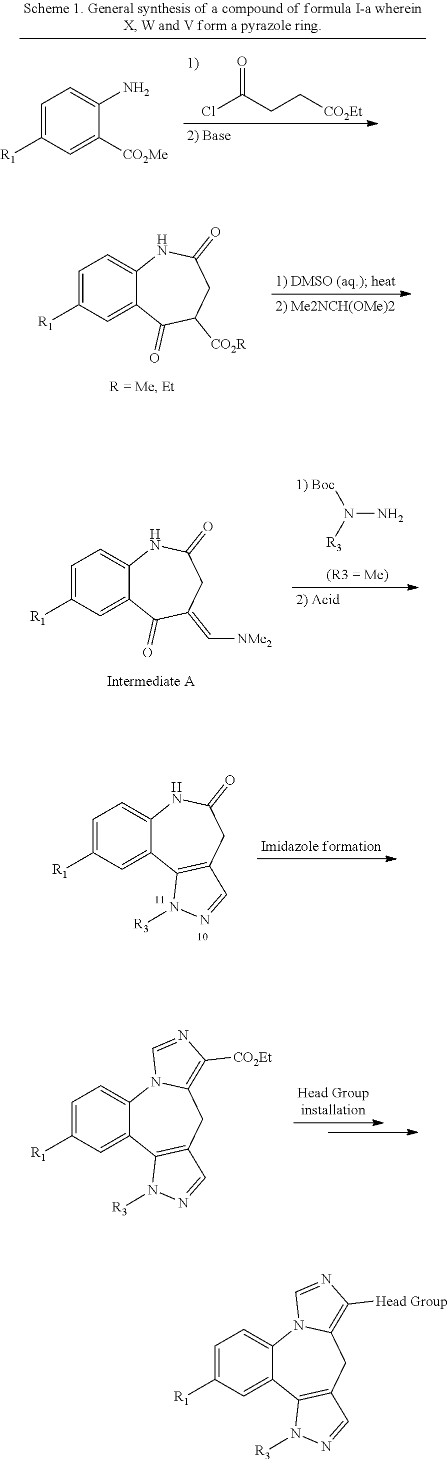

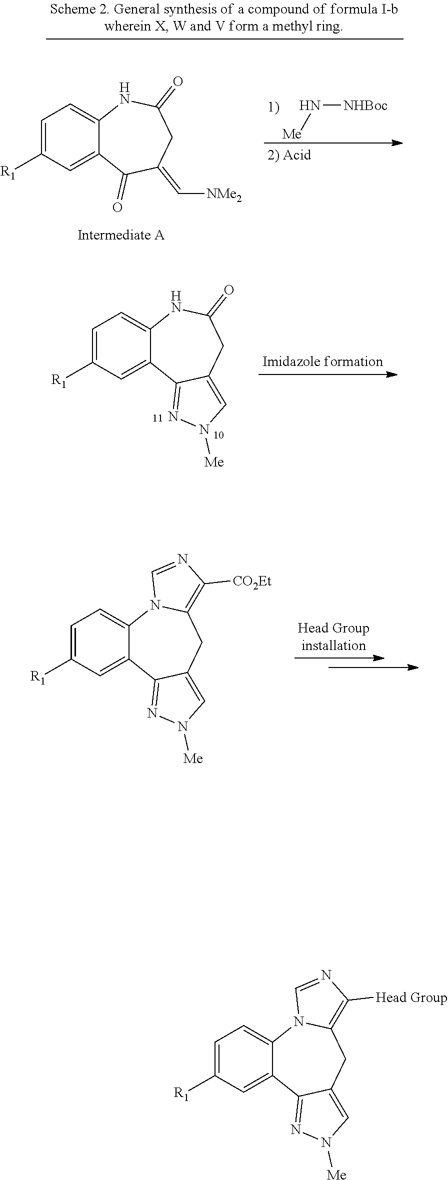

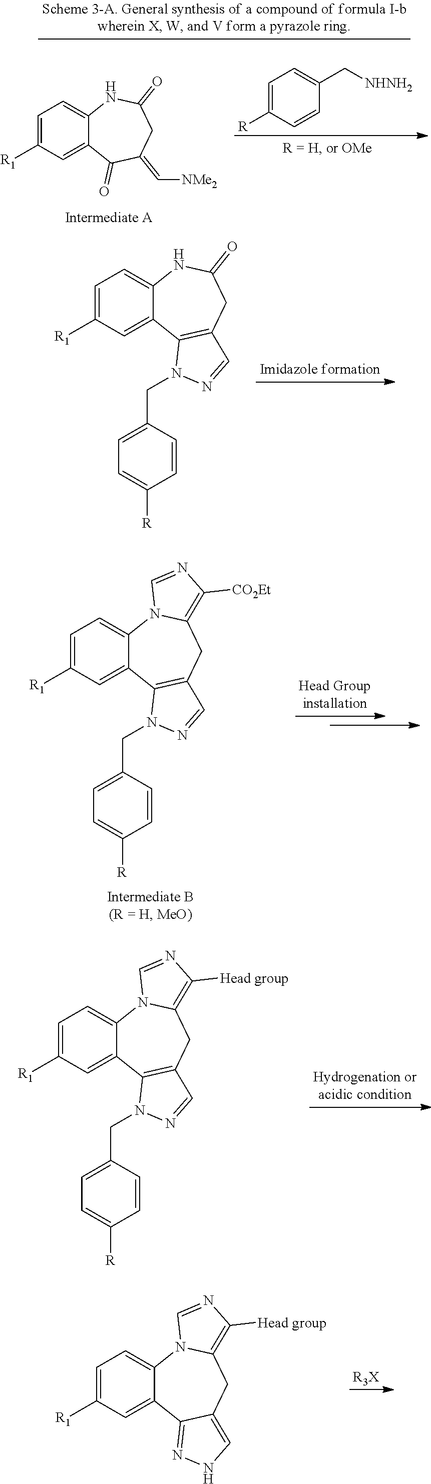

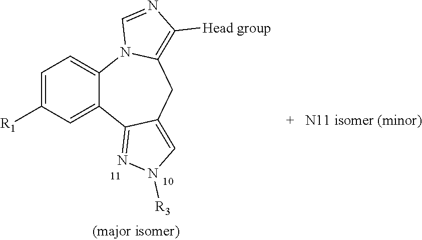

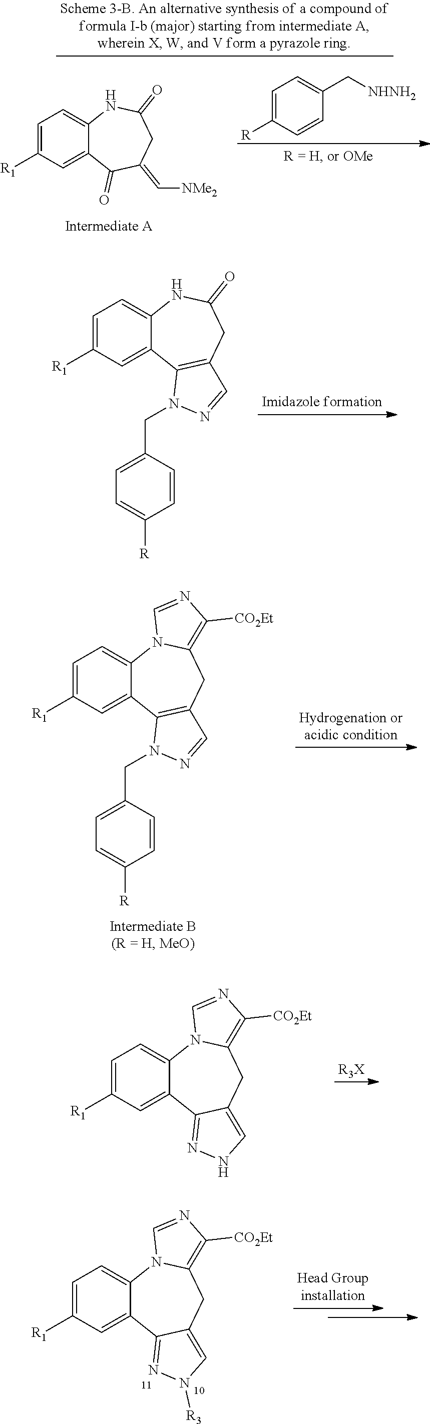

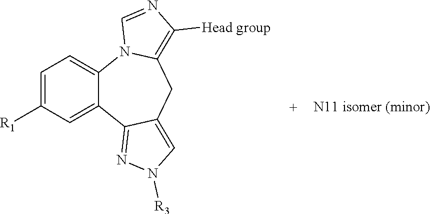

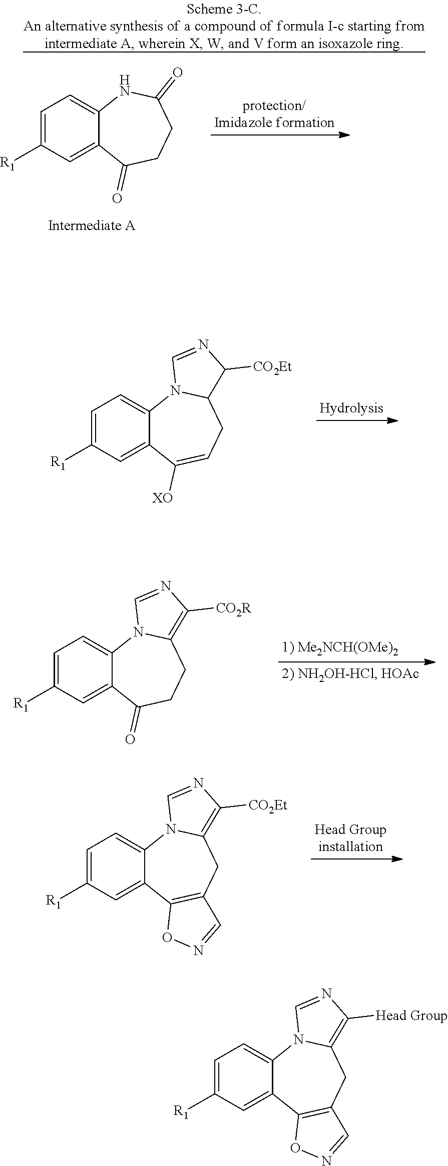

As would be recognized by skilled practitioners, compounds of formulae I, I-a, I-b, I-c, I-d, I-e and I-f with variables other than those depicted above may be prepared by varying the chemical reagents or the synthetic routes.

Predictive Risk Factors of Cognitive Decline or Cognitive Impairment in Subjects Displaying or Presenting with Cognitive Performance within the Normal Range for the Subject's Age.

In some embodiments, the subject to be treated in the methods and uses of this disclosure (a subject displaying or presenting with cognitive performance within the normal range for the subject's age) is at risk of developing cognitive decline or cognitive impairment, where the risk is associated with aging.

In some embodiments of the methods of this disclosure, the subject to be treated is at risk of developing cognitive decline or cognitive impairment, wherein the risk is associated with the presence of altered hippocampal functional connectivity in the subject. In some embodiments of the methods of this disclosure, the subject to be treated is at risk of developing cognitive decline or cognitive impairment, wherein the risk is associated with the presence of increased hippocampal functional connectivity in the subject.

In some embodiments, the subject to be treated in the methods and uses of this disclosure (a subject displaying or presenting with cognitive performance within the normal range for the subject's age) is at risk of developing cognitive decline or cognitive impairment, wherein the risk is a genetic risk associated with the presence of one or more genomic variants, mutations, or polymorphs associated with a change in the expression of genes selected from the group consisting of ABCA7, CLU, CR1, PICALM, PLD3, TREM2, and SORL1 in the genome of the subject. In some embodiments, the subject is at risk of developing cognitive decline or cognitive impairment, wherein the risk is associated with the presence of one or more genomic variants, mutations, or polymorphs associated with ABCA7 in the genome of the subject. In some embodiments, the subject is at risk of developing cognitive decline or cognitive impairment, wherein the risk is associated with the presence of one or more genomic variants, mutations, or polymorphs associated with CLU in the genome of the subject. In some embodiments, the subject is at risk of developing cognitive decline or cognitive impairment, wherein the risk is associated with the presence of one or more genomic variants, mutations, or polymorphs associated with CR1 in the genome of the subject. In some embodiments, the subject is at risk of developing cognitive decline or cognitive impairment, wherein the risk is associated with the presence of one or more genomic variants, mutations, or polymorphs associated with PICALM in the genome of the subject. In some embodiments, the subject is at risk of developing cognitive decline or cognitive impairment, wherein the risk is associated with the presence of one or more genomic variants, mutations, or polymorphs associated with PLD3 in the genome of the subject. In some embodiments, the subject is at risk of developing cognitive decline or cognitive impairment, wherein the risk is associated with the presence of one or more genomic variants, mutations, or polymorphs associated with TREM2 in the genome of the subject. In some embodiments, the subject is at risk of developing cognitive decline or cognitive impairment, wherein the risk is associated with the presence of one or more genomic variants, mutations, or polymorphs associated with SORL1 in the genome of the subject.

In some embodiments, the subject is at risk of developing cognitive decline or cognitive impairment, wherein the risk is associated with the presence of at least one allele of the APOE4 gene in the genome of the subject. In some embodiments, the subject is at risk of developing cognitive decline or cognitive impairment, wherein the risk is associated with the presence of one allele of the APOE4 gene in the genome of the subject. In some embodiments, the subject is at risk of developing cognitive decline or cognitive impairment, wherein the risk is associated with the presence of both APOE4 alleles in the genome of the subject.

In some embodiments, the subject is at risk of developing cognitive decline or cognitive impairment, wherein the risk is associated with the presence of one of more biofluid biomarkers selected from the group consisting of p-tau, t-tau, and amyloid β42 in the subject. In some embodiments, the subject is at risk of developing cognitive decline or cognitive impairment, wherein the risk is associated with the presence of the biofluid biomarker p-tau in the subject. In some embodiments, the subject is at risk of developing cognitive decline or cognitive impairment, wherein the risk is associated with the presence of the biofluid biomarker t-tau in the subject. In some embodiments, the subject is at risk of developing cognitive decline or cognitive impairment, wherein the risk is associated with the presence of the biofluid biomarker amyloid β42 in the subject.

### EXAMPLES

### Example 1: Compositions of Levetiracetam

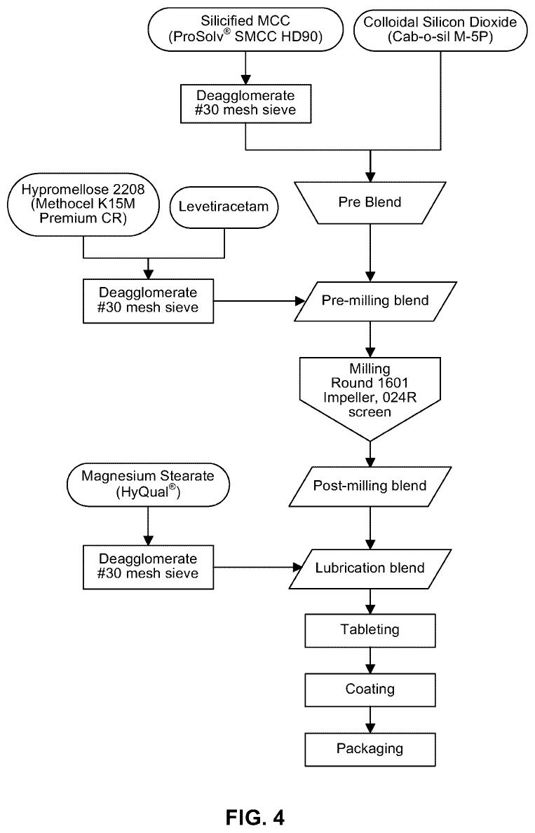

Compositions comprising levetiracetam can be made through the process exemplified in the flow diagram of FIG. 4. In brief, Silicified Microcrystalline Cellulose ProSolv™ SMCC HD90 (or Encompress, Anhydrous dicalcium phosphate) is sifted through deagglomerate #30 U.S. mesh sieve, and then blended with Colloidal Silicon Dioxide (16 qt V-shell blender; 75 rev±5 rev). The blended sample then goes through Round 1601 Impeller (2A024R screen). 220 mg of levetiracetam and hypromellose 2208 (Methocel™ K15M Premium CR) (or Methocel™ K100M Premium CR) are also sifted through deagglomerate #30 U.S. mesh sieve, and then blended in a 1 ft³ Slant Cone Blender (250 rev±5 rev) with the ground Silicified Microcrystalline Cellulose ProSolv™ HD90 and Colloidal Silicon Dioxide. This blended sample then goes through Round 1601 Impeller (2A024R screen) and then is blended in a 1 ft³ Slant Cone Blender (125 rev±5 rev) with sieved Magnesium Stearate (HyQual®) (sieved through deagglomerate #30 U.S. mesh sieve). The blended samples are compressed into tablets. Optionally, the tablets are further film coated with a hypromellose-based (HPMC-based) coating, such as Opadry® complete film coating system. A similar process can be used for compositions comprising 190 mg of levetiracetam.

### Example 2: Evaluation of Levetiracetam Blood Plasma Levels

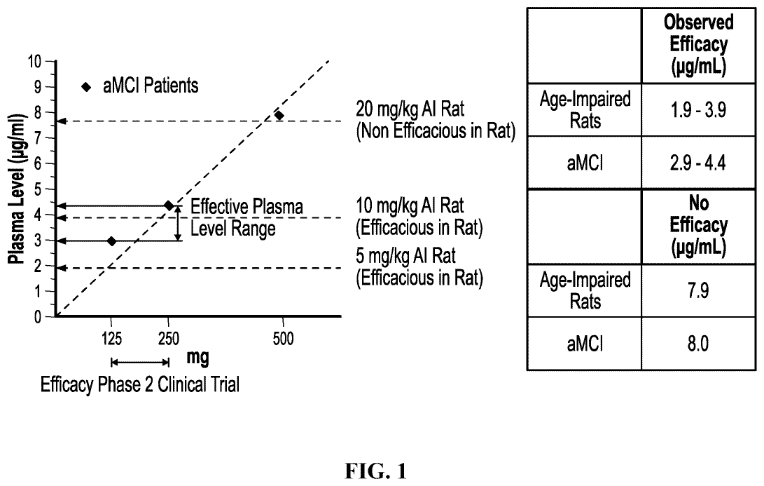

Steady state levetiracetam plasma concentration ranges effective for treating cognitive impairment were previously established using the aMCI clinical trials and aged rat studies disclosed in WO2016191288, which is incorporated by reference herein in its entirety. In one embodiment, the concentration range of the levetiracetam is between 2.9 and 4.4 μg/mL. In another embodiment, the concentration range of the levetiracetam is between 1.9 and 4.4 μg/mL. In another aspect, the effective plasma concentration is between 1.9 and 3.9 μg/mL. See FIG. 1.

This example further describes a two-group, single-dose, two-period, two-way crossover, food-effect study of two extended release levetiracetam formulations, i.e., the 190 mg Tablet A of Table 1 and the 220 mg Tablet D of Table 2.

### Study Design

This is an open label, randomized, two-group, single-dose, two-period crossover, food-effect study. Fifty-six (56) healthy subjects are enrolled. Subjects who successfully complete the screening process check into the research center the evening before first dose. Subjects who continue to meet inclusion/exclusion criteria the morning of dose are assigned a subject number, based on the order in which they successfully complete the required screening process and procedures. Dosing days are separated by a washout period of at least 7 days. Subjects are randomly assigned to one of two groups:
Group 1: Subjects (n=28) received extended-release Tablet A of Table 1 (190 mg).
Treatment A: Tablet A
   Dose=1×190 mg tablet, orally administered under fasted conditions
Treatment B: Tablet A
   Dose=1×190 mg tablet, orally administered under fed conditions
Group 2: Subjects (n=28) received extended-release Tablet D of Table 2 (220 mg).
Treatment A: Tablet D
   Dose=1×220 mg tablet, orally administered under fasted conditions
Treatment B: Tablet D
   Dose=1×220 mg tablet, orally administered under fed conditions

### Clinical Procedures Summary

During each study period, 6 mL blood samples are obtained prior to each dosing and following each dose at selected times through 24 hours post-dose. A total of 34 pharmacokinetic blood samples are to be collected from each subject, 17 samples in each study period. In addition, blood is drawn and urine is collected for clinical laboratory testing at screening and study exit.

In each study period, subjects are admitted to the study unit in the evening prior to the scheduled dose. Subjects are confined to the research center during each study period until completion of the 24-hour blood collection and other study procedures.

### Procedures for Collecting Samples for Pharmacokinetic Analysis

Blood samples (1×6 mL) are collected in vacutainer tubes containing K2-EDTA as a preservative at pre-dose (0) and at 1.0, 2.0, 3.0, 4.0, 4.5, 5.0, 5.5, 6.0, 6.5, 7.0, 8.0, 9.0, 10, 12, 18, and 24 hours after dosing.

### Bioanalytical Summary

Plasma samples are analyzed for levetiracetam using a validated LC MS MS procedure. The method is validated for a range of 0.0500 to 30.0 μg/mL for levetiracetam, based on the analysis of 0.200 mL of human EDTA plasma. Data are stored in Watson Laboratory Information Management System (LIMS; Version 7.2.0.03, Thermo Fisher Scientific).

### Pharmacokinetic Analysis

Data are analyzed by noncompartmental methods in WinNonlin. Concentration time data that are below the limit of quantification (BLQ) are treated as zero in the data summarization and descriptive statistics. In the pharmacokinetic analysis, BLQ concentrations are treated as zero from time-zero up to the time at which the first quantifiable concentration is observed; embedded and/or terminal BLQ concentrations are treated as “missing”. Actual sample times are used for all pharmacokinetic and statistical analyses.

The following pharmacokinetic parameters are calculated: peak concentration in plasma (Cmax), time to peak concentration (Tmax), elimination rate constant (λz), terminal half-life (T½), area under the concentration-time curve from time-zero to the time of the last quantifiable concentration (AUClast), and area under the plasma concentration time curve from time-zero extrapolated to infinity (AUCinf). Additionally, Cmax, AUClast, and AUCinf are dose-normalized.

### Steady State Modeling

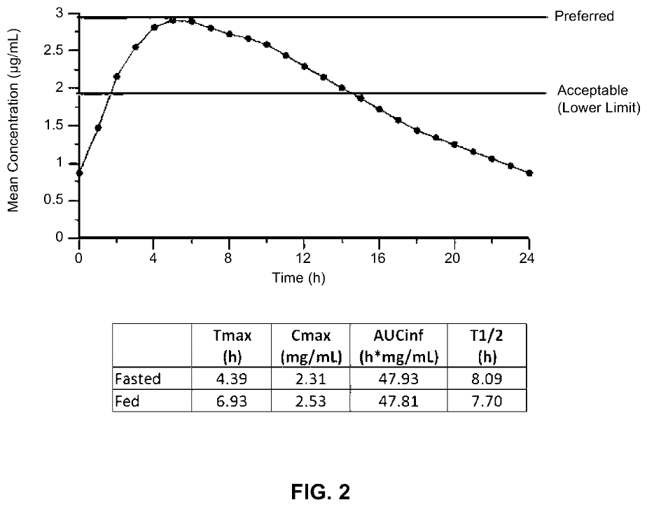

According to the steady state modeling of PK profile for the 190 mg Tablet A, the plasma concentrations of the levetiracetam were between 1.9 and 4.4 μg/mL for substantial parts of the 24-hour period after administration. See FIG. 2.

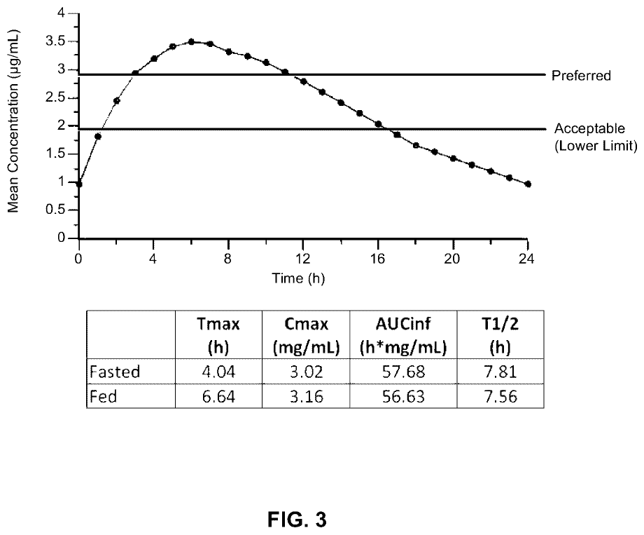

According to the steady state modeling of PK profile for the 220 mg Tablet D, the plasma concentrations of the levetiracetam were between 2.9 and 4.4 μg/mL for substantial parts of the 24-hour period after administration. See FIG. 3.

### Example 3: Effect of Levetiracetam on Hippocampal Functional Connectivity: An Alzheimer's Disease-Associated LEV-HFC Study

### Subjects

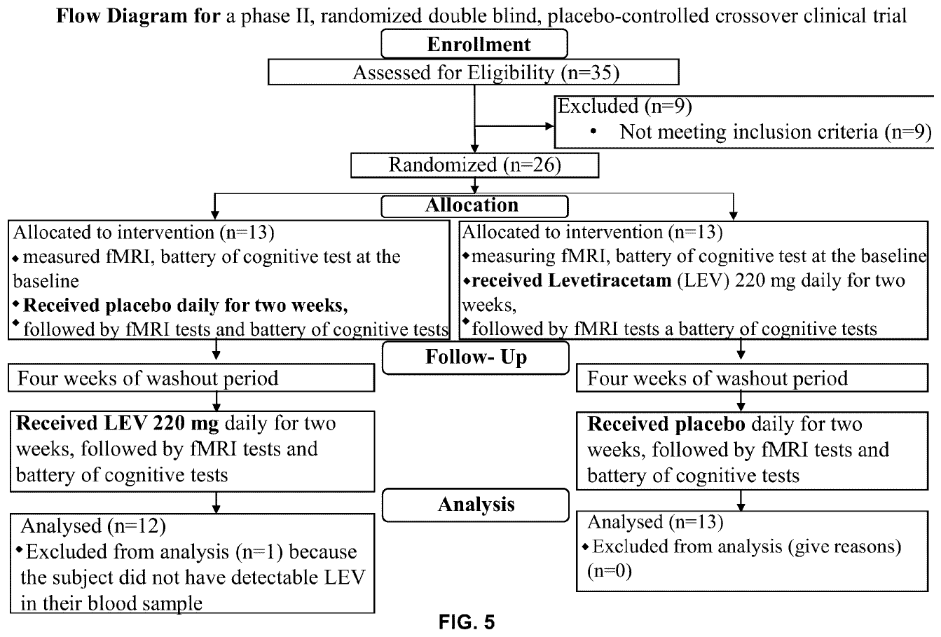

A phase II, randomized, double-blind, placebo-controlled crossover clinical trial of 26 CN subjects (ranging in age from 55 to 75 years) to assess the potential effect of levetiracetam on hippocampal functional connectivity (a risk marker for cognitive decline and cognitive impairment and progression to AD) was conducted. 35 patients were recruited and assessed for eligibility and the 26 eligible subjects were separated into Group A or Group B treatment groups. Group A subjects received an oral placebo tablet daily for two weeks followed by a four-week washout period; then, the subjects received an oral levetiracetam tablet daily for two weeks (i.e., two-week placebo treatment, four-week washout period, and two-week levetiracetam treatment). Group B subjects received treatment using the reverse sequence (i.e., two-week levetiracetam treatment, four-week washout period, and two-week placebo treatment). The tablet was characterized by 220 mg levetiracetam in extended release form, i.e. Tablet D (Table 2) above. A flow diagram of the study design is shown in FIG. 5.

### MRI Protocol

For each scanning session, imaging was carried out on a 3T GE MR750 whole-body scanner (GE Healthcare, Waukesha WI) with a body transmit and a 32-channel head receive coil (Nova Medical, Wilmington MA). Imaging acquisition included T1- and T2-weighted high resolution, anatomical images (TR=2521.27 ms, TE=3.596 ms, Voxel resolution: 0.5×0.5×0.8 mm3, FOV: 25.6 cm, Flip angle: 8°). Functional images included episodic memory (300 s duration), finger tapping (192 s duration) and two resting states (480 s duration, one A-P axial direction and one P-A axial direction scanning). Functional Imaging parameters included: TE: 33.5 ms, TR: 800 ms, a 50° flip angle, 128×128 acquisition matrix, 20.8 cm axial FOV, 2 mm thick slices, 8-band simultaneous multi-slice acceleration, ⅓ blipped-CAIPI field of view shift and 72 total slices, harmonized with the Lifespan Human Connectome Project acquisition protocol. T2-flair: Voxel resolution: 0.5×0.5×0.8 mm3, FOV: 25.6 cm, TR: 4002 ms, TE: 161.893 ms, a 90° flip angle. Blood flow image (pcASL) included: 72 frames, Voxel resolution: 1.875×1.875×4 mm3, 24 cm FOV, TR: 4667 ms, TE: 56.32 ms.

### Resting-State Image Preprocessing

Image pre-processings were conducted using Analysis of Functional NeuroImages (AFNI) software (http://afni.nimh.nih.gov/afni/), SPM8 (Wellcome Trust, London, United Kingdom), and MATLAB (MathWorks, Natick, Massachusetts). The preprocessing allows for T1-equilibration (removing the first 15 seconds of R-fMRI data); slice-acquisition-dependent time shift correction (3dTshift); motion correction (3dvolreg); detrending (3dDetrend); despiking (3dDespike); spatial normalization (original space to the Montreal Neurological Institute [MNI] space, SPM8); averaging white matter and cerebrospinal-fluid (CSF) signal retrieval (3dROIstats) using standard SPM white matter and CSF mask in the MNI space; white matter, CSF signal, and motion effect removal (3dDeconvolve); global signal removal necessity check (the global signal being if necessary) (see, e.g., Chen et al, 2012); and low-frequency band-pass filtering (3dFourier, 0.015-0.1 Hz).

### Total Functional Connectivity Indices

Total Functional connectivity indices (TFCI) were obtained based on the traditional region of interest (ROI)-based method (Chen et al, 2011) in which individual cerebrums were automatically parcellated into 90 regions, per the Automated Anatomical Labeling (AAL) toolbox (3), and the FCI was calculated as the summation of Pearson cross-correlation coefficients between the seed region and all other regions in the cerebrum. Specifically, first, the whole cerebral cortex was separated into 90 regions based on the AAL template, and the blood oxygen level dependent (BOLD) time series of each region was extracted using the AAL template mask from the preprocessed resting-state data set as published in Tzourio-Mazoyer et al (2002). Second, functional connectivity between each ROI and the other brain regions was calculated using the Pearson cross-correlation analysis. Thus, a vector consisting of 89 cross-correlation coefficient (CC) values for each ROI was obtained. Finally, each ROI's FCI value was calculated by summating 89 CC values within each ROI vector. In addition to eliminate the outlier effect, Median Functional connectivity indices (MFCI) were calculated by the median value of 89 CC values within each ROI vector. TFCI and MFCI were calculated for left, right and bilateral regions.

### Results

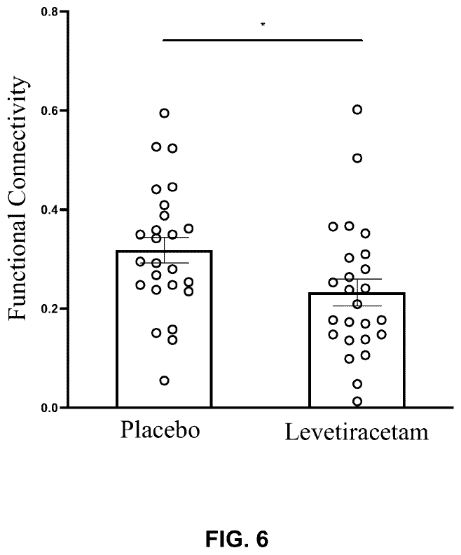

Of the 35 CN subjects assessed for eligibility, 9 did not meet eligibility criteria and were not enrolled. One enrolled subject did not have detectable levetiracetam in their blood sample and was discarded in analysis. A total of 25 participants (15 women with a mean [SD] age of 65.9 [3.8] years and 10 men with a mean [SD] age of 66.7 [5.5] years) were enrolled, randomized (12 participants to group A and 13 participants to group B), and completed the study. See Table 3. Overall, treatment with levetiracetam compared with placebo significantly decreased the functional connectivity of the hippocampus network (paired t-test, t=−2.8044, p<0.0098; FIG. 6).

TABLE 3
Demographic characteristics and functional connectivity strengths

| | | | | | | | Period | Period | LEV |
|---|---|---|---|---|---|---|---|---|---|
| Subject | | | | | APOE | Baseline | I | II | [μg] in |
| ID | Gender | Age | Education | MOCA | Type | FC | FC | FC | blood |
| 1 | M | 56 | 18 | 27 | E3/E4 | 0.08 | 0.055* | 0.106 | 2 |
| 4 | M | 73 | 18 | 27 | E3/E3 | 0.185 | 0.292* | 0.28 | 4.4 |
| 5 | F | 66 | 16 | 26 | E3/E3 | 0.044 | 0.151* | 0.177 | 8.2 |
| 6 | F | 70 | 20 | 29 | E3/E3 | 0.441 | 0.209 | 0.441* | 3 |
| 7 | F | 69 | 16 | 29 | E3/E3 | 0.551 | 0.602 | 0.35* | 4.5 |
| 8 | F | 66 | 16 | 30 | E3/E3 | 0.324 | 0.248* | 0.264 | 4.8 |
| 9 | F | 70 | 12 | 29 | E3/E3 | 0.361 | 0.35* | 0.238 | 6.1 |
| 10 | M | 69 | 18 | 30 | E3/E3 | 0.582 | 0.148 | 0.595* | 2.9 |
| 11 | F | 67 | 18 | 28 | E3/E4 | 0.227 | 0.241 | 0.409* | 4.1 |
| 12 | F | 64 | 16 | 26 | E3/E3 | 0.317 | 0.138 | 0.362* | 4.1 |
| 13 | F | 65 | 16 | 29 | E3/E4 | 0.06 | 0.099 | 0.342* | 5.1 |
| 16 | F | 71 | 16 | 28 | E3/E3 | 0.328 | 0.254* | 0.366 | 4.4 |
| 17 | F | 63 | 12 | 26 | E3/E3 | 0.083 | 0.295* | 0.013 | 6.3 |
| 18 | M | 70 | 18 | 28 | E3/E3 | 0.205 | 0.136 | 0.235* | 4.9 |
| 22 | F | 59 | 18 | 30 | E3/E2 | 0.404 | 0.504 | 0.388* | 4.2 |
| 23 | F | 67 | 16 | 27 | E3/E3 | 0.406 | 0.253 | 0.238* | 3.1 |
| 24 | M | 64 | 16 | 25 | E4/E4 | 0.439 | 0.446* | 0.352 | 2.8 |
| 25 | M | 68 | 12 | 30 | E3/E3 | 0.223 | 0.268* | 0.31 | 3.8 |
| 26 | M | 62 | 18 | 28 | E3/E3 | 0.337 | 0.137* | 0.048 | 3.8 |
| 27 | F | 61 | 16 | 27 | E3/E3 | 0.42 | 0.367 | 0.524* | 5.8 |
| 28 | M | 73 | 18 | 28 | E3/E4 | 0.244 | 0.173 | 0.248* | 5 |
| 29 | M | 62 | 16 | 29 | E3/E3 | 0.489 | 0.303 | 0.527* | 3.2 |
| 30 | M | 70 | 16 | 26 | E3/E3 | 0.232 | 0.359* | 0.177 | 3.4 |
| 32 | F | 70 | 16 | 30 | E3/E2 | 0.174 | 0.158* | 0.17 | 4 |
| 33 | F | 60 | 20 | 27 | E3/E3 | 0.149 | 0.148 | 0.28* | 4.7 |
| AVG | | 66.2 | 16.5 | 28 | | 0.292 | | | 4.3 |
| STD | | 4.5 | 2.1 | 1.5 | | 0.152 | | | 1.3 |

In Table 3, in the columns Period I and II, the numbers with * indicate the functional connectivity from subjects who received two-weeks placebo treatment, all other numbers indicate the functional connectivity from subjects who received two-weeks levetiracetam treatment.

Table 3 demonstrates the individual scores of each participant. Briefly, the table column indicates from left to right the Subject ID number, the gender of the subject, the age of the subject, the level of education of the subject in years, the Montreal Cognitive Assessment (MoCA) Test for Dementia scores for the subject, the APOE genotype of the subject, baseline hippocampal functional connectivity of the subject, hippocampal functional connectivity during the first two week period of treatment (levetiracetam or placebo), hippocampal functional connectivity during the second two week period of treatment after washout (levetiracetam or placebo), and levels of levetiracetams in the blood after treatment with levetiracetam.

Exemplary methods and materials are described, although methods and materials similar or equivalent to those described herein can also be used in the practice or testing of the various aspects and embodiments. The materials, methods, and examples are illustrative only and not intended to be limiting.

What is claimed:

1. A method of preventing or slowing the progression of cognitive impairment or preventing the development or reducing the rate of cognitive decline in a subject, the subject displaying or presenting with cognitive performance within the normal range for the subject's age, the method comprising administering to the subject levetiracetam, brivaracetam or seletracetam, or a pharmaceutically acceptable salt thereof at a daily dose of 0.7-350 mg or comprising administering to the subject a pharmaceutical composition comprising the daily dose of the levetiracetam, brivaracetam or seletracetam or pharmaceutical salt thereof and a pharmaceutically acceptable carrier.

2. The method claim 1, wherein the daily dose of the levetiracetam or seletracetam, or a pharmaceutically acceptable salt thereof is 7-350 mg.

3. The method claim 1, wherein the daily dose of the brivaracetam, or pharmaceutically acceptable salt thereof is 0.7-180 mg.

4. The method claim 1, wherein the daily dose of the levetiracetam or seletracetam, or pharmaceutically acceptable salt thereof is 125-250 mg.

5. The method of claim 4, wherein the daily dose of the levetiracetam or seletracetam, or pharmaceutically acceptable salt thereof is 220 mg.

6. The method of claim 4, wherein the daily dose of the levetiracetam or seletracetam or pharmaceutically acceptable salt thereof is 190 mg.

7. The method of claim 1, wherein the pharmaceutical composition is formulated in one or more of an oral form, an extended release form or a single-unit-dosage-form or for once-a-day administration.

8. The method of claim 7, wherein the extended release form is a controlled release form, a prolonged release form, a sustained release form, a delayed release form, or a slow release form.

9. The method of claim 1, wherein the daily dose of the levetiracetam or pharmaceutically acceptable salt thereof in the pharmaceutical composition is 220 mg and wherein the pharmaceutical composition further comprises 280 mg-350 mg of hydroxypropyl methylcellulose, 1.2 mg-1.4 mg of colloidal silicon dioxide, 92.8 mg-119.2 mg of silicified microcrystalline cellulose, and 6.0 mg-6.7 mg of magnesium stearate.

10. The method of claim 9, wherein the pharmaceutical composition comprises 280 mg of hydroxypropyl methylcellulose, 1.2 mg of colloidal silicon dioxide, 92.8 mg of silicified microcrystalline cellulose, and 6.0 mg of magnesium stearate.

11. The method of claim 9, wherein the pharmaceutical composition comprises 347.5 mg of hydroxypropyl methylcellulose, 1.4 mg of colloidal silicon dioxide, 119.2 mg of silicified microcrystalline cellulose, and 6.7 mg of magnesium stearate.

12. The method of claim 1, wherein the daily dose of the levetiracetam or pharmaceutically acceptable salt thereof in the pharmaceutical composition is 190 mg and wherein the pharmaceutical composition further comprises 300 mg of hydroxypropyl methylcellulose, 1.2 mg of colloidal silicon dioxide, 102.8 mg of silicified microcrystalline cellulose or anhydrous dicalcium phosphate, and 6 mg of magnesium stearate.

13. The method of any one of claims 9-12, wherein the hydroxypropyl methylcellulose is hypromellose 2208.

14. The method of any one of claims 9-13, wherein the silicified microcrystalline cellulose is silicified microcrystalline cellulose SMCC 90.

15. The method of claim 1, wherein the pharmaceutical composition comprising the daily dose of the levetiracetam or pharmaceutically acceptable salt thereof is in extended release form and provides a steady state plasma concentration of levetiracetam in a subject of between 1.9 μg/mL and 4.4 μg/mL within 3 hours after administration and extending for at least 8 hours of a 24-hour period after said administration.

16. The method of claim 15, wherein the pharmaceutical composition provides said steady state plasma concentration of levetiracetam within 2 hours after said administration and extending for at least 13 hours of a 24-hour period after said administration.

17. The method of claim 15, wherein the pharmaceutical composition provides said steady state plasma concentration of levetiracetam within 1 hour after said administration and extending for at least 13 hours of a 24-hour period after said administration.

18. The method of claim 1, wherein the pharmaceutical composition provides said steady state plasma concentration of levetiracetam within 1 hour after administration and extending for at least 13 to 16 hours of a 24-hour period after said administration.

19. The method of claim 15-18, wherein the pharmaceutical composition is formulated in one or more of an oral form, an extended release form or a single-unit-dosage-form or for once-a-day administration.

20. The method of claim 19, wherein the extended release form is a controlled release form, a prolonged release form, a sustained release form, a delayed release form, or a slow release form.

21. A method of preventing or slowing the progression of cognitive impairment or preventing the development or reducing the rate of cognitive decline in a subject, the subject displaying or presenting with cognitive performance within the normal range for the subject's age, the method comprising administering to the subject a GABA_{A} α5 receptor agonist or a pharmaceutically acceptable salt, hydrate, solvate, isomer, or polymorph thereof or administering to the subject a pharmaceutical composition comprising any of them and a pharmaceutically acceptable carrier.

22. The method of claim 21, wherein the GABA_{A} α5 receptor agonist, or the pharmaceutically acceptable salt, hydrate, solvate, isomer, or polymorph thereof or the GABA_{A} α5 receptor agonist, or the pharmaceutically acceptable salt, hydrate, solvate, isomer, or polymorph thereof of the pharmaceutical composition, is selected from the group consisting of:
i) a compound of formula II: 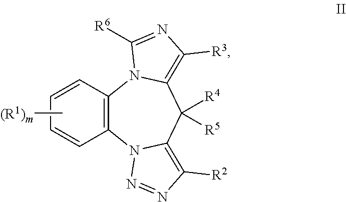
or a pharmaceutically acceptable salt, hydrate, solvate, polymorph, or isomer thereof, wherein:
m is 0-3;
each occurrence of R¹, R², R⁴, and R⁵ are each independently selected from:
halogen, —R, —OR, —NO₂, —NCS, —CN, —CF₃, —OCF₃, —SiR₃, —N(R)₂, —SR, —SOR, —SO₂R, —SO₂N(R)₂, —SO₃R, —(CR₂)₁₋₃R, —(CR₂)₁₋₃—OR, —(CR₂)₀₋₃—C(O)NR(CR₂)₀₋₃R, —(CR₂)₀₋₃—C(O)NR(CR₂) 0.3OR, —C(O)R, —C(O)C(O)R, —C(O)CH₂C(O)R, —C(S)R, —C(S)OR, —C(O)OR, —C(O)C(O)OR, —C(O)C(O)N(R)₂, —OC(O)R, —C(O)N(R)₂, —OC(O)N(R)₂, —C(S)N(R)₂, —(CR₂)₀₋₃NHC(O)R, —N(R)N(R)COR, —N(R)N(R)C(O)OR, —N(R)N(R)CON(R)₂, —N(R)SO₂R, —N(R)SO₂N(R)₂, —N(R)C(O)OR, —N(R)C(O)R, —N(R)C(S)R, —N(R)C(O)N(R)₂, —N(R)C(S)N(R)₂, —N(COR)COR, —N(OR)R, —C(═NH)N(R)₂, —C(O)N(OR)R, —C(═NOR)R, —OP(O)(OR)₂, —P(O)(R)₂, —P(O)(OR)₂, and —P(O)(H)(OR);
R³ is selected from the group consisting of:
halogen, —R, —OR, —NO₂, —NCS, —CN, —CF₃, —OCF₃, —SiR₃, —N(R)₂, —SR, —SOR, —SO₂R, —SO₂N(R)₂, —SO₃R, —(CR₂)₁₋₃R, —(CR₂)₁₋₃—OR, —(CR₂)₀₋₃—C(O)NR(CR₂)₀₋₃R, —(CR₂)₀₋₃—C(O)NR(CR₂)₀₋₃OR, —C(O)R, —C(O)C(O)R, —C(O)CH₂C(O)R, —C(S)R, —C(S)OR, —C(O)OR, —C(O)C(O)OR, —C(O)C(O)N(R)₂, —OC(O)R, —C(O)N(R)₂, —OC(O)N(R)₂, —C(S)N(R)₂, —(CR₂)₀₋₃NHC(O)R, —N(R)N(R)COR, —N(R)N(R)C(O)OR, —N(R)N(R)CON(R)₂, —N(R)SO₂R, —N(R)SO₂N(R)₂, —N(R)C(O)OR, —N(R)C(O)R, —N(R)C(S)R, —N(R)C(O)N(R)₂, —N(R)C(S)N(R)₂, —N(COR)COR, —N(OR)R, —C(═NH)N(R)₂, —C(O)N(OR)R, —C(═NOR)R, —OP(O)(OR)₂, —P(O)(R)₂, —P(O)(OR)₂, and —P(O)(H)(OR);
—C≡CH, —C≡CR⁹, —(C1-C6)alkyl-C≡C—R¹⁰, —CH₂—O—R¹⁰, —CH₂—O—CH₂-R¹⁰ 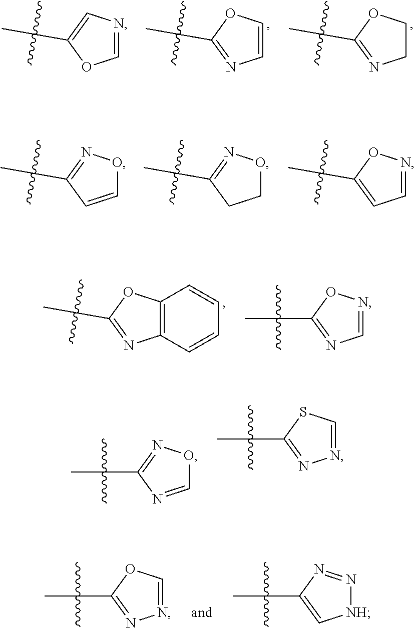
wherein each 5-member heterocycle or heteroaryl is substituted with 0-4 R₇;
wherein R³ is independently substituted with 0-5 R′;
R⁶ is selected from the group consisting of —H and —(C1-C6)alkyl;
wherein R₇ is selected from the group consisting of —(C1-C6)alkyl, —(C3-C6) cycloalkyl, -5 to 10 membered heteroaryl, —(C6-C10) aryl, (C6-C10) aryl-(C1-C6)alkyl-, -5 to 10 membered heteroaryl-(C1-C6)alkyl, and -5-10 membered heteroaryl; wherein each R₇ is independently substituted with 0-5 R′;
wherein each R⁸ is independently selected from the group consisting of —H, —(C1-C6)alkyl, —(C3-C6) cycloalkyl, —(C1-C6)alkyl-(C3-C6) cycloalkyl, —(C1-C6)alkyl-(C6-C10) aryl, —(C6-C10) aryl, -5-10 membered heteroaryl, and —(C1-C6)alkyl-5-10 membered heteroaryl;
wherein each R⁸ excluding-H and —(C1-C6)alkyl is independently substituted by 0-5 of -halogen, —(C1-C6)alkyl, —CF₃, —OCF₃, or O—(C1-C6)alkyl;
wherein R⁹ is selected from the group consisting of —H, —(C1-C6)alkyl, —(C6-C10) aryl, -5-10 membered heteroaryl, —(C1-C6)alkyl-(C6-C10) aryl, —(C1-C6)alkyl-5-10 membered heteroaryl, —(C3-C6) cycloalkyl, —(C1-C6)alkyl-(C3-C6) cycloalkyl, —C(O)—(C6-C10) aryl, 5-10 membered heterocycle, 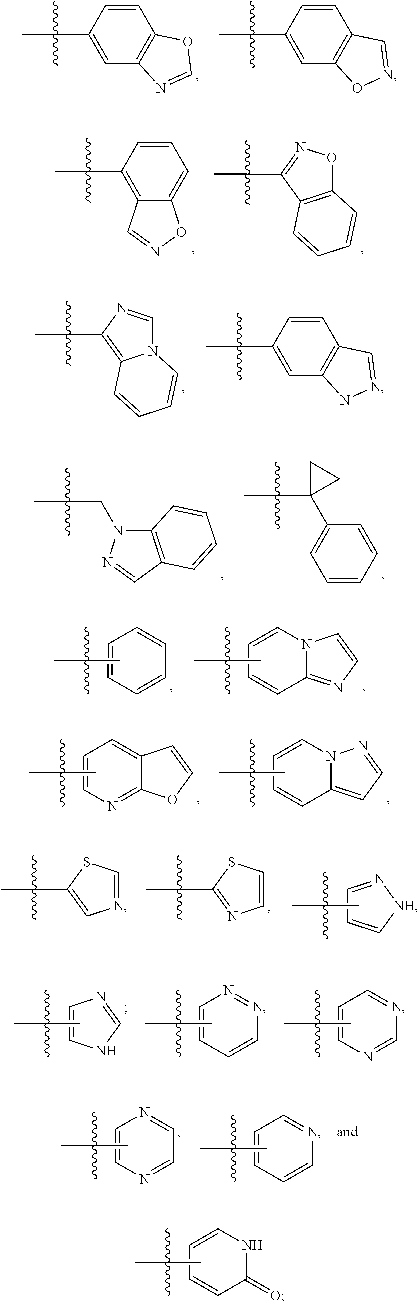
wherein each R⁹ is independently substituted with 0-5 R¹¹;
wherein R¹⁰ is selected from the group consisting of —H, halogen, —(C1-C6)alkyl, —(C6-C10) aryl, -5-10 membered heteroaryl, —(C3-C6) cycloalkyl, —CH₂—(C3-C6) cycloalkyl, —CH₂—(C6-C10) aryl, and —CH₂-5-10-membered heteroaryl,
wherein each R¹⁰ is substituted with 0-5 R′;
wherein each occurrence of R¹¹ is independently selected from the group consisting of -halogen, —CN, SCH₃, —CF₃, —OH, —OCF₃, OCHF₂, —O(C1-C6)alkyl, —(C6-C10) aryl, —(C1-C6)alkyl, and -5 to 10 membered heteroaryl;
each R is independently selected from the group consisting of:
H—,
(C1-C12)-aliphatic-,
(C3-C10)-cycloalkyl-,
(C3-C10)-cycloalkenyl-,
[(C3-C10)-cycloalkyl]-(C1-C12)-aliphatic-,
[(C3-C10)-cycloalkenyl]-(C1-C12)-aliphatic-,
[(C3-C10)-cycloalkyl]-O—(C1-C12)-aliphatic-,
[(C3-C10)-cycloalkenyl]-O—(C1-C12)-aliphatic-,
(C6-C10)-aryl-,
(C6-C10)-aryl-(C1-C12) aliphatic-,
(C6-C10)-aryl-O—(C1-C12) aliphatic-,
(C6-C10)-aryl-N(R″)—(C1-C12) aliphatic-,
3- to 10-membered heterocyclyl-,
(3- to 10-membered heterocyclyl)-(C1-C12) aliphatic-,
(3- to 10-membered heterocyclyl)-O—(C1-C12) aliphatic-,
(3- to 10-membered heterocyclyl)-N(R″)—(C1-C12) aliphatic-,
5- to 10-membered heteroaryl-,
(5- to 10-membered heteroaryl)-(C1-C12)-aliphatic-,
(5- to 10-membered heteroaryl)-O—(C1-C12)-aliphatic-; and
(5- to 10-membered heteroaryl)-N(R″)—(C1-C12)-aliphatic-;
wherein said heterocyclyl has 1-4 heteroatoms independently selected from the group consisting of N, NH, O, S, SO, and SO₂, and said heteroaryl has 1-4 heteroatoms independently selected from the group consisting of N, NH, O, and S;
wherein each occurrence of R is independently substituted with 0-5 R′;
or when two R groups bound to the same atom, the two R groups may be taken together with the atom to which they are bound to form a 3- to 10-membered aromatic or non-aromatic ring having 0-4 heteroatoms independently selected from the group consisting of N, NH, O, S, SO, and SO₂, wherein said ring is optionally substituted with 0-5 R′, and wherein said ring is optionally fused to a (C6-C10) aryl, 5- to 10-membered heteroaryl, (C3-C10) cycloalkyl, or a 3- to 10-membered heterocyclyl;
wherein each occurrence of R′ is independently selected from the group consisting of halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂;
wherein each occurrence of R″ is independently selected from the group consisting of H, —(C1-C6)-alkyl, —(C1-C6)-aliphatic, (C3-C6)-cycloalkyl, 3- to 6-membered heterocyclyl, 5- to 10-membered heteroaryl-, (C6-C10)-aryl-, (5- to 10-membered heteroaryl)-(C1-C6)-alkyl-, (C6-C10)-aryl-(C1-C6)-alkyl-, (5- to 10-membered heteroaryl)-O—(C1-C6)-alkyl-, (C6-C10)-aryl-O—(C1-C6)-alkyl-, and (C6-C10)-aryl-O—(C1-C6)-alkyl-,
wherein each occurrence of R″ is independently substituted with 0-5 substituents selected from the group consisting of: halogen, —R^{o}, —OR^{o}, oxo, —CH₂OR^{o}, —CH₂N(R^{o})₂, —C(O)N(R^{o})₂, —C(O)OR^{o}, —NO₂, —NCS, —CN, —CF₃, —OCF₃ and —N(R^{o})₂, wherein each occurrence of R^{o} is independently selected from the group consisting of: —(C1-C6)-aliphatic, (C3-C6)-cycloalkyl, 3- to 6-membered heterocyclyl, 5- to 10-membered heteroaryl-, and (C6-C10)-aryl; and
ii) a compound of formula IV: 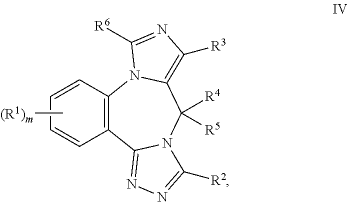
or a pharmaceutically acceptable salt, hydrate, solvate, polymorph, or isomer thereof, wherein:
m is 0-3;
each R¹, R⁴ and R⁵ is independently selected from: each occurrence of R¹, R⁴, and R⁵ are each independently selected from:
halogen, —R, —OR, —NO₂, —NCS, —CN, —CF₃, —OCF₂H—OCF₃, —SiR₃, —N(R)₂, —SR, —SOR, —SO₂R, —SO₂N(R)₂, —SO₃R, —(CR₂)₁₋₃R, —(CR₂)₁₋₃—OR, —(CR₂)₁₋₃—O(CR₂)₁₋₃—R, —(CR₂)₀₋₃—C(O)NR(CR₂)₀₋₃R, —(CR₂)₀₋₃—C(O)NR(CR₂)₀₋₃OR, —C(O)R, —C(O)C(O)R, —C(O)CH₂C(O)R, —C(S)R, —C(S)OR, —C(O)OR, —C(O)C(O)OR, —C(O)C(O)N(R)₂, —OC(O)R, —C(O)N(R)₂, —OC(O)N(R)₂, —C(S)N(R)₂, —(CR₂)₀₋₃NHC(O)R, —N(R)N(R)COR, —N(R)N(R)C(O)OR, —N(R)N(R)CON(R)₂, —N(R)SO₂R, —N(R)SO₂N(R)₂, —N(R)C(O)OR, —N(R)C(O)R, —N(R)C(S)R, —N(R)C(O)N(R)₂, —N(R)C(S)N(R)₂, —N(COR)COR, —N(OR)R, —C(═NH)N(R)₂, —C(O)N(OR)R, —C(═NOR)R, —OP(O)(OR)₂, —P(O)(R)₂, —P(O)(OR)₂, —P(O)(H)(OR), C≡C—R⁸, CH₂CF₃, or CHF₂;
R² is selected from —OR⁸, —SR⁸, —(CH₂)ₙOR⁸, —(CH₂)ₙO(CH₂)ₙR⁸, —(CH₂)ₚR⁸ or —(CH₂)ₙN(R″)R¹⁰, wherein n is an integer selected from 0-4; p is an integer selected from 2-4;
wherein R² is independently substituted with 0-5 R′;
each R³ is independently selected from:
—H, —CN, halogen, —(C1-C6) aliphatic, —CH═CR⁹, —C≡CR⁹, —SO₂((C1-C6)alkyl), —C(O)N((C1-C6)alkyl)₂), —C(O)NH((C1-C6) aliphatic), (C6-C10)-aryl-(C1-C12) aliphatic-, —C(O)((C1-C6)alkyl), —C(O)O((C1-C6)alkyl), 5- or 6-membered heterocyclyl, 5- or 6-membered heteroaryl, —(C1-C6)alkyl-C≡C—R¹⁰, —CH₂—O—R¹⁰, —CH₂—O—CH₂-R¹⁰ 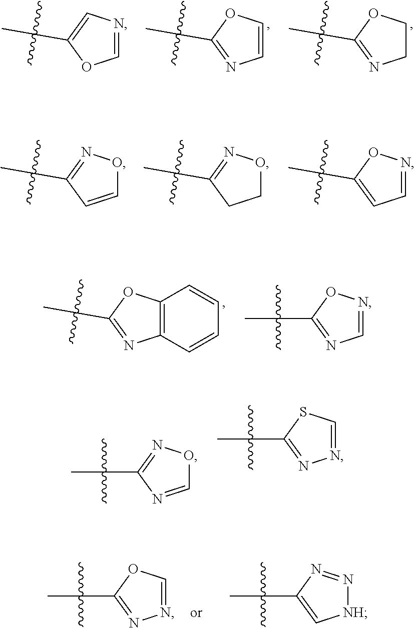
wherein each 5-10-membered heterocycle or heteroaryl are substituted with 0-3 R₇;
wherein R³ is independently substituted with 0-5 R′;
R⁶ is selected from the group consisting of —H and —(C1-C6)alkyl;
R₇ is selected from the group consisting of —(C1-C6)alkyl, —(C3-C6) cycloalkyl, -5 to 10 membered heteroaryl, —(C6-C10) aryl, —(C6-C10) aryl-(C1-C6)alkyl, and -5 to 10 membered heteroaryl-(C1-C6)alkyl, and -5-10 membered heteroaryl;
wherein each R₇ is independently substituted with 0-5 R′;
R⁸ is independently selected from the group consisting of —H, —(C1-C6)alkyl, —(C3-C10)-cycloalkyl, (C6-C10)-aryl, or 5- to 10-membered heteroaryl, 5-10 membered heteroaryl-(C1-C6)alkyl-, —(C1-C6)alkyl-(C6-C10) aryl, and —(C1-C6)alkyl-(C3-C6) cycloalkyl;
wherein each occurrence of R⁸ is independently substituted with 0-5 R′;
wherein R⁹ is selected from the group consisting of —H, —(C1-C6)alkyl, —(C3-C6) cycloalkyl, —(C1-C6)alkyl-(C3-C6) cycloalkyl, —(C1-C6)alkyl-(C6-C10) aryl, —(C6-C10) aryl, -5-10 membered heteroaryl, —(C1-C6)alkyl-5-10 membered heteroaryl, 5-10 membered heterocycle, —C(O)—(C6-C10) aryl, 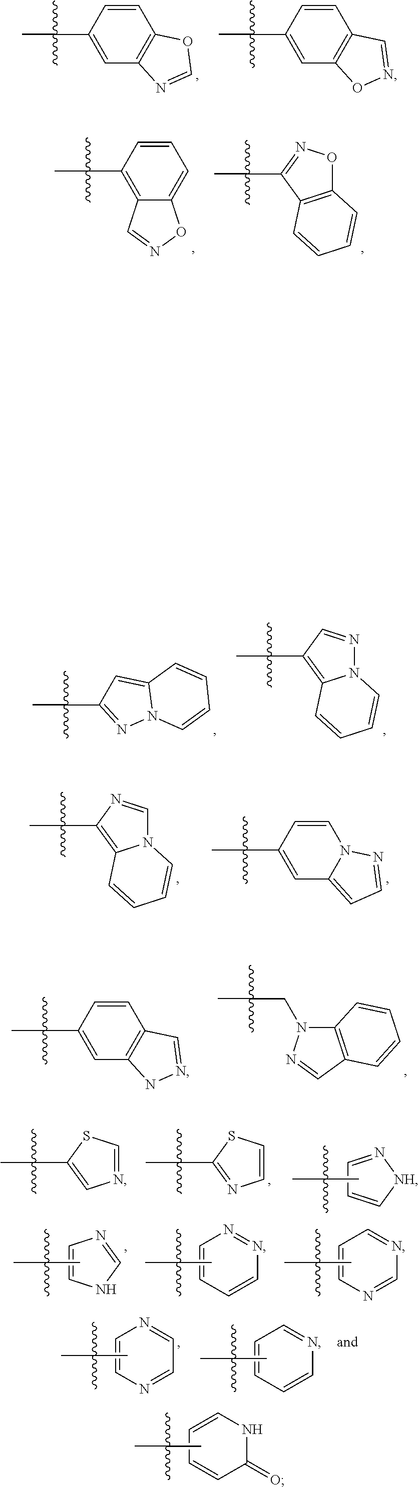
wherein each wherein each R⁹ is independently substituted with 0-5 R¹¹;
R¹⁰ is selected from the group consisting of —H, —(C1-C6)alkyl, —(C3-C10)-cycloalkyl, 3- to 10-membered heterocyclyl-, (C6-C10)-aryl, 5- to 10-membered heteroaryl, —CH₂—(C3-C6) cycloalkyl, —CH₂—(C6-C10) aryl, and —CH₂-5-10-membered heteroaryl,
wherein each occurrence of R¹⁰ is independently substituted with 0-5 R′;
wherein each occurrence of R¹¹ is independently selected from the group consisting of -halogen, —CF₃, —OCF₃, —OH, OCF₂H, —O—(C1-C6)alkyl, —(C6-C10) aryl, —(C1-C6)alkyl, —O—CH₂—(C3-C6) cycloalkyl, —CN, and -5 to 10 membered heteroaryl;
wherein each occurrence of R′ is independently selected from the group consisting of halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂;
wherein each occurrence of R″ is independently selected from the group consisting of H, —(C1-C6)-aliphatic, —(C1-C6)-alkyl, (C3-C6)-cycloalkyl, 3- to 6-membered heterocyclyl, 5- to 10-membered heteroaryl-, (C6-C10)-aryl-, (5- to 10-membered heteroaryl)-(C1-C6)-alkyl-, (C6-C10)-aryl-(C1-C6)-alkyl-, (5- to 10-membered heteroaryl)-O—(C1-C6)-alkyl-, and (C6-C10)-aryl-O—(C1-C6)-alkyl-;
wherein each occurrence of R″ is independently substituted with 0-5 R^{t} independently selected from the group consisting of: halogen, —R^{o}, —OR^{o}, oxo, —CH₂OR^{o}, —CH₂N(R^{o})₂, —C(O)N(R^{o})₂, —C(O)OR^{o}, —NO₂, —NCS, —CN, —CF₃, —OCF₃ and —N(R^{o})₂, wherein
each occurrence of R^{o} is independently selected from: —(C1-C6)-aliphatic, (C3-C6)-cycloalkyl, 3- to 6-membered heterocyclyl, 5- to 10-membered heteroaryl-, and (C6-C10)-aryl-.

23. The method of claim 22, wherein the GABA_{A} α5 receptor agonist, or the pharmaceutically acceptable salt, hydrate, solvate, isomer, or polymorph thereof or the GABA_{A} α5 receptor agonist, or the pharmaceutically acceptable salt, hydrate, solvate, isomer, or polymorph thereof of the pharmaceutical composition, is selected from the group consisting of:
i) a compound of formula II: 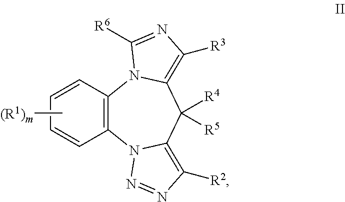
or a pharmaceutically acceptable salt, hydrate, solvate, polymorph, or isomer thereof, wherein:
m is 0-3;
each R¹ is independently selected from the group consisting of: halogen, —H, —(C1-C6)alkyl, —OH, —O((C1-C6)alkyl), —NO₂, —CN, —CF₃, —OCF₃, —OCHF₂, —OMe, —C≡C—R⁸, —CHF₂, —CH₂CF₃, —(C6-C10) aryl, —(C1-C6)alkyl-(C6-C10) aryl, -5-10 membered heteroaryl, —(C1-C6)alkyl-5-10 membered heteroaryl, and —(C3-C6) cycloalkyl; wherein R¹ is independently substituted with 0-5 R′;
R² is selected from the group consisting of:
—H, halogen, —OH, —(C1-C6) aliphatic, —O((C1-C6)alkyl), —C(O)O((C1-C6)alkyl), —C(O)NR₂, —(CR₂)₁₋₃—OR, —(CR₂)₁₋₃—O(CR₂)₁₋₃—R, —OR⁹, —C(O)R⁸, —CH₂R⁸, —CH₃, —CH₂—OR⁸,
(C6-C10)-aryl-,
(C6-C10)-aryl-(C1-C12) aliphatic-,
(C6-C10)-aryl-O—(C1-C12) aliphatic-,
(C6-C10)-aryl-N(R″)—(C1-C12) aliphatic-,
(5- to 10-membered heteroaryl)-(C1-C12) aliphatic-,
(5- to 10-membered heteroaryl)-O—(C1-C12) aliphatic-,
(5- to 10-membered heteroaryl)-N(R″)—(C1-C12) aliphatic-,
(3- to 10-membered heterocyclyl)-(C1-C12) aliphatic-,
(3- to 10-membered heterocyclyl)-O—(C1-C12) aliphatic-, and
(3- to 10-membered heterocyclyl)-N(R″)—(C1-C12) aliphatic-,
wherein R² is independently substituted with 0-5 R′;
R³ is selected from the group consisting of:
—(C1-C6)alkyl, —(C2-C6) alkenyl, —C≡CH, —C≡CR⁹, —CN, halogen, —SO₂((C6-C10)-aryl), —SO₂((C1-C6)alkyl), —C(O)N((C1-C6)alkyl)₂, —C(O)NH₂, —C(O)O((C1-C6)alkyl), —C(O)((C1-C6)alkyl), —(C6-C10) aryl, 5- to 10-membered heteroaryl, 5- to 10-membered heterocyclyl, —(C1-C6)alkyl-C≡C—R¹⁰, —CH₂—O—R¹⁰, —CH₂—O—CH₂-R¹⁰ 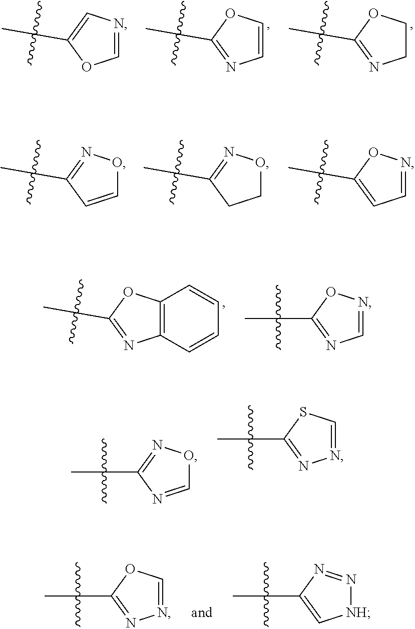
wherein each 5-member heterocycle or heteroaryl is substituted with 0-4 R₇;
wherein R³ is independently substituted with 0-5 R′;
R⁴ and R⁵ are each independently selected from the group consisting of —H, halogen, —(C1-C6)alkyl, or —(C1-C6)alkyl-(C6-C10) aryl; the (C6-C10) aryl being independently substituted with 0-5 halogen;
R⁶ is selected from the group consisting of —H and —(C1-C6)alkyl;
wherein R₇ is selected from the group consisting of (C1-C6)alkyl, —(C3-C6) cycloalkyl, -5 to 10 membered heteroaryl, —(C6-C10) aryl, (C6-C10) aryl-(C1-C6)alkyl-, -5 to 10 membered heteroaryl-(C1-C6)alkyl, and -5-10 membered heteroaryl; wherein each R₇ is independently substituted with 0-5 R′;
wherein each R⁸ is independently selected from the group consisting of —H, —(C1-C6)alkyl, —(C3-C6) cycloalkyl, —(C1-C6)alkyl-(C3-C6) cycloalkyl, —(C1-C6)alkyl-(C6-C10) aryl, —(C6-C10) aryl, -5-10 membered heteroaryl, and —(C1-C6)alkyl-5-10 membered heteroaryl;
wherein each R⁸ excluding-H and —(C1-C6)alkyl is independently substituted by 0-5 of -halogen, —(C1-C6)alkyl, —CF₃, —OCF₃, or O—(C1-C6)alkyl;
wherein R⁹ is selected from the group consisting of —H, —(C1-C6)alkyl, —(C6-C10) aryl, -5-10 membered heteroaryl, —(C1-C6)alkyl-(C6-C10) aryl, —(C1-C6)alkyl-5-10 membered heteroaryl, —(C3-C6) cycloalkyl, —(C1-C6)alkyl-(C3-C6) cycloalkyl, —C(O)—(C6-C10) aryl, 5-10 membered heterocycle, 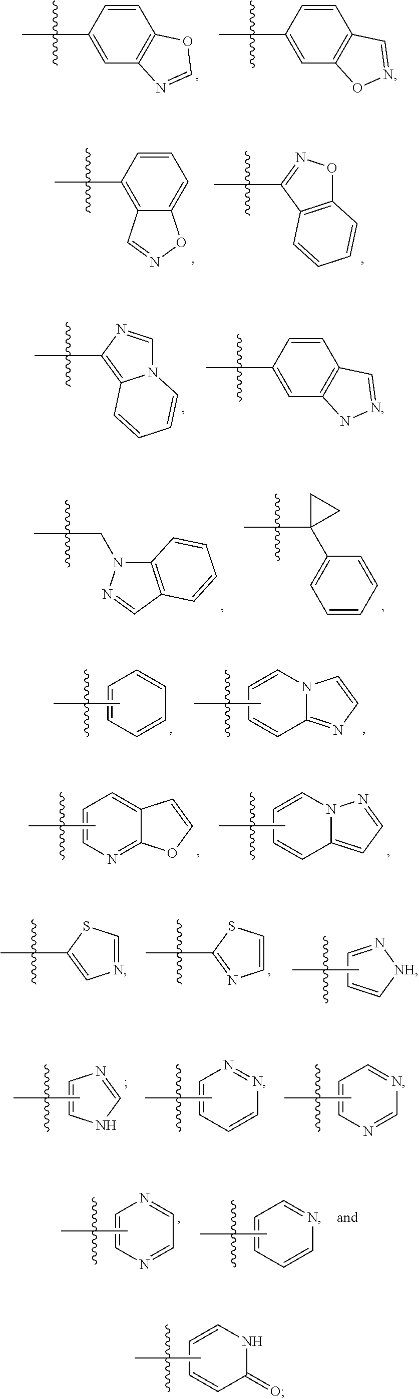
wherein each R⁹ is independently substituted with 0-5 R¹¹;
wherein R¹⁰ is selected from the group consisting of —H, halogen, —(C1-C6)alkyl, —(C6-C10) aryl, -5-10 membered heteroaryl, —(C3-C6) cycloalkyl, —CH₂—(C3-C6) cycloalkyl, —CH₂—(C6-C10) aryl, and —CH₂-5-10-membered heteroaryl,
wherein each R¹⁰ is substituted with 0-5 R′;
wherein each occurrence of R¹¹ is independently selected from the group consisting of -halogen, —CN, SCH₃, —CF₃, —OH, —OCF₃, OCHF₂, —O(C1-C6)alkyl, —(C6-C10) aryl, —(C1-C6)alkyl, and -5 to 10 membered heteroaryl;
each R is independently selected from the group consisting of:
H—,
(C1-C12)-aliphatic-,
(C3-C10)-cycloalkyl-,
(C3-C10)-cycloalkenyl-,
[(C3-C10)-cycloalkyl]-(C1-C12)-aliphatic-,
[(C3-C10)-cycloalkenyl]-(C1-C12)-aliphatic-,
[(C3-C10)-cycloalkyl]-O—(C1-C12)-aliphatic-,
[(C3-C10)-cycloalkenyl]-O—(C1-C12)-aliphatic-,
(C6-C10)-aryl-,
(C6-C10)-aryl-(C1-C12) aliphatic-,
(C6-C10)-aryl-O—(C1-C12) aliphatic-,
(C6-C10)-aryl-N(R″)—(C1-C12) aliphatic-,
3- to 10-membered heterocyclyl-,
(3- to 10-membered heterocyclyl)-(C1-C12) aliphatic-,
(3- to 10-membered heterocyclyl)-O—(C1-C12) aliphatic-,
(3- to 10-membered heterocyclyl)-N(R″)—(C1-C12) aliphatic-,
5- to 10-membered heteroaryl-,
(5- to 10-membered heteroaryl)-(C1-C12)-aliphatic-,
(5- to 10-membered heteroaryl)-O—(C1-C12)-aliphatic-; and
(5- to 10-membered heteroaryl)-N(R″)—(C1-C12)-aliphatic-;
wherein said heterocyclyl has 1-4 heteroatoms independently selected from the group consisting of N, NH, O, S, SO, and SO₂, and said heteroaryl has 1-4 heteroatoms independently selected from the group consisting of N, NH, O, and S;
wherein each occurrence of R is independently substituted with 0-5 R′;
or when two R groups bound to the same atom, the two R groups may be taken together with the atom to which they are bound to form a 3- to 10-membered aromatic or non-aromatic ring having 0-4 heteroatoms independently selected from the group consisting of N, NH, O, S, SO, and SO₂, wherein said ring is optionally substituted with 0-5 R′, and wherein said ring is optionally fused to a (C6-C10) aryl, 5- to 10-membered heteroaryl, (C3-C10) cycloalkyl, or a 3- to 10-membered heterocyclyl;
wherein each occurrence of R′ is independently selected from the group consisting of halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂;
wherein each occurrence of R″ is independently selected from the group consisting of H, —(C1-C6)-alkyl, —(C1-C6)-aliphatic, (C3-C6)-cycloalkyl, 3- to 6-membered heterocyclyl, 5- to 10-membered heteroaryl-, (C6-C10)-aryl-, (5- to 10-membered heteroaryl)-(C1-C6)-alkyl-, (C6-C10)-aryl-(C1-C6)-alkyl-, (5- to 10-membered heteroaryl)-O—(C1-C6)-alkyl-, (C6-C10)-aryl-O—(C1-C6)-alkyl-, and (C6-C10)-aryl-O—(C1-C6)-alkyl-,
wherein each occurrence of R″ is independently substituted with 0-5 substituents selected from the group consisting of: halogen, —R^{o}, —OR^{o}, oxo, —CH₂OR^{o}, —CH₂N(R^{o})₂, —C(O)N(R^{o})₂, —C(O)OR^{o}, —NO₂, —NCS, —CN, —CF₃, —OCF₃ and —N(R^{o})₂, wherein each occurrence of R^{o} is independently selected from the group consisting of: —(C1-C6)-aliphatic, (C3-C6)-cycloalkyl, 3- to 6-membered heterocyclyl, 5- to 10-membered heteroaryl-, and (C6-C10)-aryl; and
ii) a compound of formula IV: 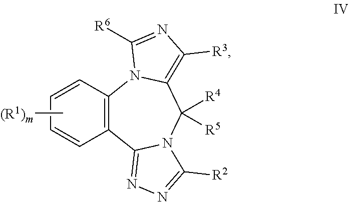
or a pharmaceutically acceptable salt, hydrate, solvate, polymorph, or isomer thereof, wherein:
m is 0-3;
each R¹ is independently selected from the group consisting of: halogen, —H, —(C1-C6)alkyl, —C≡C—R⁹, —OH, —O((C1-C6)alkyl), —NO₂, —CN, —CF₃, —OCF₃, —CHF₂, —CH₂CF₃, —(C6-C10) aryl, —(C1-C6)alkyl-(C6-C10) aryl, -5-10 membered heteroaryl, —(C1-C6)alkyl-5-10 membered heteroaryl, and —(C3-C6) cycloalkyl;
wherein R¹ is independently substituted with 0-5 R′;
R² is selected from the group consisting of —OR⁸, —SR⁸, —(CH₂)ₙOR⁸, —(CH₂)ₙO(CH₂)ₙR⁸, —(CH₂)ₚR⁸ and —(CH₂)ₙN(R″)R¹⁰, wherein n is an integer selected from 0-4; p is an integer selected from 2-4;
wherein R² is independently substituted with 0-5 R′;
each R³ is independently selected from the group consisting of:
—H, —CN, halogen, —(C1-C6) aliphatic, —CH═CR⁹, —C≡CR⁹, —SO₂((C1-C6)alkyl), —C(O)N((C1-C6)alkyl)₂), —C(O)NH((C1-C6) aliphatic), (C6-C10)-aryl-(C1-C12) aliphatic-, —C(O)((C1-C6)alkyl), —C(O)O((C1-C6)alkyl), 5- or 6-membered heterocyclyl, 5- or 6-membered heteroaryl, —CH₂—O—R¹⁰, —CH₂—O—CH₂-R¹⁰ 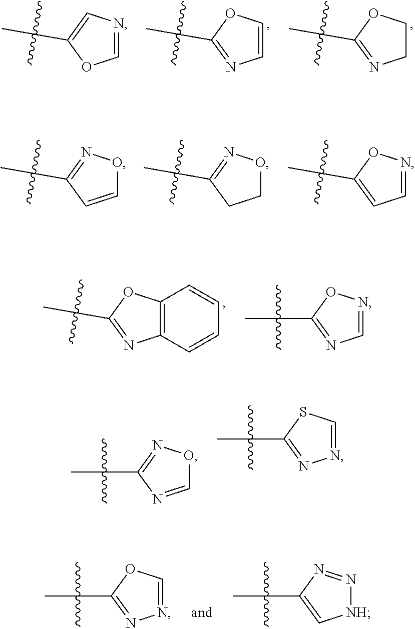
wherein each 5-10-membered heterocycle or heteroaryl are substituted with 0-3 R₇;
wherein R³ is independently substituted with 0-5 R′;
R⁴ and R⁵ are each independently selected from the group consisting of —H, halogen and —(C1-C6)alkyl;
R⁶ is selected from the group consisting of —H and —(C1-C6)alkyl;
R₇ is selected from the group consisting of —(C1-C6)alkyl, —(C3-C6) cycloalkyl, -5 to 10 membered heteroaryl, —(C6-C10) aryl, —(C6-C10) aryl-(C1-C6)alkyl, and -5 to 10 membered heteroaryl-(C1-C6)alkyl, and -5-10 membered heteroaryl;
wherein each R₇ is independently substituted with 0-5 R′;
R⁸ is independently selected from the group consisting of —H, —(C1-C6)alkyl, —(C3-C10)-cycloalkyl, (C6-C10)-aryl, or 5- to 10-membered heteroaryl, 5-10 membered heteroaryl-(C1-C6)alkyl-, —(C1-C6)alkyl-(C6-C10) aryl, and —(C1-C6)alkyl-(C3-C6) cycloalkyl;
wherein each occurrence of R⁸ is independently substituted with 0-5 R′;
wherein R⁹ is selected from the group consisting of —H, —(C1-C6)alkyl, —(C3-C6) cycloalkyl, —(C1-C6)alkyl-(C3-C6) cycloalkyl, —(C1-C6)alkyl-(C6-C10) aryl, —(C6-C10) aryl, -5-10 membered heteroaryl, —(C1-C6)alkyl-5-10 membered heteroaryl, 5-10 membered heterocycle, —C(O)—(C6-C10) aryl, 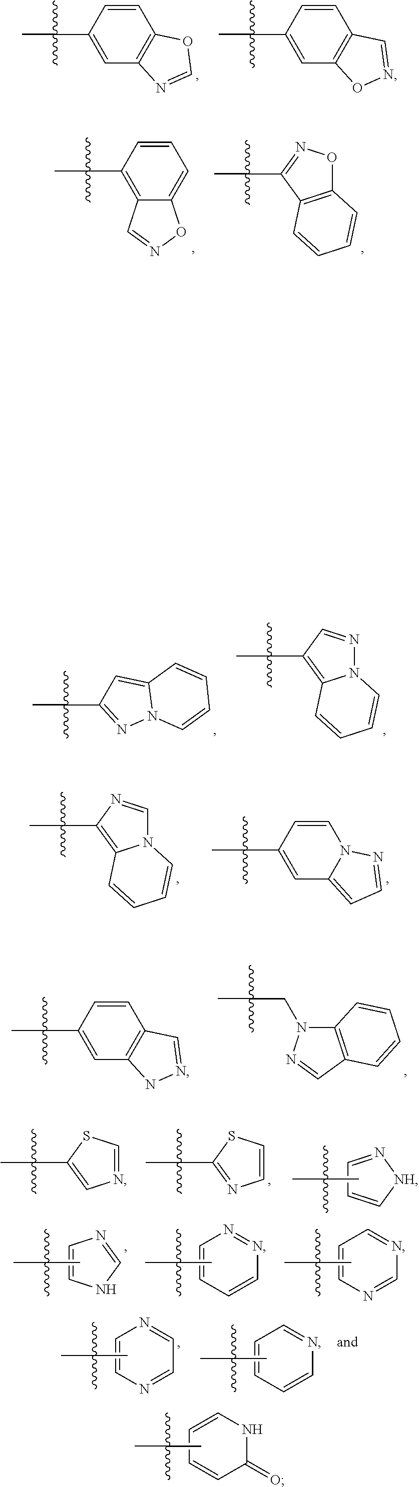
wherein each wherein each R′ is independently substituted with 0-5 R¹¹;
R¹⁰ is selected from the group consisting of —H, —(C1-C6)alkyl, —(C3-C10)-cycloalkyl, 3- to 10-membered heterocyclyl-, (C6-C10)-aryl, 5- to 10-membered heteroaryl, —CH₂—(C3-C6) cycloalkyl, —CH₂—(C6-C10) aryl, and —CH₂-5-10-membered heteroaryl,
wherein each occurrence of R¹⁰ is independently substituted with 0-5 R′;
wherein each occurrence of R¹¹ is independently selected from the group consisting of -halogen, —CF₃, —OCF₃, OCF₂H, —O—(C1-C6)alkyl, —(C6-C10) aryl, —(C1-C6)alkyl, —O—CH₂—(C3-C6) cycloalkyl, and -5 to 10 membered heteroaryl;
wherein each occurrence of R′ is independently selected from the group consisting of halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂;
wherein each occurrence of R″ is independently selected from the group consisting of H, —(C1-C6)-aliphatic, —(C1-C6)-alkyl, (C3-C6)-cycloalkyl, 3- to 6-membered heterocyclyl, 5- to 10-membered heteroaryl-, (C6-C10)-aryl-, (5- to 10-membered heteroaryl)-(C1-C6)-alkyl-, (C6-C10)-aryl-(C1-C6)-alkyl-, (5- to 10-membered heteroaryl)-O—(C1-C6)-alkyl-, and (C6-C10)-aryl-O—(C1-C6)-alkyl-;
wherein each occurrence of R″ is independently substituted with 0-5 R^{t} independently selected from the group consisting of: halogen, —R^{o}, —OR^{o}, oxo, —CH₂OR^{o}, —CH₂N(R^{o})₂, —C(O)N(R^{o})₂, —C(O)OR^{o}, —NO₂, —NCS, —CN, —CF₃, —OCF₃ and —N(R^{o})₂, wherein
each occurrence of R^{o} is independently selected from: —(C1-C6)-aliphatic, (C3-C6)-cycloalkyl, 3- to 6-membered heterocyclyl, 5- to 10-membered heteroaryl-, and (C6-C10)-aryl-.

24. The method of claim 23, wherein the GABA_{A} α5 receptor agonist, or the pharmaceutically acceptable salt, hydrate, solvate, polymorph, or isomer thereof or the GABA_{A} α5 receptor agonist, or the pharmaceutically acceptable salt, hydrate, solvate, isomer, or polymorph thereof of the pharmaceutical composition, is a compound of Formula II, or a pharmaceutically acceptable salt, hydrate, solvate, polymorph, or isomer thereof.

25. The method of claim 23, wherein the GABA_{A} α5 receptor agonist, or the pharmaceutically acceptable salt, hydrate, solvate, polymorph, or isomer thereof or the GABA_{A} α5 receptor agonist, or the pharmaceutically acceptable salt, hydrate, solvate, isomer, or polymorph thereof of the pharmaceutical composition, is a compound of Formula IV, or a pharmaceutically acceptable salt, hydrate, solvate, polymorph, or isomer thereof.

26. The method of claim 21, wherein the GABA_{A} α5 receptor agonist, or the pharmaceutically acceptable salt, hydrate, solvate, isomer, or polymorph thereof or the GABA_{A} α5 receptor agonist, or the pharmaceutically acceptable salt, hydrate, solvate, isomer, or polymorph thereof of the pharmaceutical composition is selected from the group consisting of Compounds 1-12, 44-56, 101-268, 270-644, 646-687, 689-698, 700-703, 705, 707-721, and 723-740, or a pharmaceutically acceptable salt, hydrate, solvate, isomer, or polymorph thereof.

27. The method of claim 26, wherein the GABA_{A} α5 receptor agonist, or the pharmaceutically acceptable salt, hydrate, solvate, isomer, or polymorph thereof or the GABA_{A} α5 receptor agonist, or the pharmaceutically acceptable salt, hydrate, solvate, isomer, or polymorph thereof of the pharmaceutical composition is the compound having the structure 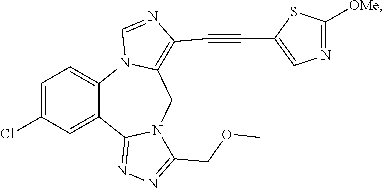 [SMILES: COCc1nnc2n1Cc1c(C#Cc3cnc(OC)s3)ncn1-c1ccc(Cl)cc1-2]
or a pharmaceutically acceptable salt, hydrate, solvate, polymorph, or isomer thereof.

28. The method of claim 27, wherein the GABA_{A} α5 receptor agonist, or the pharmaceutically acceptable salt, hydrate, solvate, isomer, or polymorph thereof or the GABA_{A} α5 receptor agonist, or the pharmaceutically acceptable salt, hydrate, solvate, isomer, or polymorph thereof of the pharmaceutical composition is a polymorph crystalline form of the compound having the structure 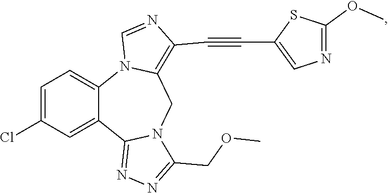 [SMILES: COCc1nnc2n1Cc1c(C#Cc3cnc(OC)s3)ncn1-c1ccc(Cl)cc1-2]
wherein the polymorph crystalline form is Form A and exhibits an XRPD comprising:
a. at least one peak selected from 3.0, and 21.0 degrees 2θ±0.2 degrees 2θ; and
b. at least one additional peak selected from the group consisting of 9.1, 10.7, 13.8, 22.0, 23.1, 23.9, 24.4, and 27.1 degrees 2θ±0.2 degrees 2θ.

29. The method of claim 27, wherein the GABA_{A} α5 receptor agonist, or the pharmaceutically acceptable salt, hydrate, solvate, isomer, or polymorph thereof or the GABA_{A} α5 receptor agonist, or the pharmaceutically acceptable salt, hydrate, solvate, isomer, or polymorph thereof of the pharmaceutical composition is a polymorph crystalline form of the compound having the structure 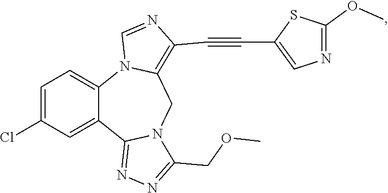 [SMILES: COCc1nnc2n1Cc1c(C#Cc3cnc(OC)s3)ncn1-c1ccc(Cl)cc1-2]
wherein the crystalline form is Form B and exhibits an XRPD comprising:
a. at least one peak selected from 13.0 and 15.3 degrees 2θ±0.2 degrees 2θ; and
b. at least one additional peak selected from the group consisting of 7.0, 9.3, 10.2, 10.4, 12.5, 13.6, 14.0, 22.0, 23.0, 23.6, and 27.3 degrees 2θ±0.2 degrees 2θ.

30. The method of claim 27, wherein the GABA_{A} α5 receptor agonist, or the pharmaceutically acceptable salt, hydrate, solvate, isomer, or polymorph thereof or the GABA_{A} α5 receptor agonist, or the pharmaceutically acceptable salt, hydrate, solvate, isomer, or polymorph thereof of the pharmaceutical composition is a solvate crystalline form of the compound having the structure 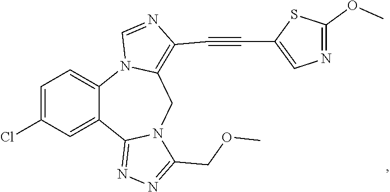 [SMILES: COCc1nnc2n1Cc1c(C#Cc3cnc(OC)s3)ncn1-c1ccc(Cl)cc1-2]
wherein the solvate crystalline form is Form C and exhibits an XRPD comprising:
a. at least one peak selected from 8.5 and 18.9 degrees 2θ±0.2 degrees 2θ; and
b. at least one additional peak selected from the group consisting of 7.1, 9.4, 10.3, 12.3, 12.5, 14.2, 20.7, 22.1, 23.2, 23.7, 24.0, and 26.4 degrees 2θ±0.2 degrees 2θ.

31. The method of claim 27, wherein the GABA_{A} α5 receptor agonist, or the pharmaceutically acceptable salt, hydrate, solvate, isomer, or polymorph thereof or the GABA_{A} α5 receptor agonist, or the pharmaceutically acceptable salt, hydrate, solvate, isomer, or polymorph thereof of the pharmaceutical composition is a polymorph crystalline form of the compound having the structure 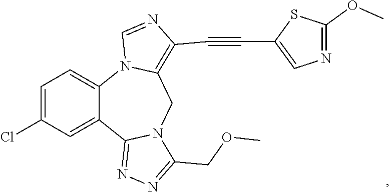 [SMILES: COCc1nnc2n1Cc1c(C#Cc3cnc(OC)s3)ncn1-c1ccc(Cl)cc1-2]
wherein the polymorph crystalline form is Form E and exhibits an XRPD comprising:
a. at least one peak selected from the group consisting of 11.4, 18.1, and 21.6 degrees 2θ±0.2 degrees 2θ; and
b. at least one additional peak selected from the group consisting of 7.2, 22.0, 23.0, 24.2, 25.0, and 26.6 degrees 2θ±0.2 degrees 2θ.

32. The method of claim 27, wherein the GABA_{A} α5 receptor agonist, or the pharmaceutically acceptable salt, hydrate, solvate, isomer, or polymorph thereof or the GABA_{A} α5 receptor agonist, or the pharmaceutically acceptable salt, hydrate, solvate, isomer, or polymorph thereof of the pharmaceutical composition is a hydrate crystalline form of the compound having the structure 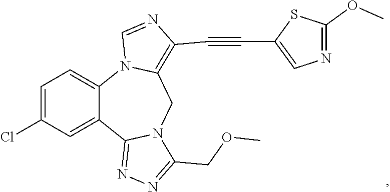 [SMILES: COCc1nnc2n1Cc1c(C#Cc3cnc(OC)s3)ncn1-c1ccc(Cl)cc1-2]
wherein the hydrate crystalline form is Form F and exhibits an XRPD comprising:
a. at least one peak selected from the group consisting of 9.9, 11.9, 17.3, 19.4, and 25.7 degrees 2θ±0.2 degrees 2θ; and
b. at least one additional peak selected from the group consisting of 9.7, 12.1, 20.8, 23.2, 23.7, 24.2, 25.0, and 26.4 degrees 2θ±0.2 degrees 2θ.

33. The method of any one of claims 21-32, wherein the GABA_{A} α5 receptor agonist or the pharmaceutically acceptable salt, hydrate, solvate, isomer, or polymorph thereof, or the GABA_{A} α5 receptor agonist or the pharmaceutically acceptable salt, hydrate, solvate, isomer, or polymorph thereof of the pharmaceutical composition, is present in an amount between 5 mg and 1000 mg.

34. The method of claim 21, wherein the GABA_{A} α5 receptor agonist, or the pharmaceutically acceptable salt, hydrate, solvate, isomer, or polymorph thereof or the GABA_{A} α5 receptor agonist, or the pharmaceutically acceptable salt, hydrate, solvate, isomer, or polymorph thereof of the pharmaceutical composition, is selected from the group consisting of:
i) a compound of formula I-a: 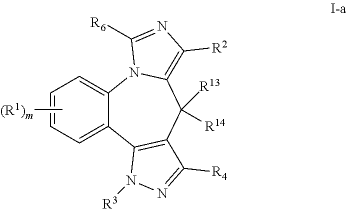
or a pharmaceutically acceptable salt, hydrate, solvate, polymorph, isomer, or combination thereof,
wherein:
m is an integer selected from 0-4;
each R¹ is independently selected from: halogen, —(C6-C10) aryl, —O(C1-C6)alkyl, —CN, —CHF₂, —CF₃, —OCF₃, —OCHF₂, CO(O)R₇, CH₂—OR⁸, —(C1-C6)alkyl-(C6-C10) aryl, -5-10 membered heteroaryl, —(C1-C6)alkyl-5-10 membered heteroaryl and —(C3-C6) cycloalkyl;
each R⁸ is independently selected from —H or —(C1-C6 alkyl)
each R² is selected from CO(O)R₇, C≡C—R⁹, —(C1-C6)alkyl-C≡C—R⁹, -(5-10 membered) heteroaryl, -(3-10 membered) heterocyclyl, (C3-C10)-cycloalkenyl 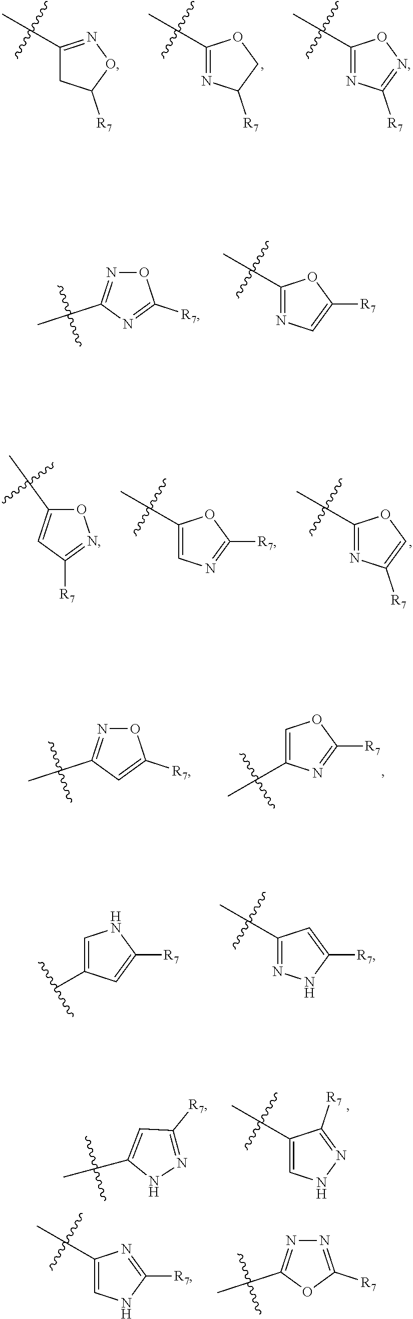
wherein each 5-6-membered heteroaryl and 3-10-membered heterocycle is substituted with 0-4 R₇;
each R⁹ is selected from: —H, —(C1-C6)alkyl, (5- to 10-membered heteroaryl), -(3-10 membered) heterocyclyl, —(C6-C10) aryl, —(C1-C6)alkyl-(C6-C10) aryl 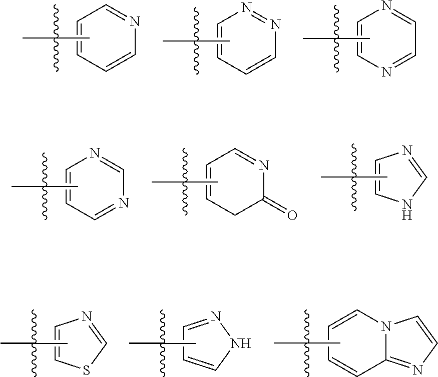
wherein each occurrence of R⁹ is independently substituted by 0-5 R¹¹;
wherein each occurrence of R¹¹ is independently selected from —(C1-C6)alkyl, —O—(C1-C6)alkyl, -halogen, —CF₃, —OCF₃, —(C6-C10) aryl and -5 to 10 membered heteroaryl;
R³ is independently selected from: —H, —(C1-C6)alkyl, -5 to 10 membered heteroaryl, -(3-10 membered) heterocyclyl, —(C1-C6)alkyl-(C3-C6) cycloalkyl and —(C1-C6)alkyl-(C6-C10) aryl, wherein R³ is independently substituted with 0-5 R¹²;
wherein each R¹² is independently selected from: —H, -halogen, —OR^{o}, R^{o}, oxo, —CH₂OR^{o}, —CH₂N(R^{o})₂, —C(O)N(R^{o})₂, —C(O)OR^{o}, —CF₃, —OCF₃ and —N(R^{o})₂, wherein each occurrence of R^{o} is independently selected from: —(C1-C6)-aliphatic, (C3-C6)-cycloalkyl, -(3-10 membered) heterocyclyl, and (C6-C10)-aryl;
wherein each R₇ is selected from —H, —CF₃, —(C1-C6)alkyl, —(C1-C6)alkyloxy, (C1-C6) alkylamino, —(C3-C6) cycloalkyl, -5 to 10 membered heteroaryl, —(C6-C10) aryl, —CH₂—(C6-C10) aryl, -5 to 10 membered heteroaryl-(C1-C6)alkyl, and -3-10 membered heterocyclyl, wherein each R₇ is independently substituted with 0-5 R′;
or when two R₇ groups bound to the same atom, the two R₇ groups may be taken together with the atom to which they are bound to form a 3-10-membered aromatic or non-aromatic ring having 0-4 heteroatoms independently selected from N, NH, O, S, SO, and SO₂, wherein said ring is optionally substituted with 0-5 R′;
each occurrence of R′ is wherein each occurrence of R′ is independently selected from halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂, wherein R″ is selected from —Cl, —F, —(C1-C6)alkyl, —OMe, and —(C6-C10) aryl;
R″ is independently substituted with 1-3 substituents wherein the substituents are selected from: halogen, —CF₃, —OCF₃, —O—(C1-C6)-aliphatic or —(C1-C6)-aliphatic;
each R⁴ is selected from —H or —(C1-C6)alkyl;
each R⁶ is selected from —H or —(C1-C6)alkyl;
each R¹³ and R¹⁴ is independently selected from H—, (C1-C3)-aliphatic-, or (C3-C6)-cycloalkyl;
ii) a compound of formula I-b: 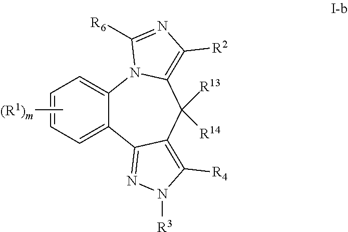
or a pharmaceutically acceptable salt, hydrate, solvate, polymorph, isomer, or combination thereof,
m is an integer selected from 0-4;
wherein:
each R¹ is independently selected from -halogen, —(C6-C10) aryl, —Ome, —CN, —CHF₂, —CF₃, —OCF₃, —OCHF₂, CO(O)R₇, CH₂—OR⁸, —(C1-C6)alkyl-(C6-C10) aryl, -5-10 membered heteroaryl, —(C1-C6)alkyl-5-10 membered heteroaryl and —(C3-C6) cycloalkyl;
each R⁸ is independently selected from —H or —(C1-C6 alkyl);
each R² is selected from CO(O)R₇, C≡C—R⁹, —(C1-C6)alkyl-C≡C—R⁹, -(5-10 membered) heteroaryl, -(3-10 membered) heterocyclyl, 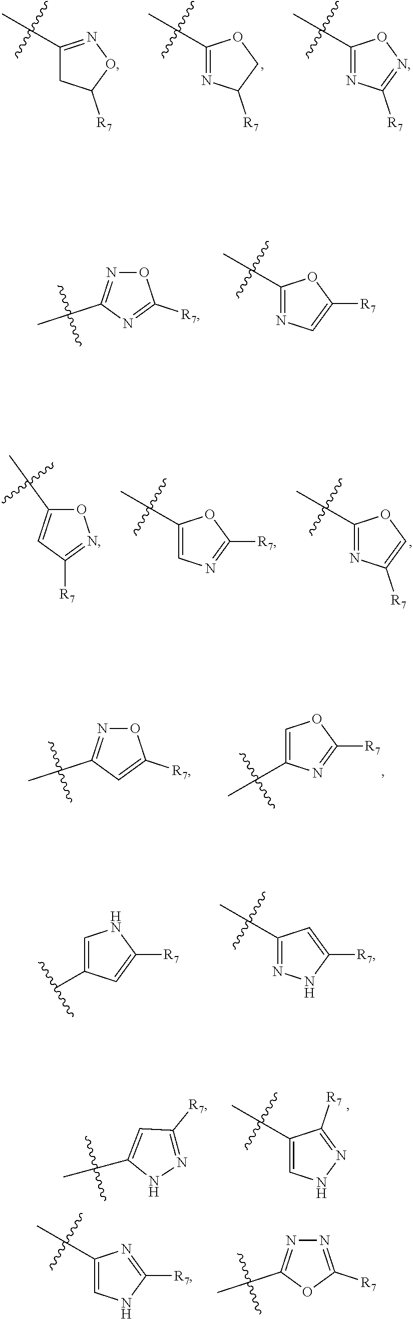
wherein each 5-6 membered heteroaryl or 3-10 membered heterocycle is substituted with 0-4 R₇;
each R⁹ is selected from: —H, —(C1-C6)alkyl, (5- to 10-membered heteroaryl), -(3-10 membered) heterocyclyl, —(C6-C10) aryl, —(C1-C6)alkyl-(C6-C10) aryl 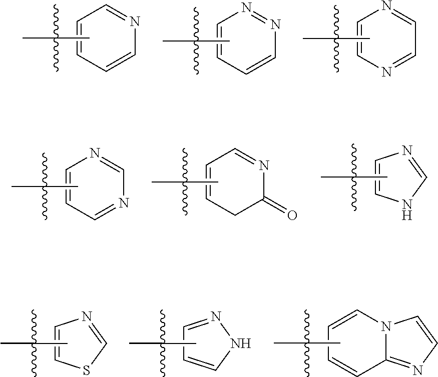
wherein each occurrence of R⁹ is independently substituted by 0-5 R¹¹
wherein each occurrence of R¹¹ is independently selected from —(C1-C6)alkyl, —O—(C1-C6)alkyl, -halogen, —CF₃, —OCF₃, —OMe, —(C6-C10) aryl and -5 to 10 membered heteroaryl;
R³ is independently selected from: —H, —(C1-C6)alkyl, —(C1-C6)alkyl-(C3-C6) cycloalkyl, —(C1-C6)alkyl-OR¹², —(C1-C6)alkyl-N(R¹²)₂, —(C1-C6)alkyl-(C6-C10) aryl, —(C1-C6) alkyl-5-10 membered heteroaryl, -3 to 10 membered heterocyclyl and -5-10 membered heteroaryl, wherein R³ is independently substituted with 0-5 R¹²;
wherein each R¹² is independently selected from: —H, -halogen, —OR^{o}, R^{o}, oxo, —CH₂OR^{o}, —CH₂N(R^{o})₂, —C(O)N(R^{o})₂, —C(O)OR^{o}, —NO₂, —NCS, —CN, —CF₃, —OCF₃ and —N(R^{o})₂,
wherein each occurrence of R^{o} is independently selected from: —(C1-C6)-aliphatic, (C3-C6)-cycloalkyl, 3- to 6-membered heterocyclyl, 5- to 10-membered heteroaryl-, and (C6-C10)-aryl,
wherein each R₇ is selected from —H, —CF₃, —(C1-C6)alkyl, —(C1-C6)alkyloxy, (C1-C6) alkylamino, —(C3-C6) cycloalkyl, -5 to 10 membered heteroaryl, —(C6-C10) aryl, (C6-C10) aryl-(C1-C6)alkyl-, —(C1-C6)alkyl-5 to 10 membered heteroaryl and -3-10 membered heterocyclyl, wherein each R₇ is independently substituted with 0-5 R′;
or when two R₇ groups bound to the same atom, the two R₇ groups may be taken together with the atom to which they are bound to form a 3-10-membered aromatic or non-aromatic ring having 0-4 heteroatoms independently selected from N, NH, O, S, SO, and SO₂, wherein said ring is optionally substituted with 0-5 R′;
each occurrence of R′ is wherein each occurrence of R′ is independently selected from halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂;
wherein R″ is selected from —Cl, —F, —(C1-C6)alkyl, —OMe, —(C1-C6)alkyl-5 to 10 membered heteroaryl, -5 to 10 membered heteroaryl, -3-10 membered heterocyclyl, —(C3-C6) cycloalkyl and —(C6-C10) aryl;
R″ is independently substituted with 1-3 substituents wherein the substituents are selected from: halogen, —CF₃, —OCF₃, —O(C1-C6)-aliphatic, —(C1-C6)-aliphatic and -5 to 10 membered heteroaryl;
each R⁴ and R⁶ is independently selected from —H or —(C1-C6)alkyl;
each R¹³ and R¹⁴ is independently selected from H—, (C1-C3)-aliphatic-, or (C3-C6)-cycloalkyl;
iii) a compound of formula I-c: 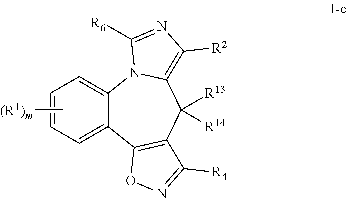
or a pharmaceutically acceptable salt, hydrate, solvate, polymorph, isomer, or combination thereof,
m is an integer selected from 0-4;
each R¹ is independently selected from -halogen, —(C6-C10) aryl, —OMe, —CN, —CHF₂, —CF₃, —OCF₃, —OCHF₂, CO(O)R₇, CH₂—OR⁸, —(C1-C6)alkyl-(C6-C10) aryl, -5-10 membered heteroaryl, —(C1-C6)alkyl-5-10 membered heteroaryl and —(C3-C6) cycloalkyl;
each R⁸ is independently selected from —H or —(C1-C6 alkyl);
each R² is selected from CO(O)R₇, C≡C—R⁹, —(C1-C6)alkyl-C≡C—R⁹, -(5-10 membered) heteroaryl, -(3-10 membered) heterocyclyl, 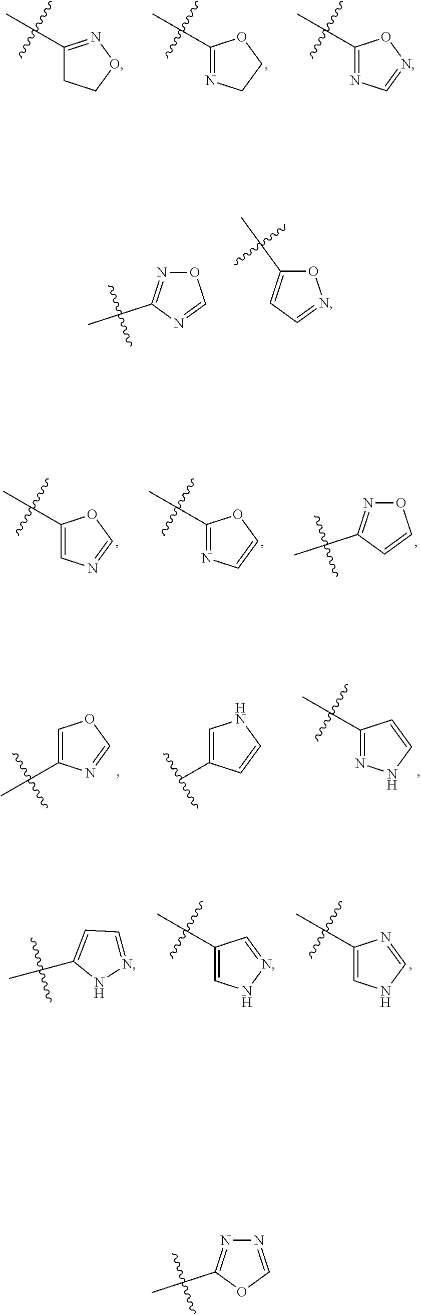
wherein each 5-membered heterocycle or heteroaryl is substituted with 0-4 R₇;
each R⁹ is selected from: —H, —(C1-C6)alkyl, (5- to 10-membered heteroaryl), -(3-10 membered) heterocyclyl 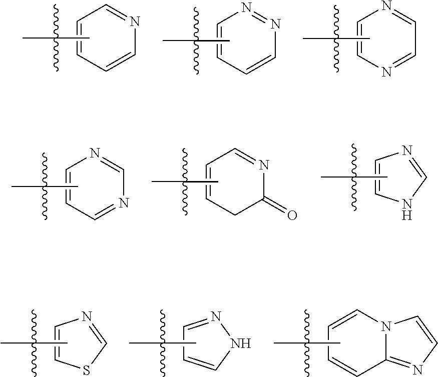
each occurrence of R⁹ is independently substituted by 0-5 R¹¹;
each occurrence of R¹¹ is independently selected from —(C1-C6)alkyl, —O—(C1-C6)alkyl, -halogen, —CF₃, —OCF₃, —OMe, —(C6-C10) aryl and -5 to 10 membered heteroaryl;
each occurrence of R₇ is selected from —CF₃, —(C1-C6)alkyl, —(C3-C6) cycloalkyl, -5 to 10 membered heteroaryl, —(C6-C10) aryl, (C6-C10) aryl-(C1-C6)alkyl-, —(C1-C6)alkyl-5 to 10 membered heteroaryl and -3-10 membered heterocyclyl, wherein each R₇ is independently substituted with 0-5 R′;
each occurrence of R′ is wherein each occurrence of R′ is independently selected from halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂,
wherein each occurrence of R″ is selected from —Cl, —F, —(C1-C6)alkyl, —OMe, —(C1-C6)alkyl-5 to 10 membered heteroaryl, -5 to 10 membered heteroaryl, -3-10 membered heterocyclyl, —(C3-C6) cycloalkyl and —(C6-C10) aryl, and R″ is independently substituted with 1-3 substituents wherein the substituents are selected from: halogen, —CF₃, —OCF₃, —(C1-C6)-aliphatic and -5 to 10 membered heteroaryl;
each R⁴ and R⁶ is independently selected from —H or (C1-C6)alkyl;
each R¹³ and R¹⁴ is independently selected from H—, (C1-C3)-aliphatic-, or (C3-C6)-cycloalkyl;
iv) a compound of formula I-d: 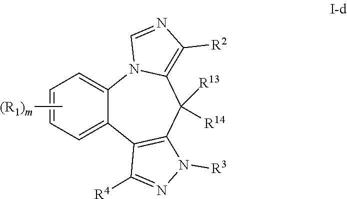
or a pharmaceutically acceptable salt, hydrate, solvate, polymorph, isomer, or combination thereof,
m is an integer selected from 0-4;
wherein:
each R¹ is independently selected from -halogen, —(C6-C10) aryl, —OMe, —CN, —CHF₂, —CF₃, —OCF₃, —OCHF₂, CO(O)R₇, CH₂—OR⁸, —(C1-C6)alkyl-(C6-C10) aryl, -5-10 membered heteroaryl, —(C1-C6)alkyl-5-10 membered heteroaryl and —(C3-C6) cycloalkyl;
each R⁸ is independently selected from —H or —(C1-C6 alkyl);
each R² is selected from CO(O)R₇, C≡C—R⁹, —(C1-C6)alkyl-C≡C—R⁹, -(5-10 membered) heteroaryl, -(3-10 membered) heterocyclyl, 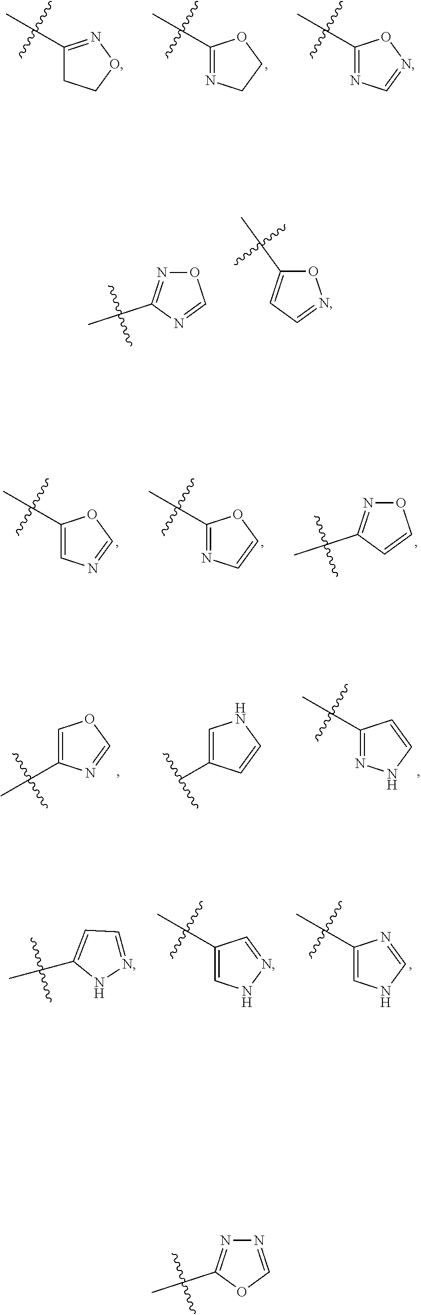
wherein each 5-membered heterocycle or heteroaryl is substituted with 0-4 R₇;
each R⁹ is selected from: —H, —(C1-C6)alkyl, (5- to 10-membered heteroaryl), -(3-10 membered) heterocyclyl, —(C6-C10) aryl, —(C1-C6)alkyl-(C6-C10) aryl 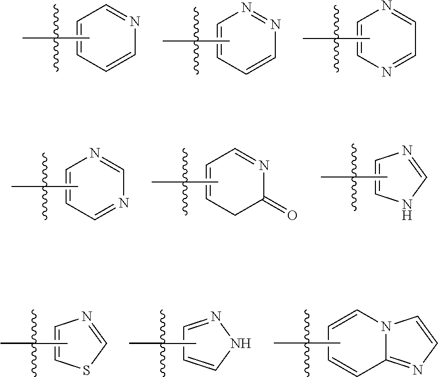
wherein each occurrence of R⁹ is independently substituted by 0-5 R¹¹
wherein each occurrence of R¹¹ is independently selected from —(C1-C6)alkyl, —O—(C1-C6)alkyl, -halogen, —CF₃, —OCF₃, —OMe, —(C6-C10) aryl and -5 to 10 membered heteroaryl;
R³ is independently selected from: —H, —(C1-C6)alkyl, —(C1-C6)alkyl-(C3-C6) cycloalkyl, —(C1-C6)alkyl-OR¹², —(C1-C6)alkyl-N(R¹²)₂, —(C1-C6)alkyl-(C6-C10) aryl, —(C1-C6) alkyl-5-10 membered heteroaryl wherein R³ is independently substituted with 0-5 R¹²;
wherein each R¹² is independently selected from: —H, -halogen, —OR^{o}, R^{o}, oxo, —CH₂OR^{o}, —CH₂N(R^{o})₂, —C(O)N(R^{o})₂, —C(O)OR^{o}, —NO₂, —NCS, —CN, —CF₃, —OCF₃ and —N(R^{o})₂,
wherein each occurrence of R^{o} is independently selected from: —(C1-C6)-aliphatic, (C3-C6)-cycloalkyl, 3- to 6-membered heterocyclyl, 5- to 10-membered heteroaryl-, and (C6-C10)-aryl.
Wherein R₇ is selected from —CF₃, —(C1-C6)alkyl, —(C3-C6) cycloalkyl, -5 to 10 membered heteroaryl, —(C6-C10) aryl, (C6-C10) aryl-(C1-C6)alkyl-, —(C1-C6)alkyl-5 to 10 membered heteroaryl and -3-10 membered heterocyclyl, wherein each R₇ is independently substituted with 0-5 R′;
each occurrence of R′ is wherein each occurrence of R′ is independently selected from halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂;
wherein R″ is selected from —Cl, —F, —(C1-C6)alkyl, —OMe, and —(C6-C10) aryl;
each R⁴ is selected from —H or —(C1-C6)alkyl;
each R⁶ is selected from —H or —(C1-C6)alkyl;
each R¹³ and R¹⁴ is independently selected from H—, (C1-C3)-aliphatic-, or (C3-C6)-cycloalkyl;
v) a compound of formula I-e: 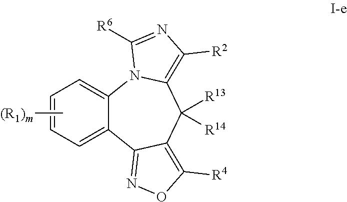
or a pharmaceutically acceptable salt, hydrate, solvate, polymorph, isomer, or combination thereof,
m is an integer selected from 0-4;
each R¹ is independently selected from -halogen, —(C6-C10) aryl, —OMe, —CN, —CHF₂, —CF₃, —OCF₃, —OCHF₂, CO(O)R₇, CH₂—OR⁸, —(C1-C6)alkyl-(C6-C10) aryl, -5-10 membered heteroaryl, —(C1-C6)alkyl-5-10 membered heteroaryl and —(C3-C6) cycloalkyl;
each R⁸ is independently selected from —H or —(C1-C6 alkyl);
each R² is selected from CO(O)R₇, C≡C—R⁹, —(C1-C6)alkyl-C≡C—R⁹, -(5-10 membered) heteroaryl, -(3-10 membered) heterocyclyl, 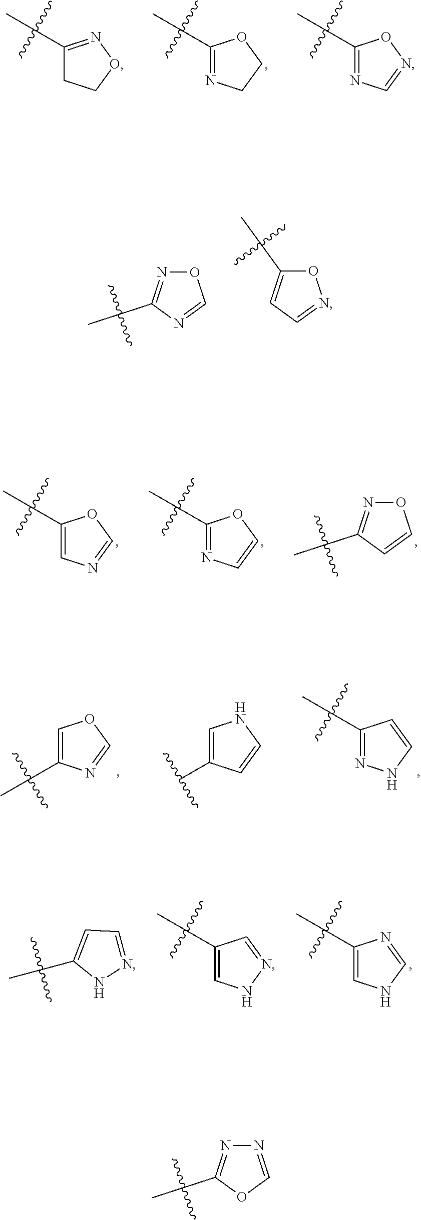
wherein each 5-membered heterocycle or heteroaryl is substituted with 0-4 R₇;
each R⁹ is selected from: —H, —(C1-C6)alkyl, (5- to 10-membered heteroaryl), -(3-10membered) heterocyclyl 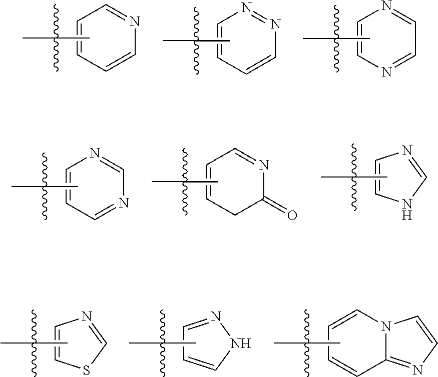
each occurrence of R⁹ is independently substituted by 0-5 R¹¹, wherein each occurrence of R¹¹ is independently selected from —(C1-C6)alkyl, —O—(C1-C6)alkyl, -halogen, —CF₃, —OCF₃, —OMe, —(C6-C10) aryl and -5 to 10 membered heteroaryl;
wherein R₇ is selected from —(C1-C6)alkyl, —(C3-C6) cycloalkyl, -5 to 10 membered heteroaryl, —(C6-C10) aryl, —(C6-C10) aryl-(C1-C6)alkyl, and -5 to 10 membered heteroaryl-(C1-C6)alkyl, and -3-10 membered heterocyclyl, wherein each R₇ is independently substituted with 0-5 R′;
each occurrence of R′ is wherein each occurrence of R′ is independently selected from halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂, wherein each R′ is independently substituted with 0-5 R″;
wherein R″ is selected from —Cl, —F, —(C1-C6)alkyl, —OMe, and —(C6-C10) aryl;
each R⁴ is independently-H or (C1-C6)alkyl;
each R⁶ is independently-H or —(C1-C6)alkyl.
Each R¹³ and R¹⁴ is independently selected from H—, (C1-C3)-aliphatic-, or (C3-C6)-cycloalkyl; and
vi) a compound of formula I-f: 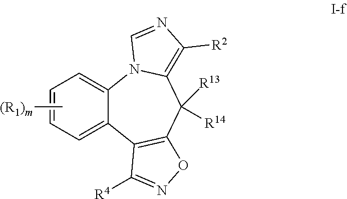
or a pharmaceutically acceptable salt, hydrate, solvate, polymorph, isomer, or combination thereof,
m is an integer selected from 0-4;
each R¹ is independently selected from -halogen, —(C6-C10) aryl, —OMe, —CN, —CHF₂, —CF₃, —OCF₃, —OCHF₂, CO(O)R₇, CH₂—OR⁸, —(C1-C6)alkyl-(C6-C10) aryl, -5-10 membered heteroaryl, —(C1-C6)alkyl-5-10 membered heteroaryl and —(C3-C6) cycloalkyl;
each R⁸ is independently selected from —H or —(C1-C6 alkyl);
each R² is selected from CO(O)R₇, C≡C—R⁹, —(C1-C6)alkyl-C≡C—R⁹, -(5-10 membered) heteroaryl, -(3-10 membered) heterocyclyl, 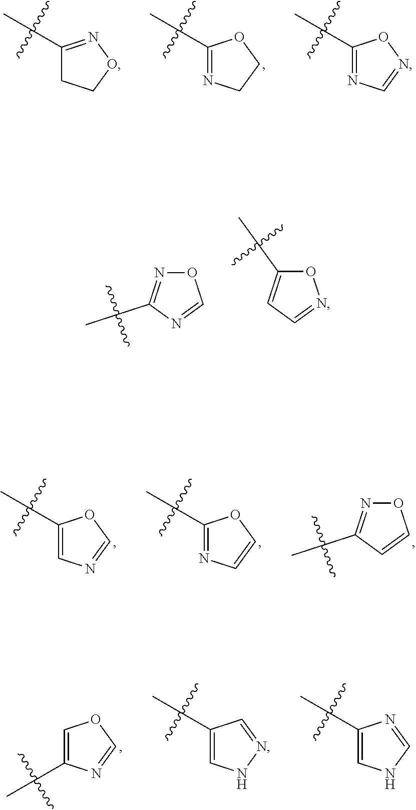
wherein each 5-membered heterocycle or heteroaryl is substituted with 0-4 R₇;
each R⁹ is selected from: —H, —(C1-C6)alkyl, (5- to 10-membered heteroaryl), -(3-10 membered) heterocyclyl 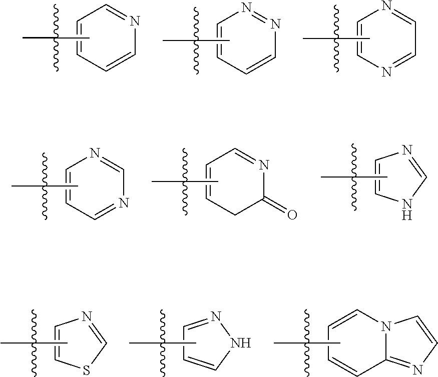
each occurrence of R⁹ is independently substituted by 0-5 R¹¹, wherein each occurrence of R¹¹ is independently selected from —(C1-C6)alkyl, —O—(C1-C6)alkyl, -halogen, —CF₃, —OCF₃, —OMe, —(C6-C10) aryl and -5 to 10 membered heteroaryl;
wherein R₇ is selected from —(C1-C6)alkyl, —(C3-C6) cycloalkyl, -5 to 10 membered heteroaryl, —(C6-C10) aryl, —(C6-C10) aryl-(C1-C6)alkyl, and -5 to 10 membered heteroaryl-(C1-C6)alkyl, and -3-10 membered heterocyclyl, wherein each R₇ is independently substituted with 0-5 R′;
each occurrence of R′ is wherein each occurrence of R′ is independently selected from halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂;
wherein each R′ is independently substituted with 0-5 R″, wherein R″ is selected from —Cl, —F, —(C1-C6)alkyl, —OMe, and —(C6-C10) aryl;
R⁴ is —H or (C1-C6)alkyl;
R⁶ is —H or —(C1-C6)alkyl;
each R¹³ and R¹⁴ is independently selected from H—, (C1-C3)-aliphatic-, or (C3-C6)-cycloalkyl.

35. The method of claim 34, wherein the GABA_{A} α5 receptor agonist, or the pharmaceutically acceptable salt, hydrate, solvate, polymorph, or isomer thereof or the GABA_{A} α5 receptor agonist, or the pharmaceutically acceptable salt, hydrate, solvate, isomer, or polymorph thereof of the pharmaceutical composition, is a compound selected from the group consisting of: Formula I-a, Formula I-b, Formula I-c, Formula I-d, Formula I-e, and Formula I-f, or a pharmaceutically acceptable salt, hydrate, solvate, polymorph, isomer, or combinations thereof., or a pharmaceutically acceptable salt, hydrate, solvate, polymorph, or isomer thereof.

36. The method of claim 21, wherein the GABA_{A} α5 receptor agonist, or the pharmaceutically acceptable salt, hydrate, solvate, isomer, or polymorph thereof or the GABA_{A} α5 receptor agonist, or the pharmaceutically acceptable salt, hydrate, solvate, isomer, or polymorph thereof of the pharmaceutical composition is selected from the group consisting of Compounds 742-755, 758-763, 765-779, 781-795, 797-810, 813-828, 830, 831, 833-846, 848-891, 893-903, 905, 907-977, 979-1012, or a pharmaceutically acceptable salt, hydrate, solvate, isomer, or polymorph thereof.

37. A method of preventing or slowing the progression of cognitive impairment or preventing the development or reducing the rate of cognitive decline in a subject, the subject displaying or presenting with cognitive performance within the normal range for the subject's age, the method comprising administering to the subject
A) levetiracetam, brivaracetam or seletracetam, or a pharmaceutically acceptable salt thereof or a pharmaceutical composition comprising levetiracetam, brivaracetam or seletracetam, or a pharmaceutically acceptable salt thereof and a pharmaceutically acceptable carrier; and
B) a GABA_{A} α5 receptor agonist, or a pharmaceutically acceptable salt, hydrate, solvate, isomer, or polymorph thereof or a pharmaceutical composition comprising a GABA_{A} α5 receptor agonist, or a pharmaceutically acceptable salt, hydrate, solvate, isomer, or polymorph thereof and a pharmaceutically acceptable carrier, or
C) a pharmaceutical composition comprising A and B and a pharmaceutically acceptable carrier.

38. The method of claim 37, wherein the levetiracetam, seletracetam, or brivaracetam, or a pharmaceutically acceptable salt thereof or the pharmaceutical composition comprising the levetiracetam, brivaracetam or seletracetam, or pharmaceutically acceptable salt thereof of A and the GABA_{A} α5 receptor agonist, or pharmaceutically acceptable salt, hydrate, solvate, isomer, or polymorph thereof or the pharmaceutical composition comprising the GABA_{A} α5 receptor agonist, or pharmaceutically acceptable salt, hydrate, solvate, isomer, or polymorph thereof of B, or the pharmaceutical composition of C, is administered orally.

39. The method of claim 37, wherein the levetiracetam, seletracetam, or brivaracetam, or a pharmaceutically acceptable salt thereof or the pharmaceutical composition comprising the levetiracetam, brivaracetam or seletracetam, or pharmaceutically acceptable salt thereof of A and the GABA_{A} α5 receptor agonist, or a pharmaceutically acceptable salt, hydrate, solvate, isomer, or polymorph thereof or the pharmaceutical composition comprising the GABA_{A} α5 receptor agonist, or pharmaceutically acceptable salt, hydrate, solvate, isomer, or polymorph thereof of B, or the pharmaceutical composition of C, is administered once daily.

40. The method of claim 37, wherein the levetiracetam, seletracetam, or brivaracetam, or a pharmaceutically acceptable salt thereof or the pharmaceutical composition comprising the levetiracetam, brivaracetam or seletracetam, or pharmaceutically acceptable salt thereof of A and the GABA_{A} α5 receptor agonist, or a pharmaceutically acceptable salt, hydrate, solvate, isomer, or polymorph thereof or the pharmaceutical composition comprising the GABA_{A} α5 receptor agonist, or pharmaceutically acceptable salt, hydrate, solvate, isomer, or polymorph thereof of B, or the pharmaceutical composition of C, is administered twice daily.

41. The method of claim 37, wherein the levetiracetam, seletracetam, or brivaracetam, or a pharmaceutically acceptable salt thereof or the pharmaceutical composition comprising the levetiracetam, brivaracetam or seletracetam, or pharmaceutically acceptable salt thereof of A and the GABA_{A} α5 receptor agonist, or a pharmaceutically acceptable salt, hydrate, solvate, isomer, or polymorph thereof or the pharmaceutical composition comprising the GABA_{A} α5 receptor agonist, or pharmaceutically acceptable salt, hydrate, solvate, isomer, or polymorph thereof of B are administered simultaneously.

42. The method of claim 37, wherein the levetiracetam, seletracetam, or brivaracetam, or a pharmaceutically acceptable salt thereof or the pharmaceutical composition comprising the levetiracetam, brivaracetam or seletracetam, or pharmaceutically acceptable salt thereof of A and the GABA_{A} α5 receptor agonist, or a pharmaceutically acceptable salt, hydrate, solvate, isomer, or polymorph thereof or the pharmaceutical composition comprising the GABA_{A} α5 receptor agonist, or pharmaceutically acceptable salt, hydrate, solvate, isomer, or polymorph thereof of B are administered sequentially.

43. The method of claim 37, wherein the levetiracetam, brivaracetam or seletracetam, or pharmaceutically acceptable salt thereof of A or C is administered at a daily dose of 0.7-350 mg.

44. The method of claim 43, wherein the levetiracetam or seletracetam, or pharmaceutically acceptable salt thereof of A or C is administered at a daily dose of 125-250 mg.

45. The method of claim 44, wherein the levetiracetam or seletracetam, or pharmaceutically acceptable salt thereof of A or Cis administered at a daily dose of 220 mg.

46. The method of claim 44, wherein the levetiracetam or seletracetam, or pharmaceutically acceptable salt thereof of A or C is administered at a daily dose of 190 mg.

47. The method of claim 43, wherein the brivaracetam, or pharmaceutically acceptable salt thereof of A or C is administered at a daily dose of is 0.7-180 mg.

48. The method of claim 43, wherein the levetiracetam or seletracetam, or pharmaceutically acceptable salt thereof of A is administered at a daily dose of is 7-350 mg.

49. The method of claim 37, wherein the pharmaceutical composition of A and the pharmaceutical composition of B, or the pharmaceutical composition of C, is formulated in one or more of an oral form, an extended release form or a single-unit-dosage-form or for once-a-day administration.

50. The method of claim 49, wherein the extended release form is a controlled release form, a prolonged release form, a sustained release form, a delayed release form, or a slow release form.

51. The method of claim 37, wherein the levetiracetam or pharmaceutically acceptable salt thereof in the pharmaceutical composition is present in an amount of 220 mg and wherein the pharmaceutical composition further comprises 280 mg-350 mg of hydroxypropyl methylcellulose, 1.2 mg-1.4 mg of colloidal silicon dioxide, 92.8 mg-119.2 mg of silicified microcrystalline cellulose, and 6.0 mg-6.7 mg of magnesium stearate.

52. The method of claim 51, wherein the pharmaceutical composition comprises 280 mg or 347.5 mg of hydroxypropyl methylcellulose, 1.2 mg of colloidal silicon dioxide, 92.8 mg or 119.2 mg of silicified microcrystalline cellulose, and 6.0 mg or 6.7 mg of magnesium stearate.

53. The method of claim 37, the levetiracetam or pharmaceutically acceptable salt in the pharmaceutical composition is present in the amount of 190 mg and wherein the pharmaceutical composition further comprises 300 mg of hydroxypropyl methylcellulose, 1.2 mg of colloidal silicon dioxide, 102.8 mg of silicified microcrystalline cellulose or anhydrous dicalcium phosphate, and 6 mg of magnesium stearate.

54. The method of any one of claims 51-53, wherein the hydroxypropyl methylcellulose is hypromellose 2208.

55. The method of any one of claims 51-54, wherein the silicified microcrystalline cellulose is silicified microcrystalline cellulose SMCC 90.

56. The method of claim 37, wherein the pharmaceutical composition comprising the levetiracetam or pharmaceutically acceptable salt thereof of A or C is in extended release form and provides a steady state plasma concentration of levetiracetam in a subject of between 1.9 μg/mL and 4.4 μg/mL within 3 hours after administration and extending for at least 8 hours of a 24-hour period after said administration, within 2 hours after said administration and extending for at least 13 hours of a 24-hour period after said administration, within 1 hour after said administration and extending for at least 13 hours of a 24-hour period after said administration, or within 1 hour after administration and extending for at least 13 to 16 hours of a 24 hour period after said administration.

57. The method of any one of claims 37-56, wherein the GABA_{A} α5 receptor agonist, or the pharmaceutically acceptable salt, hydrate, solvate, isomer, or polymorph thereof, or the GABA_{A} α5 receptor agonist or the pharmaceutically acceptable salt, hydrate, solvate, isomer, or polymorph thereof of the pharmaceutical composition is present in an amount between 5 mg and 1000 mg.

58. The method of any of claims 1-57, wherein the subject is at risk of developing cognitive decline or impairment, wherein the risk is associated with the presence of altered hippocampal functional connectivity in the subject.

59. The method of any of claims 1-57, wherein the subject is at risk of developing cognitive decline or impairment, wherein the risk is associated with the presence of one or more genomic variants, mutations, or polymorphs associated with a change in the expression of the genes selected from the group consisting of ABCA7, CLU, CR1, PICALM, PLD3, TREM2, and SORL1 in the genome of the subject.

60. The method of any of claims 1-57, wherein the subject is at risk of developing cognitive decline or impairment, wherein the risk is associated with the presence of one or more genomic variants, mutations, or polymorphs associated with a change in the expression of TREM2 in the genome of the subject.

61. The method of any of claims 1-57, wherein the subject is at risk of developing cognitive decline or cognitive impairment, wherein the risk is associated with the presence of at least one allele of the APOE4 gene in the genome of the subject.

62. The method of any of claims 1-57, wherein the subject is at risk of developing cognitive decline or cognitive impairment, wherein the risk is associated with the presence of one of more biofluid biomarkers selected from the group consisting of p-tau, t-tau, and amyloid β42 in the subject.

63. The method of any one of claims 1-62, wherein the subject is a human.
